(19) United States
(12) Patent Application Publication
(10) Pub. No.: US 2025/0263680 A1
(43) Pub. Date: Aug. 21, 2025

(54) NUCLEOBASE EDITORS COMPRISING GEOCAS9 AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Yongjoo Kim, Cambridge, MA (US)

(73) Assignees: The Broad Institute, Inc., Cambridge, MA (US); President and Fellows of Harvard College, Cambridge, MA (US)

(21) Appl. No.: 19/073,658

(22) Filed: Mar. 7, 2025

Related U.S. Application Data
(62) Division of application No. 17/289,665, filed on Apr. 28, 2021, now Pat. No. 12,281,338.
(60) Provisional application No. 62/752,225, filed on Oct. 29, 2018.

(51) Int. Cl. C12N 9/22; C12N 9/24; C12N 9/78

(52) U.S. Cl. CPC C12N 9/22; C12N 9/2497; C12N 9/78; C12Y 305/04004; C12Y 305/04005

(57) ABSTRACT

Some aspects of this disclosure provide strategies, systems, reagents, methods, and kits that are useful for the targeted editing of nucleic acids or the modification of nucleic acids or proteins, including editing a single site within the genome of a cell or subject, e.g., within the human genome. In some embodiments, fusion proteins of nucleic acid programmable DNA binding proteins e.g., GeoCas9 or variants thereof, and effector domains, e.g., deaminase domains, are provided. In some embodiments, methods for targeted nucleic acid editing or protein modification are provided. In some embodiments, reagents and kits for the generation of targeted nucleic acid editing proteins, e.g., fusion proteins of a GeoCas9 and effector domains, are provided.

### RELATED APPLICATIONS

This application is a division of and claims priority under 35 U.S.C. § 120 to U.S. patent application U.S. Ser. No. 17/289,665, filed Apr. 28, 2021, which is a national stage filing under 35 U.S.C. § 371 of International PCT Application, PCT/US2019/058678, filed Oct. 29, 2019, which claims priority under 35 U.S.C. § 119 (e) to U.S. Provisional Application, U.S. Ser. No. 62/752,225, filed on Oct. 29, 2018, each of which is incorporated herein by reference.

### REFERENCE TO AN ELECTRONIC SEQUENCE LISTING

The contents of the electronic sequence listing (B119570047US02-SUBSEQ-TNG.xml; Size: 590,708 bytes; and Date of Creation: May 9, 2025) are herein incorporated by reference in its entirety.

### BACKGROUND OF THE INVENTION

Targeted editing of nucleic acid sequences, for example, the targeted cleavage or the targeted introduction of a specific modification into genomic DNA, is a highly promising approach for the study of gene function and also has the potential to provide new therapies for human genetic diseases. An ideal nucleic acid editing technology possesses three characteristics: (1) high efficiency of installing the desired modification; (2) minimal off-target activity; and (3) the ability to be programmed to edit precisely any site in a given nucleic acid, e.g., any site within the human genome. Current genome engineering tools, including engineered zinc finger nucleases (ZFNs), transcription activator like effector nucleases (TALENs), and most recently, the RNA-guided DNA endonuclease Cas9 (e.g., Cas9 from S. pyogenes), affect sequence-specific DNA cleavage in a genome.

CRISPR-Cas (CRISPR associated) systems are protein-RNA complexes that use an RNA molecule (sgRNA) as a guide to localize the complex to a target DNA sequence via base-pairing. In the natural systems, a Cas protein then acts as an endonuclease to cleave the targeted DNA sequence. The target DNA sequence must be both complementary to the sgRNA, and also contain a “protospacer-adjacent motif” (PAM) at the 3′-end of the complementary region in order for the system to function.

Among the known Cas proteins, S. pyogenes Cas9 (spCas9) has been mostly widely used as a tool for genome engineering. However there are disadvantages to using spCas9 for nucleic acid engineering. For example, spCas9 protein is a large, multi-domain protein, and it requires a specific protospacer-adjacent motif (e.g., 5′-NGG-3′) sequence to bind and cleave a particular nucleic acid sequence.

This programmable cleavage can result in mutation of the DNA at the cleavage site via non-homologous end joining (NHEJ) or replacement of the DNA surrounding the cleavage site via homology-directed repair (HDR). Some drawbacks to the current technologies include the large size of engineered nucleases, such as Cas9, used for the modification of nucleic acids, the requirement of Cas9 proteins to bind adjacent to a specific protospacer-adjacent motif (PAM) sequence, and the stability of Cas9 proteins under different temperature conditions.

Accordingly, genome engineering tools with improved properties are desired.

### SUMMARY OF THE INVENTION

Some aspects of the disclosure are based on the recognition that fusion proteins comprising Cas9 proteins with desired properties are useful for nucleic acid engineering applications. For example, advantages of using GeoCas9 as a recognition agent include (1) the sequence specificity of GeoCas9 can be easily altered by simply changing the sgRNA sequence; (2) GeoCas9 binds to its target sequence by denaturing the dsDNA, resulting in a stretch of DNA that is single-stranded and therefore a viable substrate for the deaminase; (3) GeoCas9 is a thermostable hololog of other Cas9 proteins; (4) GeoCas9 is smaller than many homologs of Cas9 proteins from other organisms; and (5) GeoCas9 recognizes and can bind to nucleic acid sequences comprising the PAM with differing specificity relative to a wild type GeoCas9.

Accordingly, some aspects of the disclosure provide fusion proteins comprising: (i) a GeoCas9; (ii) a cytidine deaminase domain; and (iii) a uracil glycosylase inhibitor (UGI) domain. As one example, the GeoCas9 may be a GeoCas9 nickase (“nGeoCas9” or “GeoCas9n”). The GeoCas9n may be configured to cut a nucleotide target strand of a nucleotide duplex, wherein the nucleotide target strand is the strand that binds to a gRNA of the GeoCas9 nickase. In some embodiments, nicking one of the DNA strands facilitates base editing efficiency. In other embodiments, the fusion proteins comprise a second UGI domain, which may further improve base editing efficiency. The disclosure further contemplates compositions comprising any of the fusion proteins provided herein and a guide RNA (gRNA) molecule, which may be used to target a specific nucleobase within a DNA molecule (e.g., genomic DNA molecule).

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF THE DRAWINGS

FIG. 1 shows a homology model of a Geobacillus stearothermophilus Cas9 (GeoCas9) docked with DNA (Harrington et al.).

FIG. 2 shows schematic representatins of base editors containing GeoCas9. The top schematic shows a base editor 3 (BE3) with GeoCas9, referred to as GeoBE3, which comprises an N-terminal APOBEC1 cytidine deaminase linked to GeoCas9 via an XTEN linker, and a C-terminal UGI domain that is linked to the GeoCas9. The bottom schematic shows a base editor 4 (BE4) with GeoCas9, referred to as GeoBE4, which comprises an N-terminal APOBEC1 cytidine deaminase linked to GeoCas9 via a GGS-XTEN-GGS linker (SEQ ID NO: 436), and a two C-terminal UGI domains.

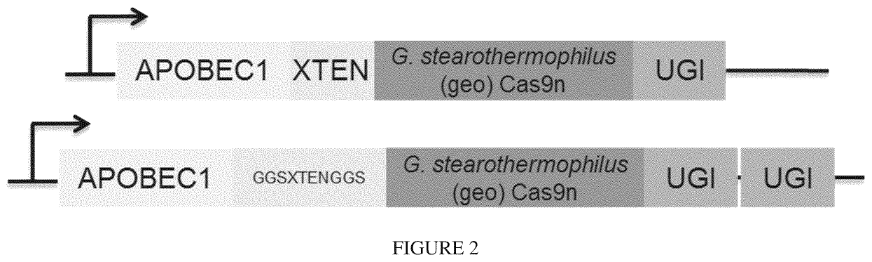

FIG. 3 shows base editing data for GeoBE3 and GeoBE4 on selected DNA sequences. Sequences FANCF Geo4-22 (SEQ ID NO: 406); FANCF Geo5-23 (SEQ ID NO: 407); HEK2 Geo6-22 (SEQ ID NO: 408); HEK2 Geo7-23 (SEQ ID NO: 409); EMX Geo2-23 (SEQ ID NO: 410); EMX Geo2-22 (SEQ ID NO: 411); EMX Geo1-22 (SEQ ID NO: 412), shown from top to bottom, were edited using GeoBE3 and GeoBE4. The PAM sequence for each of the DNA target sequences is indicated by underlining. The PAM consensus sequence is 5′-NNNNCRAA-3′ (SEQ ID NO: 79), where N is any nucleotide (e.g., A, T, C, or G), and R is A or G, as indicated by the IUPAC nucleotide code. The base editing results, indicating the percent of total sequencing reads where the indicated C is converted to T, is shown in the chart (bottom). For all target C positions, the PAM has been set as positions 21-28 (N21NNNCRAA28). The target sequences are numbered in relation to the PAM. For Example, the numbering convention for FANCF Geo4-22 is as follows: T₋₃C₋₂T₋₁C₁T₂G₃C₄G₅T₆A₇C₈T₉G₁₀A₁₁T₁₂T₁₃G₁₄G₁₅A₁₆A₁₇C₁₈A₁₉T₂₀C₂₁C₂₂G₂₃C₂₄G₂₅A₂₆A₂₇A₂₈ (SEQ ID NO: 406). There are targeted Cs further downstream, which are labeled as negative positions. For each base before position 1, the numbering continues as −1, −2, −3, etc.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

a/an/the

As used herein and in the claims, the singular forms “a,” “an,” and “the” include the singular and the plural reference unless the context clearly indicates otherwise. Thus, for example, a reference to “an agent” includes a single agent and a plurality of such agents.

### Base Editor (BE)

The term “base editor (BE),” or “nucleobase editor (NBE),” as used herein, refers to an agent comprising a polypeptide that is capable of making a modification to a base (e.g., A, T, C, G, or U) within a nucleic acid sequence (e.g., DNA or RNA). In some embodiments, the base editor is capable of deaminating a base within a nucleic acid. In some embodiments, the base editor is capable of deaminating a base within a DNA molecule. In some embodiments, the base editor is capable of deaminating a cytosine (C) in DNA. In some embodiments, the base editor is a fusion protein comprising a GeoCas9 fused to a cytidine deaminase domain. In some embodiments, the base editor comprises a GeoCas9 (e.g., catalytically inactive dGeoCas9 or GeoCas9 nickase). In some embodiments, the base editor comprises a GeoCas9 nickase (GeoCas9n) fused to a cytidine deaminase. In some embodiments, the base editor comprises a nuclease-inactive GeoCas9 (dGeoCas9) fused to a cytidine deaminase. In some embodiments, the base editor is fused to an inhibitor of base excision repair, for example, a UGI domain. In some embodiments, the base editor comprises a UGI protein fused to a cytidine deaminase. Where the base editor comprises a GeoCas9, the base editor may be referred to as a “GeoCas9 base editor” or simply as a “base editor.” In addition, since the GeoCas9 base editors comprise fusion proteins, they may also be referred to as “GeoCas9 base editor fusion proteins.”

### Cas9

The term “Cas9” or “Cas9 nuclease” refers to an RNA-guided protein comprising a Cas9 domain, or a fragment thereof (e.g., a protein comprising an active, inactive, or partially active DNA cleavage domain of Cas9, and/or the gRNA binding domain of Cas9). A Cas9 nuclease is also referred to sometimes as a casn1 nuclease or a CRISPR (clustered regularly interspaced short palindromic repeat)-associated nuclease. CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, Geobacillus stearothermophilus, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 has an inactive (e.g., an inactivated) DNA cleavage domain, that is, the Cas9 is a nuclease inactive Cas9. In some embodiments, a Cas9 has a partially inactive DNA cleavage domain, that is, the Cas9 is a nickase that cleaves one strand of a DNA duplex.

An exemplary wild type Cas9 from Streptococcus pyogenes corresponds to the amino acid sequence of SEQ ID NO: 1 (Uniprot Reference Sequence: Q99ZW2).

(SEQ ID NO: 1)
MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

dCas9 or dGeoCas9

A “nuclease-inactivated Cas9 protein” may interchangeably be referred to as a “dCas9” protein (for nuclease “dead” Cas9). A “nuclease-inactivated GeoCas9” is also referred to herein as “dGeoCas9”. Methods for generating a Cas9 protein (or a fragment thereof) having an inactive DNA cleavage domain are known (See, e.g., Jinek et al., Science. 337:816-821 (2012); Qi et al., “Repurposing CRISPR as an RNA-Guided Platform for Sequence-Specific Control of Gene Expression” (2013) Cell. 28; 152 (5): 1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D8A and H582A, with respect to SEQ ID NO: 5 or SEQ ID NO: 6 inactivate the nuclease activity of GeoCas9. As an additional example the mutations D7A and H581A, with respect to SEQ ID NO: 3 inactivate the nuclease activity of GeoCas9. In some embodiments, the dCas9 protein is nuclease inactive GeoCas9 (dGeoCas9).

In some embodiments, dCas9 corresponds to, or comprises in part or in whole, a Cas9 amino acid sequence having one or more mutations that inactivate the Cas9 nuclease activity. For example, in some embodiments, a dCas9 comprises a D7A and/or a H581A mutation in the amino acid sequence provided in SEQ ID NO: 3, or at corresponding positions in any GeoCas9, for example, any one of the amino acid sequences provided in SEQ ID NOs: 5-8. For example, a dCas9 or Cas9 nickase comprises a D7A and/or a H581A mutation in the amino acid sequence provided in SEQ ID NO: 3. A skilled artisan would appreciate how to perform a sequene alignment of GeoCas9 sequences to identify corresponding amino acid sequences in GeoCas9 proteins.

In some embodiments, proteins comprising fragments of GeoCas9 are provided. For example, in some embodiments, a protein comprises one of two GeoCas9 proteins: (1) the gRNA binding domain of GeoCas9; or (2) the DNA cleavage domain of GeoCas9. In some embodiments, “GeoCas9 variants” are provided, which include mutant forms of GeoCas9, and/or fragments thereof. In some embodiments, a GeoCas9 variant shares homology to GeoCas9, or a fragment thereof. For example, in some embodiments, a GeoCas9 variant is at least 70% identical, at least 80% identical, at least 90% identical, at least 95% identical, at least 96% identical, at least 97% identical, at least 98% identical, at least 99% identical, at least 99.5% identical, or at least 99.9% identical to a wild type GeoCas9, such as any one of the GeoCas9 amino acid sequences set forth in any one of SEQ ID NOs: 2, 3, or 5-8. In some embodiments, the GeoCas9 variant has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild-type GeoCas9, such as any one of the GeoCas9 amino acid sequences set forth in any one of SEQ ID NOs: 2, 3, or 5-8. In some embodiments, the GeoCas9 variant comprises a fragment of a GeoCas9 protein (e.g., a gRNA binding domain and/or a DNA-cleavage domain), such that the fragment is at least 70% identical, at least 80% identical, at least 90% identical, at least 95% identical, at least 96% identical, at least 97% identical, at least 98% identical, at least 99% identical, at least 99.5% identical, or at least 99.9% identical to the corresponding fragment of wild-type GeoCas9. In some embodiments, the fragment is is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type GeoCas9, and is capable of associating with (e.g., forming a complex with) a nucleic acid (e.g., RNA), such as a guide nucleic acid (gRNA). In some embodiments, the GeoCas9 fragment is at least 100 amino acids in length. In some embodiments, the fragment is at least 100, at least 150, at least 200, at least 250, at least 300, at least 350, at least 400, at least 450, at least 500, at least 550, at least 600, at least 650, at least 700, at least 750, at least 800, at least 850, at least 900, at least 950, at least 1000, at least 1050, at least 1060, at least 1070, or at least 1080 amino acids in length, and is capable of associating with (e.g., forming a complex with) a nucleic acid (e.g., RNA), such as a guide nucleic acid (gRNA). In some embodiments, Cas9 corresponds to the GeoCas9 of any one of SEQ ID NOs: 2, 3, or 5-8. In some embodiments, Cas9 corresponds to GeoCas9 of SEQ ID NO: 2. In some embodiments, wild-type Cas9 corresponds to GeoCas9 of SEQ ID NO: 3.

In some embodiments, GeoCas9 comprises a D7A mutation, while the residue at position 581 remains a histidine in the amino acid sequence provided in SEQ ID NO: 3 or at corresponding positions in any GeoCas9, for example, any one of the amino acid sequences provided in SEQ ID NOs: 5-8. Without wishing to be bound by any particular theory, the presence of the catalytic residue H581, allows GeoCas9 to cleave the non-edited (e.g., non-deaminated) strand containing a G opposite the targeted C. Restoration of H581 (e.g., from A581) in a GeoCas9 D7A and H581A mutant does not result in the cleavage of the target strand containing the C. Such GeoCas9 variants are able to generate a single-strand DNA break (nick) at a specific location based on the gRNA-defined target sequence, leading to repair of the non-edited strand, ultimately resulting in a G to A change on the non-edited strand. Briefly, the C of a C-G basepair can be deaminated to a U by a deaminase, e.g., an APOBEC deamonase. Nicking the non-edited strand, having the G, facilitates removal of the G via mismatch repair mechanisms. UGI inhibits UDG, which prevents removal of the U.

In other embodiments, dGeoCas9 or GeoCas9 nickase variants having mutations other than D7A and H581A, corresponding to SEQ ID NO: 3 are provided, which, e.g., result in nuclease inactivated GeoCas9 (dGeoCas9) or GeoCas9 nickase. Such mutations, by way of example, include other amino acid substitutions at D7 and/or H581, in the amino acid sequence provided in SEQ ID NO: 3 or at corresponding positions in any GeoCas9, for example, any one of the amino acid sequences provided in SEQ ID NOs: 5-8. Additional examples include other mutations within the nuclease domains of GeoCas9 (e.g., substitutions in the HNH nuclease subdomain and/or the RuvC1 subdomain).

In some embodiments, variants or homologues of GeoCas9 (e.g., variants of any one of SEQ ID NOs: 2, 3, or 5-8) are provided which are at least 70% identical, at least 80% identical, at least 90% identical, at least 95% identical, at least 98% identical, at least 99% identical, at least 99.5% identical, or at least 99.9% identical to any one of SEQ ID NOs: 2, 3, or 5-8. In some embodiments, variants of GeoCas9 (e.g., variants of any one of SEQ ID NOs: 2, 3, or 5-8) are provided having amino acid sequences which are shorter, or longer than any one of SEQ ID NOs: 2, 3, or 5-8, by at least 5 amino acids, by at least 10 amino acids, by at least 15 amino acids, by at least 20 amino acids, by at least 25 amino acids, by at least 30 amino acids, by at least 40 amino acids, by at least 50 amino acids, by at least 75 amino acids, by at least 100 amino acids or more.

In some embodiments, GeoCas9 fusion proteins as provided herein comprise the full-length amino acid sequence of a GeoCas9 protein, e.g., any one of the GeoCas9 sequences provided herein. In other embodiments, however, fusion proteins as provided herein do not comprise a full-length GeoCas9 sequence, but only a fragment thereof. For example, in some embodiments, a GeoCas9 fusion protein provided herein comprises a GeoCas9 fragment, wherein the fragment is capable of binding a crRNA and tracrRNA or sgRNA, but does not comprise a functional nuclease domain, e.g., in that it comprises only a truncated version of a nuclease domain or no nuclease domain at all. Exemplary amino acid sequences of suitable GeoCas9 proteins and GeoCas9 fragments are provided herein, and additional suitable sequences of GeoCas9 proteins and fragments thereof will be apparent to those of skill in the art.

### CRISPR

CRISPR is a family of DNA sequences (i.e., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA.

Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species—the guide RNA. See, e.g., Jinek M., Chylinski K., Fonfara I., Hauer M., Doudna J. A., Charpentier E. Science 337:816-821 (2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an M1 strain of Streptococcus pyogenes.” Ferretti et al., J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In certain types of CRISPR systems (e.g., type II CRISPR systems), correct processing of pre-crRNA requires a trans-encoded small RNA (tracrRNA), endogenous ribonuclease 3 (rnc), and a Cas9 protein. The tracrRNA serves as a guide for ribonuclease 3-aided processing of pre-crRNA. Subsequently, Cas9/crRNA/tracrRNA endonucleolytically cleaves linear or circular nucleic acid target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gRNA”) can be engineered so as to incorporate embodiments of both the crRNA and tracrRNA into a single RNA species—the guide RNA.

In general, a “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. The tracrRNA of the system is complementary (fully or partially) to the tracr mate sequence present on the guide RNA.

### Deaminase

The term “deaminase” or “deaminase domain,” as used herein, refers to a protein or enzyme that catalyzes a deamination reaction. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism, that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism.

In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the cytidine deaminase catalyzes the hydrolytic deamination of cytidine or cytosine in deoxyribonucleic acid (DNA). In some embodiments, the cytidine deaminase domain comprises the amino acid sequence of any one disclosed herein. In some embodiments, the cytidine deaminase or cytidine deaminase domain is a naturally-occurring cytidine deaminase from an organism, such as a human, chimpanzee, gorilla, monkey, cow, dog, rat, or mouse. In some embodiments, the cytidine deaminase or cytidine deaminase domain is a variant of a naturally-occurring cytidine deaminase from an organism that does not occur in nature. For example, in some embodiments, the cytidine deaminase or cytidine deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring cytidine deaminase from an organism, such as a human, chimpanzee, gorilla, monkey, cow, dog, rat, or mouse.

In some embodiments, the deaminase or deaminase domain is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as E. coli, S. aureus, S. typhi, S. putrefaciens, H. influenzae, or C. crescentus.

### Effective Amount

The term “effective amount,” as used herein, refers to an amount of a biologically active agent that is sufficient to elicit a desired biological response. For example, in some embodiments, an effective amount of a nuclease may refer to the amount of the nuclease that is sufficient to induce cleavage of a target site specifically bound and cleaved by the nuclease. In some embodiments, an effective amount of a fusion protein provided herein, e.g., of a fusion protein comprising a GeoCas9 and a nucleic acid editing domain (e.g., a deaminase domain), may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a recombinase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

### Effector Domain

The term, as used herein, “effector domain,” or the equivalent terms “nucleobase modification moiety” or “nucleic acid effector domain,” embraces any protein, enzyme, or polypeptide (or functional fragment thereof) which is capable of modifying a DNA or RNA molecule. Nucleobase modification moieties can be naturally occurring, or can be recombinant. For example, a nucleobase modification moiety can include one or more DNA repair enzymes, for example, and an enzyme or protein involved in base excision repair (BER), nucleotide excision repair (NER), homology-dependent recombinational repair (HR), non-homologous end-joining repair (NHEJ), microhomology end-joining repair (MMEJ), mismatch repair (MMR), direct reversal repair, or other known DNA repair pathway. A nucleobase modification moiety can have one or more types of enzymatic activities, including, but not limited to endonuclease activity, polymerase activity, ligase activity, replication activity, proofreading activity, or a nuclease, a nickase, a recombinase, a methyltransferase, a methylase, an acetylase, an acetyltransferase, a transcriptional activator, or a transcriptional repressor domain.

### Functional Equivalent

The term “functional equivalent” refers to a second biomolecule that is equivalent in function, but not necessarily equivalent in structure to a first biomolecule. For example, a “GeoCas9 equivalent” refers to a protein that has the same or substantially the same functions as GeoCas9, but not necessarily the same amino acid sequence. In the context of the disclosure, the specification refers throughout to “a protein X, or a functional equivalent thereof.” In this context, a “functional equivalent” of protein X embraces any homolog, paralog, fragment, naturally occurring, engineered, mutated, or synthetic version of protein X which bears an equivalent function.

### Fusion Protein

The term “fusion protein” as used herein refers to a hybrid polypeptide which comprises protein domains from at least two different proteins (e.g., a GeoCas9 fusion protein, which comprises a GeoCas9 domain fused to a nucleic acid effector domain). One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of GeoCas9 that directs the binding of the protein to a target site) and a nucleic acid cleavage domain or a catalytic domain of a nucleic-acid editing protein. Another example includes a GeoCas9 or equivalent thereof fused to a deaminase enzyme. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

### GeoCas9

The term “GeoCas9” refers to an RNA-guided protein from a bacterium of the genus Geobacillus (e.g., G. stearothermophilus), including variants and/or fragments thereof, that are capable of associating with a nucleic acid (e.g., RNA), such as a guide nucleic acid (gRNA). In some embodiments, the GeoCas9 comprises a variant of the amino acid sequence of any one of SEQ ID NOs: 2, 3, or 5-8 that is capable of associating with a nucleic acid (e.g., RNA), such as a guide nucleic acid (gRNA). In some embodiments, the GeoCas9 comprises a fragment of the amino acid sequence of any one of SEQ ID NOs: 2, 3, or 5-8 that is capable of associating with a nucleic acid (e.g., RNA), such as a guide nucleic acid (gRNA). In some embodiments, the GeoCas9 comprises the amino acid sequence of any one of SEQ ID NOS: 2, 3, or 5-8.

In some embodiments, the GeoCas9 is from the genus Geobacillus (e.g., G. stearothermophilus, G. thermodenitrificans, G. caldoxylosilyticus, G. galactosidasius, G. icigianus, G. jurassicus, G. kaustophilus, G. lituanicus, G. subterraneus, G. thermantarcticus, G. thermocatenulatus, G. thermoglucosidasius, G. thermoleovorans, G. toebii, G. uzenensis, or G. vulcani), or variant thereof that binds to a guide RNA in order to bind a target nucleic acid (e.g., DNA) sequence. In some embodiments, the GeoCas9 is from Geobacillus stearothermophilus. GeoCas9 from Geobacillus stearothermophilus catalyzes RNA-guided DNA cleavage at elevated temperatures (e.g., up to 70° C.), and this thermostablility and apparent resistance to degradation in vivo may provide expanded utility when GeoCas9 is incorporated into the CRISPR-Cas9 system (see, e.g., “A thermostable Cas9 with increased lifetime in human plasma” Harrington L. B., Paez-Espino D., Staahl B. T., Chen J. C., Ma E., Kyrpides N. C., Doudna J. A. Nature Communications 8, 1424 (2017); “Characterizing a thermostable Cas9 for bacterial genome editing and silencing” Mougiakos I., Mohanraju P., Bosma E. F., Vrouwe V., Bou M. F., Naduthodi M. I. S., Gussak A., Brinkman R. B. L., van Kranenburg R., van der Oost J. Nature Communications 8, 1647 (2017); and “Type II-C CRIPSR-Cas9 Biology, Mechanism, and Application” Mir A., Edraki A., Lee J., Sontheimer E. J. ACS Chemical Biology doi: 10.1021/acschembio.7b00855 (2017), the entire contents of each of which are incorporated herein by reference).

### Gene of Interest

The term “gene of interest,” as used herein, refers to a nucleic acid construct comprising a nucleotide sequence encoding a gene product of interest, for example, a gene product (e.g., a genome editor or component/domain thereof) to be evolved in a continuous evolution process as provided herein.

### Gene Function

The term “function of a gene of interest,” as interchangeably used with the term “gene function” or “activity of a gene of interest,” refers to a function or activity of a gene product, for example, a nucleic acid, or a protein, encoded by the gene of interest. For example, a function of a gene of interest may be an enzymatic activity (e.g., an enzymatic activity resulting in the generation of a reaction product, phosphorylation activity, phosphatase activity, etc.), an ability to activate transcription (e.g., transcriptional activation activity targeted to a specific promoter sequence), a bond-forming activity, (e.g., an enzymatic activity resulting in the formation of a covalent bond), or a binding activity (e.g., a protein, DNA, or RNA binding activity).

### Linker

The term “linker,” as used herein, refers to a chemical group or a molecule linking two molecules or moieties, e.g., two domains of a fusion protein, such as, for example, a GeoCas9 and a nucleic acid editing domain (e.g., a deaminase domain). A linker may be, for example, an amino acid sequence, a peptide, or a polymer of any length and composition. In some embodiments, a linker joins an RNA-guided protein, including a GeoCas9, and a deaminase domain (e.g., a cytidine deaminase domain). In some embodiments, a linker joins a GeoCas9 and a UGI domain. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 1-100 amino acids in length, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

### Mutation

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). Mutations can include a variety of categories, such as single base polymorphisms, microduplication regions, indel, and inversions, and is not meant to be limiting in any way. Mutations can include “loss-of-function” mutations which is the normal result of a mutation that reduces or abolishes a protein activity. Most loss-of-function mutations are recessive, because in a heterozygote the second chromosome copy carries an unmutated version of the gene coding for a fully functional protein whose presence compensates for the effect of the mutation. There are some exceptions where a loss-of-function mutation is dominant, one example being haploinsufficiency, where the organism is unable to tolerate the approximately 50% reduction in protein activity suffered by the heterozygote. This is the explanation for a few genetic diseases in humans. Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively the mutation could lead to overexpression of one or more genes involved in control of the cell cycle, thus leading to uncontrolled cell division and hence to cancer. Because of their nature, gain-of-function mutations are usually dominant.

napDNAbp

As used herein, the term “nucleic acid programmable DNA binding protein” or “napDNAbp,” of which Cas9 and GeoCas9 are examples, refers to a protein which use RNA:DNA hybridization to target and bind to specific sequences in a DNA molecule. Each napDNAbp is associated with at least one guide nucleic acid (e.g., guide RNA), which localizes the napDNAbp to a DNA sequence that comprises a DNA strand (i.e., a target strand) that is complementary to the guide nucleic acid, or a portion thereof (e.g., the protospacer of a guide RNA). In other words, the guide nucleic-acid “programs” the napDNAbp (e.g., Cas9, GeoCas9, or equivalent) to localize and bind to a complementary sequence.

Without being bound by theory, the binding mechanism of a napDNAbp-guide RNA complex, in general, includes the step of forming an R-loop whereby the napDNAbp induces the unwinding of a double-strand DNA target, thereby separating the strands in the region bound by the napDNAbp. The guideRNA protospacer then hybridizes to the “target strand.” This displaces a “non-target strand” that is complementary to the target strand, which forms the single strand region of the R-loop. In some embodiments, the napDNAbp includes one or more nuclease activities, which then cut the DNA leaving various types of lesions. For example, the napDNAbp may comprises a nuclease activity that cuts the non-target strand at a first location, and/or cuts the target strand at a second location. Depending on the nuclease activity, the target DNA can be cut to form a “double-stranded break” whereby both strands are cut. In other embodiments, the target DNA can be cut at only a single site, i.e., the DNA is “nicked” on one strand. Exemplary napDNAbp with different nuclease activities include “Cas9 nickase” (“nCas9”) and a deactivated Cas9 having no nuclease activities (“dead Cas9” or “dCas9”). Exemplary sequences for these and other napDNAbp are provided herein.

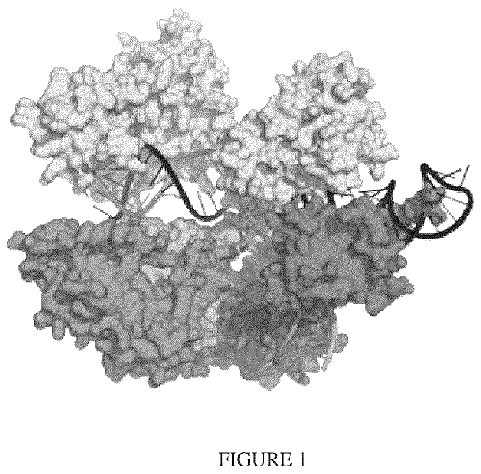

The related term “RNA-programmable nuclease,” and “RNA-guided nuclease” are used interchangeably herein and refer to a nuclease that forms a complex with (e.g., binds or associates with) one or more RNA that is not a target for cleavage (e.g., a Cas9 or homolog or variant thereof). In some embodiments, an RNA-programmable nuclease, when in a complex with an RNA, may be referred to as a nuclease: RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeabley to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al., Science 337:816-821 (2012), the entire contents of which is incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease: RNA complex. In some embodiments, the RNA-programmable nuclease is the (CRISPR-associated system) Cas9 endonuclease, for example Cas9 (Csn1) from Streptococcus pyogenes (see, e.g., “Complete genome sequence of an M1 strain of Streptococcus pyogenes.” Ferretti J. J., McShan W. M., Ajdic D. J., Savic D. J., Savic G., Lyon K., Primeaux C., Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., Mclaughlin R. E., Proc. Natl. Acad. Sci. U.S.A. 98:4658-4663 (2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607 (2011); and “A programmable dual-RNA-guided DNA endonuclease in adaptive bacterial immunity.” Jinek M., Chylinski K., Fonfara I., Hauer M., Doudna J. A., Charpentier E. Science 337:816-821 (2012), the entire contents of each of which are incorporated herein by reference.

Because RNA-programmable nucleases (e.g., Cas9 or GeoCas9) use RNA: DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823-826 (2013); Hwang, W. Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

### Non-Naturally Occurring

The terms “non-naturally occurring” or “engineered” are used interchangeably and indicate the involvement of the hand of man. The terms, when referring to nucleic acid molecules or polypeptides (e.g., GeoCas9 or single-stranded DNA binding protein) mean that the nucleic acid molecule or the polypeptide is at least substantially free from at least one other component with which they are naturally associated in nature and/or as found in nature (e.g., an amino acid sequence not found in nature).

### Nickase

The term “nickase” refers to a napDNAbp (e.g., GeoCas9) with one of the two nuclease domains inactivated. This enzyme is capable of cleaving only one strand of a target DNA.

nCas9 or nGeoCas9

The term “Cas9 nickase,” as used herein, refers to a Cas9 protein that is capable of cleaving only one strand of a duplexed nucleic acid molecule (e.g., a duplexed DNA molecule). In some embodiments, the Cas9 nickase is a GeoCas9 nickase. In some embodiments, a GeoCas9 nickase comprises a D7A mutation and has a histidine at position H581 of SEQ ID NO: 3, or a corresponding mutation in any GeoCas9, such as any one of SEQ ID NOs: 5-8. For example, a GeoCas9 nickase may comprise the amino acid sequence as set forth in SEQ ID NO: 8. Such a GeoCas9 nickase (GeoCas9n) has an active HNH nuclease domain and is able to cleave the non-targeted strand of DNA, i.e., the strand bound by the gRNA in a GeoCas9n: gRNA complex. Further, such a GeoCas9 nickase has an inactive RuvC nuclease domain and is not able to cleave the targeted strand of the DNA, i.e., the strand where base editing is desired. In some embodiments, the Cas9 nickase comprises the amino acid sequence of SEQ ID NO: 2 or a variant thereof, for example any variant thereof provided herein.

GeoCas9n (H8A):
(SEQ ID NO: 8)
MKYKIGLAIGITSIGWAVINLDIPRIEDLGVRIFDRAENPKTGESLALPR
RLARSARRRLRRRKHRLERIRRLFVREGILTKEELNKLFEKKHEIDVWQL
RVEALDRKLNNDELARILLHLAKRRGFRSNRKSERTNKENSTMLKHIEEN
QSILSSYRTVAEMVVKDPKFSLHKRNKEDNYTNTVARDDLEREIKLIFAK
QREYGNIVCTEAFEHEYISIWASQRPFASKDDIEKKVGFCTFEPKEKRAP
KATYTFQSFTVWEHINKLRLVSPGGIRALTDDERRLIYKQAFHKNKITFH
DVRTLLNLPDDTRFKGLLYDRNTTLKENEKVRFLELGAYHKIRKAIDSVY
GKGAAKSFRPIDFDTFGYALTMFKDDTDIRSYLRNEYEQNGKRMENLADK
VYDEELIEELLNLSFSKFGHLSLKALRNILPYMEQGEVYSTACERAGYTF
TGPKKKQKTVLLPNIPPIANPVVMRALTQARKVVNAIIKKYGSPVSIHIE
LARELSQSFDERRKMQKEQEGNRKKNETAIRQLVEYGLTLNPTGLDIVKF
KLWSEQNGKCAYSLQPIEIERLLEPGYTEVDHVIPYSRSLDDSYTNKVLV
LTKENREKGNRTPAEYLGLGSERWQQFETFVLTNKQFSKKKRDRLLRLHY
DENEENEFKNRNLNDTRYISRFLANFIREHLKFADSDDKQKVYTVNGRIT
AHLRSRWNFNKNREESNLHHAVDAAIVACTTPSDIARVTAFYQRREQNKE
LSKKTDPQFPQPWPHFADELQARLSKNPKESIKALNLGNYDNEKLESLQP
VFVSRMPKRSITGAAHQETLRRYIGIDERSGKIQTVVKKKLSEIQLDKTG
HFPMYGKESDPRTYEAIRQRLLEHNNDPKKAFQEPLYKPKKNGELGPIIR
TIKIIDTTNQVIPLNDGKTVAYNSNIVRVDVFEKDGKYYCVPIYTIDMMK
GILPNKAIEPNKPYSEWKEMTEDYTFRFSLYPNDLIRIEFPREKTIKTAV
GEEIKIKDLFAYYQTIDSSNGGLSLVSHDNNFSLRSIGSRTLKRFEKYQV
DVLGNIYKVRGEKRVGVASSSHSKAGETIRPL
(D8A mutation shown in single underline,
H582 shown in bold and underlining)

### Nuclear Localization Sequence (NLS)

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al., International PCT application, PCT/EP2000/011690, filed Nov. 23, 2000, published as WO/2001/038547 on May 31, 2001, the contents of which are incorporated herein by reference for its disclosure of exemplary nuclear localization sequences. In some embodiments, a NLS comprises the amino acid sequence PKKKRKV (SEQ ID NO: 75), MDSLLMNRRKFLYQFKNVRWAKGRRETYLC (SEQ ID NO: 76), KRTADGSEFESPKKKRKV (SEQ ID NO: 374), or KRTADGSEFEPKKKRKV (SEQ ID NO: 375).

### Nucleic Acid Molecule

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

### Nucleic Acid Effector Domain

The term “nucleic acid effector domain,” as used herein (or “nucleic acid editing domain”) refers to a protein or enzyme capable of making one or more modifications (e.g., deamination of a cytidine residue) to a nucleic acid (e.g., DNA or RNA) or a protein associated with a nucleic acid (e.g., a histone protein). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain. In some embodiments the nucleic acid editing domain is a protein or enzyme capable of making one or more modifications (e.g., deamination of a cytidine residue) to a nucleic acid (e.g., DNA or RNA). In some embodiments the nucleic acid editing domain is a deaminase (e.g., a cytidine deaminase, such as an APOBEC or an AID deaminase).

### Oligonucleotide

As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O (6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

### Pharmaceutical Composition

The term “pharmaceutical composition,” as used herein, refers to a composition that can be administrated to a subject in the context of treatment of a disease or disorder. In some embodiments, a pharmaceutical composition comprises an active ingredient, e.g., a base editor or a nucleic acid encoding a base editor, and a pharmaceutically acceptable excipient.

### Proliferative Disease

The term “proliferative disease,” as used herein, refers to any disease in which cell or tissue homeostasis is disturbed in that a cell or cell population exhibits an abnormally elevated proliferation rate. Proliferative diseases include hyperproliferative diseases, such as pre-neoplastic hyperplastic conditions and neoplastic diseases. Neoplastic diseases are characterized by an abnormal proliferation of cells and include both benign and malignant neoplasias. Malignant neoplasia is also referred to as cancer.

### Promoter

The term “promoter” is art-recognized and refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active in the presence of a specific condition. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

### Protein, Peptide, Polypeptide

The terms “protein,” “peptide,” and “polypeptide” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a nucleic-acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4^{th} ed., Cold Spring Harbor Laboratory Press, Cold Spring Harbor, N.Y. (2012)), the entire contents of which are incorporated herein by reference.

### Protospacer

As used herein, the term “protospacer” refers to the sequence (˜20 bp) in DNA adjacent to the PAM (protospacer adjacent motif) sequence which is complementary to the spacer sequence of the guide RNA. The guide RNA anneals to the protospacer sequence on the target DNA (specifically, one strand thereof, i.e, the “target strand” versus the “non-target strand” of the target sequence). In order for Cas9 to function it also requires a specific protospacer adjacent motif (PAM) that varies depending on the bacterial species of the Cas9 gene. The most commonly used Cas9 nuclease, derived from S. pyogenes, recognizes a PAM sequence of NGG that is found directly downstream of the target sequence in the genomic DNA, on the non-target strand. The skilled person will appreciate that the literature in the state of the art sometimes refers to the “protospacer” as the ˜20-nt target-specific guide sequence on the guide RNA itself, rather than referring to it as a “spacer.” Thus, in some cases, the term “protospacer” as used herein may be used interchangeably with the term “spacer.” The context of the discription surrounding the appearance of either “protospacer” or “spacer” will help inform the reader as to whether the term is refence to the gRNA or the DNA target. Both usages of these terms are acceptable since the state of the art uses both terms in each of these ways.

### Protospacer Adjacent Motif (PAM)

As used herein, the term “protospacer adjacent sequence” or “PAM” refers to an approximately 2-6 base pair DNA sequence (or a 2-, 3-, 4-, 5-, 6-, 7-, 8-, 9-, 10-, 11-, 12-long nucleotide sequence) that is an important targeting component of a Cas9 nuclease. Typically, the PAM sequence is on either strand, and is downstream in the 5′ to 3′ direction of Cas9 cut site. The canonical PAM sequence (i.e., the PAM sequence that is associated with the Cas9 nuclease of Streptococcus pyogenes or SpCas9) is 5′-NGG-3′ wherein “N” is any nucleobase followed by two guanine (“G”) nucleobases. Different PAM sequences can be associated with different Cas9 nucleases or equivalent proteins from different organisms. In addition, any given Cas9 nuclease, e.g., GeoCas9, may be modified to alter the PAM specificity of the nuclease such that the nuclease recognizes alternative PAM sequence.

For example, with reference to the canonical SpCas9 amino acid sequence is SEQ ID NO: 1, the PAM sequence can be modified by introducing one or more mutations, including (a) D1135V, R1335Q, and T1337R “the VQR variant”, which alters the PAM specificity to NGAN or NGNG, (b) D1135E, R1335Q, and T1337R “the EQR variant”, which alters the PAM specificity to NGAG, and (c) D1135V, G1218R, R1335E, and T1337R “the VRER variant”, which alters the PAM specificity to NGCG. In addition, the D1135E variant of canonical SpCas9 still recognizes NGG, but it is more selective compared to the wild type SpCas9 protein. The corresponding or equivalent amino acid positions in any GeoCas9 amino acid sequence could be altered similary to achieve an altered PAM specificity (e.g., by altering SEQ ID NOs: 2, 3, or 5-8).

It will also be appreciated that Cas9 enzymes from different bacterial species (i.e., Cas9 orthologs) can have varying PAM specificities. For example, Cas9 from Staphylococcus aureus (SaCas9) recognizes NGRRT or NGRRN. In addition, Cas9 from Neisseria meningitis (NmCas) recognizes NNNNGATT. In another example, Cas9 from Streptococcus thermophilis (StCas9) recognizes NNAGAAW. In still another example, Cas9 from Treponema denticola (TdCas) recognizes NAAAAC. These are example are not meant to be limiting. It will be further appreciated that non-SpCas9s bind a variety of PAM sequences, which makes them useful when no suitable SpCas9 PAM sequence is present at the desired target cut site. Furthermore, non-SpCas9s may have other characteristics that make them more useful than SpCas9. For example, Cas9 from Staphylococcus aureus (SaCas9) is about 1 kilobase smaller than SpCas9, so it can be packaged into adeno-associated virus (AAV). Further reference may be made to Shah et al., “Protospacer recognition motifs: mixed identities and functional diversity,” RNA Biology, 10 (5): 891-899 (which is incorporated herein by reference). The corresponding or equivalent amino acid positions in any GeoCas9 amino acid sequence could be altered similary to achieve an altered PAM specificity (e.g., by altering SEQ ID NOs: 2, 3, or 5-8).

### Recombinant

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

### Recombinase

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, ϕC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The Gin recombinase referred to herein may be any Gin recombinase known in the art including, but not limited to, the Gin recombinases presented in T. Gaj et al., A comprehensive approach to zinc-finger recombinase customization enables genomic targeting in human cells. Nucleic Acids Research 41, 3937-3946 (2013), incorporated herein by reference in its entirety. Exemplary recombinase sequences are disclosed herein.

### RNA-Guided Protein

The term “RNA-guided protein” refers to a protein (e.g., an RNA-guided nuclease) that forms a complex with (e.g., binds or associates with) one or more RNA(s) that is not a target for cleavage. In some embodiments, an RNA-guided protein, when in a complex with an RNA, may be referred to as a RNA-guided-protein: RNA complex. In some embodiments, the RNA-guided protein: RNA complex binds to a target nucleic acid (e.g., DNA) sequence, such as a sequence within a genome of an organism, via complementary binding of a portion of the RNA to the nucleic acid. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is also used to refer to guide RNAs that exist as either single molecules or as a complex of two or more molecules. Typically, gRNAs that exist as a single RNA species comprise two domains: (1) a domain that shares homology to a target nucleic acid (i.e., directs binding of a Cas9 complex to the target); and (2) a domain that binds a Cas9 protein. In some embodiments, domain (2) corresponds to a sequence known as a tracrRNA and comprises a stem-loop structure. In some embodiments, domain (2) is identical or homologous to a tracrRNA as provided in Jinek et al., Science 337:816-821 (2012), the entire contents of which are incorporated herein by reference. Other examples of gRNAs (e.g., those including domain 2) can be found in U.S. Provisional Patent Application, U.S. Ser. No. 61/874,682, filed Sep. 6, 2013, entitled “Switchable Cas9 Nucleases And Uses Thereof,” and U.S. Provisional Patent Application, U.S. Ser. No. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease: RNA complex.

In some embodiments, the gRNA backbone sequence is a gRNA backbone sequence that binds to a GeoCas9 protein. gRNA backbone sequences that bind to GeoCas9 are known in the art and have been described previously, for example, in Harrington, L. B. et al., A thermostable Cas9 with increased lifetime in human plasma” Nat. Commun., 2017 Nov. 10; 8 (1): 1424. The entire contents of which are incorporated herein by reference. In some embodiments, the gRNA sequence comprises the nucleic acid sequence 5′-GUCAUAGUUCCCCUGAGAAAUCAGGGUUACUAUGAUAAGGGCUUUCUGCCUA AGGCAGACUGACCCGCGGCGUUGGGGAUCGCCUGUCGCCCGCUUUUGGCGGGC AUUCCCCAUCCUUUUUUU-3′ (SEQ ID NO: 77), or a variant thereof that binds to a GeoCas9. For example, the gRNA may comprise a nucleic acid sequence that has 1, 2, 3, 4, 5, 6, 7, 8, 9, 10, 11, 12, 13, 14, 15, 16, 17, 18, 19, or 20 nucleic acid changes relative to (SEQ ID NO: 77) and is capable of binding to a GeoCas9. In some embodiments, the gRNA comprises a nucleotide sequence that complements a target site, which mediates binding of the nuclease/RNA complex to said target site, providing the sequence specificity of the nuclease: RNA complex. In some embodiments, the nucleotide sequence that complements a target site, comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides that are complementary to a target site.

For example the gRNAsequence may comprise the structure 5′-[nucleotide sequence that complements a target site]-GUCAUAGUUCCCCUGAGAAAUCAGGGUUACUAUGAUAAGGGCUUUCUGCCUA AGGCAGACUGACCCGCGGCGUUGGGGAUCGCCUGUCGCCCGCUUUUGGCGGGC AUUCCCCAUCCUUUUUUU-3′ (SEQ ID NO: 77). In some embodiments, the nucleotide sequence that complements a target site comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 contiguous nucleotides. In some embodiments, the RNA-guided protein is a nuclease, such as a Cas9 (e.g., GeoCas9) that cleaves one or both strands of a duplexed DNA molecule. In some embodiments, the Cas9 is a fully active GeoCas9 that cleaves both strands of a duplexed DNA molecule. In some embodiments, the Cas9 is a GeoCas9 nickase (GeoCas9n) tha cleaves one strand of a duplexed DNA molecule. In some embodiments, the RNA-guided protein is a GeoCas9 that has been engineered (e.g., mutated) to silence nuclease activity.

Because RNA-guided nucleases (e.g., Cas9) use RNA: DNA hybridization to target DNA cleavage sites, these proteins are able to target, in principle, any sequence specified by the guide RNA. Methods of using RNA-guided nucleases, such as Cas9, for site-specific cleavage (e.g., to modify a genome) are known in the art (see e.g., Cong, L. et al., Multiplex genome engineering using CRISPR/Cas systems. Science 339, 819-823 (2013); Mali, P. et al., RNA-guided human genome engineering via Cas9. Science 339, 823-826 (2013); Hwang, W. Y. et al., Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J. E. et al., Genome engineering in Saccharomyces cerevisiae using CRISPR-Cas systems. Nucleic Acids Research (2013); Jiang, W. et al., RNA-guided editing of bacterial genomes using CRISPR-Cas systems. Nature Biotechnology 31, 233-239 (2013); the entire contents of each of which are incorporated herein by reference).

### Spacer Sequence

As used herein, the term “spacer sequence” in connection with a guide RNA refers to the portion of the guide RNA of about 20 nucleotides (or from about 5, 6, 7, 8, 9, 11, 12 13, 14, 15, 16, 17, 18, 19, 20, 21, 22, 23, 24, 25, 26, 27, 28, 29, or 30, or more) which contains a nucleotide sequence that is complementary to the protospacer sequence in the target DNA sequence. The spacer sequence anneals to the protospacer sequence to form a ssRNA/ssDNA hybrid structure at the target site and a corresponding R loop ssDNA structure of the endogenous DNA strand that is complementary to the protospacer sequence.

### Subject

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is a healthy volunteer.

### Target Site

The term “target site” refers to a nucleobase or a sequence within a nucleic acid molecule that is targeted and/or modified by a nucleic acid editing domain. In some embodiments, a target site refers to a cytidine within a nucleic acid molecule that is deaminated by a deaminase or a fusion protein comprising a deaminase, (e.g., a GeoCas9-deaminase fusion protein provided herein).

### Transitions

As used herein, “transitions” refer to the interchange of purine nucleobases (A↔G) or the interchange of pyrimidine nucleobases (C↔T). This class of interchanges involves nucleobases of similar shape. The compositions and methods disclosed herein are capable of inducing one or more transitions in a target DNA molecule. The compositions and methods disclosed herein are also capable of inducing both transitions and transversion in the same target DNA molecule. These changes involve A↔G, G↔A, C↔T, or T↔C. In the context of a double-strand DNA with Watson-Crick paired nucleobases, transversions refer to the following base pair exchanges: A:T↔G:C, G:G↔A:T, C:G↔T:A, or T:A↔C:G. The compositions and methods disclosed herein are capable of inducing one or more transitions in a target DNA molecule. The compositions and methods disclosed herein are also capable of inducing both transitions and transversion in the same target DNA molecule, as well as other nucleotide changes, including deletions and insertions.

### Transversions

As used herein, “transversions” refer to the interchange of purine nucleobases for pyrimidine nucleobases, or in the reverse and thus, involve the interchange of nucleobases with dissimilar shape. These changes involve T↔A, T↔G, C↔G, C↔A, A↔T, A↔C, G↔C, and G↔T. In the context of a double-strand DNA with Watson-Crick paired nucleobases, transversions refer to the following base pair exchanges: T:A↔A:T, T:A↔G:C, C:G↔G:C, C:G↔A:T, A:T↔T:A, A:T↔C:G, G:C↔C:G, and G:C↔T:A. The compositions and methods disclosed herein are capable of inducing one or more transversions in a target DNA molecule. The compositions and methods disclosed herein are also capable of inducing both transitions and transversion in the same target DNA molecule, as well as other nucleotide changes, including deletions and insertions.

### Treatment

The terms “treatment,” “treat,” and “treating” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

### Uracil Glycosylase Inhibitor

The term “uracil glycosylase inhibitor” or “UGI,” as used herein, refers to a protein that is capable of inhibiting a uracil-DNA glycosylase base-excision repair enzyme. In some embodiments, a UGI domain comprises a wild-type UGI or a UGI as set forth in SEQ ID NO: 60-74. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 60-74. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 60-74. In some embodiments, a UGI comprises an amino acid sequence homologous to the amino acid sequence set forth in SEQ ID NO: 60-74, or an amino acid sequence homologous to a fragment of the amino acid sequence set forth in SEQ ID NO: 60-74. In some embodiments, proteins comprising UGI or fragments of UGI or homologs of UGI or UGI fragments are referred to as “UGI variants.” A UGI variant shares homology to UGI, or a fragment thereof. For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 60-74. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 60-74.

In some embodiments, the UGI comprises the amino acid sequence of SEQ ID NO: 60, as set forth below. Exemplary Uracil-DNA glycosylase inhibitor (UGI;

>sp|P14739|UNGI_BPPB2)
(SEQ ID NO: 60)
MTNLSDIIEKETGKQLVIQESILMLPEEVEEVIGNKPESDILVHTAYDES
TDENVMLLTSDAPEYKPWALVIQDSNGENKIKML.

### DETAILED DESCRIPTION OF CERTAIN EMBODIMENTS OF THE INVENTION

Some aspects of this disclosure provide fusion proteins that comprise a domain capable of binding to a nucleotide sequence (e.g., a GeoCas9 protein) and a nucleic acid effector domain, for example, a DNA-editing domain, such as a deaminase domain, recominase domain, or methyltransferase domain. All such fusion proteins may be referred to herein as “GeoCas9 base editors” or “GeoCas9 base editor fusion proteins.” In some cases, these proteins may be referred to simply as “fusion proteins.”

For example, in the case where the nucleic acid effector domain is a deaminase, the deamination of a nucleobase by a deaminase can lead to a point mutation at the respective residue, which is referred to herein as nucleic acid editing or “base editing.” Fusion proteins comprising a GeoCas9 variant and a DNA editing domain can thus be used for the targeted editing of nucleic acid sequences.

Such GeoCas9 base editor fusion proteins are useful for targeted editing of DNA in vitro, e.g., for the generation of mutant cells or animals; for the introduction of targeted mutations, e.g., for the correction of genetic defects in cells ex vivo, e.g., in cells obtained from a subject that are subsequently re-introduced into the same or another subject; and for the introduction of targeted mutations, e.g., the correction of genetic defects or the introduction of deactivating mutations in disease-associated genes in a subject. Typically, the GeoCas9 of the fusion proteins described herein does not have full nuclease activity but instead is a dGeoCas9 protein or a GeoCas9 nickase (GeoCas9n). Other aspects of the invention provide fusion proteins that comprise (i) a domain capable of binding to a nucleic acid sequence (e.g., a GeoCas9 or fragment thereof) and (ii) an enzyme domain, for example, a DNA-editing domain (e.g., a deaminase domain). In various embodiments, the GeoCas9 base editor fusion proteins may also comprise (iii) one or more uracil glycosylase inhibitor (UGI) domains. The presence of at least one UGI domain increases base editing efficiency compared to fusion proteins without a UGI domain. In some embodiments, a fusion protein comprising two UGI domains further increases base editing efficiency and product purity compared to fusion proteins with one UGI domain or without a UGI domain. Methods for the use of GeoCas9 fusion proteins as described herein are also provided.

### I. GeoCas9 Base Editors

In various aspects, the disclosure provides GeoCas9 base editor fusion proteins for use in base editing of target DNA molecule (e.g., a genome). In various embodiments, the GeoCas9 base editor fusion proteins are complexed with a guide RNA that programs the GeoCas9 base editor to target a specific nucleotide sequence. The various components of the GeoCas9 base editor fusion proteins (e.g., the GeoCas9 domain and the one or more effector domains) and the guide RNAs are described further herein. In various embodiments, the GeoCas9 base editor fusion proteins comprises a GeoCas9 domain and an effector domain, wherein the effector domain can include, but are not limited to, a deaminase (e.g., a cytidine deaminase or an adenosine deaminase), a nuclease, a nickase, a recombinase, a methyltransferase, a methylase, an acetylase, an acetyltransferase, a transcriptional activator, or a transcriptional repressor domain.

### A. GeoCas9 Proteins

The GeoCas9 base editors may comprise any suitable GeoCas9 protein. The GeoCas9 proteins may be a naturally occurring GeoCas9 protein. The GeoCas9 may also be a variant of a naturally occurring GeoCas9 protein. Such variants may comprise one or more mutations. Such mutations may be introduced using recombinant techniques, mutagenesis, or sequence evolution techniques (e.g., PACE or PANCE). Variants may also include GeoCas9 fragments which main some of all of the function of a complete GeoCas9 protein. Such variants are described further herein. In some embodiments, such variants may be those sequences with are at least 70%, at least 75%, at least 80%, at least 85%, at least 90%, at least 95%, at least 96%, at least 97%, at least 98%, or at least 99% similar to any of the GeoCas9 sequences provided herein.

Non-limiting, exemplary GeoCas9 proteins are provided herein. The GeoCas9 may be a nuclease active GeoCas9, a nucleasae inactive GeoCas9 (dGeoCas9), or a GeoCas9 nickase (GeoCas9n). In some embodiments, the GeoCas9 is a nuclease active GeoCas9. For example, the GeoCas9 may be a GeoCas9 that cuts both strands of a duplexed nucleic acid (e.g., both strands of a duplexed DNA molecule). In some embodiments the GeoCas9 comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any naturally-occurring GeoCas9 to any one of the amino acid sequences set forth in SEQ ID NOs: 2, 3, or 5-8. In some embodiments, the GeoCas9 comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any naturally-occurring GeoCas9 or to any one of the amino acid sequences set forth in SEQ ID NOs: 2, 3, or 5-8. In some embodiments, the GeoCas9 comprises an amino acid sequence that has at least 10, at least 15, at least 20, at least 30, at leat 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1050, at least 1060, at least 1070, or at least 1080 identical contiguous amino acid residues as compared to any naturally-occurring GeoCas9 or to any one of the amino acid sequences set forth in SEQ ID NOs: 2, 3, or 5-8. In some embodiments, the GeoCas9 comprises any one of the amino acid sequences as set forth in SEQ ID NOs: 2, 3, or 5-8.

In some embodiments, the GeoCas9 is a nuclease-inactive GeoCas9 (dGeoCas9). For example, the dGeoCas9 may bind to a duplexed nucleic acid molecule (e.g., via a gRNA molecule) without cleaving either strand of the duplexed nucleic acid molecule. In some embodiments, the nuclease-inactive dGeoCas9 comprises a D7X₁ mutation and a H581X₂ mutation of the amino acid sequence set forth in SEQ ID NO: 3, or a corresponding mutation in any GeoCas9, such as any one of the amino acid sequences provided in SEQ ID NOs: 2, 3, or 5-8, where X₁ is any amino acid except D and where X₂ is any amino acid except H. In some embodiments, the nuclease-inactive dGeoCas9 comprises a D7A mutation and a H581A mutation of the amino acid sequence set forth in SEQ ID NO: 3, or a corresponding mutation in any GeoCas9, such as any one of the amino acid sequences provided in SEQ ID NOs: 2, 3, or 5-8. As one example, a nuclease-inactive GeoCas9d comprises the amino acid sequence set forth in SEQ ID NO: 7.

Additional suitable nuclease-inactive dGeoCas9 proteins will be apparent to those of skill in the art based on this disclosure and knowledge in the field, and are within the scope of this disclosure. Examples of additional nuclease-inactive GeoCas9 proteins include, but are not limited to, D8A/H582A, D8A/D581A/H582A, and D8A/D581A/H582A/N605A mutations of the amino acid sequence set forth in SEQ ID NO: 3, or a corresponding mutation in any GeoCas9, such as any one of the amino acid sequences provided in SEQ ID NOs: 2, 3, or 5-8 (See, e.g., Prashant et al., CAS9 transcriptional activators for target specificity screening and paired nickases for cooperative genome engineering. Nature Biotechnology. 2013; 31 (9): 833-838, the entire contents of which are incorporated herein by reference). In some embodiments the dGeoCas9 comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any of the GeoCas9 proteins provided herein, but is not capable of cleaving a strand of a nucleic acid (e.g., DNA or RNA) molecule. In some embodiments, the GeoCas9 comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any naturally-occurring GeoCas9, or any one of the amino acid sequences set forth in SEQ ID NOs: 2, 3, or 5-8. In some embodiments, the GeoCas9 comprises an amino acid sequence that has at least 10, at least 15, at least 20, at least 30, at leat 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1050, at least 1060, at least 1070, or at least 1080 identical contiguous amino acid residues as compared to any GeoCas9, such as any one of the amino acid sequences set forth in SEQ ID NOs: 2, 3, or 5-8.

In some embodiments, the GeoCas9 is a GeoCas9 nickase (GeoCas9n). The GeoCas9 nickase may be a GeoCas9 protein that is capable of cleaving only one strand of a duplexed nucleic acid molecule (e.g., a duplexed DNA molecule). In some embodiments the GeoCas9 nickase cleaves the target strand of a duplexed nucleic acid molecule, meaning that the GeoCas9 nickase cleaves the strand that is base paired to (complementary to) a gRNA (e.g., an sgRNA) that is bound to the GeoCas9 nickase. In some embodiments, a GeoCas9 nickase comprises a D7A mutation and has a histidine at position 581 of SEQ ID NO: 3, or a corresponding mutation in any GeoCas9, such as any one of the GeoCas9 proteins provided in SEQ ID NOs: 2, 3, or 5-8. For example, a GeoCas9 nickase may comprise the amino acid sequence as set forth in SEQ ID NO: 2. In some embodiments the GeoCas9 nickase cleaves the non-target, non-base-edited strand of a duplexed nucleic acid molecule, meaning that the GeoCas9 nickase cleaves the strand that is not base paired to a gRNA (e.g., a sgRNA) that is bound to the Cas9. In some embodiments, a GeoCas9 nickase comprises an H581A mutation and has an aspartic acid residue at position 7 of SEQ ID NO: 3, or a corresponding mutation in any GeoCas9, such as the GeoCas9 of any one of SEQ ID NOs: 2, 3, or 5-8. In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any GeoCas9, such as the GeoCas9 of any one of SEQ ID NOs: 2, 3, or 5-8, and is capable of cleaving one, but not both, strands of a duplexed nucleic acid (e.g., DNA or RNA) molecule. Additional suitable Cas9 nickases will be apparent to those of skill in the art based on this disclosure and knowledge in the field, and are within the scope of this disclosure.

Exemplary GeoCas9 sequences for use in the this disclosure are as follows:

dGeoCas9 (D8A and H582A):
(SEQ ID NO: 7)
MKYKIGLAIGITSIGWAVINLDIPRIEDLGVRIFDRAENPKTGESLALPRRLAR
SARRRLRRRKHRLERIRRLFVREGILTKEELNKLFEKKHEIDVWQLRVEALDRKLNN
DELARILLHLAKRRGFRSNRKSERTNKENSTMLKHIEENQSILSSYRTVAEMVVKDP
KFSLHKRNKEDNYTNTVARDDLEREIKLIFAKQREYGNIVCTEAFEHEYISIWASQRP
FASKDDIEKKVGFCTFEPKEKRAPKATYTFQSFTVWEHINKLRLVSPGGIRALTDDER
RLIYKQAFHKNKITFHDVRTLLNLPDDTRFKGLLYDRNTTLKENEKVRFLELGAYHK
IRKAIDSVYGKGAAKSFRPIDFDTFGYALTMFKDDTDIRSYLRNEYEQNGKRMENLA
DKVYDEELIEELLMLSFSKFGHLSLKALRNILPYMEQGEVYSTACERAGYTFTGPKKK
QKTVLLPNIPPIANPVVMRALTQARKVVNAIIKKYGSPVSIHIELARELSQSFDERRKM
QKEQEGNRKKNETAIRQLVEYGLTLNPTGLDIVKFKLWSEQNGKCAYSLQPIEIERLL
EPGYTEVDAVIPYSRSLDDSYTNKVLVLTKENREKGNRTPAEYLGLGSERWQQFETF
VLTNKQFSKKKRDRLLRLHYDENEENEFKNRNLNDTRYISRFLANFIREHLKFADSD
DKQKVYTVNGRITAHLRSRWNFNKNREESNLHHAVDAAIVACTTSDIARVTAFYQ
RREQNKELSKKTDPQFPQPWPHFADELQARLSKNPKESIKALNLGNYDNEKLESLQP
VFVSRMPKRSITGAAHQETLRRYIGIDERSGKIQTVVKKKLSEIQLDKTGHFPMYGKE
SDPRTYEAIRQRLLEHNNDPKKAFQEPLYKPKKNGELGPIIRTIKIIDTTNQVIPLNDGK
TVAYNSNIVRRVDVFEKDGKYYCVPIYTIDMMKGILPNKAIEPNKPYSEWKEMTEDYT
FRFSLYPNDLIRIEFPREKTIKTAVGEEIKIKDLFAYYQTIDSSNGGLSLVSHDNNFSLR
SIGSRTLKRFEKYQVDVLGNIYKVRGEKRVGVASSSHSKAGETIRPL
(D8A and H582A mutations shown in bold and underlining)
GeoCas9 nickase (D8A) - Used in GeoBE3 and GeoBE4 Constructs
(SEQ ID NO: 2)
RYKIGLAIGITSVGWAVMNLDIPRIEDLGVRIFDRAENPQTGESLALPRRLAR
SARRRLRRRKHRLERIRRLVIREGILTKEELDKLFEEKHIEDVWQLRVEALDRKLNND
ELARVLLHLAKRRGFKSMRKSERSNKENSTMLKHIEENRAILSSYRTVGEMIVKDPK
FALHKRNKGENYTNTIARDDLEREIRLIFSKQREFGNMSCTEEFENEYITIWASQRPV
ASKDDIEKKVGFCTFEPKEKRAPKATYTFQSFIAWEHINKLRLISPSGARGLTDEERRL
LYEQAFQKNKITYHDIRTLLHLPDDTYFKGIVYDRGESRKQNENIRFLELDAYHQIRK
AVDKVYGKGKSSSFLPIDFDTFGYALTLFKDDADIHSYLRNEYEQNGKRMPMLALK
VYDNELIEELLNLSFTKFGHLSLKALRSILPYMEQGEVYSSACERAGYTFTGPKKKQK
TMPLLPNIPPIANPVVMRALTQARKVVNAIIKKYGSPVSIHIELARDLSQTFDERRKTKK
EQDENRKKNETAIRQLMEYGLTLNPTGHDIVKFKLWSEQNGRCAYSLQPIEIERLLEP
GYVEVDHVIPYSRSLDDSYTNKVLVLTRENREKGNRIPAEYLGVGTERWQQFETFVL
TNKQFSKKKDRLLRLHYDENEETEFKNRNLNFTRYISRFFANFIREHLKFAESDDK
QKVYTVNGRVTAHLRSRWEFNKNREESDLHHAVDAVIVACTTPSDIAKVTAFYQRR
EQNKELAKKTEPHFPQPWPHFADELRARLSKHPKESIKALNLGNYDDQKLESLQPVF
VSRMPKRSVTGAAHQETLRRYVGIDERSGKIQTVVKTKLSEIKLDASGHFPMYGKES
DPRTYEAIRQRLLEHNNDPKKAFQEPLYKPKKNGEPGPVIRTVKIIDTKNQVIPLNDG
KTVAYNSNIVRVDVFEKDGKYYCVPVYTMDIMKGILPNKAIEPNKPYSEWKEMTED
YTFRFSLYPNDLIRIELPREKTVKTAAGEEINVKDVFVYYKTIDSANGGLELISHDHRF
SLRGVGSRTLKRFEKYQVDVLGNIYKVRGEKRVGLASSAHSKPGKTIRPLQSTRD
GeoCas9 wt - Wild type version of the nickase used in GeoBE3
and GeoBE4 Consturcts
(SEQ ID NO: 3)
RYKIGLDIGITSVGWAVMNLDIPRIEDLGVRIFDRAENPQTGESLALPRRLAR
SARRRLRRRKHRLERIRRLVIREGILTKEELDKLFEEKHEIDVWQLRVEALDRKLNND
ELARVLLHLAKRRGFKSNRKSERSNKENSTMLKHIEENRAILSSYRTVGEMIVKDPK
FALHKRNKGENYTNTIARDDLEREIRLIFSKQREFGNMSCTEEFENEYITIWASQRPV
ASKDDIEKKVGFCTFEPKEKRAPKATYTFQSFIAWEHINKLRLISPSGARGLTDEERRL
LYEQAGQKNKITYHDIRTLLHLPDDTYFKGIVYDRGESRKQNENIRFLELDAYHQIRK
AVDKVYGKGKSSSFLPIDFDTFGYALTLFKDDADIHSYLRNEYEQNGKRMPNLANK
VYDNELIEELLNLSFTKFGHLSLKALRSILPYMEQGEVYSSACERAGYTFTGPKKKQK
TMLLPNIPPIANPVVMRALTQARKVVNAIIKKYGSPVSIHIELARDLSQTFDERRKTKK
EQDENRKKNETAIRQLMEYGLTLNPTGHDIVKFKLWSEQNGRCAYSLQPIEIERLLEP
GYVEVDHVIPYSRSLDDSYTNKVLVLTRENREKGNRIPAEYLGVGTERWQQFETFVL
TNKQFSKKKRDRLLRLHYDENEETEFKNRNLNDTRYISRFFANFIREHLKFAESDDK
QKVYTVNGRVTAHLRSRWEFNKNREESDLHHAVDAVIVACTTPSDIAKVTAFYQRR
EQNKELAKKTEPHFPQPWPHFADELRARLSKHPKESIKALNLGNYDDQKLESLQPVF
VSRMPKRSVTGAAHQETLRRYVGIDERSGKIQTVVKTKLSEIKLDASGHFPMYGKES
DPRTYEAIRQRLLEHNNDPKKAFQEPLYKPKKNGEPGPVIRTVKIIDTKNQVIPLNDG
KTVAYNSNIVRVDVFEKDGKYYCVPVYTMDIMKGILPNKAIEPNKPYSEWKEMTED
YTFRFSLYPNDLIRIELPREKTVKTAAGEEINVKDVFVYYKTIDSANGGLELISHDHRF
SLRGVGSRTLKRFEKYQVDVLGNIYKVRGEKRVGLASSAHSKPGKTIRPLQSTRD
In some embodiements, GeoCas9 corresponds to Cas9 from Geobacillus
stearothermophilus (>tr|A0A178TEJ9|A0A178TEJ9_GEOSE CRISPR-associated
endonuclease Cas9 OS = Geobacillus stearothermophilus GN = cas9 PE =
3 SV = 1; SEQ ID NO: 6)
(SEQ ID NO: 6)
MRYKIGLDIGITSVGWAVMNLDIPRIEDLGVRIFDRAENPQTGESLALPRRLA
RSARRRLRRRKHRLERIRRLVIREGILTKEELDKLFEEKHEIDVWQLRVEALDRKLNN
DELARVLLHLAKRRGFKSNRKSERSNKENSTMLKHIEENRAILSSYRTVGEMIVKDP
KFALHKRNKGENYTNTIARDDLEREIRLIFSKQREFGNMSCTEEFENEYITIWASQRP
VASKDDIEKKVGFCTFEPKEKRAPKATYTFQSFIAWEHINKLRLISPSGARGLTDEER
RLLYEQAFQKNKITYHDIRTLLHLPDDTYFKGIVYDRGESRKQNENIRFLELDAYHQI
RKAVDKVYGKGKSSSFLPIDFDTFGYALTLFKDDADIHSYLRNEYEQNGKRMPNLA
NKVYDNELIEELLNLSFTKFGHLSLKALRSILPYMEQGEVYSSACERAGYTFTGPKKK
QKTMLLPNIPPIANPVVMRALTQARKVVNAIIKKYGSPVSIHIELARDLSQTFDERRKT
KKEQDENRKKNETAIRQLMEYGLTLNPTGHDIVKFKLWSEQNGRCAYSLQPIEIERL
LEPGYVEVDHVIPYSRSLDDSYTNKVLVLTRENREKGNRIPAEYLGVGTERWQQFET
FVLTNKQFSKKKRDRLLRLHYDENEETEFKNRNLNDTRYISRFFANFIREHLKFAESD
DKQKVYTVNGRVTAHLRSRWEFNKNREESDLHHAVDAAIVACTTPSDIAKVTAFYQ
RREQNKELAKKTEPHFPQPWPHFADELRARLSKHPKESIKALNLGNYDDQKLESLQP
VFVSRMPKRSVTGAAHQETLRRYVGIDERSGKIQTVVKTKLSEIKLDASGHFPMYGK
ESDPRTYEAIRQRLLEHNNDPKKAFQEPLYKPKKNGEPGPVIRTVKIIDTKNQVIPLND
GKTVAYNSNIVRVDVFEKDGKYYCVPVYTMDIMKGILPNKAIEPNKPYSEWKEMTE
DYTFRFSLYPNDLIRIELPREKTVKTAAGEEINVKDFVYYKTIDSANGGLELISHDHR
FSLRGVGSRTLKRFEKYQVDVLGNIYKVRGEKRVGLASSAHSKTGETVRPLQSTRD
In some embodiments, GeoCas9 corresponds to Cas9 from Geobacillus
thermodenitrificans (NCBI Reference Sequence: WP_087959824.1
SEQ ID NO: 4 (nucleotide); SEQ ID NO: 5 (amino acid)).
(SEQ ID NO: 4)
atgaagtataaaatcggtcttgctatcggcattacgtctatcggttgggctgtcattaatttggac
attcctcgcatcgaagatttaggtgtccgcatttttgacagagcggaaaacccgaaaaccggggag
tcactagctcttccacgtcgcctcgcccgctccgcccgacgtcgtctgcggcgtcgcaaacatcga
ctggagcgcattcgccgcctgttcgtccgcgaaggaattttaacgaaggaagagctgaacaagctg
tttgaaaaaaagcacgaaatcgacgtctggcagcttcgtgttgaagcactggatcgaaaactaaat
aacgatgaattagcccgcatccttcttcatctggctaaacggcgtggatttagatccaaccgcaag
agtgagcgcaccaacaaagaaaacagtacgatgctcaaacatattgaagaaaaccaatccattctt
tcaagttaccgaacggttgcagaaatggttgtcaaggatccgaaattttccctgcacaagcgtaat
aaagaggataattacaccaacactgttgcccgcgacgatcttgaacgggaaatcaaactgattttc
gccaaacagcgcgaatatgggaacatcgtttgcacagaagcatttgaacacgagtatatttccatt
tgggcatcgcaacgcccttttgcttctaaggatgatatcgagaaaaaagtcggtttctgtacgttt
gagcctaaagaaaaacgcgcgccaaaagcaacatacacattccagtccttcaccgtctgggaacat
attaacaaacttcgtcttgtctccccgggaggcatccgggcactaaccgatgatgaacgtcgtctt
atatacaagcaagcatttcataaaaataaaatcaccttccatgatgttcgaacattgcttaacttg
cctgacgacacccgttttaaaggtcttttatatgaccgaaacaccacgctgaaggaaaatgagaaa
gttcgcttccttgaactcggcgcctatcataaaatacggaaagcgatcgacagcgtctatggcaaa
ggagcagcaaaatcatttcgtccgattgattttgatacatttggctacgcattaacgatgtttaaa
gacgacaccgacattcgcagttacttgcgaaacgaatacgaacaaaatggaaaacgaatggaaaat
ctagcggataaagtctatgatgaagaattgattgaagaacttttaaacttatcgttttctaagttt
ggtcatctatcccttaaagcgcttcgcaacatccttccatatatggaacaaggcgaagtctactca
accgcttgtgaacgagcaggatatacatttacagggccaaagaaaaaacagaaaacggtattgctg
ccgaacattccgccgatcgccaatccggtcgtcatgcgcgcactgacacaggcacgcaaagtggtc
aatgccattatcaaaaagtacggctcaccggtctccatccatatcgaactggcccgggaactatca
caatcctttgatgaacgacgtaaaatgcagaaagaacaggaaggaaaccgaaagaaaaacgaaact
gccattcgccaacttgttgaatatgggctgacgctcaatccaactgggcttgacattgtgaaattc
aaactatggagcgaacaaaacggaaaatgtgcctattcactccaaccgatcgaaatcgagcggttg
ctcgaaccaggctatacagaagtcgacgctgtgattccatacagccgaagcttggacgatagctat
accaataaagttcttgtgttgacaaaggagaaccgtgaaaaaggaaaccgcaccccagctgaatat
ttaggattaggctcagaacgttggcaacagttcgagacgtttgtcttgacaaataagcagttttcg
aaaaagaagcgggatcgactccttcggcttcattacgatgaaaacgaagaaaatgagtttaaaaat
cgtaatctaaatgatacccgttatatctcacgcttcttggctaactttattcgcgaacatctcaaa
ttcgccgacagcgatgacaaacaaaaagtatacacggtcaacggccgtattaccgcccatttacgc
agccgttggaattttaacaaaaaccgggaagaatcgaatttgcatcatgccgtcgatgctgccatc
gtcgcctgcacaacgccgagcgatatcgcccgagtcaccgccttctatcaacggcgcgaacaaaac
aaagaactgtccaaaaagacggatccgcagtttccgcagccttggccgcactttgctgatgaactg
caggcgcgtttatcaaaaaatccaaaggagagtataaaagctctcaatcttggaaattatgataac
gagaaactcgaatcgttgcagccggtttttgtctcccgaatgccgaagcggagcataacaggagcg
gctcatcaagaaacattgcggcgttatatcggcatcgacgaacggagcggaaaaatacagacggtc
gtcaaaaagaaactatccgagatccaactggataaaacaggtcatttcccaatgtacgggaaagaa
agcgatccaaggacatatgaagccattcgccaacggttgcttgaacataacaatgacccaaaaaag
gcgtttcaagagcctctgtataaaccgaagaagaacggagaactaggtcctatcatccgaacaatc
aaaatcatcgatacgacaaatcaagttattccgctcaacgatggcaaaacagtcgcctacaacagc
aacatcgtgcgggtcgacgtctttgagaaagatggcaaatattattgtgtccctatctatacaata
gatatgatgaaagggatcttgccaaacaaggcgatcgagccgaacaaaccgtactctgagtggaag
gaaatgacggaggactatacattccgattcagtctatacccaaatgatcttatccgtatcgaattt
ccccgagaaaaaacaataaagactgctgtgggggaagaaatcaaaattaaggatctgttcgcctat
tatcaaaccatcgactcctccaatggagggttaagtttggttagccatgataacaacttttcgctc
cgcagcatcggttcaagaaccctcaaacgattcgagaaataccaagtagatgtgctaggcaacatc
tacaaagtgagaggggaaaagagagttggggtggcgtcatcttctcattcgaaagccggggaaact
atccgtccgttataa
(SEQ ID NO: 5)
MKYKIGLDIGITSIGWAVINLDIPRIEDLGVRIFDRAENPKTGESLALPRRLAR
SARRRLRRRKHRLERIRRLFVREGILTKEELNKLFEKKHEIDVWQLRVEALDRKLNN
DELARILLHLAKRRGFRSNRKSERTNKENSTMLKHIEENQSILSSYRTVAEMVVKDP
KFSLHKRNKEDNYTNTVARDDLEREIKLIFAKQREYGNIVCTEAFEHEYISIWASQRP
FASKDDIEKKVGFCTFEPKEKRAPKATYTFQSFTVWEHINKLRLVSPGGIRALTDDER
RLIYKQAFHKNKITFHDVRTLLNLPDDTRFKGLLYDRNTTLKENEKVRFLELGAYHK
IRKAIDSVYGKGAAKSFRPIDFDTFGYALTMFKDDTDIRSYLRNEYEQNGKRMENLA
DKVYDEELIEELLNLSFSKFGHLSLKALRNILPYMEQGEVYSTACERAGYTFTGPKKK
QKTVLLPNIPPIANPVVMRALTQARKVVNAIIKKYGSPVSIHIELARELSQSFDERRKM
QKEQEGNRKKNETAIRQLVEYGLTLNPTGLDIVKFKLWSEQNGKCAYSLQPIEIERLL
EPGYTEVDHVIPYSRSLDDSYTNKVLVLTKENREKGNRTPAEYLGLGSERWQQFETF
VLTNKQFSKKKRDRLLRLHYDENEENEFKNRNLNDTRYISRFLANFIREHLKFADSD
DKQKVYTVNGRITAHLRSRWNFNKNREESNLHHAVDAAIVACTTPSDIARVTAFYQ
RREQNKELSKKTDPQFPQPWPHFADELQARLSKNPKESIKALNLGNYDNEKLESLQP
VFVSRMPKRSITGAAHQETLRRYIGIDERSGKIQTVVKKKLSEIQLDKTGHFPMYGKE
SDPRTYEAIRQRLLEHNNDPKKAFQEPLYKPKKNGELGPIIRTIKIIDTTNQVIPLNDGK
TVAYNSNIVRVDVFEKDGKYYCVPIYTIDMMKGILPNKAIEPNKPYSEWKEMTEDYT
FRFSLYPNDLIRIEFPREKTIKTAVGEEIKIKDLFAYYQTIDSSNGGLSLVSHDNNFSLR
SIGSRTLKRFEKYQVDVLGNIYKVRGEKRVGVASSSHSKAGETIRPL

B. GeoCas9 with Altered PAM Specificities

The GeoCas9 base editors may also comprise GeoCas9 proteins with altered PAM specificities.

Some aspects of the disclosure provide GeoCas9 proteins that have Protospacer Adjacent Motif (PAM) specificities that differ from other GeoCas9 proteins. A PAM sequence is typically a 2-6 base pair DNA sequence that immediately follows the DNA sequence targeted by a Cas9 nuclease in the CRISPR bacterial adaptive immune system. Typically Cas9 will not bind to or cleave a target DNA sequence if it is not followed by the PAM sequence. Thus, PAM requirements may limit the ability to edit desired bases within a genome. For example spCas9 proteins may be limited to binding to locations of a nucleic acid molecule having an adjacent 5′-NGG-3′ sequence. In some embodiments, the base editing fusion proteins provided herein may need to be placed at a precise location, for example where a target base is placed within a 4 base region (e.g., a “deamination window”), which is approximately 15 bases upstream of the PAM. See Komor, A. C., et al., “Programmable editing of a target base in genomic DNA without double-stranded DNA cleavage” Nature 533, 420-424 (2016), the entire contents of which are hereby incorporated by reference. In some embodiments, a target base is placed within a base region from 1 to 10 nucleotides in length. In some embodiments, a target base is placed within a base region that is at least 1, at lest 2, at least 3, at least 4, at least 5, at least 6, at least 7, at least 8, at least 9, or at least 10 nucleotides in length. In some embodiments, the target base is from 5 to 25 bases upstream of the PAM. In some embodiments, the target base is 5, 6, 7, 8, 9, 10, 11, 12, 13, 14, 15, 16, 17, 18, 19, 20, 21, 22, 23, 24, or 25 bases upstream of the PAM.

In some embodiments, any of the fusion proteins provided herein may contain a GeoCas9 that is capable of binding a nucleotide sequence having the PAM sequence 5′-NNNNCRAA-3′ (SEQ ID NO: 79), where N is A, T, C, or G, and R is A or G as indicated by the IUPAC nucleotide code naming system. In some embodiments, the GeoCas9 is capable of binding a nucleotide sequence having the PAM sequence 5′-NIN2N3N4CRAA-3′, where N1, N2, N3, and/or N4 is A. In some embodiments, the GeoCas9 is capable of binding a nucleotide sequence having the PAM sequence 5′-N₁N₂N₃N₄CRAA-3′, where N₁, N₂, N₃, and/or N₄ is T. In some embodiments, the GeoCas9 is capable of binding a nucleotide sequence having the PAM sequence 5′-N₁N₂N₃N₄CRAA-3′, where N₁, N₂, N₃, and/or N₄ is C. In some embodiments, the GeoCas9 is capable of binding a nucleotide sequence having the PAM sequence 5′-N₁N₂N₃N₄CRAA-3′, where N₁, N₂, N₃, and/or N₄ is G. It should be appreciated that N₁N₂N₃N₄ may be a four nucleotide sequence having any combination of neucleotides selected from the group consisting of A, T, C, and G. GeoCas9 proteins that bind to 5′-NNNNCRAA-3′ (SEQ ID NO: 79) PAM sequences have been described in Harrington L. B., Paez-Espino D., Staahl B. T., Chen J. C., Ma E., Kyrpides N.C., Doudna J. A. Nature Communications 8, 1424 (2017)); the entire contents of each are hereby incorporated by reference.

In some embodiments, the GeoCas9 or variant thereof is a GeoCas9 or variant thereof from the genus Geobacillus. In some embodiments, the GeoCas9 or variant thereof is from a species selected from the group consisting of G. stearothermophilus, G. thermodenitrificans, G. caldoxylosilyticus, G. galactosidasius, G. icigianus, G. jurassicus, G. kaustophilus, G. lituanicus, G. subterraneus, G. thermantarcticus, G. thermocatenulatus, G. thermoglucosidasius, G. thermoleovorans, G. toebii, G. uzenensis, and G. vulcani. In some embodiments, the GeoCas9 of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of SEQ ID NOs: 2, 3, or 5-8. In some embodiments, the GeoCas9 of any of the fusion proteins provided herein comprises the amino acid sequence of any one of SEQ ID NOs: 2, 3, or 5-8. In some embodiments, the GeoCas9 protein of any of the fusion proteins provided herein consists of the amino acid sequence of any one of SEQ ID NOs: 2, 3, or 5-8.

Additional suitable GeoCas9 proteins, variants, and sequences will also be apparent to those of skill in the art. Examples of such additional suitable GeoCas9 proteins include, but are not limited to, D8A, D8A/D581A/H582A, and D8A/D581A/H582A/N605A mutant domains (see, e.g., Prashant et al., CAS9 transcriptional activators for target specificity screening and paired nickases for cooperative genome engineering. Nature Biotechnology. 2013; 31 (9): 833-838 the entire contents of which are incorporated herein by reference). In some embodiments, the GeoCas9 comprises a histidine residue at position 582 of the amino acid sequence provided in SEQ ID NO: 3, or a corresponding mutation in any of the amino acid sequences provided in SEQ ID NOs: 2, 3, 5-8.

### C. Nucleic Acid Effector Domain

The GeoCas9 base editor fusion proteins may comprise one or more nucleic acid effector domains. Exemplary nucleic acid effector domains include, but are not limited to a deaminase (e.g., a cytidine deaminase or an adenosine deaminase), a nuclease, a nickase, a recombinase, a methyltransferase, a methylase, an acetylase, an acetyltransferase, a transcriptional activator, or a transcriptional repressor domain. In some embodiments the nucleic acid effector domain is a protein or enzyme capable of making one or more modifications (e.g., deamination of a cytidine residue) to a nucleic acid (e.g., DNA or RNA). In some embodiments the nucleic acid editing domain is a deaminase (e.g., a cytidine deaminase, such as an APOBEC or an AID deaminase). Additional suitable nucleic-acid effector enzyme sequences that can be used according to aspects of this invention, e.g., that can be fused to a GeoCas9 protein, will be apparent to those of skill in the art based on this disclosure. In some embodiments, such additional enzyme sequences include deaminase enzyme or deaminase domain sequences that are at least 70%, at least 75%, at least 80%, at least 85%, at least 90%, at least 95%, at least 96%, at least 97%, at least 98%, or at least 99% similar to the sequences provided herein.

In some embodiments, the nucleic acid effector domain is a deaminase domain. In some embodiments, the deaminase is a cytosine deaminase or a cytidine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the demianse is a rat APOBEC1 (SEQ ID NO: 43). In some embodiments, the deminase is a human APOBEC1 (SEQ ID NO: 42). In some embodiments, the deaminase is a Petromyzon marinus cytidine deaminase 1 (pmCDA1) (SEQ ID NO: 53). In some embodiments, the deminase is a human APOBEC3G (SEQ ID NO: 34). In some embodiments, the deaminase is a fragment of the human APOBEC3G (SEQ ID NO: 58). In some embodiments, the deaminase is a human APOBEC3G variant comprising a D316R_D317R mutation (SEQ ID NO: 57). In some embodiments, the deaminase is a frantment of the human APOBEC3G and comprising mutations corresponding to the D316R_D317R mutations in SEQ ID NO: 34 (SEQ ID NO: 59).

One exemplary suitable type of nucleic acid effector domain is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner.²⁹ One family member, activation-induced cytidine deaminase (AID), is responsible for the maturation of antibodies by converting cytosines in ssDNA to uracils in a transcription-dependent, strand-biased fashion.³⁰ The apolipoprotein B editing complex 3 (APOBEC3) enzyme provides protection to human cells against a certain HIV-1 strain via the deamination of cytosines in reverse-transcribed viral ssDNA.31 These proteins all require a Zn²⁺-coordinating motif (His-X-Glu-X₂₃₋₂₆-Pro-Cys-X₂₋₄-Cys; SEQ ID NO: 383) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F.³² A recent crystal structure of the catalytic domain of APOBEC3G revealed a secondary structure comprised of a five-stranded β-sheet core flanked by six α-helices, which is believed to be conserved across the entire family.³³ The active center loops have been shown to be responsible for both ssDNA binding and in determining “hotspot” identity.³⁴ Overexpression of these enzymes has been linked to genomic instability and cancer, thus highlighting the importance of sequence-specific targeting.³⁵

Some aspects of this disclosure relate to the recognition that the activity of cytidine deaminase enzymes such as APOBEC enzymes can be directed to a specific site in genomic DNA. Without wishing to be bound by any particular theory, advantages of using GeoCas9 as a recognition agent include (1) the sequence specificity of GeoCas9 can be easily altered by simply changing the sgRNA sequence; and (2) GeoCas9 binds to its target sequence by denaturing the dsDNA, resulting in a stretch of DNA that is single-stranded and therefore a viable substrate for the deaminase; (3) GeoCas9 is a thermostable hololog of other Cas9 proteins; (4) GeoCas9 is smaller than many homologs of Cas9 proteins from other organisms; and (5) GeoCas9 recognizes and can bind to nucleic acid sequences comprising the PAM sequence 5′-NNNNCRAA-3′ (SEQ ID NO: 79). It should be understood that other catalytic domains, or catalytic domains from other deaminases, can also be used to generate GeoCas9 base editor fusion proteins with GeoCas9, and that the disclosure is not limited in this regard.

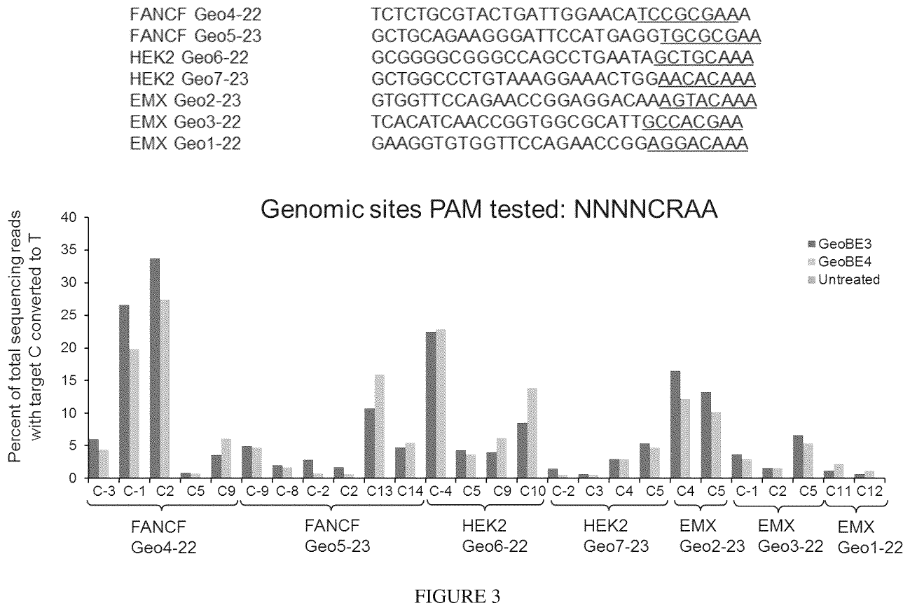

Some aspects of this disclosure are based on the recognition that Cas9: deaminase fusion proteins can efficiently deaminate nucleotides at positions-15 to 15 according to the numbering scheme in FIG. 3. In view of the results provided herein regarding the nucleotides that can be targeted by Cas9: deaminase fusion proteins, a person of skill in the art will be able to design suitable guide RNAs to target the GeoCas9 base editor fusion proteins to a target sequence that comprises a nucleotide to be deaminated. In some embodiments, the deamination window is from position −15 to 15. In some embodiments, the deamination window is from position −15 to 10, from −15 to 5, from −15 to 0, from −15 to −5, from −15 to −10, from −10 to 15, from −10 to 10, from −10 to 5, from −10 to 0, from −10 to −5, from −5 to 15, from −5 to 10, from −5 to 5, from −5 to 0, from 0 to 15, from 0 to 10, from 0 to 5, from 5 to 15, from 5 to 10, or from 10 to 15 according to the numbering scheme in SEQ ID NO: 406.

### D. Cytidine Deaminases

In some embodiments, the GeoCas9 base editors may comprise a cytidine deaminase as a nucleic acid effector domain. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1. In some embodiments, the deaminase is an activation-induced cytidine deaminase (AID). In some embodiments, the deaminase is a cytidine deaminase 1 (CDA1). In some embodiments, the deaminase is a Petromyzon marinus cytidine deaminase 1 (pmCDA1). In some embodiments, the deminase is a human APOBEC3G (SEQ ID NO: 34). In some embodiments, the deaminase is a fragment of the human APOBEC3G (SEQ ID NO: 58). In some embodiments, the deaminase is a human APOBEC3G variant comprising a D316R_D317R mutation (SEQ ID NO: 57). In some embodiments, the deaminase is a frantment of the human APOBEC3G and comprising mutations corresponding to the D316R_D317R mutations in SEQ ID NO: 34 (SEQ ID NO: 59).

In some embodiments, the nucleic acid editing domain is at least 80%, at least 85%, at least 90%, at least 92%, at least 95%, at least 96%, at least 97%, at least 98%, at least 99%, or at least 99.5% identical to the deaminase domain of any one of SEQ ID NOs: 25-59. In some embodiments, the nucleic acid editing domain comprises the amino acid sequence of any one of SEQ ID NOs: 25-59.

A cytidine deaminase domain may also be referred to interchangeably as a cytosine deaminase domain. In some embodiments, the cytidine deaminase catalyzes the hydrolytic deamination of cytidine (C) or deoxycytidine (dC) to uridine (U) or deoxyuridine (dU), respectively. In some embodiments, the cytidine deaminase domain catalyzes the hydrolytic deamination of cytosine (C) to uracil (U). In some embodiments, the cytidine deaminase catalyzes the hydrolytic deamination of cytidine or cytosine in deoxyribonucleic acid (DNA). Without wishing to be bound by any particular theory, fusion proteins comprising a cytidine deaminase are useful inter alia for targeted editing, referred to herein as “base editing,” of nucleic acid sequences in vitro and in vivo.

One exemplary suitable type of cytidine deaminase is a cytidine deaminase, for example, of the APOBEC family. The apolipoprotein B mRNA-editing complex (APOBEC) family of cytidine deaminase enzymes encompasses eleven proteins that serve to initiate mutagenesis in a controlled and beneficial manner (see, e.g., Conticello S G. The AID/APOBEC family of nucleic acid mutators. Genome Biol. 2008; 9 (6): 229). One family member, activation-induced cytidine deaminase (AID), is responsible for the maturation of antibodies by converting cytosines in ssDNA to uracils in a transcription-dependent, strand-biased fashion (see, e.g., Reynaud C A, et al. What role for AID: mutator, or assembler of the immunoglobulin mutasome, Nat Immunol. 2003; 4 (7): 631-638). The apolipoprotein B editing complex 3 (APOBEC3) enzyme provides protection to human cells against a certain HIV-1 strain via the deamination of cytosines in reverse-transcribed viral ssDNA (see, e.g., Bhagwat A S. DNA-cytosine deaminases: from antibody maturation to antiviral defense. DNA Repair (Amst). 2004; 3 (1): 85-89). These proteins all require a Zn2+-coordinating motif (His-X-Glu-X23-26-Pro-Cys-X2-4-Cys; SEQ ID NO: 383) and bound water molecule for catalytic activity. The Glu residue acts to activate the water molecule to a zinc hydroxide for nucleophilic attack in the deamination reaction. Each family member preferentially deaminates at its own particular “hotspot”, ranging from WRC (W is A or T, R is A or G) for hAID, to TTC for hAPOBEC3F (see, e.g., Navaratnam N and Sarwar R. An overview of cytidine deaminases. Int J Hematol. 2006; 83 (3): 195-200). A recent crystal structure of the catalytic domain of APOBEC3G revealed a secondary structure comprised of a five-stranded β-sheet core flanked by six α-helices, which is believed to be conserved across the entire family (see, e.g., Holden L G, et al. Crystal structure of the anti-viral APOBEC3G catalytic domain and functional implications. Nature. 2008; 456 (7218): 121-4). The active center loops have been shown to be responsible for both ssDNA binding and in determining “hotspot” identity (see, e.g., Chelico L, et al. Biochemical basis of immunological and retroviral responses to DNA-targeted cytosine deamination by activation-induced cytidine deaminase and APOBEC3G. J Biol Chem. 2009; 284 (41). 27761-5). Overexpression of these enzymes has been linked to genomic instability and cancer, thus highlighting the importance of sequence-specific targeting (see, e.g., Pham P, et al. Reward versus risk: DNA cytidine deaminases triggering immunity and disease. Biochemistry. 2005; 44 (8): 2703-15).

Some aspects of this disclosure relate to the recognition that the activity of cytidine deaminase enzymes such as APOBEC enzymes can be directed to a specific site in genomic DNA. Without wishing to be bound by any particular theory, advantages of using a nucleic acid programmable binding protein (e.g., a Cas9 domain) as a recognition agent include (1) the sequence specificity of nucleic acid programmable binding protein (e.g., a Cas9 domain) can be easily altered by simply changing the sgRNA sequence; and (2) the nucleic acid programmable binding protein (e.g., a Cas9 domain) may bind to its target sequence by denaturing the dsDNA, resulting in a stretch of DNA that is single-stranded and therefore a viable substrate for the deaminase. It should be understood that other catalytic domains of napDNAbps, or catalytic domains from other nucleic acid editing proteins, can also be used to generate fusion proteins with Cas9, and that the disclosure is not limited in this regard.

In view of the results provided herein regarding the nucleotides that can be targeted by Cas9: deaminase fusion proteins, a person of ordinary skill in the art will be able to design suitable guide RNAs to target the fusion proteins to a target sequence that comprises a nucleotide to be deaminated.

In some embodiments, the cytidine deaminase is an apolipoprotein B mRNA-editing complex (APOBEC) family deaminase. In some embodiments, the cytidine deaminase is an APOBEC1 deaminase. In some embodiments, the cytidine deaminase is an APOBEC2 deaminase. In some embodiments, the cytidine deaminase is an APOBEC3 deaminase. In some embodiments, the cytidine deaminase is an APOBEC3A deaminase. In some embodiments, the cytidine deaminase is an APOBEC3B deaminase. In some embodiments, the cytidine deaminase is an APOBEC3C deaminase. In some embodiments, the cytidine deaminase is an APOBEC3D deaminase. In some embodiments, the cytidine deaminase is an APOBEC3E deaminase. In some embodiments, the cytidine deaminase is an APOBEC3F deaminase. In some embodiments, the cytidine deaminase is an APOBEC3G deaminase. In some embodiments, the cytidine deaminase is an APOBEC3H deaminase. In some embodiments, the cytidine deaminase is an APOBEC4 deaminase. In some embodiments, the cytidine deaminase is an activation-induced deaminase (AID). In some embodiments, the cytidine deaminase is a vertebrate cytidine deaminase. In some embodiments, the cytidine deaminase is an invertebrate cytidine deaminase. In some embodiments, the cytidine deaminase is a human, chimpanzee, gorilla, monkey, cow, dog, rat, or mouse deaminase. In some embodiments, the cytidine deaminase is a human cytidine deaminase. In some embodiments, the cytidine deaminase is a rat cytidine deaminase, e.g., rAPOBEC1. In some embodiments, the cytidine deaminase is a Petromyzon marinus cytidine deaminase 1 (pmCDA1) (SEQ ID NO: 56). In some embodiments, the cytidine deaminase is a human APOBEC3G (SEQ ID NO: 34). In some embodiments, the cytidine deaminase is a fragment of the human APOBEC3G. In some embodiments, the deaminase is a human APOBEC3G variant comprising a D316R and D317R mutation. In some embodiments, the deaminase is a fragment of the human APOBEC3G and comprising mutations corresponding to the D316R and D317R mutations in SEQ ID NO: 59.

In some embodiments, the nucleic acid editing domain is at least 80%, at least 85%, at least 90%, at least 92%, at least 95%, at least 96%, at least 97%, at least 98%, at least 99%, or at least 99.5% identical to the cytidine deaminase domain of any one of SEQ ID NOs: 25-59. In some embodiments, the nucleic acid editing domain comprises the amino acid sequence of any one of SEQ ID NOs: 25-59.

Some exemplary suitable nucleic-acid editing domains, e.g., cytidine deaminases and cytidine deaminase domains, that can be fused to a GeoCas9 domain according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal). The following are exemplary cytidine deaminases that may be used in the GeoCas9 base editor fusion protein disclosed herein.

Human AID:
(SEQ ID NO: 25)
MDSLLMNRRKFLYQFKNVRWAKGRRETYLCYVVKRRDSATSFSLDFGYL
RNKNGCHVELLFLRYISDWDLDPGRCYRVTWFTSWSPCYDCARHVADFLRGNPNLS
LRIFTARLYFCEDRKAEPEGLRRLHRAGVQIAIMTFKDYFYCWNTFVENHERTFKAW
EGLHENSVRLSRQLRRILLPLYEVDDLRDAFRTLGL
(underline: nuclear localization sequence;
double underline: nuclear export signal)
Mouse AID:
(SEQ ID NO: 26)
MDSLLMKQKKFLYHFKNVRWAKGRHETYLCYVVKRRDSATSCSLDFGH
LRNKSGCHVELLFLRYISDWDLDPGRCYRVTWFTSWSPCYDCARHVAEFLRWNPNL
SLRIFTARLYFCEDRKAEPEGLRRLHRAGVQIGIMTFKDYFYCWNTFVENRERTFKA
WEGLHENSVRLTRQLRRILLPLYEVDDLRDAFRMLGF
(underline: nuclear localization sequence;
double underline: nuclear export signal)
Dog AID:
(SEQ ID NO: 27)
MDSLLMKQRKFLYHFKNVRWAKGRHETYLCYVVKRRDSATSFSLDFGHL
RNKSGCHVELLFLRYISDWDLDPGRCYRVTWFTSWSPCYDCARHVADFLRGYPNLS
LRIFAARLYFCEDRKAEPEGLRRLHRAGVQIAIMTFKDYFYCWNTFVENREKTFKAW
EGLHENSVRLSRQLRRILLPLYEVDDLRDAFRTLGL
(underline: nuclear localization sequence;
double underline: nuclear export signal)
Bovine AID:
(SEQ ID NO: 28)
MDSLLKKQRQFLYQFKNVRWAKGRHETYLCYVVKRRDSPTSFSLDFGHL
RNKAGCHVELLFLRYISDWDLDPGRCYRVTWFTSWSPCYDCARHVADFLRGYPNLS
LRIFTARLYFCDKERKAEPEGLRRLHRAGVQIAIMTFKDYFYCWNTFVENHERTFKA
WEGLHENSVRLSRQLRRILLPLYEVDDLRDAFRTLGL
(underline: nuclear localization sequence;
double underline: nuclear export signal)
Rat AID:
(SEQ ID NO: 44)
MAVGSKPKAALVGPHWERERIWCFLCSTGLGTQQTGQTSRWLRPAATQD
PVSPPRSLLMKQRKFLYHFKNVRWAKGRHETYLCYVVKRRDSATSFSLDFGYLRNK
SGCHVELLFLRYISDWDLDPGRCYRVTWFTSWSPCYDCARHVADFLRGNPNLSLRIF
TARLTGWGALPAGLMSPARPSDYFYCWNTFVENHERTFKAWEGLHENSVRLSRRL
RRILLPLYEVDDLRDAFRTLGL
(underline: nuclear localization sequence;
double underline: nuclear export signal)
Mouse APOBEC-3:
(SEQ ID NO: 29)
MGPFCLGCSHRKCYSPIRNLISQETFKFHFKNLGYAKGRKDTFLCYEVTRK
DCDSPVSLHHGVFKNKDNIHAEICFLYWFHDKVLKVLSPREEFKITWYMSWSPCFEC
AEQIVRFLATHHNLSLDIFSSRLYNVQDPETQQNLCRLVQEGAQVAAMDLYEFKKC
WKKFVDNGGRRFRPWKRLLTNFRYQDSKLQEILRPCYIPVPSSSSSTLSNICLTKGLP
ETRFCVEGRRMDPLSEEEFYSQFYNQRVKHLCYYHRMKPYLCYQLEQFNGQAPLKG
CLLSEKGKQHAEILFLDKIRSMELSQVTITCYLTWSPCPNCAWQLAAFKRDRPDLILH
IYTSRLYFHWKRPFQKGLCSLWQSGILVDVMDLPQFTDCWTNFVNPKRPFWPWKGL
EIISRRTQRRLRRIKESWGLQDLVNDFGNLQLGPPMS
(italic: nucleic acid editing domain)
Rat APOBEC-3:
(SEQ ID NO: 30)
MGPFCLGCSHRKCYSPIRNLISQETFKFHFKNLRYAIDRKDTFLCYEVTRKD
CDSPVSLHHGVFKNKDNIHAEICFLYWFHDKVLKVLSPREEFKITWYMSWSPCFECA
EQVLRFLATHHNLSLDIFSSRLYNIRDPENQQNLCRLVQEGAQVAAMDLYEFKKCW
KKFVDNGGRRFRPWKKLLTNFRYQDSKLQEILRPCYIPVPSSSSSTLSNICLTKGLPET
RFCVERRRVHLLSEEEFYSQFYNQRVKHLCYYHGVKPYLCYQLEQFNGQAPLKGCL
LSEKGKQHAEILFLDKIRSMELSQVIITCYLTWSPCPNCAWQLAAFKRDRPDLILHIYT
SRLYFHWKRPFQKGLCSLWQSGILVDVMDLPQFTDCWTNFVNPKRPFWPWKGLEII
SRRTQRRLHRIKESWGLQDLVNDFGNLQLGPPMS
(italic: nucleic acid editing domain)
Rhesus macaque APOBEC-3G:
(SEQ ID NO: 31)
MVEPMDPRTFVSNFNNRPILSGLNTVWLCCEVKTKDPSGPPLDAKIFQGKV
YSKAKYHPEMRFLRWFHKWRQLHHDQEYKVTWYVSWSPCTRCANSVATFLAKDP
KVTLTIFVARLYYFWKPDYQQALRILCQKRGGPHATMKIMNYNEFQDCWNKFVDG
RGKPFKPRNNLPKHYTLLQATLGELLRHLMDPGTFTSNFNNKPWVSGQHETYLCYK
VERLHNDTWVPLNQHRGFLRNQAPNIHGFPKGRHAELCFLDLIPFWKLDGQQYRVT
CFTSWSPCFSCAQEMAKFISNNEHVSLCIFAARIYDDQGRYQEGLRALHRDGAKIAM
MNYSEFEYCWDTFVDRQGRPFQPWDGLDEHSQALSGRLRAI
(italic: nucleic acid editing domain;
underline: cytoplasmic localization signal)
Chimpanzee APOBEC-3G:
(SEQ ID NO: 32)
MKPHFRNPVERMYQDTFSDNFYNRPILSHRNTVWLCYEVKTKGPSRPPLD
AKIFRGQVYSKLKYHPEMRFFHWFSKWRKLHRDQEYEVTWYISWSPCTKCTRDVA
TFLAEDPKVTLTIFVARLYYFWDPDYQEALRSLCQKRDGPRATMKIMNYDEFQHCW
SKFVYSQRELFEPWNNLPKYYILLHIMLGEILRHSMDPPTFTSNFNNELWVRGRHET
YLCYEVERLHNDTWVLLNQRRGFLCNQAPHKHGFLEGRHAELCFLDVIPFWKLDLH
QDYRVTCFTSWSPCFSCAQEMAKFISNNKHVSLCIFAARIYDDQGRCQEGLRTLAKA
GAKISIMTYSEFKHCWDTFVDHQGCPFQPWDGLEEHSQALSGRLRAILQNQGN
(italic: nucleic acid editing domain;
underline: cytoplasmic localization signal)
Green monkey APOBEC-3G:
(SEQ ID NO: 33)
MNPQIRNMVEQMEPDIFVYYFNNRPILSGRNTVWLCYEVKTKDPSGPPLD
ANIFQGKLYPEAKDHPEMKFLHWFRKWRQLHRDQEYEVTWYVSWSPCTRCANSVA
TFLAEDPKVTLTIFVARLYYFWKPDYQQALRILCQERGGPHATMKIMNYNEFQHCW
NEFVDGQGKPFKPRKNLPKHYTLLHATLGELLRHVMDPGTFTSNFNNKPWVSGQRE
TYLCYKVERSHNDTWVLLNQHRGFLRNQAPDRHGFPKGRHAELCFLDLIPFWKLDD
QQYRVTCFTSWSPCFSCAQKMAKFISNNKHVSLCIFAARIYDDQGRCQEGLRTLHRD
GAKIAVMNYSEFEYCWDTFVDRQGRPFQPWDGLDEHSQALSGRLRAI
(italic: nucleic acid editing domain;
underline: cytoplasmic localization signal)
Human APOBEC-3G:
(SEQ ID NO: 34)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKCTRDMA
TFLAEDPKVTLTIFVARLYYFWDPDYQEALRSLCQKRDGPRATMKIMNYDEFQHCW
SKFVYSQRELFEPWNNLPKYYILLHIMLGEILRHSMDPPTFTFNFNNEPWVRGRHETY
LCYEVERMHNDTWVLLNQRRGFLCNQAPHKHGFLEGRHAELCFLDVIPFWKLDLD
QDYRVTCFTSWSPCFSCAQEMAKFISKNKHVSLCIFTARIYDDQGRCQEGLRTLAEA
GAKISIMTYSEFKHCWDTFVDHQGCPFQPWDGLDEHSQDLSGRLRAILQNQEN
(italic: nucleic acid editing domain;
underline: cytoplasmic localization signal)
Human APOBEC-3F:
(SEQ ID NO: 35)
MKPHFRNTVERMYRDTFSYNFYNRPILSRRNTVWLCYEVKTKGPSRPRLD
AKIFRGQVYSQPEHHAEMCFLSWFCGNQLPAYKCFQITWFVSWTPCPDCVAKLAEF
LAEHPNVTLTISAARLYYYWERDYRRALCRLSQAGARVKIMDDEEFAYCWENFVYS
EGQPFMPWYKFDDNYAFLHRTLKEILRNPMEAMYPHIFYFHFKNLRKAYGRNESWL
CFTMEVVKHHSPVSWKRGVFRNQVDPETHCHAERCFLSWFCDDILSPNTNYEVTWY
TSWSPCPECAGEVAEFLARHSNVNLTIFTARLYYFWDTDYQEGLRSLSQEGASVEIM
GYKDFKYCWENFVYNDDEPFKPWKGLKYNFLFLDSKLQEILE
(italic: nucleic acid editing domain)
Human APOBEC-3B:
(SEQ ID NO: 36)
MNPQIRNPMERMYRDTFYDNFENEPILYGRSYTWLCYEVKIKRGRSNLLW
DTGVFRGQVYFKPQYHAEMCFLSWFCGNQLPAYKCFQITWFVSWTPCPDCVAKLA
EFLSEHPNVTLTISAARLYYYWERDYRRALCRLSQAGARVTIMDYEEFAYCWENFV
YNEGQQFMPWYKFDENYAFLHRTLKEILRYLMDPDTFTFNFNNDPLVLRRRQTYLC
YEVERLDNGTWVLMDQHMGFLCNEAKNLLCGFYGRHAELRFLDLVPSLQLDPAQI
YRVTWFISWSPCFSWGCAGEVRAFLQENTHVRLRIFAARIYDYDPLYKEALQMLRD
AGAQVSIMTYDEFEYCWDTFVYRQGCPFQPWDGLEEHSQALSGRLRAILQNQGN
(italic: nucleic acid editing domain)
Rat APOBEC-3B:
(SEQ ID NO: 48)
MQPQGLGPNAGMGPVCLGCSHRRPYSPIRNPLKKLYQQTFYFHFKNVRYA
WGRKNNFLCYEVNGMDCALPVPLRQGVFRKQGHIHAELCFIYWFHDKVLRVLSPM
EEFKVTWYMSWSPCSKCAEQVARFLAAHRNLSLAIFSSRLYYYLRNPNYQQKLCRLI
QEGVHVAAMDLPEFKKCWNKFVDNDGQPFRPWMRLRINFSFYDCKLQEIFSRMNLL
REDVFYLQFNNSHRVKPVQNRYYRRKSYLCYQLERANGQEPLKGYLLYKKGEQHV
EILFLEKMRSMELSQVRITCYLTWSPCPNCARQLAAFKKDHPDLILRIYTSRLYFYWR
KKFQKGLCTLWRSGIHVDVMDLPQFADCWTNFVNPQRPFRPWNELEKNSWRIQRR
LRRIKESWGL
Bovine APOBEC-3B:
(SEQ ID NO: 49)
DGWEVAFRSGTVLKAGVLGVSMTEGWAGSGHPGQGACVWTPGTRNTM
NLLREVLFKQQFGNQPRVPAPYYRRKTYLCYQLKQRNDLTLDRGCFRNKKQRHAEI
RFIDKINSLDLNPSQSYKIICYITWSPCPNCANELVNFITRNNHLKLEIFASRLYFHWIK
SFKMGLQDLQNAGISVAVMTHTEFEDCWEQFVDNQSRPFQPWDKLEQYSASIRRRL
QRILTAPI
Chimpanzee APOBEC-3B:
(SEQ ID NO: 50)
MNPQIRNPMEWMYQRTFYYNFENEPILYGRSYTWLCYEVKIRRGHSNLL
WDTGVFRGQMYSQPEHHAEMCFLSWFCGNQLSAYKCFQITWFVSWTPCPDCVAKL
AKFLAEHPNVTLTISAARLYYYWERDYRRALCRLSQAGARVKIMDDEEFAYCWENF
VYNEGQPFMPWYKFDDNYAFLHRTLKEIIRHLMDPDTFTFNFNNDPLVLRRHQTYL
CYEVERLDNGTWVLMDQHMGFLCNEAKNLLCGFYGRHAELRFLDLVPSLQLDPAQI
YRVTWFISWSPCFSWGCAGQVRAFLQENTHVRLRIFAARIYDYDPLYKEALQMLRD
AGAQVSIMTYDEFEYCWDTFVYRQGCPFQPWDGLEEHSQALSGRLRAILQVRASSL
CMVPHRPPPPPQSPGPCLPLCSEPPLGSLLPTGRPAPSLPFLLTASFSFPPPASLPPLPSL
SLSPGHLPVPSFHSLTSCSIQPPCSSRIRETEGWASVSKEGRDLG
Human APOBEC-3C:
(SEQ ID NO: 37)
MNPQIRNPMKAMYPGTFYFQFKNLWEANDRNETWLCFTVEGIKRRSVVS
WKTGVFRNQVDSETHCHAERCFLSWFCDDILSPNTKYQVTWYTSWSPCPDCAGEV
AEFLARHSNVNLTIFTARLYYFQYPCYQEGLRSLSQEGVAVEIMDYEDFKYCWENFV
YNDNEPFKPWKGLKTNFRLLKRRLRESLQ
(italic: nucleic acid editing domain)
Gorilla APOBEC3C:
(SEQ ID NO: 45)
MNPQIRNPMKAMYPGTFYFQFKNLWEANDRNETWLCFTVEGIKRRSVVSWKTGVF
RNQVDSETHCHAERCFLSWFCDDILSPNTNYQVTWYTSWSPCPECAGEVAEFLARH
SNVNLTIFTARLYYFQDTDYQEGLRSLSQEGVAVKIMDYKDFKYCWENFVYNDDEP
FKPWKGLKYNFRFLKRRLQEILE
(italic: nucleic acid editing domain)
Human APOBEC-3A:
(SEQ ID NO: 38)
MEASPASGPRHLMDPHIFTSNFNNGIGRHKTYLCYEVERLDNGTSVKMDQ
HRGFLHNQAKNLLCGFYGRHAELRFLDLVPSLQLDPAQIYRVTWFISWSPCFSWGCA
GEVRAFLQENTHVRLRIFAARIYDYDPLYKEALQMLRDAGAQVSIMTYDEFKHCWD
TFVDHQGCPFQPWDGLDEHSQALSGRLRAILQNQGN
(italic: nucleic acid editing domain)
Rhesus macaque APOBEC-3A:
(SEQ ID NO: 46)
MDGSPASRPRHLMDPNTFTFNFNNDLSVRGRHQTYLCYEVERLDNGTWVPMDERR
GFLCNKAKNVPCGDYGCHVELRFLCEVPSWQLDPAQTYRVTWFISWSPCFRRGCAG
QVRVFLQENKHVRLRIFAARIYDYDPLYQEALRTLRDAGAQVSIMTYEEFKHCWDT
FVDRQGRPFQPWDGLDEHSQALSGRLRAILQNQGN
(italic: nucleic acid editing domain)
Bovine APOBEC-3A:
(SEQ ID NO: 47)
MDEYTFTENFNNQGWPSKTYLCYEMERLDGDATIPLDEYKGFVRNKGLDQPEKPCH
AELYFLGKIHSWNLDRNQHYRLTCFISWSPCYDCAQKLTTFLKENHHISLHILASRIY
THNRFGCHQSGLCELQAAGARITIMTFEDFKHCWETFVDHKGKPFQPWEGLNVKSQ
ALCTELQAILKTQQN
(italic: nucleic acid editing domain)
Human APOBEC-3H:
(SEQ ID NO: 39)
MALLTAETFRLQFNNKRRLRRPYYPRKALLCYQLTPQNGSTPTRGYFENK
KKCHAEICFINEIKSMGLDETQCYQVTCYLTWSPCSSCAWELVDFIKAHDHLNLGIFA
SRLYYHWCKPQQKGLRLLCGSQVPVEVMGFPKFADCWENFVDHEKPLSFNPYKML
EELDKNSRAIKRRLERIKIPGVRAQGRYMDILCDAEV
(italic: nucleic acid editing domain)
Rhesus macaque APOBEC-3H:
(SEQ ID NO: 51)
MALLTAKTFSLQFNNKRRVNKPYYPRKALLCYQLTPQNGSTPTRGHLKNK
KKDHAEIRFINKIKSMGLDETQCYQVTCYLTWSPCPSCAGELVDFIKAHRHLNLRIFA
SRLYYHWRPNYQEGLLLLCGSQVPVEVMGLPEFTDCWENFVDHKEPPSFNPSEKLE
ELDKNSQAIKRRLERIKSRSVDVLENGLRSLQLGPVTPSSSIRNSR
Human APOBEC-3D:
(SEQ ID NO: 40)
MNPQIRNPMERMYRDTFYDNFENEPILYGRSYTWLCYEVKIKRGRSNLLW
DTGVFRGPVLPKRQSNHRQEVYFRFENHAEMCFLSWFCGNRLPANRRFQITWFVSW
NPCLPCVVKVTKFLAEHPNVTLTISAARLYYYRDRDWRWVLLRLHKAGARVKIMD
YEDFAYCWENFVCNEGQPFMPWYKFDDNYASLHRTLKEILRNPMEAMYPHIFYFHF
KNLLKACGRNESWLCFTMEVTKHHSAVFRKRGVFRNQVDPETHCHAERCFLSWFC
DDILSPNTNYEVTWYTSWSPCPECAGEVAEFLARHSNVNLTIFTARLCYFWDTDYQE
GLCSLSQEGASVKIMGYKDFVSCWKNFVYSDDEPFKPWKGLQTNFRLLKRRLREILQ
(italic: nucleic acid editing domain)
Human APOBEC-1:
(SEQ ID NO: 41)
MTSEKGPSTGDPTLRRRIEPWEFDVFYDPRELRKEACLLYEIKWGMSRKIW
RSSGKNTTNHVEVNFIKKFTSERDFHPSMSCSITWFLSWSPCWECSQAIREFLSRHPG
VTLVIYVARLFWHMDQQNRQGLRDLVNSGVTIQIMRASEYYHCWRNFVNYPPGDE
AHWPQYPPLWMMLYALELHCIILSLPPCLKISRRWQNHLTFFRLHLQNCHYQTIPPHI
LLATGLIHPSVAWR
Mouse APOBEC-1:
(SEQ ID NO: 42)
MSSETGPVAVDPTLRRRIEPHEFEVFFDPRELRKETCLLYEINWGGRHSVW
RHTSQNTSNHVEVNFLEKFTTERYFRPNTRCSITWFLSWSPCGECSRAITEFLSRHPY
VTLFIYIARLYHHTDQRNRQGLRDLISSGVTIQIMTEQEYCYCWRNFVNYPPSNEAY
WPRYPHLWVKLYVLELYCIILGLPPCLKILRRKQPQLTFFTITLQTCHYQRIPPHLLWA
TGLK
RatAPOBEC-1:
(SEQ ID NO: 43)
MSSETGPVAVDPTLRRRIEPHEFEVFFDPRELRKETCLLYEINWGGRHSIWR
HTSQNTNKHVEVNFIEKFTTERYFCPNTRCSITWFLSWSPCGECSRAITEFLSRYPHVT
LFIYIARLYHHADPRNRQGLRDLISSGVTIQIMTEQESGYCWRNFVNYSPSNEAHWPR
YPHLWVRLYVLELYCIILGLPPCLNILRRKQPQLTFFTIALQSCHYQRLPPHILWATGLK
Human APOBEC-2:
(SEQ ID NO: 52)
MAQKEEAAVATEAASQNGEDLENLDDPEKLKELIELPPFEIVTGERLPANF
FKFQFRNVEYSSGRNKTFLCYVVEAQGKGGQVQASRGYLEDEHAAAHAEEAFFNTI
LPAFDPALRYNVTWYVSSSPCAACADRIIKTLSKTKNLRLLILVGRLFMWEEPEIQAA
LKKLKEAGCKLRIMKPQDFEYVWQNFVEQEEGESKAFQPWEDIQENFLYYEEKLAD
ILK
Mouse APOBEC-2:
(SEQ ID NO: 53)
MAQKEEAAEAAAPASQNGDDLENLEDPEKLKELIDLPPFEIVTGVRLPVNF
FKFQFRNVEYSSGRNKTFLCYVVEVQSKGGQAQATQGYLEDEHAGAHAEEAFFNTI
LPAFDPALKYNVTWYVSSSPCAACADRILKTLSKTKNLRLLILVSRLFMWEEPEVQA
ALKKLKEAGCKLRIMKPQDFEYIWQNFVEQEEGESKAFEPWEDIQENFLYYEEKLAD
ILK
Rat APOBEC-2:
(SEQ ID NO: 54)
MAQKEEAAEAAAPASQNGDDLENLEDPEKLKELIDLPPFEIVTGVRLPVNF
FKFQFRNVEYSSGRNKTFLCYVVEAQSKGGQVQATQGYLEDEHAGAHAEEAFFNTI
LPAFDPALKYNVTWYVSSSPCAACADRILKTLSKTKNLRLLILVSRLFMWEEPEVQA
ALKKLKEAGCKLRIMKPQDFEYLWQNFVEQEEGESKAFEPWEDIQENFLYYEEKLA
DILK
Bovine APOBEC-2:
(SEQ ID NO: 55)
MAQKEEAAAAAEPASQNGEEVENLEDPEKLKELIELPPFEIVTGERLPAHYFKFQFRN
VEYSSGRNKTFLCYVVEAQSKGGQVQASRGYLEDEHATNHAEEAFFNSIMPTFDPA
LRYMVTWYVSSSPCAACADRIVKTLNKTKNLRLLILVGRLFMWEEPEIQAALRKLKE
AGCRLRIMKPQDFEYIWQNFVEQEEGESKAFEPWEDIQENFLYYEEKLADILK
Petromyzon marinus CDA1 (pmCDA1):
(SEQ ID NO: 56)
MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADCAEKILEWY
NQELRGNGHTLKIWACKLYYEKNARNQIGLWNLRDNGVGLNVMVSEHYQCCRKIF
IQSSHNQLNENRWLEKTLKRAEKRRSELSIMIQVKILHTTKSPAV
Human APOBEC3G D316R D317R:
(SEQ ID NO: 57)
MKPHFRNTVERMYRDTFSYNFYNRPILSRRNTVWLCYEVKTKGPSRPPLD
AKIFRGQVYSELKYHPEMRFFHWFSKWRKLHRDQEYEVTWYISWSPCTKCTRDMA
TFLAEDPKVTLTIFVARLYYFWDPDYQEALRSLCQKRDGPRATMKIMNYDEFQHCW
SKFVYSQRELFEPWNNLPKYYILLHIMLGEILRHSMDPPTFTFNFNNEPWVRGRHETY
LCYEVERMHNDTWVLLNQRRGFLCNQAPHKHGFLEGRHAELCFLDVIPFWKLDLD
QDYRVTCFTSWSPCFSCAQEMAKFISKNKHVSLCIFTARIYRRQGRCQEGLRTLAEA
GAKISIMTYSEFKHCWDTFVDHQGCPFQPWDGLDEHSQDLSGRLRAILQNQEN
Human APOBEC3G chain A:
(SEQ ID NO: 58)
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQEMAKFISKN
KHVSLCIFTARIYDDQGRCQEGLRTLAEAGAKISIMTYSEFKHCWDTFVDHQGCPFQ
PWDGLDEHSQDLSGRLRAILQ
Human APOBEC3G chain A D120R_D121R:
(SEQ ID NO: 59)
MDPPTFTFNFNNEPWVRGRHETYLCYEVERMHNDTWVLLNQRRGFLCNQ
APHKHGFLEGRHAELCFLDVIPFWKLDLDQDYRVTCFTSWSPCFSCAQEMAKFISKN
KHVSLCIFTARIYRRQGRCQEGLRTLAEAGAKISIMTYSEFKHCWDTFVDHQGCPFQ
PWDGLDEHSQDLSGRLRAILQ

Some aspects of the disclosure are based on the recognition that modulating the deaminase domain catalytic activity of any of the fusion proteins provided herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deaminataion window may prevent unwanted deamination of residues adjacent of specific target residues, which may decrease or prevent off-target effects.

In some embodiments, any of the fusion proteins provided herein comprise a deaminase domain (e.g., a cytidine deaminase domain) that has reduced catalytic deaminase activity. In some embodiments, any of the fusion proteins provided herein comprise a deaminase domain (e.g., a cytidine deaminase domain) that has a reduced catalytic deaminase activity as compared to an appropriate control. For example, the appropriate control may be the deaminase activity of the deaminase prior to introducing one or more mutations into the deaminase. In other embodiments, the appropriate control may be a wild-type deaminase. In some embodiments, the appropriate control is a wild-type apolipoprotein B mRNA-editing complex (APOBEC) family deaminase. In some embodiments, the appropriate control is an APOBEC1 deaminase, an APOBEC2 deaminase, an APOBEC3A deaminase, an APOBEC3B deaminase, an APOBEC3C deaminase, an APOBEC3D deaminase, an APOBEC3F deaminase, an APOBEC3G deaminase, or an APOBEC3H deaminase. In some embodiments, the appropriate control is an activation induced deaminase (AID). In some embodiments, the appropriate control is a cytidine deaminase 1 from Petromyzon marinus (pmCDA1). In some embodiments, the deaminse domain may be a deaminase domain that has at least 1%, at least 5%, at least 15%, at least 20%, at least 25%, at least 30%, at least 40%, at least 50%, at least 60%, at lest 70%, at least 80%, at least 90%, or at least 95% less catalytic deaminase activity as compared to an appropriate control.

In some embodiments, any of the fusion proteins provided herein comprise an APOBEC deaminase comprising one or more mutations selected from the group consisting of H121X, H122X, R126X, R126X, R118X, W90X, W90X, and R132X of rAPOBEC1 (SEQ ID NO: 43), or one or more corresponding mutations in another APOBEC deaminase, wherein X is any amino acid. In some embodiments, any of the fusion proteins provided herein comprise an APOBEC deaminase comprising one or more mutations selected from the group consisting of H121R, H122R, R126A, R126E, R118A, W90A, W90Y, and R132E of rAPOBEC1 (SEQ ID NO: 43), or one or more corresponding mutations in another APOBEC deaminase.

In some embodiments, any of the fusion proteins provided herein comprise an APOBEC deaminase comprising one or more mutations selected from the group consisting of D316X, D317X, R320X, R320X, R313X, W285X, W285X, R326X of hAPOBEC3G (SEQ ID NO: 34), or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G (SEQ ID NO: 34), or one or more corresponding mutations in another APOBEC deaminase.

In some embodiments, any of the fusion proteins provided herein comprise an APOBEC deaminase comprising a H121R and a H122Rmutation of rAPOBEC1 (SEQ ID NO: 43), or one or more corresponding mutations in another APOBEC deaminase. In some embodiments, any of the fusion proteins provided herein comprise an APOBEC deaminase comprising a R126A mutation of rAPOBEC1 (SEQ ID NO: 43), or one or more corresponding mutations in another APOBEC deaminase. In some embodiments, any of the fusion proteins provided herein comprise an APOBEC deaminase comprising a R126E mutation of rAPOBEC1 (SEQ ID NO: 43), or one or more corresponding mutations in another APOBEC deaminase. In some embodiments, any of the fusion proteins provided herein comprise an APOBEC deaminase comprising a R118A mutation of rAPOBEC1 (SEQ ID NO: 43), or one or more corresponding mutations in another APOBEC deaminase. In some embodiments, any of the fusion proteins provided herein comprise an APOBEC deaminase comprising a W90A mutation of rAPOBEC1 (SEQ ID NO: 43), or one or more corresponding mutations in another APOBEC deaminase. In some embodiments, any of the fusion proteins provided herein comprise an APOBEC deaminase comprising a W90Y mutation of rAPOBEC1 (SEQ ID NO: 43), or one or more corresponding mutations in another APOBEC deaminase. In some embodiments, any of the fusion proteins provided herein comprise an APOBEC deaminase comprising a R132E mutation of rAPOBEC1 (SEQ ID NO: 43), or one or more corresponding mutations in another APOBEC deaminase. In some embodiments, any of the fusion proteins provided herein comprise an APOBEC deaminase comprising a W90Y and a R126E mutation of rAPOBEC1 (SEQ ID NO: 43), or one or more corresponding mutations in another APOBEC deaminase. In some embodiments, any of the fusion proteins provided herein comprise an APOBEC deaminase comprising a R126E and a R132E mutation of rAPOBEC1 (SEQ ID NO: 43), or one or more corresponding mutations in another APOBEC deaminase. In some embodiments, any of the fusion proteins provided herein comprise an APOBEC deaminase comprising a W90Y and a R132E mutation of rAPOBEC1 (SEQ ID NO: 43), or one or more corresponding mutations in another APOBEC deaminase. In some embodiments, any of the fusion proteins provided herein comprise an APOBEC deaminase comprising a W90Y, R126E, and R132E mutation of rAPOBEC1 (SEQ ID NO: 43), or one or more corresponding mutations in another APOBEC deaminase.

In some embodiments, any of the fusion proteins provided herein comprise an APOBEC deaminase comprising a D316R and a D317R mutation of hAPOBEC3G (SEQ ID NO: 34), or one or more corresponding mutations in another APOBEC deaminase. In some embodiments, any of the fusion proteins provided herein comprise an APOBEC deaminase comprising a R320A mutation of hAPOBEC3G (SEQ ID NO: 34), or one or more corresponding mutations in another APOBEC deaminase. In some embodiments, any of the fusion proteins provided herein comprise an APOBEC deaminase comprising a R320E mutation of hAPOBEC3G (SEQ ID NO: 34), or one or more corresponding mutations in another APOBEC deaminase. In some embodiments, any of the fusion proteins provided herein comprise an APOBEC deaminase comprising a R313A mutation of hAPOBEC3G (SEQ ID NO: 34), or one or more corresponding mutations in another APOBEC deaminase. In some embodiments, any of the fusion proteins provided herein comprise an APOBEC deaminase comprising a W285A mutation of hAPOBEC3G (SEQ ID NO: 34), or one or more corresponding mutations in another APOBEC deaminase. In some embodiments, any of the fusion proteins provided herein comprise an APOBEC deaminase comprising a W285Y mutation of hAPOBEC3G (SEQ ID NO: 34), or one or more corresponding mutations in another APOBEC deaminase. In some embodiments, any of the fusion proteins provided herein comprise an APOBEC deaminase comprising a R326E mutation of hAPOBEC3G (SEQ ID NO: 34), or one or more corresponding mutations in another APOBEC deaminase. In some embodiments, any of the fusion proteins provided herein comprise an APOBEC deaminase comprising a W285Y and a R320E mutation of hAPOBEC3G (SEQ ID NO: 34), or one or more corresponding mutations in another APOBEC deaminase. In some embodiments, any of the fusion proteins provided herein comprise an APOBEC deaminase comprising a R320E and a R326E mutation of hAPOBEC3G (SEQ ID NO: 34), or one or more corresponding mutations in another APOBEC deaminase. In some embodiments, any of the fusion proteins provided herein comprise an APOBEC deaminase comprising a W285Y and a R326E mutation of hAPOBEC3G (SEQ ID NO: 34), or one or more corresponding mutations in another APOBEC deaminase. In some embodiments, any of the fusion proteins provided herein comprise an APOBEC deaminase comprising a W285Y, R320E, and R326E mutation of hAPOBEC3G (SEQ ID NO: 34), or one or more corresponding mutations in another APOBEC deaminase.

### E. Adenosine Deaminases

In still other embodiments, the GeoCas9 base editors may comprise an adenosine deaminase as the nucleic acid effector domain.

Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the GeoCas9 base editors may comprise 2, 3, 4 or 5 adenosine deaminases. In some embodiments, any of the GeoCas9 base editors provided herein comprise two adenosine deaminases. Exemplary, non-limiting, embodiments of adenosine deaminases are provided herein. It should be appreciated that the mutations provided herein (e.g., mutations in ecTadA) may be applied to adenosine deaminases in other adenosine base editors, for example those provided in U.S. Patent Publication No. 2018/0073012, published Mar. 15, 2018, which issued as U.S. Pat. No. 10,113,163, on Oct. 30, 2018; U.S. Patent Publication No. 2017/0121693, published May 4, 2017, which issued as U.S. Pat. No. 10,167,457 on Jan. 1, 2019; International Publication No. WO 2017/070633, published Apr. 27, 2017; U.S. Patent Publication No. 2015/0166980, published Jun. 18, 2015; U.S. Pat. No. 9,840,699, issued Dec. 12, 2017; and U.S. Pat. No. 10,077,453, issued Sep. 18, 2018, all of which are incorporated herein by reference in their entireties.

In some embodiments, any of the adenosine deaminases provided herein is capable of deaminating adenine. In some embodiments, the adenosine deaminases provided herein are capable of deaminating adenine in a deoxyadenosine residue of DNA. The adenosine deaminase may be derived from any suitable organism (e.g., E. coli). In some embodiments, the adenosine deaminase is a naturally-occurring adenosine deaminase that includes one or more mutations corresponding to any of the mutations provided herein (e.g., mutations in ecTadA). One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any one of SEQ ID NOs: 413-434, or to any of the adenosine deaminases provided herein. It should be appreciated that adenosine deaminases provided herein may include one or more mutations (e.g., any of the mutations provided herein). The disclosure provides adenosine deaminases with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to any one of the amino acid sequences set forth in SEQ ID NOs: 413-434, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences set forth in SEQ ID NOs: 413-434, or any of the adenosine deaminases provided herein.

In some embodiments, the adenosine deaminase comprises an E59X mutation in ecTadA SEQ ID NO: 422, or a corresponding mutation in another adenosine deaminase, where X indicates any amino acid other than the corresponding amino acid in the wild-type adenosine deaminase. In particular embodiments, the adenosine deaminase comprises a E59A mutation in SEQ ID NO: 422, or a corresponding mutation in another adenosine deaminase.

In some embodiments, the adenosine deaminase comprises a D108X mutation in ecTadA SEQ ID NO: 422, or a corresponding mutation in another adenosine deaminase, where X indicates any amino acid other than the corresponding amino acid in the wild-type adenosine deaminase. In some embodiments, the adenosine deaminase comprises a D108W, D108Q, D108F, D108K, or D108M mutation in SEQ ID NO: 422, or a corresponding mutation in another adenosine deaminase. In particular embodiments, the adenosine deaminase comprises a D108W mutation in SEQ ID NO: 422, or a corresponding mutation in another adenosine deaminase. It should be appreciated, however, that additional deaminases may similarly be aligned to identify homologous amino acid residues that may be mutated as provided herein.

In some embodiments, the adenosine deaminase comprises TadA 7.10, whose sequence is provided as SEQ ID NO: 423, or a variant thereof. TadA7.10 comprises the following mutations in ecTadA: W23R, H36L, P48A, R51L, L84F, A106V, D108N, H123Y, S146C, D147Y, R152P, E155V, 1156F, K157N.

In particular embodiments, the adenosine deaminase comprises an N108W mutation in SEQ ID NO: 423, an embodiment also referred to as TadA 7.10 (N108W). Its sequence is provided as SEQ ID NO: 425.

In some embodiments, the adenosine deaminase comprises an A106X mutation in ecTadA SEQ ID NO: 422, or a corresponding mutation in another adenosine deaminase, where X indicates any amino acid other than the corresponding amino acid in the wild-type adenosine deaminase. In some embodiments, the adenosine deaminase comprises an A106V mutation in SEQ ID NO: 422, or a corresponding mutation in another adenosine deaminase. In some embodiments, the adenosine deaminase comprises an A106Q, A106F, A106W, or A106M mutation in SEQ ID NO: 422, or a corresponding mutation in another adenosine deaminase.

In particular embodiments, the adenosine deaminase comprises a V106W mutation in SEQ ID NO: 423, an embodiment also referred to as TadA 7.10 (V106W). Its sequence is provided as SEQ ID NO: 424.

In some embodiments, the adenosine deaminase comprises a R47X mutation in SEQ ID NO: 425, or a corresponding mutation in another adenosine deaminase, where the presence of X indicates any amino acid other than the corresponding amino acid in the wild-type adenosine deaminase. In some embodiments, the adenosine deaminase comprises a R47Q, R47F, R47W, or R47M mutation in SEQ ID NO: 425, or a corresponding mutation in another adenosine deaminase.

In particular embodiments, the adenosine deaminase comprises a R47Q, R47F, R47W, or R47M mutation in SEQ ID NO: 425.

In particular embodiments, the adenosine deaminase comprises a V106Q mutation and an N108W mutation in SEQ ID NO: 425. In particular embodiments, the adenosine deaminase comprises a V106W mutation, an N108W mutation and an R47Z mutation, wherein Z is selected from the residues consisting of Q, F, W and M, in SEQ ID NO: 425.

It should be appreciated that any of the mutations provided herein (e.g., based on the ecTadA amino acid sequence of SEQ ID NO: 422) may be introduced into other adenosine deaminases, such as S. aureus TadA (saTadA), or other adenosine deaminases (e.g., bacterial adenosine deaminases), such as those sequences provided below. It would be apparent to the skilled artisan how to identify amino acid residues from other adenosine deaminases that are homologous to the mutated residues in ecTadA. Thus, any of the mutations identified in ecTadA may be made in other adenosine deaminases that have homologous amino acid residues. It should also be appreciated that any of the mutations provided herein may be made individually or in any combination in ecTadA or another adenosine deaminase. For example, an adenosine deaminase may contain a D108N, an A106V, and/or a R47Q mutation in ecTadA SEQ ID NO: 422, or a corresponding mutation in another adenosine deaminase.

In some embodiments, the adenosine deaminase comprises one, two, or three mutations selected from the group consisting of D108, A106, and R47 in SEQ ID NO: 422, or a corresponding mutation or mutations in another adenosine deaminase.

In other aspects, the disclosure provides adenine base editors with broadened target sequence compatibility. In general, native ecTadA deaminates the adenine in the sequence UAC (e.g., the target sequence) of the anticodon loop of tRNAArg. Without wishing to be bound by any particular theory, in order to expand the utility of ABEs comprising one or more ecTadA deaminases, such as any of the adenosine deaminases provided herein, the adenosine deaminase proteins were optimized to recognize a wide variety of target sequences within the protospacer sequence without compromising the editing efficiency of the adenosine nucleobase editor complex. In some embodiments, the target sequence is an A in the middle of a 5′-NAN-3′ sequence, wherein N is T, C, G, or A. In some embodiments, the target sequence comprises 5′-TAC-3′. In some embodiments, the target sequence comprises 5′-GAA-3′.

In some embodiments, the adenosine deaminase is an N-terminal truncated E. coli TadA. In certain embodiments, the adenosine deaminase comprises the amino acid sequence:

(SEQ ID NO: 413)
MSEVEFSHEYWMRHALTLAKRAWDEREVPVGAVLVHNNRVIGEGWNRPI
GRHDPTAHAEIMALRQGGLVMQNYRLIDATLYVTLEPCVMCAGAMIHSR
IGRVVFGARDAKTGAAGSLMDVLHHPGMNHRVEITEGILADECAALLSD
FFRMRRQEIKAQKKAQSSTD.

In some embodiments, the TadA deaminase is a full-length E. coli TadA deaminase (ecTadA). For example, in certain embodiments, the adenosine deaminase comprises the amino acid sequence:

(SEQ ID NO: 414)
MRRAFITGVFFLSEVEFSHEYWMRHALTLAKRAWDEREVPVGAVLVHNN
RVIGEGWNRPIGRHDPTAHAEIMALRQGGLVMQNYRLIDATLYVTLEPC
VMCAGAMIHSRIGRVVFGARDAKTGAAGSLMDVLHHPGMNHRVEITEGI
LADECAALLSDFFRMRRQEIKAQKKAQSSTD

It should be appreciated, however, that additional adenosine deaminases useful in the present application would be apparent to the skilled artisan and are within the scope of this disclosure. For example, the adenosine deaminase may be a homolog of an ADAT. Exemplary ADAT homologs include, without limitation:

Staphylococcus aureus TadA:
(SEQ ID NO: 415)
MGSHMTNDIYFMTLAIEEAKKAAQLGEVPIGAIITKDDEVIARAHNLRETL
QQPTAHAEHIAIERAAKVLGSWRLEGCTLYVTLEPCVMCAGTIVMSRIPRVVYGADD
PKGGCSGSLMNLLQQSNFNHRAIVDKGVLKEACSTLLTTFFKNLRANKKSTN
Bacillus subtilis TadA:
(SEQ ID NO: 416)
MTQDELYMKEAIKEAKKAEEKGEVPIGAVLVINGEIIARAHNLRETEQRSI
AHAEMLVIDEACKALGTWRLEGATLYVTLEPCPMCAGAVVLSRVEKVVFGAFDPK
GGCSGTLMNLLQEERFNHQAEVVSGVLEEECGGMLSAFFRELRKKKKAARKNLSE
Salmonella typhimurium (S. typhimurium) TadA:
(SEQ ID NO: 417)
MPPAFITGVTSLSDVELDHEYWMRHALTLAKRAWDEREVPVGAVLVHNH
RVIGEGWNRPIGRHDPTAHAEIMALRQGGLVLQNYRLLDTTLYVTLEPCVMCAGAM
VHSRIGRVVFGARDAKTGAAGSLIDVLHHPGMNHRVEIIEGVLRDECATLLSDFFRM
RRQEIKALKKADRAEGAGPAV
Shewanella putrefaciens (S. putrefaciens) TadA:
(SEQ ID NO: 418)
MDEYWMQVAMQMAEKAEAAGEVPVGAVLVKDGQQIATGYNLSISQHDP
TAHAEILCLRSAGKKLENYRLLDATLYITLEPCAMCAGAMVHSRIARVVYGARDEK
TGAAGTVVNLLQHPAFNHQVEVTSGVLAEACSAQLSRFFKRRRDEKKALKLAQRAQ
QGIE
Haemophilus influenzae F3031 (H. influenzae) TadA:
(SEQ ID NO: 419)
MDAAKVRSEFDEKMMRYALELADKAEALGEIPVGAVLVDDARNIIGEGW
NLSIVQSDPTAHAEIIALRNGAKNIQNYRLLNSTLYVTLEPCTMCAGAILHSRIKRLVF
GASDYKTGAIGSRFHFFDDYKMNHTLEITSGVLAEECSQKLSTFFQKRREEKKIEKAL
LKSLSDK
Caulobacter crescentus (C. crescentus) TadA:
(SEQ ID NO: 420)
MRTDESEDQDHRMMRLALDAARAAAEAGETPVGAVILDPSTGEVIATAG
NGPIAAHDPTAHAEIAAMRAAAAKLGNYRLTDLTLVVTLEPCAMCAGAISHARIGR
VVFGADDPKGGAVVHGPKFFAQPTCHWRPEVTGGVLADESADLLRGFFRARRKAKI
Geobacter sulfurreducens (G. sulfurreducens) TadA:
(SEQ ID NO: 421)
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIILARLERVV
FGCYDPKGGAAGSLYDLSADPRLNHQVRLSPGVCQEECGTMLSDFFRDLRRRKKAK
ATPALFIDERKVPPEP

Exemplary adenosine deaminase variants of the disclosure are described below. In certain embodiments, the adenosine deaminase has a sequence with at least 80%, at least 85%, at least 90%, at least 95%, at least 98%, at least 99%, or at least 99.5% sequence identity to one of the following:

(Ec)TadA, catalytically inactive (E59A)
(SEQ ID NO: 422)
MSEVEFSHEYWMRHALTLAKRAWDEREVPVGAVLVHNNRVIGEGWNRPI
GRHDPTAHAAIMALRQGGLVMQNYRLIDATLYVTLEPCVMCAGAMIHSRIGRVVFG
ARDAKTGAAGSLMDVLHHPGMNHRVEITEGILADECAALLSDFFRMRRQEIKAQKK
AQSSTD
TadA 7.10
(SEQ ID NO: 423)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQK
KAQSSTD
TadA 7.10 (V106W)
(SEQ ID NO: 424)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
WRNAKTGAAGSLMDVLHYPGMNHRVEITEGILADECAALLCYFFRMPRQVFNAQK
KAQSSTD
TadA 7.10 (N108W)
(SEQ ID NO: 425)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
VRWAKTGAAGSLMDVLHYPGMNHRVEITEGILADECAALLCYFFRMPRQVFNAQK
KAQSSTD
TadA 7.10 (N108Q)
(SEQ ID NO: 426)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
VRQAKTGAAGSLMDVLHYPGMNHRVEITEGILADECAALLCYFFRMPRQVFNAQK
KAQSSTD
TadA 7.10 (V106F)
(SEQ ID NO: 427)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
FRNAKTGAAGSLMDVLHYPGMNHRVEITEGILADECAALLCYFFRMPRQVFNAQKK
AQSSTD
TadA 7.10 (V106Q)
(SEQ ID NO: 428)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
QRNAKTGAAGSLMDVLHYPGMNHRVEITEGILADECAALLCYFFRMPRQVFNAQK
KAQSSTD
TadA 7.10 (V106M)
(SEQ ID NO: 429)
MSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRIGRVVFG
MRNAKTGAAGSLMDVLHYPGMNHRVEITEGILADECAALLCYFFRMPRQVFNAQK
KAQSSTD
TadA 7.10 (R47F)
(SEQ ID NO: 430)
MSEVEFSHEYWMRHALTLAKRARDEREVPVGAVLVLNNRVIGEGWNFAI
GLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQK
KAQSSTD
TadA 7.10 (R47W)
(SEQ ID NO: 431)
MSEVEFSHEYWMRHALTLAKRARDEREVPVGAVLVLNNRVIGEGWNWAI
GLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQK
KAQSSTD
TadA 7.10 (R47Q)
(SEQ ID NO: 432)
MSEVEFSHEYWMRHALTLAKRARDEREVPVGAVLVLNNRVIGEGWNQAI
GLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQK
KAQSSTD
TadA 7.10 (R47M)
(SEQ ID NO: 433)
MSEVEFSHEYWMRHALTLAKRARDEREVPVGAVLVLNNRVIGEGWNMAI
GLHDPTAHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQVFNAQK
KAQSSTD
TadA (E59Q)
(SEQ ID NO: 434)
MSEVEFSHEYWMRHALTLAKRAWDEREVPVGAVLVHNNRVIGEGWNRPI
GRHDPTAHAQIMALRQGGLVMQNYRLIDATLYVTLEPCVMCAGAMIHSRIGRVVFG
ARDAKTGAAGSLMDVLHHPGMNHRVEITEGILADECAALLSDFFRMRRQEIKAQKK
AQSSTD

Any two or more of the adenosine deaminases described herein may be connected to one another (e.g. by a linker) within an adenosine deaminase domain of the fusion proteins provided herein. For instance, the fusion proteins provided herein may contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase. In some embodiments, the fusion protein comprises two adenosine deaminases (e.g., a first adenosine deaminase and a second adenosine deaminase). In some embodiments, the fusion protein comprises a first adenosine deaminase and a second adenosine deaminase. In some embodiments, the first adenosine deaminase is N-terminal to the second adenosine deaminase in the fusion protein. In some embodiments, the first adenosine deaminase is C-terminal to the second adenosine deaminase in the fusion protein. In some embodiments, the first adenosine deaminase and the second deaminase are fused directly or via a linker.

In particular embodiments, the base editors disclosed herein comprise a heterodimer of a first adenosine deaminase that is N-terminal to a second adenosine deaminase, wherein the first adenosine deaminase comprises a sequence with at least 80%, 85%, 90%, 95%, 98%, 99%, or 99.5% sequence identity to SEQ ID NO: 413-434; and the second adenosine deaminase comprises a sequence with at least 80%, 85%, 90%, 95%, 98%, 99%, or 99.5% sequence identity to SEQ ID NO: 413-434.

In other embodiments, the second adenosine deaminase of the base editors provided herein comprises a sequence with at least 80%, 85%, 90%, 95%, 98%, 99%, or 99.5% sequence identity to SEQ ID NO: 423 (TadA 7.10), wherein any sequence variation may only occur in amino acid positions other than R47, V106 or N108 of SEQ ID NO: 423. In other words, these embodiments must contain amino acid substitutions at R47, V106 or N108 of SEQ ID NO: 423.

In other embodiments, the second adenosine deaminase of the heterodimer comprises a sequence with at least 80%, 85%, 90%, 95%, 98%, 99%, or 99.5% sequence identity to SEQ ID NO: 413-434.

### F. Recombinases

In other embodiments, the GeoCas9 base editors may comprise a recombinase as the nucleic acid effector domain.

The term “recombinase,” as used herein, refers to a site-specific enzyme that mediates the recombination of DNA between recombinase recognition sequences, which results in the excision, integration, inversion, or exchange (e.g., translocation) of DNA fragments between the recombinase recognition sequences. Recombinases can be classified into two distinct families: serine recombinases (e.g., resolvases and invertases) and tyrosine recombinases (e.g., integrases). Examples of serine recombinases include, without limitation, Hin, Gin, Tn3, β-six, CinH, ParA, γδ, Bxb1, ϕC31, TP901, TG1, φBT1, R4, φRV1, φFC1, MR11, A118, U153, and gp29. Examples of tyrosine recombinases include, without limitation, Cre, FLP, R, Lambda, HK101, HK022, and pSAM2. The Gin recombinase referred to herein may be any Gin recombinase known in the art including, but not limited to, the Gin recombinases presented in T. Gaj et al., A comprehensive approach to zinc-finger recombinase customization enables genomic targeting in human cells. Nucleic Acids Research 41, 3937-3946 (2013), incorporated herein by reference in its entirety. In certain embodiments, the Gin recombinase catalytic domain has greater than 85%, 90%, 95%, 98%, or 99% sequence identity with the amino acid sequence shown in SEQ ID NO: 384. In another embodiment, the amino acid sequence of the Gin recombinase catalytic domain comprises a mutation corresponding to H106Y, and/or I127L, and/or I136R and/or G137F. In yet another embodiment, the amino acid sequence of the Gin recombinase catalytic domain comprises a mutation corresponding to H106Y, I127L, 1136R, and G137F.

In a further embodiment, the amino acid sequence of the Gin recombinase has been further mutated. In a specific embodiment, the amino acid sequence of the Gin recombinase catalytic domain comprises SEQ ID NO: 384. Gin recombinases bind to gix target sites (also referred to herein as “gix core,” “minimal gix core,” or “gix-related core” sequences). The minimal gix core recombinase site is NNNNAAASSWWSSTTTNNNN (SEQ ID NO: 384), wherein N is defined as any amino acid, W is an A or a T, and S is a G or a C. The gix target site may include any other mutations known in the art. In certain embodiments, the gix target site has greater than 90%, 95%, or 99% sequence identity with the amino acid sequence shown in SEQ ID NO: 384. The distance between the gix core or gix-related core sequence and at least one gRNA binding site may be from 1 to 10 base pairs, from 3 to 7 base pairs, from 5 to 7 base pairs, or from 5 to 6 base pairs. The distance between the gix core or gix-related core sequence and at least one gRNA binding site may be 1, 2, 3, 4, 5, 6, 7, 8, 9, or 10 base pairs.

The serine and tyrosine recombinase names stem from the conserved nucleophilic amino acid residue that the recombinase uses to attack the DNA and which becomes covalently linked to the DNA during strand exchange. Recombinases have numerous applications, including the creation of gene knockouts/knock-ins and gene therapy applications. See, e.g., Brown et al., “Serine recombinases as tools for genome engineering.” Methods. 2011; 53 (4): 372-9; Hirano et al., “Site-specific recombinases as tools for heterologous gene integration.” Appl. Microbiol. Biotechnol. 2011; 92 (2): 227-39; Chavez and Calos, “Therapeutic applications of the ΦC31 integrase system.” Curr. Gene Ther. 2011; 11 (5): 375-81; Turan and Bode, “Site-specific recombinases: from tag-and-target-to tag-and-exchange-based genomic modifications.” FASEB J. 2011; 25 (12): 4088-107; Venken and Bellen, “Genome-wide manipulations of Drosophila melanogaster with transposons, Flp recombinase, and @C31 integrase.” Methods Mol. Biol. 2012; 859:203-28; Murphy, “Phage recombinases and their applications.” Adv. Virus Res. 2012; 83:367-414; Zhang et al., “Conditional gene manipulation: Creating a new biological era.” J. Zhejiang Univ. Sci. B. 2012; 13 (7): 511-24; Karpenshif and Bernstein, “From yeast to mammals: recent advances in genetic control of homologous recombination.” DNA Repair (Amst). 2012; 1; 11 (10): 781-8; the entire contents of each are hereby incorporated by reference in their entirety. The recombinases provided herein are not meant to be exclusive examples of recombinases that can be used in embodiments of the invention. The methods and compositions of the invention can be expanded by mining databases for new orthogonal recombinases or designing synthetic recombinases with defined DNA specificities (See, e.g., Groth et al., “Phage integrases: biology and applications.” J. Mol. Biol. 2004; 335, 667-678; Gordley et al., “Synthesis of programmable integrases.” Proc. Natl. Acad. Sci. USA. 2009; 106, 5053-5058; the entire contents of each are hereby incorporated by reference in their entirety).

Other examples of recombinases that are useful in the methods and compositions described herein are known to those of skill in the art, and any new recombinase that is discovered or generated is expected to be able to be used in the different embodiments of the invention. In some embodiments, the catalytic domains of a recombinase are fused to a nuclease-inactivated RNA-programmable nuclease (e.g., dCas9, or a fragment thereof), such that the recombinase domain does not comprise a nucleic acid binding domain or is unable to bind to a target nucleic acid that subsequently results in enzymatic catalysis (e.g., the recombinase domain is engineered such that it does not have specific DNA binding activity). Recombinases lacking part of their DNA binding activity and those that act independently of accessory proteins and methods for engineering such are known, and include those described by Klippel et al., “Isolation and characterisation of unusual gin mutants.” EMBO J. 1988; 7:3983-3989: Burke et al., “Activating mutations of Tn3 resolvase marking interfaces important in recombination catalysis and its regulation. Mol Microbiol. 2004; 51:937-948; Olorunniji et al., “Synapsis and catalysis by activated Tn3 resolvase mutants.” Nucleic Acids Res. 2008; 36:7181-7191; Rowland et al., “Regulatory mutations in Sin recombinase support a structure-based model of the synaptosome.” Mol Microbiol. 2009; 74:282-298; Akopian et al., “Chimeric recombinases with designed DNA sequence recognition.” Proc Natl Acad Sci USA. 2003; 100:8688-8691; Gordley et al., “Evolution of programmable zinc finger-recombinases with activity in human cells. J Mol Biol. 2007; 367:802-813; Gordley et al., “Synthesis of programmable integrases.” Proc Natl Acad Sci USA. 2009; 106:5053-5058; Arnold et al., “Mutants of Tn3 resolvase which do not require accessory binding sites for recombination activity.” EMBO J. 1999; 18:1407-1414; Gaj et al., “Structure-guided reprogramming of serine recombinase DNA sequence specificity.” Proc Natl Acad Sci USA. 2011; 108 (2): 498-503; and Proudfoot et al., “Zinc finger recombinases with adaptable DNA sequence specificity.” PLOS One. 2011; 6 (4): e19537; the entire contents of each are hereby incorporated by reference.

For example, serine recombinases of the resolvase-invertase group, e.g., Tn3 and γδ resolvases and the Hin and Gin invertases, have modular structures with partly autonomous catalytic and DNA-binding domains (See, e.g., Grindley et al., “Mechanism of site-specific recombination.” Ann Rev Biochem. 2006; 75:567-605, the entire contents of which are incorporated by reference). The catalytic domains of these recombinases are therefore amenable to being recombined with nuclease-inactivated RNA-programmable nucleases (e.g., dCas9, or a fragment thereof) as described herein, e.g., following the isolation of ‘activated’ recombinase mutants which do not require any accessory factors (e.g., DNA binding activities) (See, e.g., Klippel et al., “Isolation and characterisation of unusual gin mutants.” EMBO J. 1988; 7:3983-3989: Burke et al., “Activating mutations of Tn3 resolvase marking interfaces important in recombination catalysis and its regulation. Mol Microbiol. 2004; 51:937-948; Olorunniji et al., “Synapsis and catalysis by activated Tn3 resolvase mutants.” Nucleic Acids Res. 2008; 36:7181-7191; Rowland et al., “Regulatory mutations in Sin recombinase support a structure-based model of the synaptosome.” Mol Microbiol. 2009; 74:282-298; Akopian et al., “Chimeric recombinases with designed DNA sequence recognition.” Proc Natl Acad Sci USA. 2003; 100:8688-8691). The GeoCas9 base editor fusion proteins contemplated herein may comprise any of these recombinase proteins.

Additionally, many other natural serine recombinases having an N-terminal catalytic domain and a C-terminal DNA binding domain are known (e.g., phiC31 integrase, TnpX transposase, IS607 transposase), and their catalytic domains can be co-opted to engineer programmable site-specific recombinases as described herein (See, e.g., Smith et al., “Diversity in the serine recombinases.” Mol Microbiol. 2002; 44:299-307, the entire contents of which are incorporated by reference). Similarly, the core catalytic domains of tyrosine recombinases (e.g., Cre, 2 integrase) are known, and can be similarly co-opted to engineer programmable site-specific recombinases as described herein (See, e.g., Guo et al., “Structure of Cre recombinase complexed with DNA in a site-specific recombination synapse.” Nature. 1997; 389:40-46; Hartung et al., “Cre mutants with altered DNA binding properties.” J Biol Chem 1998; 273:22884-22891; Shaikh et al., “Chimeras of the Flp and Cre recombinases: Tests of the mode of cleavage by Flp and Cre. J Mol Biol. 2000; 302:27-48; Rongrong et al., “Effect of deletion mutation on the recombination activity of Cre recombinase.” Acta Biochim Pol. 2005; 52:541-544; Kilbride et al., “Determinants of product topology in a hybrid Cre-Tn3 resolvase site-specific recombination system.” J Mol Biol. 2006; 355:185-195; Warren et al., “A chimeric cre recombinase with regulated directionality.” Proc Natl Acad Sci USA. 2008 105:18278-18283; Van Duyne, “Teaching Cre to follow directions.” Proc Natl Acad Sci USA. 2009 Jan. 6; 106 (1): 4-5; Numrych et al., “A comparison of the effects of single-base and triple-base changes in the integrase arm-type binding sites on the site-specific recombination of bacteriophage 2.” Nucleic Acids Res. 1990; 18:3953-3959; Tirumalai et al., “The recognition of core-type DNA sites by λ integrase.” J Mol Biol. 1998; 279:513-527; Aihara et al., “A conformational switch controls the DNA cleavage activity of A integrase.” Mol Cell. 2003; 12:187-198; Biswas et al., “A structural basis for allosteric control of DNA recombination by λ integrase.” Nature. 2005; 435:1059-1066; and Warren et al., “Mutations in the amino-terminal domain of λ-integrase have differential effects on integrative and excisive recombination.” Mol Microbiol. 2005; 55:1104-1112; the entire contents of each are incorporated by reference). The GeoCas9 base editor fusion proteins contemplated herein may comprise any of these recombinase proteins.

The recombinase catalytic domain for use in the compositions and methods of the instant disclosure may be from any recombinase. Suitable recombinases catalytic domains for use in the disclosed methods and compositions may be obtained from, for example, and without limitation, tyrosine recombinases and serine recombinases. Some exemplary suitable recombinases provided herein include, for example, and without limitation, Gin recombinase (acting on gix sites), Hin recombinase (acting on hix sites), β recombinase (acting on six sites), Sin recombinase (acting on resH sites), Tn3 recombinase (acting on res sites), γδ recombinase (acting on res sites), Cre recombinase from bacteriophage P1 (acting on LoxP sites); FLP recombinases of fungal origin (acting on FTR sites); and phiC31 integrase (acting on att sites). Non-limiting sequences of exemplary suitable recombinases may be found below.

Cre recombinase
(SEQ ID NO: 385)
MSNLLTVHQNLPALPVDATSDEVRKNLMDMFRDRQAFSEHTWKMLLSVC
RSWAAWCKLNNRKWFPAEPEDVRDYLLYLQARGLAVKTIQQHLGQLNMLHRRSGL
PRPSDSNAVSLVMRRIRKENVDAGERAKQALAFERTDFDQVRSLMENSDRCQDIRN
LAFLGIAYNTLLRIAEIARIRVKDISRTDGGRMLIHIGRTKTLVSTAGVEKALSLGVTK
LVERWISVSGVADDPNNYLFCRVRKNGVAAPSATSQLSTRALEGIFEATHRLIYGAK
DDSGQRYLAWSGHSARVGAARDMARAGVSIPEIMQAGGWTNVNIVMNYIRNLDSE
TGAMVRLLEDGD.
FLP recombinase
(SEQ ID NO: 386)
MPQFGILCKTPPKVLVRQFVERFERPSGEKIALCAAELTYLCWMITHNGTAI
KRATFMSYNTIISNSLSFDIVNKSLQFKYKTQKATILEASLKKLIPAWEFTIIPYYGQKH
QSDITDIVSSLQLQFESSEEADKGNSHSKKMLKALLSEGESIWEITEKILNSFEYTSRFT
KTKTLYQFLFLATFINCGRFSDIKNVDPKSFKLVQNKYLGVIIQCLVTETKTSVSRHIY
FFSARGRIDPLVYLDEFLRNSEPVLKRVNRTGNSSSNKQEYQLLKDNLVRSYNKALK
KNAPYSIFAIKNGPKSHIGRHLMTSFLSMKGLTELTNVVGNWSDKRASAVARTTYTH
QITAIPDHYFALVSRYYAYDPISKEMIALKDETNPIEEWQHIEQLKGSAEGSIRYPAW
NGIISQEVLDYLSSYINRRI.
γδ recombinase (Gamma Delta resolvase)
(SEQ ID NO: 387)
MRLFGYARVSTSQQSLDIQVRALKDAGVKANRIFTDKASGSSSDRKGLDL
LRMKVEEGDVILVKKLDRLGRDTADMIQLIKEFDAQGVSIRFIDDGISTDGEMGKMV
VTILSAVAQAERQRILERTNEGRQEAMAKGVVFGRKR
γδ recombinase (E124Q mutation)
(SEQ ID NO: 388)
MRLFGYARVSTSQQSLDIQVRALKDAGVKANRIFTDKASGSSSDRKGLDL
LRMKVEEGDVILVKKLDRLGRDTADMIQLIKEFDAQGVSIRFIDDGISTDGEMGKMV
VTILSAVAQAERQRILQRTNEGRQEAMAKGVVFGRKR.
γδ recombinase (E102Y/E124Q mutation)
(SEQ ID NO: 389)
MRLFGYARVSTSQQSLDIQVRALKDAGVKANRIFTDKASGSSSDRKGLDL
LRMKVEEGDVILVKKLDRLGRDTADMIQLIKEFDAQGVSIRFIDDGISTDGYMGKMV
VTILSAVAOAERORILORTNEGROEAMAKGVVFGRKR.
β recombinase
(SEQ ID NO: 390)
MAKIGYARVSSKEQNLDRQLQALQGVSKVFSDKLSGQSVERPQLQAMLN
YIREGDIVVVTELDRLGRNNKELTELMNAIQQKGATLEVLDLPSMNGIEDENLRRLIN
NLVIELYKYQAESERKRIKERQAQGIEIAKSKGKFKGRQH.
β recombinase (N95D mutation)
(SEQ ID NO: 391)
MAKIGYARVSSKEQNLDRQLQALQGVSKVFSDKLSGQSVERPQLQAMLNYIREGDI
VVVTELDRLGRNNKELTELMNAIQQKGATLEVLDLPSMDGIEDENLRRLINNLVIEL
YKYQAESERKRIKERQAQGIEIAKSKGKFKGRQH.
Sin recombinase
(SEQ ID NO: 392)
MIIGYARVSSLDQNLERQLENLKTFGAEKIFTEKQSGKSIENRPILQKALNF
VRMGDRFIVESIDRLGRNYNEVIHTVNYLKDKEVQLMITSLPMMNEVIGNPLLDKFM
KDLIIQILAMVSEQERNESKRRQAQGIQVAKEKGVYKGRPL.
Sin recombinase (Q87R/Q115R mutations)
(SEQ ID NO: 393)
MIIGYARVSSLDQNLERQLENLKTFGAEKIFTEKQSGKSIENRPILQKALNF
VRMGDRFIVESIDRLGRNYNEVIHTVNYLKDKEVRLMITSLPMMNEVIGNPLLDKFM
KDLIIRILAMVSEQERNESKRRQAQGIQVAKEKGVYKGRPL.
Tn3 recombinase
(SEQ ID NO: 394)
MRLFGYARVSTSQQSLDLQVRALKDAGVKANRIFTDKASGSSTDREGLDL
LRMKVKEGDVILVKKLDRLGRDTADMLQLIKEFDAQGVAVRFIDDGISTDGDMGQ
MVVTILSAVAQAERRRILERTNEGRQEAKLKGIKFGRRR.
Tn3 recombinase (G70S/D102Y, E124Q mutations)
(SEQ ID NO: 395)
MRLFGYARVSTSQQSLDLQVRALKDAGVKANRIFTDKASGSSTDREGLDL
LRMKVKEGDVILVKKLDRLSRDTADMLQLIKEFDAQGVAVRFIDDGISTDGYMGQM
VVTILSAVAQAERRRILQRTNEGRQEAKLKGIKFGRRR.
Hin recombinase
(SEQ ID NO: 396)
MATIGYIRVSTIDQNIDLQRNALTSANCDRIFEDRISGKIANRPGLKRALKY
VNKGDTLVVWKLDRLGRSVKNLVALISELHERGAHFHSLTDSIDTSSAMGRFFFHV
MSALAEMERELIVERTLAGLAAARAQGRLGGRPV.
Hin recombinase (H107Y mutation)
(SEQ ID NO: 397)
MATIGYIRVSTIDQNIDLQRNALTSANCDRIFEDRISGKIANRPGLKRALKY
VNKGDTLVVWKLDRLGRSVKNLVALISELHERGAHFHSLTDSIDTSSAMGRFFFYV
MSALAEMERELIVERTLAGLAAARAQGRLGGRPV.
PhiC31 recombinase
(SEQ ID NO: 398)
MDTYAGAYDRQSRERENSSAASPATQRSANEDKAADLQREVERDGGRFR
FVGHFSEAPGTSAFGTAERPEFERILNECRAGRLNMIIVYDVSRFSRLKVMDAIPIVSE
LLALGVTIVSTQEGVFRQGNVMDLIHLIMRLDASHKESSLKSAKILDTKNLQRELGG
YVGGKAPYGFELVSETKEITRNGRMVNVVINKLAHSTTPLTGPFEFEPDVIRWWWRE
IKTHKHLPFKPGSQAAIHPGSITGLCKRMDADAVPTRGETIGKKTASSAWDPATVMR
ILRDPRIAGFAAEVIYKKKPDGTPTTKIEGYRIQRDPITLRPVELDCGPIIEPAEWYELQ
AWLDGRGRGKGLSRGQAILSAMDKLYCECGAVMTSKRGEESIKDSYRCRRRKVVD
PSAPGQHEGTCNVSMAALDKFVAERIFNKIRHAEGDEETLALLWEAARRFGKLTEAP
EKSGERANLVAERADALNALEELYEDRAAGAYDGPVGRKHFRKQQAALTLRQQGA
EERLAELEAAEAPKLPLDQWFPEDADADPTGPKSWWGRASVDDKRVFVGLFVDKIV
VTKSTTGRGQGTPIEKRASITWAKPPTDDDEDDAQDGTEDVAATGA.

Recombinases for use with the GeoCas9 base editor fusion proteins may also include further mutations. Some aspects of this disclosure provide recombinases comprising an amino acid sequence that is at least 70%, at least 80%, at least 90%, at least 95%, or at least 97% identical to the sequence of the recombinase sequence discussed herein, wherein the amino acid sequence of the recombinase comprises at least one mutation as compared to the sequence of the recombinase sequence discussed herein. In some embodiments, the amino acid sequence of the recombinase comprises at least 2, at least 3, at least 4, at least 5, at least 6, at least 7, at least 8, at least 9, at least 10, at least 11, at least 12, at least 13, at least 14, or at least 15 mutations as compared to the sequence of the recombinase sequence discussed herein.

For example, the γδ recombinase may comprise one or more mutations from the list: R2A, E56K, G101S, E102Y, M103I, or E124Q. In one embodiment, the γδ recombinase may comprise an E102Y mutation, an E124Q mutation, or both an E102Y and E124Q mutation. In another embodiment, the β recombinase may comprise one or more mutations including, but not limited to N95D. See, for example, Sirk et al., “Expanding the zinc-finger recombinase repertoire: directed evolution and mutational analysis of serine recombinase specificity determinants” Nucl Acids Res (2014) 42 (7): 4755-4766. In another embodiment, the Sin recombinase may have one or more mutations including, but not limited to: Q87R, Q115R, or Q87R and Q115R. In another embodiment, the Tn3 recombinase may have one or more mutations including, but not limited to: G70S, D102Y, E124Q, and any combination thereof. In another embodiment, the Hin recombinase may have one or more mutations including, but not limited to: H107Y. In another embodiment, the Sin recombinase may have one or more mutations including, but not limited to: H107Y. Any of the recombinase catalytic domains for use with the disclosed compositions and methods may have greater than 85%, 90%, 95%, 98%, or 99% sequence identity with the native (or wild type) amino acid sequence. For example, in certain embodiments, the Gin recombinase catalytic domain has greater than 85%, 90%, 95%, 98%, or 99% sequence identity with the amino acid sequence shown in SEQ ID NO: 384. In another embodiment, the amino acid sequence of the Gin recombinase catalytic domain comprises a mutation corresponding to H106Y, and/or I127L, and/or I136R and/or G137F. In yet another embodiment, the amino acid sequence of the Gin recombinase catalytic domain comprises a mutation corresponding to H106Y, I127L, 1136R, and G137F. In a further embodiment, the amino acid sequence of the Gin recombinase has been further mutated. In a specific embodiment, the amino acid sequence of the Gin recombinase catalytic domain comprises SEQ ID NO: 384.

In some embodiments, only a portion of the recombinase is used in the GeoCas9 fusion proteins and methods described herein. As a non-limiting embodiment, only the C-terminal portion of the recombinase may be used in the fusion proteins and methods described herein. In a specific embodiment, the 25 kDa carboxy-terminal domain of Cre recombinase may be used in the compositions and methods. See, for example, Hoess et al, “DNA Specificity of the Cre Recombinase Resides in the 25 kDa Carboxyl Domain of the Protein,” J. Mol. Bio. 1990 Dec. 20, 216 (4): 873-82, which is incorporated by reference herein for all purposes. The 25 kDa carboxy-terminal domain of Cre recombinase is the portion stretching from R118 to the carboxy terminus of the protein. In some embodiments, the 25 kDa carboxy-terminal domain of Cre recombinase for use in the instant fusion proteins and methods may differ from the canonical 25 kDa carboxy-terminal domain of Cre recombinase by at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 amino acids. In some embodiments, the 25 kDa carboxy-terminal domain of Cre recombinase for use in the instant fusion proteins and methods may differ from the canonical 25 kDa carboxy-terminal domain of Cre recombinase by 1, 2, 3, 4, 5, 6, 7, 8, 9, 10, 11, 12, 13, 14, 15, 16, 17, 18, 19, or 20 amino acids. In certain embodiments, only a portion of the 25 kDa carboxy-terminal domain of Cre recombinase may be used in the fusion proteins and methods described herein. For example, the portion of Cre recombinase used may be R130 to the carboxy terminus of the protein, T140 to the carboxy terminus of the protein, E150 to the carboxy terminus of the protein, N160 to the carboxy terminus of the protein, T170 to the carboxy terminus of the protein, 1180 to the carboxy terminus of the protein, G190 to the carboxy terminus of the protein, T200 to the carboxy terminus of the protein, E210 to the carboxy terminus of the protein, L220 to the carboxy terminus of the protein, V230 to the carboxy terminus of the protein, C240 to the carboxy terminus of the protein, P250 to the carboxy terminus of the protein, A260 to the carboxy terminus of the protein, R270 to the carboxy terminus of the protein, G280 to the carboxy terminus of the protein, S290 to the carboxy terminus of the protein, A300 to the carboxy terminus of the protein, or M310 to the carboxy terminus of the protein. As another set of non-limiting examples, the portion of Cre recombinase used may be R118-E340, R118-S330, R118-1320, R118-M310, R118-A300, R118-S290, R118-G280, R118-R270, R118-A260, R118-P250, R118-C240, R118-V230, R118-L220, or R118-E210. As a further set of non-limiting examples, the portion of Cre recombinase used may be R118-E210, G190-R270, E210-S290, P250-M310, or R270 to the carboxy terminus of the protein.

In some embodiments, the Cre recombinase used in the fusion proteins and methods described herein may be truncated at any position. In a specific embodiment, the Cre recombinase used in the fusion proteins and methods described herein may be truncated such that it begins with amino acid R118, A127, E138, or R154) (preceded in each case by methionine). In another set of non-limiting embodiments, the Cre recombinase used in the fusion proteins and methods described herein may be truncated within 10 amino acids, 9 amino acids, 8 amino acids, 7 amino acids, 6 amino acids, 5 amino acids, 4 amino acids, 3 amino acids, 2 amino acids, or 1 amino acid of R118, A127, E138, or R154.

In some embodiments, the recombinase target sequence is between 10-50 nucleotides long. In some embodiments, the recombinase is a Cre recombinase, a Hin recombinase, or a FLP recombinase. In some embodiments, the canonical recombinase target sequence is a LoxP site (5′-ATAACTTCGTATA GCATACAT TATACGAAGTTAT-3′ (SEQ ID NO: 399). In some embodiments, the canonical recombinase target sequence is an FRT site (5′-GAAGTTCCTATTCTCTAGAAA GTATAGGAACTTC-3′) (SEQ ID NO: 400). In some embodiments, the amino acid sequence of the evolved recombinase comprises at least 2, at least 3, at least 4, at least 5, at least 6, at least 7, at least 8, at least 9, at least 10, at least 11, at least 12, at least 13, at least 14, or at least 15 mutations as compared to the sequence of the wild-type recombinase. In some embodiments, the evolved recombinase recognizes a DNA recombinase target sequence that comprises a left half-site, a spacer sequence, and a right half-site, and wherein the left half-site is not a palindrome of the right half-site.

In some embodiments, the evolved recombinase recognizes a DNA recombinase target sequence that comprises a naturally occurring sequence. In some embodiments, the evolved recombinase recognizes a DNA recombinase target sequence that is comprised in the genome of a mammal. In some embodiments, the evolved recombinase recognizes a DNA recombinase target sequence comprised in the genome of a human. In some embodiments, the evolved recombinase recognizes a DNA recombinase target sequence that occurs only once in the genome of a mammal. In some embodiments, the evolved recombinase recognizes a DNA recombinase target sequence in the genome of a mammal that differs from any other site in the genome by at least 1, at least 2, at least 3, at least 4, at least 5, at least 6, at least 7, at least 8, at least 9, at least 10, at least 11, at least 12, at least 13, at least 14, or at least 15 nucleotide(s). In some embodiments, the evolved recombinase recognizes a DNA recombinase target sequence located in a safe harbor genomic locus. In some embodiments, the safe harbor genomic locus is a Rosa26 locus. In some embodiments, the evolved recombinase recognizes a DNA recombinase target sequence located in a genomic locus associated with a disease or disorder.

In certain embodiments, the evolved recombinase may target a site in the Rosa locus of the human genome (e.g., 36C6). A non-limiting set of such recombinases may be found, for example, in International PCT Publication, WO 2017/015545A1, published Jan. 26, 2017, entitled “Evolution of Site Specific Recombinases,” which is incorporated by reference herein for this purpose. In some embodiments, the amino acid sequence of the evolved recombinase comprises at least 2, at least 3, at least 4, at least 5, at least 6, at least 7, at least 8, at least 9, at least 10, at least 11, at least 12, at least 13, at least 14, or at least 15 mutations as compared to the sequence of the wild-type recombinase. The nucleotide sequence encoding 36C6 is shown below in bold; those encoding GGS linkers are shown in italics; those encoding dCas9 linkers are black; those encoding the FLAG tag and NLS are underlined and in lowercase, respectively.

### G. Other Effector Domains

In addition to cytidine deaminases, adenosine deaminasws GeoCas9 base editor fusion proteins may comprise one or more nucleic acid effector domains. Exemplary nucleic acid effector domains include, but are not limited to a deaminase (e.g., a cytidine deaminase or an adenosine deaminase), a nuclease, a nickase, a recombinase, a methyltransferase, a methylase, an acetylase, an acetyltransferase, a transcriptional activator, or a transcriptional repressor domain. In some embodiments the nucleic acid effector domain is a protein or enzyme capable of making one or more modifications (e.g., deamination of a cytidine residue) to a nucleic acid (e.g., DNA or RNA). In some embodiments the nucleic acid editing domain is a deaminase (e.g., a cytidine deaminase, such as an APOBEC or an AID deaminase). Additional suitable nucleic-acid effector enzyme sequences that can be used according to aspects of this invention, e.g., that can be fused to a GeoCas9 protein, will be apparent to those of skill in the art based on this disclosure. In some embodiments, such additional enzyme sequences include deaminase enzyme or deaminase domain sequences that are at least 70%, at least 75%, at least 80%, at least 85%, at least 90%, at least 95%, at least 96%, at least 97%, at least 98%, or at least 99% similar to the sequences provided herein.

In some embodiments, the GeoCas9 base editors may comprise a Gam protein as the nucleic acid effector domain.

The term “Gam proteins,” as used herein, refers generally to proteins capable of binding to one or more ends of a double strand break of a double stranded nucleic acid (e.g., double stranded DNA). In some embodiments, the Gam protein prevents or inhibits degradation of one or more strands of a nucleic acid at the site of the double strand break. In some embodiments, a Gam protein is a naturally-occurring Gam protein from bacteriophage Mu, or a non-naturally occurring variant thereof. Gam Proteins are described in PCT Publication No. WO2019/139645, the contents of each of which are incorporated herein by reference.

Gam is related to Ku70 and Ku80, two eukaryotic proteins involved in non-homologous DNA end-joining (Cui et al., 2016). Gam has sequence homology with both subunits of Ku (Ku70 and Ku80), and can have a similar structure to the core DNA-binding region of Ku. Orthologs to Mu Gam are present in the bacterial genomes of Haemophilus influenzae, Salmonella typhi, Neisseria meningitidis and the enterohemorrhagic 0157: H7 strain of E. coli (d'Adda di Fagagna et al., 2003). Gam proteins have been described previously, for example, in COX, Proteins pinpoint double strand breaks. eLife.2013; 2: e01561.; CUI et al., Consequences of Cas9 cleavage in the chromosome of Escherichia coli. Nucleic Acids Res. 2016 May 19; 44 (9): 4243-51. doi: 10.1093/nar/gkw223. Epub 2016 Apr. 8.; D′ADDA DI FAGAGNA et al., The Gam protein of bacteriophage Mu is an orthologue of eukaryotic Ku. EMBO Rep. 2003 January; 4 (1): 47-52.; and SHEE et al., Engineered proteins detect spontaneous DNA breakage in human and bacterial cells. Elife. 2013 Oct. 29; 2: e01222. doi: 10.7554/eLife.01222; the contents of each of which are incorporated herein by reference.

In some embodiments, the Gam protein comprises the amino acid sequence of the Gam form bacteriophage Mu:

(SEQ ID NO: 401)
AKPAKRIKSAAAAYVPQNRDAVITDIKRIGDLQREASRLETEMNDAIAEI
TEKFAARIAPIKTDIETLSKGVQGWCEANRDELTNGGKVKTANLVTGDVS
WRVRPPSVSIRGMDAVMETLERLGLQRFIRTKQEINKEAILLEPKAVAGV
AGITVKSGIEDFSIIPFEQEAGI

In other embodiments, the Gam protein comprises the amino acid sequences as follows:

WP_001107930.1 MULTISPECIES: host-nuclease
inhibitor protein Gam [Enterobacteriaceae]
(SEQ ID NO: 402)
MAKPAKRIKSAAAAYVPQNRDAVITDIKRIGDLQREASRLETEMNDAIAE
ITEKFAARIAPIKTDIETLSKGVQGWCEANRDELTNGGKVKTANLVTGDV
SWRVRPPSVSIRGMDAVMETLERLGLQRFIRTKQEINKEAILLEPKAVAG
VAGITVKSGIEDFSIIPFEQEAGI
CAA27978.1 unnamed protein product
[Escherichia virus Mu]
(SEQ ID NO: 403)
MAKPAKRIKSAAAAYVPQNRDAVITDIKRIGDLQREASRLETEMNDAIAE
ITEKFAARIAPIKTDIETLSKGVQGWCEANRDELTNGGKVKTANLVTGDV
SWRVRPPSVSIRGMDAVMETLERLGLQRFVRTKQEINKEAILLEPKAVAG
VAGITVKSGIEDFSIIPFEQEAGI
WP_001107932.1 host-nuclease inhibitor protein
Gam [Escherichia coli]
(SEQ ID NO: 404)
MAKPAKRIKSAAAAYVPQNRDAVITDIKRIGDLQREASRLETEMNDAIAE
ITEKFAARIAPLKTDIETLSKGVQGWCEANRDELTNGGKVKTANLVTGDV
SWRVRPPSVSIRGMDAVMETLERLGLQRFIRTKQEINKEAILLEPKAVAG
VAGITVKSGIEDFSIIPFEQEAGI
WP_061335739.1 host-nuclease inhibitor protein
Gam [Escherichia coli]
(SEQ ID NO: 405)
MAKPAKRIKSAAAAYVPQNRDAVITDIKRIGDLQREASRLETEMNDAIAE
ITEKFAARIAPIKTDIETLSKGVQGWCEANRDELTNGGKVKTANLITGDV
SWRVRPPSVSIRGMDAVMETLERLGLQRFIRTKQEINKEAILLEPKAVAG
VAGITVKSGIEDFSIIPFEQEAGI

### H. Linkers

In various embodiments, the GeoCas9 base editors comprise one or more linkers that join or fuse the various functional domains of the base editors, e.g., the GeoCas9 domain and the one or more nucleic acid effector domains (e.g., a cytidine deaminase or adenosine deaminase domain).

In some embodiments, the linker comprises (GGGS)n (SEQ ID NO: 9), (GGGGS)n (SEQ ID NO: 10), (G)n (SEQ ID NO: 11), (EAAAK)n (SEQ ID NO: 12), (GGS)n (SEQ ID NO: 13), (SGGS)n (SEQ ID NO: 14), SGSETPGTSESATPES (SEQ ID NO: 15), SGGS (GGS)n (SEQ ID NO: 16), SGGSSGGSSGSETPGTSESATPESSGGSSGGS (SEQ ID NO: 17), or (XP)n (SEQ ID NO: 18) motif, or a combination of any of these, wherein n is independently an integer between 1 and 30, and wherein X is any amino acid. In some embodiments, the linker comprises a (GGS)n (SEQ ID NO: 13) motif, wherein n is 1, 3, or 7. In some embodiments, the linker comprises a (GGS)n (SEQ ID NO: 13) motif, wherein n is 1, 2, 3, 4, 5, 6, 7, 8, 9, 10, 11, 12, 13, 14, or 15. In some embodiments, the linker comprises the amino acid sequence SGGS (GGS)n, (SEQ ID NO: 16), wherein n is 1, 2, 3, 4, 5, 6, 7, 8, 9, or 10. In some embodiments, the linker comprises the amino acid sequence SGGS (GGS)n, (SEQ ID NO: 16), wherein n is 2. In some embodiments, the linker comprises an amino acid sequence of SGSETPGTSESATPES (SEQ ID NO: 15), also referred to as the “XTEN linker”. In some embodiments, the linker comprises the amino acid sequence SGGSSGGSSGSETPGTSESATPESSGGSSGGS (SEQ ID NO: 17), also referred to as the 32 amino acid linker. The length of the linker can influence the base to be edited. For example, when the GeoCas9 is fused to a cytidine deaminase, a 16-amino-acid linker (e.g., the XTEN linker) may give a −3 to 15, a −3 to 13, a −3 to 11, a −3 to 9, a −3 to 7, a −3 to 5, a −3 to 3, a −1 to 15, a −1 to 13, a −1 to 11, a −1 to 9, a −1 to 7, a −1 to 5, a −1 to 3, a 2 to 15, a 2 to 13, a 2 to 11, a 2 to 9, a 2 to 7, a 2 to 5, a 4 to 15, a 4 to 13, a 4 to 11, a 4 to 9, a 4 to 7, a 6 to 15, a 6 to 13, a 6 to 11, or a 6 to 9, a −3 to 7, a −3 to 5, a −3 to 3, base editing window relative to the PAM sequence (e.g., a NNNNCRAA (SEQ ID NO: 79) PAM sequence having positions 21-28). As another example, when the GeoCas9 is fused to a cytidine deaminase, a 32-amino-acid linker (e.g., the (SGGS) 2XTEN (SGGS) 2 linker) (SEQ ID NO: 17) may give a −3 to 15, a −3 to 13, a −3 to 11, a −3 to 9, a −3 to 7, a −3 to 5, a −3 to 3, a −1 to 15, a −1 to 13, a −1 to 11, a −1 to 9, a −1 to 7, a −1 to 5, a −1 to 3, a 2 to 15, a 2 to 13, a 2 to 11, a 2 to 9, a 2 to 7, a 2 to 5, a 4 to 15, a 4 to 13, a 4 to 11, a 4 to 9, a 4 to 7, a 6 to 15, a 6 to 13, a 6 to 11, or a 6 to 9, a −3 to 7, a −3 to 5, a −3 to 3, base editing window relative to the PAM sequence (e.g., a NNNNCRAA (SEQ ID NO: 79) PAM sequence having positions 21-28). Varying linker length may allow GeoCas9 fusion proteins of the disclosure to edit nucleobases different distances from the PAM sequence, which affords siginicant clinical importance, since a PAM sequence may be of varying distance to the disease-causing mutation to be modified in a gene. It is to be understood that the linker lengths described as examples herein are not meant to be limiting.

Additional suitable linker motifs and linker configurations will be apparent to those of skill in the art. In some embodiments, suitable linker motifs and configurations include those described in Chen et al., Fusion protein linkers: property, design and functionality. Adv Drug Deliv Rev. 2013; 65 (10): 1357-69, the entire contents of which are incorporated herein by reference. Additional suitable linker sequences will be apparent to those of skill in the art based on the instant disclosure.

### I. NLS and Other Sequence Tags

The GeoCas9 base editor fusion proteins of the present disclosure may also comprise one or more additional sequence features. For example, in some embodiments, the GeoCas9 base editor fusion proteins may comprise one or more nuclear localization sequences (NLS). In some embodiments, the NLS of the GeoCas9 base editor fusion proteins is localized between the nucleic acid editing domain and the GeoCas9 domain. In some embodiments, the NLS of the GeoCas9 base editor fusion protein is localized C-terminal to the GeoCas9 domain.

Other exemplary features that may be present are localization sequences, such as cytoplasmic localization sequences, export sequences, such as nuclear export sequences, or other localization sequences, as well as sequence tags that are useful for solubilization, purification, or detection of the GeoCas9 base editor fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the GeoCas9 base editor fusion protein comprises one or more His tags.

In some embodiments, the deaminase domain and the GeoCas9 are fused to each other via a linker. Various linker lengths and flexibilities between the deaminase domain (e.g., AID) and the Cas9 domain can be employed (e.g., ranging from very flexible linkers of the form (GGGS)ₙ (SEQ ID NO: 9) (GGGGS)ₙ (SEQ ID NO: 10), (GGS), (SEQ ID NO: 13), and (G)ₙ (SEQ ID NO: 11) to more rigid linkers of the form (EAAAK)ₙ (SEQ ID NO: 12), (SGGS)ₙ (SEQ ID NO: 14), SGSETPGTSESATPES (SEQ ID NO: 15) (see, e.g., Guilinger J P, Thompson D B, Liu D R. Fusion of catalytically inactive Cas9 to FokI nuclease improves the specificity of genome modification. Nat. Biotechnol. 2014; 32 (6): 577-82; the entire contents are incorporated herein by reference) and (XP)n) (SEQ ID NO: 18) in order to achieve the optimal length for deaminase activity for the specific application. In some embodiments, the linker comprises a (GGS)ₙ (SEQ ID NO: 13) motif, wherein n is 1, 3, or 7. In some embodiments, the linker comprises a (an SGSETPGTSESATPES (SEQ ID NO: 15) motif.

Some exemplary suitable nucleic-acid editing domains, e.g., deaminases and deaminase domains that can be fused to GeoCas9 according to aspects of this disclosure are provided below. It should be understood that, in some embodiments, the active domain of the respective sequence can be used, e.g., the domain without a localizing signal (nuclear localization sequence, without nuclear export signal, cytoplasmic localizing signal). Examples of cytidine deaminase amino acid sequences with an NLS sequence (single underline) and/or nuclear export signals (double underline) are shown, as follows:

Human AID:
(SEQ ID NO: 25)
MDSLLMNRRKFLYQFKNVRWAKGRRETYLCYVVKRRDSATSFSLDFGYLRNKNGC
HVELLFLRYISDWDLDPGRCYRVTWFTSWSPCYDCARHVADFLRGNPNLSLRIFTAR
LYFCEDRKAEPEGLRRLHRAGVQIAIMTFKDYFYCWNTFVENHERTFKAWEGLHEN
SVRLSROLRRILLPLYEVDDLRDAFRTLGL
(underline: nuclear localization sequence; double
underline: nuclear export signal)
Mouse AID:
(SEQ ID NO: 26)
MDSLLMKQKKFLYHFKNVRWAKGRHETYLCYVVKRRDSATSCSLDFGHLRNKSGC
HVELLFLRYISDWDLDPGRCYRVTWFTSWSPCYDCARHVAEFLRWNPNLSLRIFTAR
LYFCEDRKAEPEGLRRLHRAGVQIGIMTFKDYFYCWNTFVENRERTFKAWEGLHEN
SVRLTRQLRRILLPLYEVDDLRDAFRMLGF
(underline: nuclear localization sequence; double
underline: nuclear export signal)
Dog AID:
(SEQ ID NO: 27)
MDSLLMKQRKFLYHFKNVRWAKGRHETYLCYVVKRRDSATSFSLDFGHLRNKSGC
HVELLFLRYISDWDLDPGRCYRVTWFTSWSPCYDCARHVADFLRGYPNLSLRIFAAR
LYFCEDRKAEPEGLRRLHRAGVQIAIMTFKDYFYCWNTFVENREKTFKAWEGLHEN
SVRLSRQLRRILLPLYEVDDLRDAFRTLGL
(underline: nuclear localization sequence; double
underline: nuclear export signal)
Bovine AID:
(SEQ ID NO: 28)
MDSLLKKQRQFLYQFKNVRWAKGRHETYLCYVVKRRDSPTSFSLDFGHLRNKAGC
HVELLFLRYISDWDLDPGRCYRVTWFTSWSPCYDCARHVADFLRGYPNLSLRIFTAR
LYFCDKERKAEPEGLRRLHRAGVQIAIMTFKDYFYCWNTFVENHERTFKAWEGLHE
NSVRLSRQLRRILLPLYEVDDLRDAFRTLGL
(underline: nuclear localization sequence; double
underline: nuclear export signal)
Rat AID:
(SEQ ID NO: 44)
MAVGSKPKAALVGPHWERERIWCFLCSTGLGTQQTGQTSRWLRPAATQDPVSPPRS
LLMKQRKFLYHFKNVRWAKGRHETYLCYVVKRRDSATSFSLDFGYLRNKSGCHVE
LLFLRYISDWDLDPGRCYRVTWFTSWSPCYDCARHVADFLRGNPNLSLRIFTARLTG
WGALPAGLMSPARPSDYFYCWNTFVENHERTFKAWEGLHENSVRLSRRLRRILLPL
YEVDDLRDAFRTLGL
(underline: nuclear localization sequence; double
underline: nuclear export signal)
Rhesus macaque APOBEC-3G:
(SEQ ID NO: 31)
MVEPMDPRTFVSNFNNRPILSGLNTVWLCCEVKTKDPSGPPLDAKIFQGKVYSKAKY
HPEMRFLRWFHKWRQLHHDQEYKVTWYVSWSPCTRCANSVAEFEAKDPKVEEE1FVA
RLYYFWKPDYQQALRILCQKRGGPHATMKIMNYNEFQDCWNKFVDGRGKPFKPRN
NLPKHYTLLQATLGELLRHLMDPGTFTSNFNNKPWVSGQHETYLCYKVERLHNDT
WNPENQHRGFERNQAPNIHGFPKGRHAELCFLDLIPFWKLDGQQYRVTCFTSWSPCFS
CAQEMAKFISNNEHVSLCIFAARIYDDQGRYQEGLRALHRDGAKIAMMNYSEFEYC
WDTFVDRQGRPFQPWDGLDEHSQALSGRLRAI
(italic: nucleic acid editing domain; underline:
cytoplasmic localization signal)
Chimpanzee APOBEC-3G:
(SEQ ID NO: 32)
MKPHFRNPVERMYQDTFSDNFYNRPILSHRNTVWLCYEVKTKGPSRPPLDAKIFRGQ
VYSKLKAHPEMRFFHWFSKWRKLHRDQEYEVTWYISWSPCTKCTRDVAEFEAEDPKV
TLTIFVARLYYFWDPDYQEALRSLCQKRDGPRATMKIMNYDEFQHCWSKFVYSQRE
LFEPWNNLPKYYILLHIMLGEILRHSMDPPTFTSNFNNELWVRGRHETYLCYEVERL
HNDTWVLLNQRRGFLCNQAPHKHGFLEGRHAELCFLDVIPFWKLDLHQDYRVTCFTS
WSPCFSCAQEMAKFISNNKHVSLCIFAARIYDDQGRCQEGLRTLAKAGAKISIMTYSE
FKHCWDTFVDHQGCPFQPWDGLEEHSQALSGRLRAILQNQGN
(italic: nucleic acid editing domain; underline:
cytoplasmic localization signal)
Green monkey APOBEC-3G:
(SEQ ID NO: 33)
MNPQIRNMVEOMEPDIFVYYFNNRPILSGRNTVWLCYEVKTKDPSGPPLDANIFQGK
EYPEAKDHPEMKFLHWFRKWRQLHRDQEYEVTWYVSWSPCTRCANSVAEFEAEDPKV
TLTIFVARLYYFWKPDYQQALRILCQERGGPHATMKIMNYNEFQHCWNEFVDGQG
KPFKPRKNLPKHYTLLHATLGELLRHVMDPGTFTSNFNNKPWVSGQRETYLCYKVE
RSHNDTWVLLNQHRGFLRNQAPDRHGFPKGRHAELCFLDLIPFWKLDDOQYRVTCFT
SWSPCFSCAQKMAKFISNNKHVSLCIFAARIYDDQGRCQEGLRTLHRDGAKIAVMNY
SEFEYCWDTFVDRQGRPFQPWDGLDEHSQALSGRLRAI
(italic: nucleic acid editing domain; underline:
cytoplasmic localization signal)
Human APOBEC-3G:
(SEQ ID NO: 34)
MKPHFRNTVERMYRDTFSYNFYNRPILSRRNTVWLCYEVKTKGPSRPPLDAKIFRGO
VYSELKYHPEMRFFHWFSKWRKLHRDQEYEVTWYISWSPCTKCTRDMATFLAEDPKV
TLTIFVARLYYFWDPDYQEALRSLCQKRDGPRATMKIMNYDEFQHCWSKFVYSQRE
LFEPWNNLPKYYILLHIMLGEILRHSMDPPTFTFNFNNEPWVRGRHETYLCYEVERM
HNDTWVLLNQRRGFLCNQAPHKHGFLEGRHAELCFLDVIPFWKLDLDODYRVTCFTS
WSPCFSCAQEMAKFISKNKHVSLCIFTARIYDDQGRCQEGLRTLAEAGAKISIMTYSE
FKHCWDTFVDHQGCPFQPWDGLDEHSQDLSGRLRAILQNQEN
(italic: nucleic acid editing domain; underline:
cytoplasmic localization signal)
Human APOBEC-3F:
(SEQ ID NO: 35)
MKPHFRNTVERMYRDTFSYNFYNRPILSRRNTVWLCYEVKTKGPSRPRLDAKIFRGQ
VYSQPEHHAEMCFLSWFCGNQLPAYKCFQITWFVSWTPCPDCVAKEAEEEAEHPNVYE
TISAARLYYYWERDYRRALCRLSQAGARVKIMDDEEFAYCWENFVYSEGQPFMPW
YKFDDNYAFLHRTLKEILRNPMEAMYPHIFYFHFKNLRKAYGRNESWLCFTMEVVK
HHSPNSWKRGNERNQNDPEYHCHAERCFLSWFCDDILSPNTNYEVTWYTSWSPCPECA
GEVAEFLARHSNVNLTIFTARLYYFWDTDYQEGLRSLSQEGASVEIMGYKDFKYCW
ENFVYNDDEPFKPWKGLKYNFLFLDSKLQEILE
(italic: nucleic acid editing domain)

### J. UGI Domains

In some aspects of the disclosure, the GeoCas9 base editor fusion proteins may comprise one or more uracil glycosylase inhibitor (UGI) domains. In some embodiments, the GeoCas9 domain (e.g., a nuclease active GeoCas9 domain, a nuclease inactive dGeoCas9 domain, or a GeoCas9 nickase) may be fused to one or more UGI domains either directly or via a linker. Without wishing to be bound by any particular theory, cellular DNA-repair response to the presence of U: G heteroduplex DNA may be responsible for the decrease in nucleobase editing efficiency in cells. For example, uracil DNA glycosylase (UDG) catalyzes removal of U from DNA in cells, which may initiate base excision repair, with reversion of the U: G pair to a C: G pair as the most common outcome. Uracil DNA Glycosylase Inhibitor (UGI) inhibits human UDG activity. Thus, this disclosure contemplates a GeoCas9 base editor fusion protein comprising GeoCas9 domain, a nucleic acid effector domain (e.g., a cytidine or adenosine deaminase), and one or more UGI domains. Each of the domains may be joined through one or more linkers. It should be understood that the use of a UGI domain may increase the editing efficiency of a nucleic acid editing domain that is capable of catalyzing a C to U change. For example, GeoCas9 base editor fusion proteins comprising a UGI domain may be more efficient in deaminating C residues. In some embodiments, the GeoCas9 base editor fusion protein comprises the structure:
[NH²]-[deaminase domain]-[GeoCas9]-[UGI domain]-[COOH];
[NH²]-[nucleic acid effector domain]-[UGI domain]-[GeoCas9]-[COOH];
[NH²]-[UGI domain]-[nucleic acid effector domain]-[GeoCas9]-[COOH];
[NH²]-[UGI domain]-[GeoCas9]-[nucleic acid effector domain]-[COOH];
[NH²]-[GeoCas9]-[nucleic acid effector domain]-[UGI domain]-[COOH];
[NH²]-[GeoCas9]-[UGI domain]-[nucleic acid effector domain]-[COOH];
[NH²]-[nucleic acid effector domain]-[GeoCas9]-[first UGI domain]-[second UGI domain]-[COOH];
[NH²]-[nucleic acid effector domain]-[first UGI domain]-[second UGI domain]-[GeoCas9]-[COOH];
[NH²]-[first UGI domain]-[second UGI domain]-[nucleic acid effector domain]-[GeoCas9]-[COOH];
[NH²]-[first UGI domain]-[second UGI domain]-[GeoCas9]-[nucleic acid effector domain]-[COOH];
[NH²]-[GeoCas9]-[nucleic acid effector domain]-[first UGI domain]-[second UGI domain]-[COOH];
[NH²]-[GeoCas9]-[first UGI domain]-[second UGI domain]-[nucleic acid effector domain]-[COOH];
[NH²]-[NLS]-[nucleic acid effector domain]-[GeoCas9]-[UGI domain]-[COOH];
[NH²]-[NLS]-[nucleic acid effector domain]-[UGI domain]-[GeoCas9]-[COOH];
[NH²]-[NLS]-[UGI domain]-[nucleic acid effector domain]-[GeoCas9]-[COOH];
[NH²]-[NLS]-[UGI domain]-[GeoCas9 domain]-[deaminase]-[COOH];
[NH²]-[NLS]-[GeoCas9]-[nucleic acid effector domain]-[UGI domain]-[COOH];
[NH²]-[NLS]-[GeoCas9]-[UGI domain]-[nucleic acid effector domain]-[COOH];
[NH²]-[NLS]-[nucleic acid effector domain]-[GeoCas9]-[first UGI domain]-[second UGI domain]-[COOH];
[NH²]-[NLS]-[nucleic acid effector domain]-[first UGI domain]-[second UGI domain]-[GeoCas9]-[COOH];
[NH²]-[NLS]-[first UGI domain]-[second UGI domain]-[nucleic acid effector domain]-[GeoCas9]-[COOH];
[NH²]-[NLS]-[first UGI domain]-[second UGI domain]-[GeoCas9]-[nucleic acid effector domain]-[COOH];
[NH²]-[NLS]-[GeoCas9]-[nucleic acid effector domain]-[first UGI domain]-[second UGI domain]-[COOH];
[NH²]-[NLS]-[GeoCas9]-[first UGI domain]-[second UGI domain]-[nucleic acid effector domain]-[COOH];
[NH²]-[nucleic acid effector domain]-[GeoCas9]-[UGI domain]-[NLS]-[COOH];
[NH²]-[nucleic acid effector domain]-[UGI domain]-[GeoCas9]-[NLS]-[COOH];
[NH²]-[UGI domain]-[nucleic acid effector domain]-[GeoCas9]-[NLS]-[COOH];
[NH²]-[UGI domain]-[GeoCas9]-[nucleic acid effector domain]-[NLS]-[COOH];
[NH²]-[GeoCas9]-[nucleic acid effector domain]-[UGI domain]-[NLS]-[COOH];
[NH²]-[GeoCas9]-[UGI domain]-[nucleic acid effector domain]-[NLS]-[COOH];
[NH²]-[nucleic acid effector domain]-[GeoCas9]-[first UGI domain]-[second UGI domain]-[NLS]-[COOH];
[NH²]-[nucleic acid effector domain]-[first UGI domain]-[second UGI domain]-[GeoCas9]-[NLS]-[COOH];
[NH²]-[first UGI domain]-[second UGI domain]-[nucleic acid effector domain]-[GeoCas9]-[NLS]-[COOH];
[NH²]-[first UGI domain]-[second UGI domain]-[GeoCas9]-[nucleic acid effector domain]-[NLS]-[COOH];
[NH²]-[GeoCas9]-[nucleic acid effector domain]-[first UGI domain]-[second UGI domain]-[NLS]-[COOH]; or
[NH²]-[GeoCas9]-[first UGI domain]-[second UGI domain]-[nucleic acid effector domain]-[NLS]-[COOH].

In various embodiments, the nucleic acid effector domain in these constructs can be a cytidine deaminase. In other embodiment, the nucleic acid effector domain in these constructs can be an adenosine deaminase. In still other embodiments, the nucleic acid effector domain can be a recombinase domain. In yet other embodiments, the nucleic acid effector domain can be a methyltransferase, a methylase, an acetylase, an acetyltransferase, a transcriptional activator, or a transcriptional repressor domain.

In some embodiments, a UGI domain comprises a wild-type UGI or a UGI as set forth in SEQ ID NO: 60. In some embodiments, the UGI proteins provided herein include fragments of UGI and proteins homologous to a UGI or a UGI fragment. For example, in some embodiments, a UGI domain comprises a fragment of the amino acid sequence set forth in SEQ ID NO: 60. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or at least 99.5% of the amino acid sequence as set forth in SEQ ID NO: 60. In some embodiments, a UGI comprises an amino acid sequence homologous to the amino acid sequence set forth in SEQ ID NO: 60 or an amino acid sequence homologous to a fragment of the amino acid sequence set forth in SEQ ID NO: 60. In some embodiments, proteins comprising UGI or fragments of UGI or homologs of UGI or UGI fragments are referred to as “UGI variants.” A UGI variant shares homology to UGI, or a fragment thereof.

For example a UGI variant is at least 70% identical, at least 75% identical, at least 80% identical, at least 85% identical, at least 90% identical, at least 95% identical, at least 96% identical, at least 97% identical, at least 98% identical, at least 99% identical, at least 99.5% identical, or at least 99.9% identical to a wild type UGI or a UGI as set forth in SEQ ID NO: 60. In some embodiments, the UGI variant comprises a fragment of UGI, such that the fragment is at least 70% identical, at least 80% identical, at least 90% identical, at least 95% identical, at least 96% identical, at least 97% identical, at least 98% identical, at least 99% identical, at least 99.5% identical, or at least 99.9% to the corresponding fragment of wild-type UGI or a UGI as set forth in SEQ ID NO: 60. In some embodiments, the UGI comprises the following amino acid sequence:

>sp|P14739|UNGI_BPPB2 Uracil-DNA glycosylase
inhibitor
(SEQ ID NO: 60)
MTNLSDIIEKETGKQLVIQESILMLPEEVEEVIGNKPESDILVH
TAYDESTDENVMLLTSDAPEYKPWALVIQDSNGENKIKML

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171 (1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419 (1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887 (1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346 (1999), the entire contents of each are incorporated herein by reference.

It should be appreciated that additional proteins may be uracil glycosylase inhibitors. For example, other proteins that are capable of inhibiting (e.g., sterically blocking) a uracil-DNA glycosylase base-excision repair enzyme are within the scope of this disclosure. Additionally, any proteins that block or inhibit base-excision repair as also within the scope of this disclosure. In some embodiments, the GeoCas9 base editor fusion proteins described herein comprise one UGI domain. In some emobidments, the GeoCas9 base editor fusion proteins described herein comprise more than one UGI domain. In some embodiments, the GeoCas9 base editor fusion proteins described herein comprise two UGI domains. In some embodiments, the GeoCas9 base editor fusion proteins described herein comprise more than two UGI domains. In some embodiments, a protein that binds DNA is used. In another embodiment, a substitute for UGI is used. In some embodiments, a uracil glycosylase inhibitor is a protein that binds single-stranded DNA.

For example, a uracil glycosylase inhibitor may be a Erwinia tasmaniensis single-stranded binding protein. In some embodiments, the single-stranded binding protein comprises the amino acid sequence (SEQ ID NO: 61). In some embodiments, a uracil glycosylase inhibitor is a protein that binds uracil. In some embodiments, a uracil glycosylase inhibitor is a protein that binds uracil in DNA. In some embodiments, a uracil glycosylase inhibitor is a catalytically inactive uracil DNA-glycosylase protein. In some embodiments, a uracil glycosylase inhibitor is a catalytically inactive uracil DNA-glycosylase protein that does not excise uracil from the DNA. For example, a uracil glycosylase inhibitor is a UdgX. In some embodiments, the UdgX comprises the amino acid sequence (SEQ ID NO: 62). As another example, a uracil glycosylase inhibitor is a catalytically inactive UDG. In some embodiments, a catalytically inactive UDG comprises the amino acid sequence (SEQ ID NO: 63).

It should be appreciated that other uracil glycosylase inhibitors would be apparent to the skilled artisan and are within the scope of this disclosure. In some embodiments, a uracil glycosylase inhibitor is a protein that is homologous to any one of SEQ ID NOs: 60-74. In some embodiments, a uracil glycosylase inhibitor is a protein that is at least 50% identical, at least 55% identical at least 60% identical, at least 65% identical, at least 70% identical, at least 75% identical, at least 80% identical at least 85% identical, at least 90% identical, at least 95% identical, at least 96% identical, at least 98% identical, at least 99% identical, or at least 99.5% identical to any one of SEQ ID NOs: 60-74. Additional suitable UGI sequences are as follows.

Erwinia tasmaniensis SSB
(themostable single-stranded DNA binding protein)
(SEQ ID NO: 61)
MASRGVNKVILVGNLGQDPEVRYMPNGGAVANITLATSESWRDKQTGET
KEKTEWHRVVLFGKLAEVAGEYLRKGSQVYIEGALQTRKWTDQAGVEKY
TTEVVVNVGGTMQMLGGRSQGGGASAGGQNGGSNNGWGQPQQPQGGNQF
SGGAQQQARPQQQPQQNNAPANNEPPIDFDDDIP
UdgX (binds to Uracil in DNA but does not excise)
(SEQ ID NO: 62)
MAGAQDFVPHTADLAELAAAAGECRGCGLYRDATQAVFGAGGRSARIMM
IGEQPGDKEDLAGLPFVGPAGRLLDRALEAADIDRDALYVTNAVKHFKF
TRAAGGKRRIHKTPSRTEVVACRPWLIAEMTSVEPDVVVLLGATAAKAL
LGNDFRVTQHRGEVLHVDDVPGDPALVATVHPSSLLRGPKEERESAFAG
LVDDLRVAADVRP
UDG (catalytically inactive human UDG, binds to
Uracil in DNA but does not excise)
(SEQ ID NO: 63)
MIGQKTLYSFFSPSPARKRHAPSPEPAVQGTGVAGVPEESGDAAAIPAK
KAPAGQEEPGTPPSSPLSAEQLDRIQRNKAAALLRLAARNVPVGFGESW
KKHLSGEFGKPYFIKLMGFVAEERKHYTVYPPPHQVFTWTQMCDIKDVK
VVILGQEPYHGPNQAHGLCFSVQRPVPPPPSLENIYKELSTDIEDFVHP
GHGDLSGWAKQGVLLLNAVLTVRAHQANSHKERGWEQFTDAVVSWLNQN
SNGLVFLLWGSYAQKKGSAIDRKRHHVLQTAHPSPLSVYRGFFGCRHFS
KTNELLQKSGKKPIDWKEL

Additional single-stranded DNA binding proteins that can be used as a UGI are shown below. It should be appreciated that other single-stranded binding proteins may be used as a UGI, for example those described in Dickey T H, Altschuler S E, Wuttke D S. Single-stranded DNA-binding proteins: multiple domains for multiple functions. Structure. 2013 Jul. 2; 21 (7): 1074-84. doi: 10.1016/j.str.2013.05.013. Review.; Marceau A H. Functions of single-strand DNA-binding proteins in DNA replication, recombination, and repair. Methods Mol Biol. 2012; 922:1-21. doi: 10.1007/978-1-62703-032-8_1.; Mijakovic, Ivan, et al.; Bacterial single-stranded DNA-binding proteins are phosphorylated on tyrosine. Nucleic Acids Res 2006; 34 (5): 1588-1596. doi: 10.1093/nar/gkj514; Mumtsidu E, Makhov A M, Konarev P V, Svergun D I, Griffith J D, Tucker P A. Structural features of the single-stranded DNA-binding protein of Epstein-Barrvirus. J Struct Biol. 2008 February; 161 (2): 172-87. Epub 2007 Nov. 1; Nowak M, Olszewski M, Śpibida M, Kur J. Characterization of single-stranded DNA-binding proteins from the psychrophilic bacteria Desulfotalea psychrophila, Flavobacterium psychrophilum, Psychrobacter arcticus, Psychrobactercryohalolentis, Psychromonas ingrahamii, Psychroflexus torquis, and Photobacterium profundum. BMC Microbiol. 2014 Apr. 14; 14:91. doi: 10.1186/1471-2180-14-91; Tone T, Takeuchi A, Makino O. Single-stranded DNA binding protein Gp5 of Bacillus subtilis phage Φ29 is required for viral DNA replication in growth-temperature dependent fashion. Biosci Biotechnol Biochem. 2012; 76 (12): 2351-3. Epub 2012 Dec. 7; Wold. REPLICATION PROTEIN A: A Heterotrimeric, Single-Stranded DNA-Binding Protein Required for Eukaryotic DNA Metabolism. Annual Review of Biochem. 1997; 66:61-92. doi: 10.1146/annurev.biochem.66.1.61; Wu Y, Lu J, Kang T. Human single-stranded DNA binding proteins: guardians of genome stability. Acta Biochim Biophys Sin (Shanghai). 2016 July; 48 (7): 671-7. doi: 10.1093/abbs/gmw044. Epub 2016 May 23. Review; the entire contents of each are hereby incorporated by reference.

mtSSB-SSBP1 single stranded DNA binding protein 1
[Homo sapiens (human)] (UniProtKB: Q04837; NP_001243439.1)
(SEQ ID NO: 64)
MFRRPVLQVLRQFVRHESETTTSLVLERSLNRVHLLGRVGQDPVLRQVEG
KNPVTIFSLATNEMWRSGDSEVYQLGDVSQKTTWHRISVFRPGLRDVAYQYVKKGS
RIYLEGKIDYGEYMDKNNVRRQATTIIADNIIFLSDQTKEKE
Single-stranded DNA-binding protein 3 isoform A [Mus musculus]
(UniProtKB-Q9D032-1; NCBI Ref: NP_076161.2)
(SEQ ID NO: 65)
MFAKGKGSAVPSDGQAREKLALYVYEYLLHVGAQKSAQTFLSEIRWEKNI
TLGEPPGFLHSWWCVFWDLYCAAPERRDTCEHSSEAKAFHDYSAAAAPSPVLGNIPP
NDGMPGGPIPPGFFQGPPGSQPSPHAQPPPHNPSSMMGPHSQPFMSPRYAGGPRPPIR
MGNQPPGGVPGTQPLLPNSMDPTRQQGHPNMGGSMQRMNPPRGMGPMGPGPQNY
GSGMRPPPNSLGPAMPGINMGPGAGRPWPNPNSANSIPYSSSSPGTYVGPPGGGGPP
GTPIMPSPADSTNSSDNIYTMINPVPPGGSRSNFPMGPGSDGPMGGMGGMEPHHMN
GSLGSGDIDGLPKNSPNNISGISNPPGTPRDDGELGGNFLHSFQNDNYSPSMTMSV
RPA 1-Replication protein A 70 kDa DNA-binding subunit
(UniProtKB: P27694; NCBI Ref: NM_002945.3)
(SEQ ID NO: 66)
MVGQLSEGAIAAIMQKGDTNIKPILQVINIRPITTGNSPPRYRLLMSDGLNT
LSSFMLATQLNPLVEEEQLSSNCVCQIHRFIVNTLKDGRRVVILMELEVLKSAEAVGV
KIGNPVPYNEGLGQPQVAPPAPAASPAASSRPQPQNGSSGMGSTVSKAYGASKTFGK
AAGPSLSHTSGGTQSKVVPIASLTPYQSKWTICARVTNKSQIRTWSNSRGEGKLFSLE
LVDESGEIRATAFNEQVDKFFPLIEVNKVYYFSKGTLKIANKQFTAVKNDYEMTFNN
ETSVMPCEDDHHLPTVQFDFTGIDDLENKSKDSLVDIIGICKSYEDATKITVRSNNRE
VAKRNIYLMDTSGKVVTATLWGEDADKFDGSRQPVLAIKGARVSDFGGRSLSVLSS
STIIANPDIPEAYKLRGWFDAEGQALDGVSISDLKSGGVGGSNTNWKTLYEVKSENL
GQGDKPDYFSSVATVVYLRKENCMYQACPTQDCNKKVIDQQNGLYRCEKCDTEFP
NFKYRMILSVNIADFQENQWVTCFQESAEAILGQNAAYLGELKDKNEQAFEEVFQN
ANFRSFIFRVRVKVETYNDESRIKATVMDVKPVDYREYGRRLVMSIRRSALM
RPA 2-Replication protein A 32 kDa subunit (UniProtKB:
P15927; NCBI Ref: NM_002946)
(SEQ ID NO: 67)
MWNSGFESYGSSSYGGAGGYTQSPGGFGSPAPSQAEKKSRARAQHIVPCTI
SQLLSATLVDEVFRIGNVEISQVTIVGIIRHAEKAPTNIVYKIDDMTAAPMDVRQWVD
TDDTSSENTVVPPETYVKVAGHLRSFQNKKSLVAFKIMPLEDMNEFTTHILEVINAH
MVLSKANSQPSAGRAPISNPGMSEAGNFGGNSFMPANGLTVAQNQVLNLIKACPRP
EGLNFQDLKNQLKHMSVSSIKQAVDFLSNEGHIYSTVDDDHFKSTDAE
RPA 3-Replication protein A 14 kDa subunit (UniProtKB: P35244;
NCBI Ref: NM_002947.4)
(SEQ ID NO: 68)
MVDMMDLPRSRINAGMLAQFIDKPVCFVGRLEKIHPTGKMFILSDGEGKN
GTIELMEPLDEEISGIVEVVGRVTAKATILCTSYVQFKEDSHPFDLGLYNEAVKIIHDF
PQFYPLGIVQHD
ssbA-single-stranded DNA-binding protein [Bacillus subtilis
subsp. subtilis str. 168] (UniProtKB: P37455; NCBI Ref:)
(SEQ ID NO: 69)
MLNRVVLVGR LTKDPELRYT PNGAAVATFT LAVNRTFTNQ
SGEREADFINCVTWRRQAEN VANFLKKGSL AGVDGRLQTR NYENQQGQRV
FVTEVQAESVQFLEPKNGGG SGSGGYNEGN SGGGQYFGGG QNDNPFGGNQ
NNQRRNQGNSFNDDPFANDG KPIDISDDDLPF
Single-stranded DNA-binding protein 2 [Streptomyces coelicolor
A3(2)] (UniProtKB: Q9X8U3; NCBI Ref: NP_628093.1)
(SEQ ID NO: 70)
MAGETVITVVGNLVDDPELRFTPSGAAVAKFRVASTPRTFDRQTNEWKDG
ESLFLTCSVWRQAAENVAESLQRGMRVIVQGRLKQRSYEDREGVKRTVYELDVDEV
GASLRSATAKVTKTSGQGRGGQGGYGGGGGGQGGGGWGGGPGGGQQGGGAPAD
DPWATGGAPAGGQQGGGGQGGGGWGGGSGGGGGYSDEPPF
Single-stranded DNA-binding protein [Streptococcus pneumoniae R6]
(UniProtKB: P66855; NCBI Ref: NP_358988.1)
(SEQ ID NO: 71)
MINNVVLVGRMTRDAELRYTPSNVAVATFTLAVNRTFKSQNGEREADFIN
VVMWRQQAENLANWAKKGSLIGVTGRIQTRSYDNQQGQRVYVTEVVAENFQMLE
SRSVREGHTGGAYSAPTANYSAPTNSVPDFSRNENPFGATNPLDISDDDLPF
Single-stranded DNA-binding protein [Human alphaherpesvirus 1]
(UniProtKB: P04296; NCBI Ref: YP_009137104.1)
(SEQ ID NO: 72)
METKPKTATTIKVPPGPLGYVYARACPSEGIELLALLSARSGDSDVAVAPL
VVGLTVESGFEANVAVVVGSRTTGLGGTAVSLKLTPSHYSSSVYVFHGGRHLDPST
QAPNLTRLCERARRHFGFSDYTPRPGDLKHETTGEALCERLGLDPDRALLYLVVTEG
FKEAVCINNTFLHLGGSDKVTIGGAEVHRIPVYPLQLFMPDFSRVIAEPFNANHRSIGE
NFTYPLPFFNRPLNRLLFEAVVGPAAVALRCRNVDAVARAAAHLAFDENHEGAALP
ADITFTAFEASQGKTPRGGRDGGGKGPAGGFEQRLASVMAGDAALALESIVSMAVF
DEPPTDISAWPLFEGQDTAAARANAVGAYLARAAGLVGAMVFSTNSALHLTEVDD
AGPADPKDHSKPSFYRFFLVPGTHVAANPQVDREGHVVPGFEGRPTAPLVGGTQEF
AGEHLAMLCGFSPALLAKMLFYLERCDGGVIVGRQEMDVFRYVADSNQTDVPCNL
CTFDTRHACVHTTLMRLRARHPKFASAARGAIGVFGTMNSMYSDCDVLGNYAAFS
ALKRADGSETARTIMQETYRAATERVMAELETLQYVDQAVPTAMGRLETIITNREAL
HTVVNNVRQVVDREVEQLMRNLVEGRNFKFRDGLGEANHAMSLTLDPYACGPCPL
LQLLGRRSNLAVYQDLALSQCHGVFAGQSVEGRNFRNQFQPVLRRRVMDMFNNGF
LSAKTLTVALSEGAAICAPSLTAGQTAPAESSFEGDVARVTLGFPKELRVKSRVLFAG
ASANASEAAKARVASLQSAYQKPDKRVDILLGPLGFLLKQFHAAIFPNGKPPGSNQP
NPQWFWTALQRNQLPARLLSREDIETIAFIKKFSLDYGAINFINLAPNNVSELAMYYM
ANQILRYCDHSTYFINTLTAIIAGSRRPPSVQAAAAWSAQGGAGLEAGARALMDAV
DAHPGAWTSMFASCNLLRPVMAARPMVVLGLSISKYYGMAGNDRVFQAGNWASL
MGGKNACPLLIFDRTRKFVLACPRAGFVCAASSLGGGAHESSLCEQLRGIISEGGAA
VASSVFVATVKSLGPRTQQLQIEDWLALLEDEYLSEEMMELTARALERGNGEWSTD
AALEVAHEAEALVSQLGNAGEVFNFGDFGCEDDNATPFGGPGAPGPAFAGRKRAFH
GDDPFGEGPPDKKGDLTLDML
Single-stranded DNA-binding protein from Bacillus virus phi29
(UniProtKB: Q38504.1; NCBI Ref: YP_002004532.1)
(SEQ ID NO: 73)
MENTNIVKATFDTETLEGQIKIFNAQTGGGQSFKNLPDGTIIEANAIAQYKQ
VSDTYGDAKEETVTTIFAADGSLYSAISKTVAEAASDLIDLVTRHKLETFKVKVVQG
TSSKGNVFFSLQLSL
Single stranded DNA binding protein [Burkholderia virus DC1]
(UniProtKB: I6NRL7; NCBI Ref: YP_006589943.1)
(SEQ ID NO: 74)
MASVNKVILVGNLGADPETRYLPSGDAISNIRLATTDRYKDKASGEMKEST
EWHRVSFFGRLAEIVDEYLRKGAPVYIEGRIRTRKWQDNAGQDRYTTEIVAEKMQM
LGDRRDGGERQQRAPQQQQQRTQRNGYADATGRAQPSQRPAAGGGFDEMDDDIPF

Suitable UGI protein and nucleotide sequences are provided herein and additional suitable UGI sequences are known to those in the art, and include, for example, those published in Wang et al., Uracil-DNA glycosylase inhibitor gene of bacteriophage PBS2 encodes a binding protein specific for uracil-DNA glycosylase. J. Biol. Chem. 264:1163-1171 (1989); Lundquist et al., Site-directed mutagenesis and characterization of uracil-DNA glycosylase inhibitor protein. Role of specific carboxylic amino acids in complex formation with Escherichia coli uracil-DNA glycosylase. J. Biol. Chem. 272:21408-21419 (1997); Ravishankar et al., X-ray analysis of a complex of Escherichia coli uracil DNA glycosylase (EcUDG) with a proteinaceous inhibitor. The structure elucidation of a prokaryotic UDG. Nucleic Acids Res. 26:4880-4887 (1998); and Putnam et al., Protein mimicry of DNA from crystal structures of the uracil-DNA glycosylase inhibitor protein and its complex with Escherichia coli uracil-DNA glycosylase. J. Mol. Biol. 287:331-346 (1999), the entire contents of which are incorporated herein by reference. In some embodiments, the optional linker comprises a (GGS)n motif, wherein n is 1, 2, 3, 4, 5, 6, 7, 8, 9, 19, 11, 12, 13, 14, 15, 16, 17, 18, 19, or 20. In some embodiments, the optional linker comprises a (GGS), motif, wherein n is 1, 3, or 7. In some embodiments, the optional linker comprises the amino acid sequence SGSETPGTSESATPES (SEQ ID NO: 15), which is also referred to as the XTEN linker in the Examples.

### K. GeoCas9 Base Editor Fusion Proteins

In various aspects, the disclosure provide GeoCas9 base editors which are fusion proteins comprising two or more functional domains, optionally joined by one or linkers. These proteins may also be referred to as GeoCas9 base editor fusion proteins, or in some cases, fusion proteins. The functional domains can include a GeoCas9 domain (e.g., a nuclease active GeoCas9 protein, a nuclease-inactive dGeoCas9 protein, or a GeoCas9 nickase protein) and one or more nucleic acid effector domains, such as, but not limited to, a cytidine deaminase, an adenosine deaminase, a nuclease, a nickase, a recombinase, a methyltransferase, a methylase, an acetylase, an acetyltransferase, a transcriptional activator, or a transcriptional repressor domain. Any of these functional domains may be joined by one or more linkers. In addition, the GeoCas9 base editors may comprise additional sequences, including, but not limited to, one or more nuclear localization sequences, UGI domains (one or more) and other sequence tags. The one or more UGI domains may also be joined by one or more linker sequences.

The GeoCas9 base editors are not particularly limited in the arrangement of its functional domains, i.e., the linear order of functional domains from the amino-terminus to the carboxy-terminus of the GeoCas9 base editor fusion proteins.

In some embodiments, the general architecture of exemplary GeoCas9 fusion proteins with a nucleic acid effector domain (e.g., a cytidine deaminase, adenosine deaminase, recombinase, methylase, methyltransferase, etc.) comprises the structure:
[NH₂]-[NLS]-[nucleic acid effector domain]-[GeoCas9]-[COOH];
[NH₂]-[NLS]-[GeoCas9]-[nucleic acid effector domain]-[COOH];
[NH₂]-[nucleic acid effector domain]-[GeoCas9]-[NLS]-[COOH]; or
[NH₂]-[GeoCas9]-[nucleic acid effector domain]-[NLS]-[COOH];
wherein NLS is a nuclear localization sequence, NH₂ is the N-terminus of the GeoCas9 base editor fusion protein, COOH is the C-terminus of the GeoCas9 base editor fusion protein, and wherein each instance of “-” comprises an optional linker, for example any of the linkers provided herein. Nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, a NLS comprises the amino acid sequence PKKKRKV (SEQ ID NO: 75) or MDSLLMNRRKFLYQFKNVRWAKGRRETYLC (SEQ ID NO: 76).

In some embodiments, a linker is inserted between the GeoCas9 and the deaminase. In some embodiments, the NLS is located C-terminal of the GeoCas9 domain. In some embodiments, the NLS is located N-terminal of the GeoCas9 domain. In some embodiments, the NLS is located between the deaminase and the GeoCas9 domain. In some embodiments, the NLS is located N-terminal of the deaminase domain. In some embodiments, the NLS is located C-terminal of the deaminase domain.

Some aspects of this disclosure provide fusion proteins comprising (i) a GeoCas9, such as a nuclease active GeoCas9, a nuclease inactive dGeoCas9, or a GeoCas9 nickase; and (ii) an effector domain, such as a cytidine deaminase domain. In some embodiments, the GeoCas9 (e.g., GeoCas9, dGeoCas9, or GeoCas9n), comprises an amino acid sequence that is at least 80%, at least 85%, at least 90%, at least 92%, at least 95%, at least 96%, at least 97%, at least 98%, at least 99%, or at least 99.5% identical to the amino acid sequence of a GeoCas9 as provided by any one of SEQ ID NOs: 2, 3, 5-8. In some embodiments, the GeoCas9 comprises one or more mutations that inactivate or alter (e.g., yield a nickase) the nuclease activity of GeoCas9. Mutations that render one or more nuclease domains of Cas9 inactive are well-known in the art. For example, the DNA cleavage domain of GeoCas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of GeoCas9. For example, the mutations D8A and H582A could inactivate the nuclease activity of GeoCas9. In some embodiments, the dGeoCas9 of this disclosure comprises a D8A mutation of the amino acid sequence provided in SEQ ID NO: 5, or a corresponding mutation in any of the amino acid sequences provided in SEQ ID NOs: 6-8.

In some embodiments, the dGeoCas9 of this disclosure comprises a H582A mutation of the amino acid sequence provided in SEQ ID NO: 5, or a corresponding mutation in any of the amino acid sequences provided in SEQ ID NOs: 6-8. In some embodiments, the dGeoCas9 of this disclosure comprises both D8A and H582A mutations of the amino acid sequence provided in SEQ ID NO: 5, or a corresponding mutation in any of the amino acid sequences provided in SEQ ID NOs: 6-8. In some embodiments, the GeoCas9 comprises a histidine residue at position 840 of the amino acid sequence provided in SEQ ID NO: 5, or a corresponding mutation in any of the amino acid sequences provided in SEQ ID NOs: 6-8. In some embodiments, the GeoCas9 comprises an amino acid sequence of SEQ ID NO: 7. It is to be understood that other mutations that modify or inactivate the nuclease domains of GeoCas9 may also be included in the dGeoCas9 or GeoCas9n of this disclosure.

The GeoCas9, GeoCas9n, or dGeoCas9 proteins comprising the mutations disclosed herein, may be a full-length GeoCas9, or a fragment thereof. In some embodiments, proteins comprising GeoCas9, or fragments thereof, are referred to as “GeoCas9 variants.” A GeoCas9 variant shares homology to GeoCas9, or a fragment thereof. For example a GeoCas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% to a wild-type GeoCas9. In some embodiments, the GeoCas9 variant comprises a fragment of GeoCas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type GeoCas9, e.g., a GeoCas9 comprising the amino acid sequence of SEQ ID NO: 5, SEQ ID NO: 6, or any one of SEQ ID NOs 7-8.

Any of the GeoCas9 fusion proteins of this disclosure may further comprise an effector domain. In some embodiments, the effector domain is a nucleic acid editing domain (e.g., an enzyme that is capable of modifying nucleic acid, such as a deaminase). In some embodiments, the nucleic acid editing domain is a DNA-editing domain. In some embodiments, the nucleic acid editing domain has deaminase activity. In some embodiments, the nucleic acid editing domain comprises or consists of a deaminase or deaminase domain.

In some embodiments, the deaminase is a cytidine deaminase. In some embodiments, the deaminase is an apolipoprotein B mRNA-editing complex (APOBEC) family deaminase. In some embodiments, the deaminase is an APOBEC1 family deaminase. In some embodiments, the deaminase is an activation-induced cytidine deaminase (AID). Some nucleic-acid editing domains as well as Cas9 fusion proteins including such domains are described in detail herein. Additional suitable nucleic acid editing domains will be apparent to the skilled artisan based on this disclosure and knowledge in the field.

Strategies for generating GeoCas9 base editor fusion proteins comprising a GeoCas9 and an effector domain (e.g., a deaminase domain) will be apparent to those of skill in the art based on this disclosure in combination with the general knowledge in the art. Suitable strategies for generating GeoCas9 base editor fusion proteins according to aspects of this disclosure using linkers or without the use of linkers will also be apparent to those of skill in the art in view of the instant disclosure and the knowledge in the art. For example, Gilbert et al., CRISPR-mediated modular RNA-guided regulation of transcription in eukaryotes. Cell. 2013; 154 (2): 442-51, showed that C-terminal fusions of Cas9 with VP64 using 2 NLS's as a linker (SPKKKRKVEAS, SEQ ID NO: 23), can be employed for transcriptional activation. Mali et al., CAS9 transcriptional activators for target specificity screening and paired nickases for cooperative genome engineering. Nat Biotechnol. 2013; 31 (9): 833-8, reported that C-terminal fusions with VP64 without linker can be employed for transcriptional activation. And Maeder et al., CRISPR RNA-guided activation of endogenous human genes. Nat Methods. 2013; 10:977-979, reported that C-terminal fusions with VP64 using a Gly₄Ser (SEQ ID NO: 10) linker can be used as transcriptional activators. Recently, dCas9-FokI nuclease fusions have successfully been generated and exhibit improved enzymatic specificity as compared to the parental Cas9 enzyme (In Guilinger J P, Thompson D B, Liu D R. Fusion of catalytically inactive Cas9 to FokI nuclease improves the specificity of genome modification. Nat. Biotechnol. 2014; 32 (6): 577-82, and in Tsai S Q, Wyvekens N, Khayter C, Foden J A, Thapar V, Reyon D, Goodwin M J, Aryee M J, Joung J K. Dimeric CRISPR RNA-guided FokI nucleases for highly specific genome editing. Nat Biotechnol. 2014; 32 (6): 569-76. PMID: 24770325 a SGSETPGTSESATPES (SEQ ID NO: 15) or a (GGGGS)ₙ (SEQ ID NO: 10), wherein n is 1, linker was used in GeoCas9 base editor fusion proteins, respectively).

In some embodiments, the various domains of the GeoCas9 fusion proteins can be joined by various linkers that join the domains of the protein, e.g., a first linker that joins a first and second domain, a second linker that joins a second domain to a third domain, etc.

For example, the first linker can comprise an amino acid sequence of 1-50 amino acids. In some embodiments, the first linker comprises an amino acid sequence of 1-40 amino acids. In some embodiments, the first linker comprises an amino acid sequence of 1-35 amino acids. In some embodiments, the first linker comprises an amino acid sequence of 1-30 amino acids. In some embodiments, the first linker comprises an amino acid sequence of 1-20 amino acids. In some embodiments, the first linker comprises an amino acid sequence of 10-20 amino acids. In some embodiments, the first linker comprises an amino acid sequence of 30-40 amino acids. In some embodiments, the first linker comprises an amino acid sequence of 14, 16, or 18 amino acids. In some embodiments, the first linker comprises an amino acid sequence of 16 amino acids. In some embodiments, the first linker comprises an amino acid sequence of 30, 32, or 34 amino acids. In some embodiments, the first linker comprises an amino acid sequence of 32 amino acids. In some embodiments, the first linker comprises a SGSETPGTSESATPES (SEQ ID NO: 15) motif. In some embodiments, the first linker comprises a SGGSSGGSSGSETPGTSESATPESSGGSSGGS (SEQ ID NO: 17) motif.

In some embodiments, the second linker comprises comprises an amino acid sequence of 1-50 amino acids. In some embodiments, the second linker comprises an amino acid sequence of 1-40 amino acids. In some embodiments, the second linker comprises an amino acid sequence of 1-35 amino acids. In some embodiments, the second linker comprises an amino acid sequence of 1-30 amino acids. In some embodiments, the second linker comprises an amino acid sequence of 1-20 amino acids. In some embodiments, the second linker comprises an amino acid sequence of 2-20 amino acids. In some embodiments, the second linker comprises an amino acid sequence of 2-10 amino acids. In some embodiments, the second linker comprises an amino acid sequence of 10-20 amino acids. In some embodiments, the second linker comprises an amino acid sequence of 2, 4, or 6 amino acids. In some embodiments, the second linker comprises an amino acid sequence of 7, 9, or 11 amino acids. In some embodiments, the second linker comprises an amino acid sequence of 14, 16, or 18 amino acids. In some embodiments, the second linker comprises an amino acid sequence of 4 amino acids. In some embodiments, the second linker comprises an amino acid sequence of 9 amino acids. In some embodiments, the second linker comprises an amino acid sequence of 16 amino acids. In some embodiments, the second linker comprises a (SGGS)ₙ (SEQ ID NO: 14) motif, wherein n is an integer between 1 and 30, inclusive. In some embodiments, the second linker comprises a (SGGS)ₙ (SEQ ID NO: 14) motif, wherein n is 1. In some embodiments, the second linker comprises a SGGS(GGS)ₙ (SEQ ID NO: 16) motif, wherein n is an integer between 1 and 30, inclusive. In some embodiments, the second linker comprises a SGGS(GGS)ₙ (SEQ ID NO: 16) motif, wherein n is 2.

In some embodiments, the third linker comprises an amino acid sequence of 1-50 amino acids. In some embodiments, the third linker comprises an amino acid sequence of 1-40 amino acids. In some embodiments, the third linker comprises an amino acid sequence of 1-35 amino acids. In some embodiments, the third linker comprises an amino acid sequence of 1-30 amino acids. In some embodiments, the third linker comprises an amino acid sequence of 1-20 amino acids. In some embodiments, the third linker comprises an amino acid sequence of 2-20 amino acids. In some embodiments, the third linker comprises an amino acid sequence of 2-10 amino acids. In some embodiments, the third linker comprises an amino acid sequence of 10-20 amino acids. In some embodiments, the third linker comprises an amino acid sequence of 2, 4, or 6 amino acids. In some embodiments, the third linker comprises an amino acid sequence of 7, 9, or 11 amino acids. In some embodiments, the third linker comprises an amino acid sequence of 14, 16, or 18 amino acids. In some embodiments, the third linker comprises an amino acid sequence of 4 amino acids. In some embodiments, the third linker comprises an amino acid sequence of 9 amino acids. In some embodiments, the third linker comprises an amino acid sequence of 16 amino acids. In some embodiments, the third linker comprises a (SGGS)ₙ (SEQ ID NO: 14) motif, wherein n is an integer between 1 and 30, inclusive. In some embodiments, the third linker comprises a (SGGS)ₙ (SEQ ID NO: 14) motif, wherein n is 1. In some embodiments, the third linker comprises a SGGS(GGS)ₙ (SEQ ID NO: 16) motif, wherein n is an integer between 1 and 30, inclusive. In some embodiments, the third linker comprises a SGGS(GGS)ₙ (SEQ ID NO: 16) motif, wherein n is 2.

In some embodiments, the GeoCas9 base editor fusion proteins provided herein do not comprise a linker sequence. In some embodiments, one or both of the optional linker sequences are present. In some embodiments, one, two, or three of the optional linker sequences are present.

In some embodiments, the “-” used in the general architecture above indicates the presence of an optional linker sequence. In some embodiments, the GeoCas9 base editor fusion proteins comprising a UGI further comprise a nuclear targeting sequence, for example a nuclear localization sequence. In some embodiments, GeoCas9 base editor fusion proteins provided herein further comprise a nuclear localization sequence (NLS). In some embodiments, the NLS is fused to the N-terminus of the GeoCas9 base editor fusion protein. In some embodiments, the NLS is fused to the C-terminus of the GeoCas9 base editor fusion protein. In some embodiments, the NLS is fused to the N-terminus of the UGI protein. In some embodiments, the NLS is fused to the C-terminus of the UGI protein. In some embodiments, the NLS is fused to the N-terminus of the Cas9 protein. In some embodiments, the NLS is fused to the C-terminus of the Cas9 protein. In some embodiments, the NLS is fused to the N-terminus of the deaminase. In some embodiments, the NLS is fused to the C-terminus of the deaminase. In some embodiments, the NLS is fused to the N-terminus of the second Cas9. In some embodiments, the NLS is fused to the C-terminus of the second Cas9. In some embodiments, the NLS is fused to the GeoCas9 base editor fusion protein via one or more linkers. In some embodiments, the NLS is fused to the fusioin protein without a linker. In some embodiments, the NLS comprises an amino acid sequence of any one of the NLS sequences provided or referenced herein. In some embodiments, the NLS comprises an amino acid sequence as set forth in SEQ ID NO: 75 or SEQ ID NO: 76.

In a preferred embodiment, the GeoCas9 base editor fusion protein may comprise the following structures:
[NH²]-[nucleic acid effector domain]-[GeoCas9]-[UGI domain]-[COOH];
[NH²]-[nucleic acid effector domain]-[GeoCas9]-[UGI domain]-[NLS]-[COOH];
[NH²]-[nucleic acid effector domain]-[GeoCas9]-[first UGI domain]-[second UGI domain]-[COOH]; or
[NH²]-[nucleic acid effector domain]-[GeoCas9]-[first UGI domain]-[second UGI domain]-[NLS]-[COOH].

In some embodiments, the nucleic acid effector domain and the GeoCas9 are fused via a linker comprising or consisting of the amino acid sequence SGSETPGTSESATPES (SEQ ID NO: 16), also referred to as the “XTEN linker”. In some embodiments, the nucleic acid effector domain and the GeoCas9 are fused via a linker comprising or consisting of the amino acid sequence SGGSSGGSSGSETPGTSESATPESSGGSSGGS (SEQ ID NO: 17).

In some embodiments, the GeoCas9 and the UGI domain are vused via a linker comprising or consisting of the amino acid sequence SGGS (SEQ ID NO: 14). In some embodiments, the GeoCas9 and the first UGI domain are vused via a linker comprising or consisting of the amino acid sequence SGGS (SEQ ID NO: 14). In some embodiments, the first UGI domain and the second UGI domain are fused via a linker comprising or consisting of the amino acid sequence SGGSGGSGGS (SEQ ID NO: 24). In some embodiments, the cytidine deaminase domain and the GeoCas9 are fused via a linker comprising or consisting of the amino acid sequence SGSETPGTSESATPES (SEQ ID NO: 15), and the GeoCas9 and the UGI domain are fused via a linker comprising or consisting of the amino acid sequence SGGS (SEQ ID NO: 14). In some embodiments, the nucleic acid effector domain and the GeoCas9 are fused via a linker comprising or consisting of the amino acid sequence SGGSSGGSSGSETPGTSESATPESSGGSSGGS (SEQ ID NO: 17), the GeoCas9 and the first UGI domain are fused via a linker comprising or consisting of the amino acid sequence SGGS (SEQ ID NO: 14), and the first UGI domain and the second UGI domain are fused via a linker comprising or consisting of the amino acid sequence SGGSGGSGGS (SEQ ID NO: 24)

In the GeoCas9 base editor fusion protein structures provided above, NLS is a nuclear localization sequence, NH₂ is the N-terminus of the GeoCas9 base editor fusion protein, COOH is the C-terminus of the GeoCas9 base editor fusion protein, and each instance of “-” comprises an optional linker, for example any of the linkers provided herein.

It should be appreciated that any of the GeoCas9 base editor fusion proteins described above may be comprised of (i) a GeoCas9 (e.g., a nGeoCas9 or dGeoCas9); (ii) an effector domain (e.g., cytidine or adenosine deaminase); and optionally (iii) two or more UGI domains, wherein the two or more UGI domains may be adjacent (e.g., [first UGI domain]-[second UGI domain], wherein “-” is an optional linker) to one another in the construct, or the two or more UGI domains may be separated by the GeoCas9 of (i) and/or the cytidine deaminase domain of (ii) (e.g., [first UGI domain]-[deaminase domain]-[second UGI domain], [first UGI domain]-[napDNAbp]-[second UGI domain], [first UGI domain]-[deaminase domain]-[napDNAbp]-[second UGI domain], ect., wherein “-” is an optional linker).

In another aspect, the GeoCas9 base editor fusion protein comprises: (i) a GeoCas9; (ii) a nucleic acid-editing enzyme or domain (e.g., a second protein, such as a cytidine deaminase domain); (iii) a first uracil glycosylase inhibitor domain (UGI) (e.g., a third protein); and (iv) a second uracil glycosylase inhibitor domain (UGI) (e.g., a fourth protein). The first and second uracil glycosylase inhibitor domains (UGIs) may be the same or different. In some embodiments, the GeoCas9 (e.g., the first protein) and the deaminase (e.g., the second protein) are fused via a linker. In some embodiments, the GeoCas9 is fused to the C-terminus of the deaminase. In some embodiments, the GeoCas9 protein (e.g., the first protein) and the first UGI domain (e.g., the third protein) are fused via a linker (e.g., a second linker). In some embodiments, the first UGI domain is fused to the C-terminus of the GeoCas9 protein. In some embodiments, the first UGI domain (e.g., the third protein) and the second UGI domain (e.g., the forth protein) are fused via a linker (e.g., a third linker). In some embodiments, the second UGI domain is fused to the C-terminus of the first UGI domain. In some embodiments, the linker comprises a (GGGS)ₙ (SEQ ID NO: 9), (GGGGS)ₙ (SEQ ID NO: 10), a (G)ₙ (SEQ ID NO: 11), an (EAAAK)ₙ (SEQ ID NO: 12), a (GGS)ₙ (SEQ ID NO: 13), (SGGS)ₙ (SEQ ID NO: 14), a SGSETPGTSESATPES (SEQ ID NO: 15), a SGGS (GGS)ₙ (SEQ ID NO: 16), a SGGSSGGSSGSETPGTSESATPESSGGSSGGS (SEQ ID NO: 17), or an (XP)ₙ (SEQ ID NO: 18) motif, or a combination of any of these, wherein n is independently an integer between 1 and 30.

### II. Guide RNA

In various embodiments, the GeoCas9 base editors described herein may be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design embodiments of a guide sequence will depend upon the nucleotide sequence of a genomic target site of interest (i.e., the desired site to be edited) and the particular GeoCas9 protein present in the base editor, among other factors, such as PAM sequence locations, percent G/C content in the target sequence, the degree of microhomology regions, secondary structures, etc.

In general, a guide sequence is any polynucleotide sequence having sufficient complementarity with a target polynucleotide sequence to hybridize with the target sequence and direct sequence-specific binding of a GeoCas9 to a target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length.

In some embodiments, a guide sequence is less than about 75, 50, 45, 40, 35, 30, 25, 20, 15, 12, or fewer nucleotides in length. The ability of a guide sequence to direct sequence-specific binding of a base editor to a target sequence may be assessed by any suitable assay. For example, the components of a base editor, including the guide sequence to be tested, may be provided to a host cell having the corresponding target sequence, such as by transfection with vectors encoding the components of a base editor disclosed herein, followed by an assessment of preferential cleavage within the target sequence, such as by Surveyor assay as described herein. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker & Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see, e.g., A. R. Gruber et al., 2008, Cell 106 (1): 23-24; and P A Carr & GM Church, 2009, Nature Biotechnology 27 (12): 1151-62). Additional algorithms may be found in Chuai, G. et al., DeepCRISPR: optimized CRISPR guide RNA design by deep learning, Genome Biol. 19:80 (2018), and U.S. application Ser. No. 61/836,080, the entireties of each of which are incorporated herein by reference.

The guide sequence can be linked to a tracr mate sequence which in turn hybridizes to a tracr sequence. A tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a complex at a target sequence, wherein the complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence.

In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. Preferred loop forming sequences for use in hairpin structures are four nucleotides in length, and most preferably have the sequence GAAA. However, longer or shorter loop sequences may be used, as may alternative sequences. The sequences preferably include a nucleotide triplet (for example, AAA), and an additional nucleotide (for example C or G). Examples of loop forming sequences include CAAA and AAAG. In an embodiment of the disclosure, the transcript or transcribed polynucleotide sequence has at least two or more hairpins. In certain embodiments, the transcript has two, three, four or five hairpins. In a further embodiment of the disclosure, the transcript has at most five hairpins. In some embodiments, the single transcript further includes a transcription termination sequence; preferably this is a polyT sequence, for example six T nucleotides. Further non-limiting examples of single polynucleotides comprising a guide sequence, a tracr mate sequence, and a tracr sequence are as follows (listed 5′ to 3′), where “N” represents a base of a guide sequence, the first block of lower case letters represent the tracr mate sequence, and the second block of lower case letters represent the tracr sequence, and the final poly-T sequence represents the transcription terminator:

(SEQ ID NO: 376)
(1) NNNNNNNNgtttttgtactctcaagatttaGAAAtaaatcttgcag
aagctacaaagataaggcttcatgccgaaatcaacaccctgtcattttat
ggcagggtgttttcgttatttaaTTTTTT;
(SEQ ID NO: 377)
(2)NNNNNNNNNNNNNNNNNNgtttttgtactctcaGAAAtgcagaagct
acaaagataaggcttcatgccgaaatca acaccctgtcattttatggca
gggtgttttcgttatttaaTTTTTT;
(SEQ ID NO: 378)
(3)NNNNNNNNNNNNNNNNNNNNgtttttgtactctcaGAAAtgcagaag
ctacaaagataaggcttcatgccgaaatca acaccctgtcattttatgg
cagggtgtTTTTT;
(SEQ ID NO: 379)
(4)NNNNNNNNNNNNNNNNNNNNgttttagagctaGAAAtagcaagttaa
aataaggctagtccgttatcaacttgaaaa agtggcaccgagtcggtgc
TTTTTT;
(SEQ ID NO: 380)
(5)NNNNNNNNNNNNNNNNNNNNgttttagagctaGAAATAGcaagttaa
aataaggctagtccgttatcaacttgaa aaagtgTTTTTTT;
and
(SEQ ID NO: 381)
(6)NNNNNNNNNNNNNNNNNNNNgttttagagctagAAATAGcaagttaa
aataaggctagtccgttatcaTTTTT TTT.

In some embodiments, the tracr sequence is a separate transcript from a transcript comprising the tracr mate sequence.

It will be apparent to those of skill in the art that in order to target any of the herein disclosed GeoCas9 fusion proteins to a target site, e.g., a site comprising a point mutation to be edited, it is typically necessary to co-express the fusion protein together with a guide RNA. A guide RNA typically comprises a tracrRNA framework allowing for GeoCas9 binding, and a guide sequence or spacer sequence, which confers sequence specificity to the Cas9: nucleic acid editing enzyme/domain fusion protein.

In some embodiments, the guide RNA comprises a structure 5′-[guide sequence]-guuuuagagcuagaaauagcaaguuaaaauaaggcuaguccguuaucaacuugaaaaaguggcaccgagucggugcuuu uu-3′ (SEQ ID NO: 382), wherein the guide sequence comprises a sequence that is complementary to the target sequence. See U.S. Publication No. 2015/0166981, published Jun. 18, 2015, the disclosure of which is incorporated by reference herein in its entirety. The guide sequence is typically 20 nucleotides long.

The sequences of suitable guide RNAs for targeting GeoCas9: nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

Additional guide sequences are well known in the art and can be used with the base editors described herein. Additional exemplary guide sequences are disclosed in, for example, Jinek M., et al., Science 337:816-821 (2012); Mali P, Esvelt K M & Church G M (2013) Cas9 as a versatile tool for engineering biology, Nature Methods, 10, 957-963; Li J F et al., (2013) Multiplex and homologous recombination-mediated genome editing in Arabidopsis and Nicotiana benthamiana using guide RNA and Cas9, Nature Biotechnology, 31, 688-691; Hwang, W. Y. et al., Efficient genome editing in zebrafish using a CRISPR-Cas system, Nature Biotechnology 31, 227-229 (2013); Cong L et al., (2013) Multiplex genome engineering using CRIPSR/Cas systems, Science, 339, 819-823; Cho S W et al., (2013) Targeted genome engineering in human cells with the Cas9 RNA-guided endonuclease, Nature Biotechnology, 31, 230-232; Jinek, M. et al., RNA-programmed genome editing in human cells, eLife 2, e00471 (2013); Dicarlo, J. E. et al., Genome engineering in Saccharomyces cerevisiae using CRISPR-Cas systems. Nucleic Acid Res. (2013); Briner A E et al., (2014) Guide RNA functional modules direct Cas9 activity and orthogonality, Mol Cell, 56, 333-339, the entire contents of each of which are herein incorporated by reference.

### III. GeoCas9 BE/Guide RNA Complexes

Some aspects of this disclosure provide complexes comprising any of the GeoCas9 base editor fusion proteins provided herein and a guide RNA.

In various embodiments, the guide RNA is bound to a GeoCas9 protein (e.g., a dGeoCas9, a nuclease active GeoCas9, or a GeoCas9 nickase) of GeoCas9 base editor fusion protein and functions to guide the GeoCas9 fusion protein to a target nucleotide sequence.

In some embodiments, the guide RNA is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary (e.g., perfectly complementary) to a target sequence. In some embodiments, the target sequence is a DNA sequence. In some embodiments, the target sequence is a sequence in the genome of a mammal. In some embodiments, the target sequence is a sequence in the genome of a human. In some embodiments, the 3′ end of the target sequence is immediately adjacent to a PAM sequence (e.g., 5′-NNNNCRAA-3′ (SEQ ID NO: 79)), such as any of the PAM sequences provided herein. In some embodiments, the guide RNA is complementary to a sequence associated with a disease or disorder.

In some embodiments, the target sequence comprises a sequence associated with a disease or disorder. In some embodiments, the target sequence comprises a point mutation associated with a disease or disorder. In some embodiments, the target sequence comprises a T→C point mutation. In some embodiments, the complex deaminates the target C point mutation, wherein the deamination results in a sequence that is not associated with a disease or disorder. In some embodiments, the target C point mutation is present in the DNA strand that is not complementary to the guide RNA. In some embodiments, the target sequence comprises a T->A point mutation. In some embodiments, the complex deaminates the target A point mutation, and wherein the deamination results in a sequence that is not associated with a disease or disorder. In some embodiments, the target A point mutation is present in the DNA strand that is not complementary to the guide RNA.

In some embodiments, the complex edits a point mutation in the target sequence. In some embodiments, the point mutation is located between about 10 to about 20 nucleotides upstream of the PAM in the target sequence. In some embodiments, the point mutation is located between about 13 to about 17 nucleotides upstream of the PAM in the target sequence. In some embodiments, the point mutation is about 13 nucleotides upstream of the PAM. In some embodiments, the point mutation is about 14 nucleotides upstream of the PAM. In some embodiments, the point mutation is about 15 nucleotides upstream of the PAM. In some embodiments, the point mutation is about 16 nucleotides upstream of the PAM. In some embodiments, the point mutation is about 17 nucleotides upstream of the PAM.

In some embodiments, the complex exhibits increased deamination efficiency of a point mutation in a target sequence that does not comprise the canonical PAM (5′-NGG-3′) at its 3′ end as compared to the deamination efficiency of a complex comprising Streptococcus pyogenes Cas9 as provided by SEQ ID NO: 1. In some embodiments, the complex exhibits increased deamination efficiency of a point mutation in a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold increased as compared to the deamination efficiency of complex comprising the Streptococcus pyogenes Cas9 as provided by SEQ ID NO: 1 on the same target sequence. In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA. In some embodiments, deamination activity is measured using high-throughput sequencing.

In some embodiments, the complex produces fewer indels in a target sequence that does not comprise the canonical PAM (5′-NGG-3′) at its 3′ end as compared to the amount of indels produced by a complex comprising Streptococcus pyogenes Cas9 as provided by SEQ ID NO: 1. In some embodiments, the complex produces fewer indels in a target sequence having a 3′ end that is not directly adjacent to the canonical PAM sequence (5′-NGG-3′) that is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold lower as compared to the amount of indels produced by a complex comprising Streptococcus pyogenes Cas9 as provided by SEQ ID NO: 1 on the same target sequence. In some embodiments, the 3′ end of the target sequence is directly adjacent to a sequence selected from the group consisting of NGT, NGA, NGC, and NNG, wherein N is an A, G, T, or C. In some embodiments, the 3′ end of the target sequence is directly adjacent to a sequence selected from the group consisting of CGG, AGT, TGG, AGT, CGT, GGG, CGT, TGT, GGT, AGC, CGC, TGC, GGC, AGA, CGA, TGA, GGA, GAA, GAT, and CAA. In some embodiments, indels are measured using high-throughput sequencing.

In some embodiments, the complex exhibits a decreased off-target activity as compared to the off-target activity of a complex comprising Streptococcus pyogenes Cas9 as provided by SEQ ID NO: 1. In some embodiments, the off-target activity of the complex is at least 2-fold, at least 3-fold, at least 4-fold, at least 5-fold, at least 10-fold, at least 50-fold, at least 100-fold, at least 500-fold, at least 1,000-fold, at least 5,000-fold, at least 10,000-fold, at least 50,000-fold, at least 100,000-fold, at least 500,000-fold, or at least 1,000,000-fold decreased as compared to the off-target activity of a complex comprising Streptococcus pyogenes Cas9 as provided by SEQ ID NO: 1. In some embodiments, the off-target activity is determined using a genome-wide off-target analysis. In some embodiments, the off-target activity is determined using GUIDE-seq.

### IV. Methods of Using GeoCas9 Fusion Proteins

Some aspects of this disclosure provide methods of using the GeoCas9 proteins, fusion proteins, or complexes provided herein. For example, some aspects of this disclosure provide methods comprising contacting a DNA molecule (a) with any of the the GeoCas9 proteins or fusion proteins provided herein, and with at least one guide RNA, wherein the guide RNA is about 15-100 nucleotides long and comprises a sequence of at least 10 contiguous nucleotides that is complementary to a target sequence; or (b) with a GeoCas9 protein, a GeoCas9 fusion protein, or a GeoCas9 protein or fusion protein complex with at least one gRNA as provided herein. In some embodiments, the 3′ end of the target sequence is not immediately adjacent to a canonical PAM sequence (e.g., 5′-NNNNCRAA-3′ (SEQ ID NO: 79)), sush as any of the PAM sequences provided herein.

In some embodiments, the target DNA sequence comprises a sequence associated with a disease or disorder. In some embodiments, the target DNA sequence comprises a point mutation associated with a disease or disorder. In some embodiments, the activity of the Cas9 protein, the Cas9 fusion protein, or the complex results in a correction of the point mutation. In some embodiments, the target DNA sequence comprises a T->C point mutation associated with a disease or disorder, and wherein the deamination of the mutant C base results in a sequence that is not associated with a disease or disorder. In some embodiments, the target DNA sequence encodes a protein and wherein the point mutation is in a codon and results in a change in the amino acid encoded by the mutant codon as compared to the wild-type codon. In some embodiments, the deamination of the mutant C results in a change of the amino acid encoded by the mutant codon. In some embodiments, the deamination of the mutant C results in the codon encoding the wild-type amino acid. In some embodiments, the contacting is in vivo in a subject. In some embodiments, the subject has or has been diagnosed with a disease or disorder. In some embodiments, the disease or disorder is cystic fibrosis, phenylketonuria, epidermolytic hyperkeratosis (EHK), Charcot-Marie-Toot disease type 4J, neuroblastoma (NB), von Willebrand disease (vWD), myotonia congenital, hereditary renal amyloidosis, dilated cardiomyopathy (DCM), hereditary lymphedema, familial Alzheimer's disease, HIV, Prion disease, chronic infantile neurologic cutaneous articular syndrome (CINCA), desmin-related myopathy (DRM), a neoplastic disease associated with a mutant PI3KCA protein, a mutant CTNNB 1 protein, a mutant HRAS protein, or a mutant p53 protein.

In some embodiments, any of the base editors provided herein may be used to treat a disease or disorder. For example, any base editors provided herein may be used to correct one or more mutations associated with any of the diseases or disorders provided herein. Exemplary diseases or disorders that may be treated include, without limitation, those listed in Table 1 and Table 2, as well as 3-Methylglutaconic aciduria type 2, 46,XY gonadal dysgenesis, 4-Alpha-hydroxyphenylpyruvate hydroxylase deficiency, 6-pyruvoyl-tetrahydropterin synthase deficiency, achromatopsia, Acid-labile subunit deficiency, Acrodysostosis, acroerythrokeratoderma, ACTH resistance, ACTH-independent macronodular adrenal hyperplasia, Activated PI3K-delta syndrome, Acute intermittent porphyria, Acute myeloid leukemia, Adams-Oliver syndrome 1/5/6, Adenylosuccinate lyase deficiency, Adrenoleukodystrophy, Adult neuronal ceroid lipofuscinosis, Adult onset ataxia with oculomotor apraxia, Advanced sleep phase syndrome, Age-related macular degeneration, Alagille syndrome, Alexander disease, Allan-Herndon-Dudley syndrome, Alport syndrome, X-linked recessive, Alternating hemiplegia of childhood, Alveolar capillary dysplasia with misalignment of pulmonary veins, Amelogenesis imperfecta, Amyloidogenic transthyretin amyloidosis, Amyotrophic lateral sclerosis, Anemia (nonspherocytic hemolytic, due to G6PD deficiency), Anemia (sideroblastic, pyridoxine-refractory, autosomal recessive), Anonychia, Antithrombin III deficiency, Aortic aneurysm, Aplastic anemia, Apolipoprotein C2 deficiency, Apparent mineralocorticoid excess, Aromatase deficiency, Arrhythmogenic right ventricular cardiomyopathy, Familial hypertrophic cardiomyopathy, Hypertrophic cardiomyopathy, Arthrogryposis multiplex congenital, Aspartylglycosaminuria, Asphyxiating thoracic dystrophy, Ataxia with vitamin E deficiency, Ataxia (spastic), Atrial fibrillation, Atrial septal defect, atypical hemolytic-uremic syndrome, autosomal dominant CD11C+/CD1C+ dendritic cell deficiency, Autosomal dominant progressive external ophthalmoplegia with mitochondrial DNA deletions, Baraitser-Winter syndrome, Bartter syndrome, Basa ganglia calcification, Beckwith-Wiedemann syndrome, Benign familial neonatal seizures, Benign scapuloperoneal muscular dystrophy, Bernard Soulier syndrome, Beta thalassemia intermedia, Beta-D-mannosidosis, Bietti crystalline corneoretinal dystrophy, Bile acid malabsorption, Biotinidase deficiency, Borjeson-Forssman-Lehmann syndrome, Boucher Neuhauser syndrome, Bowen-Conradi syndrome, Brachydactyly, Brown-Vialetto-Van laere syndrome, Brugada syndrome, Cardiac arrhythmia, Cardiofaciocutaneous syndrome, Cardiomyopathy, Carnevale syndrome, Carnitine palmitoyltransferase II deficiency, Carpenter syndrome, Cataract, Catecholaminergic polymorphic ventricular tachycardia, Central core disease, Centromeric instability of chromosomes 1,9 and 16 and immunodeficiency, Cerebral autosomal dominant arteriopathy, Cerebro-oculo-facio-skeletal syndrome, Ceroid lipofuscinosis, Charcot-Marie-Tooth disease, Cholestanol storage disease, Chondrocalcinosis, Chondrodysplasia, Chronic progressive multiple sclerosis, Coenzyme Q10 deficiency, Cohen syndrome, Combined deficiency of factor V and factor VIII, Combined immunodeficiency, Combined oxidative phosphorylation deficiency, Combined partial 17-alpha-hydroxylase/17,20-lyase deficiency, Complement factor d deficiency, Complete combined 17-alpha-hydroxylase/17,20-lyase deficiency, Cone-rod dystrophy, Congenital contractural arachnodactyly, Congenital disorder of glycosylation, Congenital lipomatous overgrowth, Neoplasm of ovary, PIK3CA Related Overgrowth Spectrum, Congenital long QT syndrome, Congenital muscular dystrophy, Congenital muscular hypertrophy-cerebral syndrome, Congenital myasthenic syndrome, Congenital myopathy with fiber type disproportion, Eichsfeld type congenital muscular dystrophy, Congenital stationary night blindness, Corneal dystrophy, Cornelia de Lange syndrome, Craniometaphyseal dysplasia, Crigler Najjar syndrome, Crouzon syndrome, Cutis laxa with osteodystrophy, Cyanosis, Cystic fibrosis, Cystinosis, Cytochrome-c oxidase deficiency, Mitochondrial complex I deficiency, D-2-hydroxyglutaric aciduria, Danon disease, Deafness with labyrinthine aplasia microtia and microdontia (LAMM), Deafness, Deficiency of acetyl-CoA acetyltransferase, Deficiency of ferroxidase, Deficiency of UDPglucose-hexose-1-phosphate uridylyltransferase, Dejerine-Sottas disease, Desbuquois syndrome, DFNA, Diabetes mellitus type 2, Diabetes-deafness syndrome, Diamond-Blackfan anemia, Diastrophic dysplasia, Dihydropteridine reductase deficiency, Dihydropyrimidinase deficiency, Dilated cardiomyopathy, Disseminated atypical mycobacterial infection, Distal arthrogryposis, Distal hereditary motor neuronopathy, Donnai Barrow syndrome, Duchenne muscular dystrophy, Becker muscular dystrophy, Dyschromatosis universalis hereditaria, Dyskeratosis congenital, Dystonia, Early infantile epileptic encephalopathy, Ehlers-Danlos syndrome, Eichsfeld type congenital muscular dystrophy, Emery-Dreifuss muscular dystrophy, Enamel-renal syndrome, Epidermolysis bullosa dystrophica inversa, Epidermolysis bullosa herpetiformis, Epilepsy, Episodic ataxia, Erythrokeratodermia variabilis, Erythropoietic protoporphyria, Exercise intolerance, Exudative vitreoretinopathy, Fabry disease, Factor V deficiency, Factor VII deficiency, Factor xiii deficiency, Familial adenomatous polyposis, breast cancer, ovarian cancer, cold urticarial, chronic infantile neurological, cutaneous and articular syndrome, hemiplegic migraine, hypercholesterolemia, hypertrophic cardiomyopathy, hypoalphalipoproteinemia, hypokalemia-hypomagnesemia, juvenile gout, hyperlipoproteinemia, visceral amyloidosis, hypophosphatemic vitamin D refractory rickets, FG syndrome, Fibrosis of extraocular muscles, Finnish congenital nephrotic syndrome, focal epilepsy, Focal segmental glomerulosclerosis, Frontonasal dysplasia, Frontotemporal dementia, Fructose-biphosphatase deficiency, Gamstorp-Wohlfart syndrome, Ganglioside sialidase deficiency, GATA-1-related thrombocytopenia, Gaucher disease, Giant axonal neuropathy, Glanzmann thrombasthenia, Glomerulocystic kidney disease, Glomerulopathy, Glucocorticoid resistance, Glucose-6-phosphate transport defect, Glutaric aciduria, Glycogen storage disease, Gorlin syndrome, Holoprosencephaly, GRACILE syndrome, Hemorrhagic telangiectasia, Hemochromatosis, Hemoglobin H disease, Hemolytic anemia, Hemophagocytic lymphohistiocytosis, Carcinoma of colon, Myhre syndrome, leukoencephalopathy, Hereditary factor IX deficiency disease, Hereditary factor VIII deficiency disease, Hereditary factor XI deficiency disease, Hereditary fructosuria, Hereditary Nonpolyposis Colorectal Neoplasm, Hereditary pancreatitis, Hereditary pyropoikilocytosis, Elliptocytosis, Heterotaxy, Heterotopia, Histiocytic medullary reticulosis, Histiocytosis-lymphadenopathy plus syndrome, HNSHA due to aldolase A deficiency, Holocarboxylase synthetase deficiency, Homocysteinemia, Howel-Evans syndrome, Hydatidiform mole, Hypercalciuric hypercalcemia, Hyperimmunoglobulin D, Mevalonic aciduria, Hyperinsulinemic hypoglycemia, Hyperkalemic Periodic Paralysis, Paramyotonia congenita of von Eulenburg, Hyperlipoproteinemia, Hypermanganesemia, Hypermethioninemia, Hyperphosphatasemia, Hypertension, hypomagnesemia, Hypobetalipoproteinemia, Hypocalcemia, Hypogonadotropic hypogonadism, Hypogonadotropic hypogonadism, Hypohidrotic ectodermal dysplasia, Hyper-IgM immunodeficiency, Hypohidrotic X-linked ectodermal dysplasia, Hypomagnesemia, Hypoparathyroidism, Idiopathic fibrosing alveolitis, Immunodeficiency, Immunoglobulin A deficiency, Infantile hypophosphatasia, Infantile Parkinsonism-dystonia, Insulin-dependent diabetes mellitus, Intermediate maple syrup urine disease, Ischiopatellar dysplasia, Islet cell hyperplasia, Isolated growth hormone deficiency, Isolated lutropin deficiency, Isovaleric acidemia, Joubert syndrome, Juvenile polyposis syndrome, Juvenile retinoschisis, Kallmann syndrome, Kartagener syndrome, Kugelberg-Welander disease, Lattice corneal dystrophy, Leber congenital amaurosis, Leber optic atrophy, Left ventricular noncompaction, Leigh disease, Mitochondrial complex I deficiency, Leprechaunism syndrome, Arthrogryposis, Anterior horn cell disease, Leukocyte adhesion deficiency, Leukodystrophy, Leukoencephalopathy, Ovarioleukodystrophy, L-ferritin deficiency, Li-Fraumeni syndrome, Limb-girdle muscular dystrophy-dystroglycanopathy, Loeys-Dietz syndrome, Long QT syndrome, Macrocephaly/autism syndrome, Macular corneal dystrophy, Macular dystrophy, Malignant hyperthermia susceptibility, Malignant tumor of prostate, Maple syrup urine disease, Marden Walker like syndrome, Marfan syndrome, Marie Unna hereditary hypotrichosis, Mast cell disease, Meconium ileus, Medium-chain acyl-coenzyme A dehydrogenase deficiency, Melnick-Fraser syndrome, Mental retardation, Merosin deficient congenital muscular dystrophy, Mesothelioma, Metachromatic leukodystrophy, Metaphyseal chondrodysplasia, Methemoglobinemia, methylmalonic aciduria, homocystinuria, Microcephaly, chorioretinopathy, lymphedema, Microphthalmia, Mild non-PKU hyperphenylalanemia, Mitchell-Riley syndrome, mitochondrial 3-hydroxy-3-methylglutaryl-CoA synthase deficiency, Mitochondrial complex I deficiency, Mitochondrial complex III deficiency, Mitochondrial myopathy, Mucolipidosis III, Mucopolysaccharidosis, Multiple sulfatase deficiency, Myasthenic syndrome, Mycobacterium tuberculosis, Myeloperoxidase deficiency, Myhre syndrome, Myoclonic epilepsy, Myofibrillar myopathy, Myoglobinuria, Myopathy, Myopia, Myotonia congenital, Navajo neurohepatopathy, Nemaline myopathy, Neoplasm of stomach, Nephrogenic diabetes insipidus, Nephronophthisis, Nephrotic syndrome, Neurofibromatosis, Neutral lipid storage disease, Niemann-Pick disease, Non-ketotic hyperglycinemia, Noonan syndrome, Noonan syndrome-like disorder, Norum disease, Macular degeneration, N-terminal acetyltransferase deficiency, Oculocutaneous albinism, Oculodentodigital dysplasia, Ohdo syndrome, Optic nerve aplasia, Ornithine carbamoyltransferase deficiency, Orofaciodigital syndrome, Osteogenesis imperfecta, Osteopetrosis, Ovarian dysgenesis, Pachyonychia, Palmoplantar keratoderma, nonepidermolytic, Papillon-Lef\xc3\xa8vre syndrome, Haim-Munk syndrome, Periodontitis, Peeling skin syndrome, Pendred syndrome, Peroxisomal fatty acyl-coa reductase 1 disorder, Peroxisome biogenesis disorder, Pfeiffer syndrome, Phenylketonuria, Phenylketonuria, Hyperphenylalaninemia, non-PKU, Pituitary hormone deficiency, Pityriasis rubra pilaris, Polyarteritis nodosa, Polycystic kidney disease, Polycystic lipomembranous osteodysplasia, Polymicrogyria, Pontocerebellar hypoplasia, Porokeratosis, Posterior column ataxia, Primary erythromelalgia, hyperoxaluria, Progressive familial intrahepatic cholestasis, Progressive pseudorheumatoid dysplasia, Propionic acidemia, Pseudohermaphroditism, Pseudohypoaldosteronism, Pseudoxanthoma elasticum-like disorder, Purine-nucleoside phosphorylase deficiency, Pyridoxal 5-phosphate-dependent epilepsy, Renal dysplasia, retinal pigmentary dystrophy, cerebellar ataxia, skeletal dysplasia, Reticular dysgenesis, Retinitis pigmentosa, Usher syndrome, Retinoblastoma, Retinopathy, RRM2B-related mitochondrial disease, Rubinstein-Taybi syndrome, Schnyder crystalline corneal dystrophy, Sebaceous tumor, Severe congenital neutropenia, Severe myoclonic epilepsy in infancy, Severe X-linked myotubular myopathy, onychodysplasia, facial dysmorphism, hypotrichosis, Short-rib thoracic dysplasia, Sialic acid storage disease, Sialidosis, Sideroblastic anemia, Small fiber neuropathy, Smith-Magenis syndrome, Sorsby fundus dystrophy, Spastic ataxia, Spastic paraplegia, Spermatogenic failure, Spherocytosis, Sphingomyelin/cholesterol lipidosis, Spinocerebellar ataxia, Split-hand/foot malformation, Spondyloepimetaphyseal dysplasia, Platyspondylic lethal skeletal dysplasia, Squamous cell carcinoma of the head and neck, Stargardt disease, Sucrase-isomaltase deficiency, Sudden infant death syndrome, Supravalvar aortic stenosis, Surfactant metabolism dysfunction, Tangier disease, Tatton-Brown-rahman syndrome, Thoracic aortic aneurysms and aortic dissections, Thrombophilia, Thyroid hormone resistance, TNF receptor-associated periodic fever syndrome (TRAPS), Tooth agenesis, Torsades de pointes, Transposition of great arteries, Treacher Collins syndrome, Tuberous sclerosis syndrome, Tyrosinase-negative oculocutaneous albinism, Tyrosinase-positive oculocutaneous albinism, Tyrosinemia, UDPglucose-4-epimerase deficiency, Ullrich congenital muscular dystrophy, Bethlem myopathy Usher syndrome, UV-sensitive syndrome, Van der Woude syndrome, popliteal pterygium syndrome, Very long chain acyl-CoA dehydrogenase deficiency, Vesicoureteral reflux, Vitreoretinochoroidopathy, Von Hippel-Lindau syndrome, von Willebrand disease, Waardenburg syndrome, Warsaw breakage syndrome, WFS1-Related Disorders, Wilson disease, Xeroderma pigmentosum, X-linked agammaglobulinemia, X-linked hereditary motor and sensory neuropathy, X-linked severe combined immunodeficiency, and Zellweger syndrome.

Some embodiments provide methods for using the Cas9 DNA editing fusion proteins provided herein. In some embodiments, the fusion protein is used to introduce a point mutation into a nucleic acid by deaminating a target nucleobase, e.g., a C residue. In some embodiments, the deamination of the target nucleobase results in the correction of a genetic defect, e.g., in the correction of a point mutation that leads to a loss of function in a gene product. In some embodiments, the genetic defect is associated with a disease or disorder, e.g., a lysosomal storage disorder or a metabolic disease, such as, for example, type I diabetes. In some embodiments, the methods provided herein are used to introduce a deactivating point mutation into a gene or allele that encodes a gene product that is associated with a disease or disorder. For example, in some embodiments, methods are provided herein that employ a Cas9 DNA editing fusion protein to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein.

In some embodiments, the purpose of the methods provide herein is to restore the function of a dysfunctional gene via genome editing. The Cas9 deaminase fusion proteins provided herein can be validated for gene editing-based human therapeutics in vitro, e.g., by correcting a disease-associated mutation in human cell culture. It will be understood by the skilled artisan that the fusion proteins provided herein, e.g., the fusion proteins comprising a Cas9 domain and a nucleic acid deaminase domain can be used to correct any single point T->C or A->G mutation. In the first case, deamination of the mutant C back to U corrects the mutation, and in the latter case, deamination of the C that is base-paired with the mutant G, followed by a round of replication, corrects the mutation.

The successful correction of point mutations in disease-associated genes and alleles opens up new strategies for gene correction with applications in therapeutics and basic research. Site-specific single-base modification systems like the disclosed fusions of GeoCas9 and deaminase enzymes or domains also have applications in “reverse” gene therapy, where certain gene functions are purposely suppressed or abolished. In these cases, site-specifically mutating Trp (TGG), Gln (CAA and CAG), or Arg (CGA) residues to premature stop codons (TAA, TAG, TGA) can be used to abolish protein function in vitro, ex vivo, or in vivo.

The instant disclosure provides methods for the treatment of a subject diagnosed with a disease associated with or caused by a point mutation that can be corrected by a GeoCas9 base editing fusion protein provided herein. For example, in some embodiments, a method is provided that comprises administering to a subject having such a disease, e.g., a cancer, an effective amount of a GeoCas9 deaminase fusion protein that corrects the point mutation or introduces a deactivating mutation into the disease-associated gene. In some embodiments, the disease is a proliferative disease. In some embodiments, the disease is a genetic disease. In some embodiments, the disease is a neoplastic disease. In some embodiments, the disease is a metabolic disease. In some embodiments, the disease is a lysosomal storage disease. Other diseases that can be treated by correcting a point mutation or introducing a deactivating mutation into a disease-associated gene will be known to those of skill in the art, and the disclosure is not limited in this respect.

The instant disclosure provides methods for the treatment of additional diseases or disorders, e.g., diseases or disorders that are associated or caused by a point mutation that can be corrected by deaminase-mediated gene editing. Some such diseases are described herein, and additional suitable diseases that can be treated with the strategies and fusion proteins provided herein will be apparent to those of skill in the art based on the instant disclosure. Exemplary suitable diseases and disorders are provided herein. It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues. Exemplary suitable diseases and disorders include, without limitation, cystic fibrosis (see, e.g., Schwank et al., Functional repair of CFTR by CRISPR/Cas9 in intestinal stem cell organoids of cystic fibrosis patients. Cell stem cell. 2013; 13:653-658; and Wu et. al., Correction of a genetic disease in mouse via use of CRISPR-Cas9. Cell stem cell. 2013; 13:659-662, neither of which uses a deaminase fusion protein to correct the genetic defect); phenylketonuria—e.g., phenylalanine to serine mutation at position 835 (mouse) or 240 (human) or a homologous residue in phenylalanine hydroxylase gene (T>C mutation)—see, e.g., McDonald et al., Genomics. 1997; 39:402-405; Bernard-Soulier syndrome (BSS)—e.g., phenylalanine to serine mutation at position 55 or a homologous residue, or cysteine to arginine at residue 24 or a homologous residue in the platelet membrane glycoprotein IX (T>C mutation)—see, e.g., Noris et al., British Journal of Haematology. 1997; 97:312-320, and Ali et al., Hematol. 2014; 93:381-384; epidermolytic hyperkeratosis (EHK)—e.g., leucine to proline mutation at position 160 or 161 (if counting the initiator methionine) or a homologous residue in keratin 1 (T>C mutation)—see, e.g., Chipev et al., Cell. 1992; 70:821-828, see also accession number P04264 in the UNIPROT database at www [dot] uniprot [dot] org; chronic obstructive pulmonary disease (COPD)—e.g., leucine to proline mutation at position 54 or 55 (if counting the initiator methionine) or a homologous residue in the processed form of α₁-antitrypsin or residue 78 in the unprocessed form or a homologous residue (T>C mutation)—see, e.g., Poller et al., Genomics. 1993; 17:740-743, see also accession number P01011 in the UNIPROT database; Charcot-Marie-Toot disease type 4J—e.g., isoleucine to threonine mutation at position 41 or a homologous residue in FIG. 4 (T>C mutation)—see, e.g., Lenk et al., PLOS Genetics. 2011; 7: e1002104; neuroblastoma (NB)—e.g., leucine to proline mutation at position 197 or a homologous residue in Caspase-9 (T>C mutation)—see, e.g., Kundu et al., 3 Biotech. 2013, 3:225-234; von Willebrand disease (vWD)—e.g., cysteine to arginine mutation at position 509 or a homologous residue in the processed form of von Willebrand factor, or at position 1272 or a homologous residue in the unprocessed form of von Willebrand factor (T>C mutation)—see, e.g., Lavergne et al., Br. J. Haematol. 1992, see also accession number P04275 in the UNIPROT database; 82:66-72; myotonia congenital—e.g., cysteine to arginine mutation at position 277 or a homologous residue in the muscle chloride channel gene CLCN1 (T>C mutation)—see, e.g., Weinberger et al., The J. of Physiology. 2012; 590:3449-3464; hereditary renal amyloidosis—e.g., stop codon to arginine mutation at position 78 or a homologous residue in the processed form of apolipoprotein AII or at position 101 or a homologous residue in the unprocessed form (T>C mutation)—see, e.g., Yazaki et al., Kidney Int. 2003; 64:11-16; dilated cardiomyopathy (DCM)—e.g., tryptophan to Arginine mutation at position 148 or a homologous residue in the FOXD4 gene (T>C mutation), see, e.g., Minoretti et. al., Int. J. of Mol. Med. 2007; 19:369-372; hereditary lymphedema—e.g., histidine to arginine mutation at position 1035 or a homologous residue in VEGFR3 tyrosine kinase (A>G mutation), see, e.g., Irrthum et al., Am. J. Hum. Genet. 2000; 67:295-301; familial Alzheimer's disease—e.g., isoleucine to valine mutation at position 143 or a homologous residue in presenilin1 (A>G mutation), see, e.g., Gallo et. al., J. Alzheimer's disease. 2011; 25:425-431; Prion disease—e.g., methionine to valine mutation at position 129 or a homologous residue in prion protein (A>G mutation)—see, e.g., Lewis et. al., J. of General Virology. 2006; 87:2443-2449; chronic infantile neurologic cutaneous articular syndrome (CINCA)—e.g., Tyrosine to Cysteine mutation at position 570 or a homologous residue in cryopyrin (A>G mutation)—see, e.g., Fujisawa et. al. Blood. 2007; 109:2903-2911; and desmin-related myopathy (DRM)—e.g., arginine to glycine mutation at position 120 or a homologous residue in aß crystallin (A>G mutation)—see, e.g., Kumar et al., J. Biol. Chem. 1999; 274:24137-24141. The entire contents of all references and database entries is incorporated herein by reference.

Additional exemplary genes in the human genome that may be targeted by the base editors or complexes of this disclosure are provided herein in Tables 1 and 2. Table 1 includes gene mutations that may be correcteded by changing a cytosine (C) to a thymine (T), for example, using a GeoBE3 or GeoBE4 nucleobase editor. Table 2 includes gene mutations that may be corrected by changing a guanine (G) to an adenine (A), for example, using a GeoBE3 or GeoBE4. Identification of exemplary genes that may be targeted using any of the base editors provided herein were identified using the ClinVar database. The ‘gRNA’ tab shows the potential target sequence (followed by the PAM, which would not be included in the gRNA). In Table 1, which list targets for C to T changes, the target base is indicated by ‘Y’ (the corresponding gRNA would have a C here). In Table 2, which lists targets for G to A changes the target base is indicated as ‘R’ (the corresponding gRNA would have a G here). To account for a potential large window of editing for GeoBEs, include are diseases whose mutated base can be placed within position 2-12 of the window (PAM is in position 21-23). The PAM for GeoCas9 has been set to NNNNCRAA (SEQ ID NO: 79).

TABLE 1
Human gene mutations that may be corrected by changing a cytosine (C)
to a thymine (T) using GeoCas9 base editors provided herein. The gene name, gene symbol,
and dbSNP database reference number (RS#) are indicated. Also indicated are exemplary
protospacers with their PAM sequences (gRNAs) and the base to be edited, e.g., a C,
indicated by a “Y”. The “gRNAs” sequences correspond to SEQ ID NOs: 82-199. It should
be appreciated that each instance of thymine “T” in the gRNAs listed in the below tables is
uracil “U”, as uracil occurs in RNA.

| Name | RS# | | | | SEQ ID |
|---|---|---|---|---|---|
| (dbSNP) | Symbol | Gene | gRNAs | Phenotypes | NO(s): |
| NM_000071.2(CBS): | 5742905 | CBS | [‘TCTGCAGATCAYTGG | [‘Homocystinuria due to | 82 |
| c.833T > C (p.Ile278Thr) | | | GGTGGATCCCGAA’] | CBS deficiency’, | |
| | | | | ‘Homocystinuria, | |
| | | | | pyridoxine-responsive’, | |
| | | | | ‘not provided’] | |
| NM_000521.3(HEXB): | 820878 | HEXB | [‘TGCCGCTCTYGGTGA | [‘Sandhoff disease, | 83 |
| c.185C > T (p.Ser62Leu) | | | AGATGACCCCGAA’] | infantile type’] | |
| NM_139058.2(ARX): | 28936077 | ARX | [‘CATCCYGGGCCGGA | [‘Mental retardation, with | 84 |
| c.98T > C (p.Leu33Pro) | | | GGAGCCCGTGCAAA’] | or without seizures, | |
| | | | | ARX-related, X-linked’] | |
| NM_024960.4(PANK2): | 28939088 | PANK2 | [‘AYGGCATCTCGTGG | [‘Hypoprebetalipoprotein | 85, 86 |
| c.437T > C (p.Met146Thr) | | | AGATAGCACCAAA’, | emia, acanthocytosis, | |
| | | | ‘AAYGGCATCTCGTGG | retinitis pigmentosa, and | |
| | | | AGATAGCACCAAA’] | pallidal degeneration’] | |
| NM_000045.3(ARG1: | 28941474 | ARG1 | [‘TAGGGATTAYTGGA | [‘Arginase deficiency’] | 87, 87 |
| c.32T > C (p.Ile11Thr) | | | GCTCCTTTCTC AAA’]; | | |
| | | | [‘TAGGGATTAYTGGA | | |
| | | | GCTCCTTTCTCAAA’] | | |
| NM_024426.4(WT1): | 28941777 | WT1 | [‘CCAYTCCAGTGTAAA | [‘Diffuse mesangial | 88 |
| c.1351T > C (p.Phe451Leu) | | | ACTTGTCAGCGAA’] | sclerosis’] | |
| NM_000559.2(HBG1): | 35710727 | HBG1 | [‘CCCCTYCCCCACACT | [‘Fetal hemoglobin | 89 |
| c.-251T > C | | | ATCTCAATGCAAA’] | quantitative trait locus 1’] | |
| NM_000518.4(HBB): | 36015961 | HBB | [‘CTGTGTGCYGGCCCA | [‘Beta thalassemia | 90 |
| c.344T > C (p.Leu115Pro) | | | TCACTTTGGCAAA’] | intermedia’] | |
| m.3394T > C | 41460449 | MT-ND1 | [‘AATTCTAGGCYATAT | [‘Leber optic atrophy’] | 91 |
| | | | ACAACTACGCAAA’] | | |
| NM_000548.3(TSC2): | 45517281 | TSC2 | [‘TCTTCYCCAACTTCA | [‘Tuberous sclerosis | 92 |
| c.3106T > C | | | CGGCTGTCCCGAA’] | syndrome’, ‘Tuberous | |
| (p.Ser1036Pro) | | | | sclerosis 2’] | |
| NM_024514.4(C YP2R1): | 61495246 | CYP2R1 | [‘AAAGGAATGCCYTG | [‘Vitamin d | 93 |
| c.296T > C | | | TTCATCAAAGCGAA’] | hydroxylation-deficient | |
| (p.Leu99Pro) | | | | rickets, type lb’] | |
| NM_002618.3(PEX13): | 61752115 | PEX13 | [‘AGGACTTAYACCTGC | [‘Peroxisome biogenesis | 94 |
| c977T > C (p.Ile326Thr) | | | GAATTATGTCAAA’] | disorder 11B’] | |
| NM_014165.3(NDUFAF | 63751061 | NDUFAF4 | [‘CYAAAAGATGTGTA | [‘Mitochondrial complex | 97, 96, |
| 4):c.194T > C | | | TGTTGATTCCAAA’, | I deficiency’] | 97 |
| (p.Leu65Pro) | | | ‘TCYAAAAGATGTGTA | | |
| | | | TGTTGATTCCAAA’]; | | |
| | | | [‘CYAAAAGATGTGTA | | |
| | | | TGTTGATTCCAAA’] | | |
| NM_000104.3(CYP1B1): | 72549389 | CYP1B1 | [‘CCAGCAYGGGCACC | [‘Irido-corneo-trabecular | 98 |
| c.2T > C (p.Met1Thr) | | | AGCCTCAGCCCGAA’] | dysgenesis’] | |
| NM_000531.5(OTC): | 72554359 | OTC | [‘GYTTGTAAATATTTT | [‘not provided’] | 99, 100, |
| c.386 + 2T > C | | | CTTCTCTCCAAA’, | | 99, 100 |
| | | | ‘GGYTTGTAAATATTT | | |
| | | | TCTTCTCTCCAAA’]; | | |
| | | | [‘GYTTGTAAATATTTT | | |
| | | | CTTCTCTCCAAA’, | | |
| | | | ‘GGYTTGTAAATATTT | | |
| | | | TCTTCTCTCCAAA’] | | |
| NM_000098.2(CPT2): | 74315297 | CPT2 | [‘CTGCGTCCAGYTTCA | [‘CARNITINE | 101 |
| c.1342T > C (p.Phe448Leu) | | | GAGAGGAGGCAAA’] | PALMITOYLTRANSFE | |
| | | | | RASE II DEFICIENCY, | |
| | | | | LATE-ONSET’, ‘not | |
| | | | | provided’] | |
| NM_007375.3(TARDBP): | 80356744 | TARDBP | [‘AAATACAYATGTACT | [‘Amyotrophic lateral | 102 |
| c.*83T > C | | | AAGAATTTTCAAA’] | sclerosis type 10’] | |
| NM_000271.4(NPC1): | 80358259 | NPC1 | [‘CCGACTTAYAGCCA | [‘Niemann-Pick disease | 103 |
| c.3182T > C(p.Ile1061Thr) | | | GTAATGTCACCGAA’] | type C1’] | |
| NM_198159.2(MITF): | 104893744 | MITF | [‘TTAAAAGCAYCCGT | [‘Waardenburg syndrome | 104 |
| c.1051T > C(p.Ser351Pro) | | | GGACTATATCCGAA’] | type 2A’] | |
| NM_001018077.1(NR3C1): | 104893911 | NR3C1 | [‘AAGYGATTGCAGCA | [‘Pseudohermaphroditism, | 105 |
| c.1712T > C | | | GTGAAATGGGCAAA’] | female, with | |
| (p.Val571Ala) | | | | hypokalemia, due to | |
| | | | | glucocorticoid | |
| | | | | resistance’] | |
| NM_004577.3(PSPH): | 104894036 | PSPH | [‘AGCCAYGGGCGGGG | [‘Deficiency of | 106 |
| c.155T > C (p.Met52Thr) | | | CAGTGCCTTTCAAA’] | phosphoserine | |
| | | | | phosphatase’] | |
| NM_020661.2(AICDA): | 104894327 | AICDA | [‘CTGGAATACTTYTGT | [‘Immunodeficiency with | 107 |
| c.452T > C | | | AGAAAACCACGAA’] | hyper IgM type 2’] | |
| (p.Phel51Ser) | | | | | |
| NM_000432.3(MYL2): | 104894370 | MYL2 | [‘GTGYTCTCCATGTTC | [‘Familial hypertrophic | 108 |
| c.52T > C (p.Phe18Leu) | | | GAACAGACCCAAA’] | cardiomyopathy 10’] | |
| NM_000371.3(TTR): | 104894665 | TTR | [‘GCCATYTGCCTCTGG | [‘Amyloidogenic | 109 |
| c.191T > C (p.Phe64Ser) | | | GTAAGTTGCCAAA’] | transthyretin | |
| | | | | amyloidosis’, | |
| | | | | ‘AMYLOIDOSIS, | |
| | | | | LEPTOMENINGEAL, | |
| | | | | TRANSTHYRETIN- | |
| | | | | RELATED’] | |
| NM_033290.3(MID1): | 104894866 | MID1 | [‘CYGGCTCAGCAGATT | [‘Opitz-Frias syndrome’] | 110, |
| c.884T > C (p.Leu295Pro) | | | GCAAACTGCAAA’, | | 111, 112 |
| | | | ‘ACYGGCTCAGCAGAT | | |
| | | | TGCAAACTGCAAA’, | | |
| | | | ‘TTCGCAAACYGGCTC | | |
| | | | AGCAGATTGCAAA’] | | |
| NM_001109878.1(TBX22): | 104894946 | TBX22 | [‘CATTCYGCAATCCAT | [‘Cleft palate with | 113 |
| c.641T > C | | | GCATAAGTACAAA’] | ankyloglossia’] | |
| (p.Leu214Pro) | | | | | |
| NM_001065.3(TNFRSF1A): | 104895217 | TNFRSF1A | [‘TTCGATTTGCYGTAC | [‘TNF receptor- | 114 |
| c.175T > C | | | CAAGTGCCACAAA’] | associated periodic fever | |
| (p.Cys59Arg) | | | | syndrome (TRAPS)’] | |
| NM_000441.1 (SLC26A4): | 111033254 | SLC26A4 | [‘TATCYACAAAAGTA | [‘Pendred syndrome’, | 115 |
| c, 1588T > C | | | CCAAGAATTACAAA’] | ‘Enlarged vestibular | |
| (p.Tyr530His) | | | | aqueduct syndrome’] | |
| NM_206933.2(USH2A): | 111033264 | USH2A | [‘TAAACYGGAGAAAA | [‘Usher syndrome, type | 116 |
| c.10561T > C | | | CCTATACAATCAAA’] | 2A’] | |
| (p.Trp3521Arg) | | | | | |
| NM_006364.2(SEC23A): | 118204000 | SEC23A | [‘ACTTCCTTAYTCAAA | [‘Craniolenticulo sutural | 117 |
| c.H44T > C | | | CAAACTTTTCAAA’] | dysplasia’] | |
| (p.Phe382Leu) | | | | | |
| NM_000018.3(ACADV | 118204017 | ACADVL | [‘GCATCYTCCGGATCT | [‘Very long chain acyl- | 118 |
| L):c.1372T > C | | | TTGAGGGGACAAA’] | CoA dehydrogenase | |
| (p.Phe458Leu) | | | | deficiency’] | |
| NM_000123.3(ERCC5): | 121434575 | — | [‘TTATTTGCYTGGAAG | [‘Xeroderma | 119 |
| c.2573T > C | | | TGATTATACCGAA’] | pigmentosum, group G’] | |
| (p.Leu858Pro) | | | | | |
| NM_000280.4(PAX6): | 121907925 | PAX6 | [‘GGTATGGTYTTCTAA | [‘Congenital ocular | 120 |
| c.773T > C (p.Phe258Ser) | | | TCGAAGGGCCAAA’] | coloboma’, ‘Coloboma of | |
| | | | | optic disc’] | |
| NM_005857.4(ZMPSTE24): | 121908093 | ZMPSTE24 | [‘GCACYGGAAGTTGG | [‘Mandibulo acral | 121 |
| c.1018T > C | | | GACATACAGTCAAA’] | dysplasia with type B | |
| (p.Trp340Arg) | | | | lipodystrophy’, ‘not | |
| | | | | provided’] | |
| NM_031433.3(MFRP): | 121908190 | — | [‘AGACCATGCAAYAC | [‘Nanophthalmos 2’] | 122 |
| c.545T > C (p.Ile182Thr) | | | AGCTCAAGATCGAA’] | | |
| NM_133459.3(CCBE1): | 121908254 | CCBE1 | [‘TGGGAAGACAYGTA | [‘Hennekam | 123 |
| c.520T > C | | | CCAGGGGAGACAAA’] | lymphangiectasia- | |
| (p.Cys174Arg) | | | | lymphedema syndrome’] | |
| NM_014845.5(FIG4): | 121908287 | FIG4 | [‘TCTTGAAGAYTGATA | [‘Charcot-Marie-Tooth | 124 |
| c.122T > C (p.Ile41Thr) | | | GAACAGAACCAAA’] | disease, type 4J’, ‘not | |
| | | | | provided’] | |
| NM_003835.3(RGS9): | 121908449 | RGS9 | [‘GGAACGAYGGGCCT | [‘Prolonged electroretinal | 125 |
| c.895T > C (p.Trp299Arg) | | | TCAACTTCAGCGAA’] | response suppression’] | |
| NM_000492.3(CFTR): | 121909028 | CFTR | [‘TYTGGAGTGATACCA | [‘Cystic fibrosis’] | 126, |
| c.3857T > C | | | CAGGTGAGCAAA’, | | 128, |
| (p.Phe1286Ser) | | | ‘CTYTGGAGTGATACC | | 127, 128 |
| | | | ACAGGTGAGCAAA’]; | | |
| | | | [‘TYTGGAGTGATACCA | | |
| | | | CAGGTGAGCAAA’, | | |
| | | | ‘CTYTGGAGTGATACC | | |
| | | | ACAGGTGAGCAAA’] | | |
| NM_005570.3(LMAN1): | 121909253 | LMAN1 | [‘GAATCCAAGAYGGC | [‘Combined deficiency of | 129 |
| c.2T > C (p.Met1Thr) | | | GGGATCCAGGCAAA’] | factor V and factor VIII, | |
| | | | | 1’] | |
| NM_006302.2(MOGS): | 121909292 | MOGS | [‘CYTTGCAGACTTTGG | [‘Congenital disorder of | 130, 131 |
| c.1954T > C (p.Phe652Leu) | | | GAACCACACAAA’, | glycosylation type 2B’] | |
| | | | ‘TCYTTGCAGACTTTG | | |
| | | | GGAACCACACAAA’] | | |
| NM_002427.3(MMP13): | 121909497 | MMP13 | [‘TTTCTYCGGCTTAGA | [‘Spondyloepimetaphysea | 132 |
| c.224T > C (p.Phe75Ser) | | | GGTGACTGGCAAA’] | 1 dysplasia, Missouri | |
| | | | | type’] | |
| NM_002427.3(MMP13): | 121909498 | MMP13 | [‘TYCTTCGGCTTAGAG | [] | 133, 134 |
| c.221T > C (p.Phe74Ser) | | | GTGACTGGCAAA’, | | |
| | | | ‘TTYCTTCGGCTTAGA | | |
| | | | GGTGACTGGCAAA’] | | |
| NM_000488.3(SERPINC1): | 121909572 | SERPINC1 | [‘TGGGTGYCCAATAA | [‘Antithrombin III | 135 |
| c.667T > C | | | GACCGAAGGCCGAA’] | deficiency’] | |
| (p.Ser223Pro) | | | | | |
| NM_001173464.1(KIF21 | 121912587 | KIF21A | [‘CCAACAYAATGCAG | [‘Fibrosis of extraocular | 136 |
| A):c.3029T > C | | | ATGGAAGAAGCAAA’] | muscles, congenital, 1’] | |
| (p.Ile1010Thr) | | | | | |
| NM_000257.3(MYH7): | 121913654 | MYH7 | [‘TAAGGACCYGCAGC | [‘Familial hypertrophic | 137 |
| c.5378T > C | | | ACCGGCTGGACGAA’] | cardiomyopathy 1’, | |
| (p.Leu1793Pro) | | | | ‘Myosin storage | |
| | | | | myopathy’, ‘Left | |
| | | | | ventricular | |
| | | | | noncompaction 5’, | |
| | | | | ‘Cardiomyopathy’] | |
| NM_000124.3(ERCC6): | 121917905 | ERCC6 | [‘AATAGAGTGCYAAA | [‘Cerebro-oculo-facio- | 138 |
| c.2960T > C | | | AGACCCAAAACAAA’] | skeletal syndrome’] | |
| (p.Leu987Pro) | | | | | |
| NM_000371.3(TTR): | 121918083 | TTR | [‘TGAATCCAAGYGTCC | [‘Amyloidogenic | 139 |
| c.88T > C (p.Cys30Arg) | | | TCTGATGGTCAAA’] | transthyretin | |
| | | | | amyloidosis’, | |
| | | | | ‘Cardiomyopathy’] | |
| NM_000371.3(TTR): | 121918084 | TTR | [‘GATATACAAAGYGG | [‘Amyloidogenic | 140 |
| c.272T > C (p.Val91Ala) | | | AAATAGACACCAAA’] | transthyretin | |
| | | | | amyloidosis’] | |
| NM_000371.3(TTR): | 121918091 | TTR | [‘GGAGGAAYTTGTAG | [‘Amyloidogenic | 141, 141 |
| 5c.20T > C (p.Phe84Leu) | | | AAGGGATATAC AAA’]; | transthyretin | |
| | | | [‘GGAGGAAYTTGTAG | amyloidosis’] | |
| | | | AAGGGATATACAAA’] | | |
| NM_000371.3(TTR): | 121918100 | TTR | [‘GATAYACAAAGTGG | [‘AMYLOIDOSIS, | 143 |
| c.265T > C (p.Tyr89His) | | | AAATAGACACCAAA’] | LEPTOMENINGEAL, | |
| | | | | TRANSTHYRETIN- | |
| | | | | RELATED’] | |
| NM_000322.4(PRPH2): | 121918563 | PRPH2 | [‘CYGGACTTTTCCTCC | [‘Patterned dystrophy of | 144, |
| c.554T > C (p.Leu185Pro) | | | AAAGAAGTCAAA’, | retinal pigment | 145, 146 |
| | | | ‘CCYGGACTTTTCCTCC | epithelium’, ‘Retinitis | |
| | | | AAAGAAGTCAAA’, | pigmentosa 7, digenic’, | |
| | | | ‘CAATCGCTACCYGGA | ‘not provided’, ‘Leber | |
| | | | CTTTTCCTCCAAA’] | congenital amaurosis 18’] | |
| NM_001035.2(RYR2): | 121918602 | RYR2 | [‘GCCYTGATGCTCTCA | [‘Arrhythmogenic right | 147 |
| c.1298T > C (p.Leu433Pro) | | | GCAAGAAAGCGAA’] | ventricular | |
| | | | | cardiomyopathy, type 2’, | |
| | | | | ‘Catecholaminergic | |
| | | | | polymorphic ventricular | |
| | | | | tachycardia’, ‘Long QT | |
| | | | | syndrome’] | |
| NM_001159287.1(TPI1): | 121964847 | TPI1 | [‘CGAAYTCGTGGACA | [‘Triosephosphate | 148 |
| c.832T > C (p.Phe278Leu) | | | TCATCAATGCCAAA’] | isomerase deficiency’] | |
| NM_000398.6(CYB5R3): | 121965008 | CYB5R3 | [‘CAGTGGGCTGCYGG | [‘METHEMOGLOBINE | 149 |
| c.446T > C | | | TCTACCAGGGCAAA’] | MIA, TYPE 1’] | |
| (p.Leu149Pro) | | | | | |
| NM_004586.2(RPS6KA3): | 122454131 | RPS6KA3 | [‘ACACTCCCTTYCCAA | [‘Coffin-Lowry | 150 |
| c.803T > C | | | GGAAAAGATCGAA’] | syndrome’] | |
| (p.Phe268Ser) | | | | | |
| NM_001128834.2(PLP1): | 132630283 | PLP1 | [‘TGGCTCCAACCYTCT | [‘Peliz aeus-Merzb acher | 151 |
| c.671T > C | | | GTCCATCTGCAAA’] | disease’] | |
| (p.Leu224Pro) | | | | | |
| NM_000133.3(F9): | 137852269 | F9 | [‘ATGTCAACYGGATTA | [‘Hereditary factor IX | 152 |
| c.1357T > C (p.Trp453Arg) | | | AGGAAAAAACAAA’] | deficiency disease’] | |
| NM_000044.3(AR): | 137852597 | AR | [‘CCTGGACYCCGTGCA | [‘Androgen resistance | 153 |
| c.2596T > C (p.Ser866Pro) | | | GCCTGTAAGCAAA’] | syndrome’] | |
| NM_001171507.2(MCFD2): | 137852914 | MCFD2 | [‘GATACAYTGACTATG | [‘Factor v and factor viii, | 154 |
| c.407T > C | | | CTGAATTTGCAAA’] | combined deficiency of, | |
| (p.Ile136Thr) | | | | 2’] | |
| NM_001001486.1 (ATP2C1): | 137853015 | ATP2C1 | [‘AGCCAGTCGTCYGG | [‘Familial benign | 155 |
| c.1751T > C | | | GATTGTATTCCAAA’] | pemphigus’] | |
| (p.Leu584Pro) | | | | | |
| NM_001127695.1(CTSA): | 137854546 | CTSA | [‘TCTCYCCAGACCCAC | [‘Galactosialidosis, early | 156 |
| c.707T > C | | | TGCTGCTCTCAAA’] | infantile’] | |
| (p.Leu236Pro) | | | | | |
| NM_000371.3(TTR): | 138065384 | TTR | [‘GCCAYTTGCCTCTGG | [‘Cardiomyopathy’, ‘not | 157 |
| c.190T > C (p.Phe64Leu) | | | GTAAGTTGCCAAA’] | specified’] | |
| NM_004614.4(TK2): | 138439950 | TK2 | [‘ATAYTGCCCTCGACA | [‘Mitochondrial DNA | 158 |
| c.173A > G (p.Asn58Ser) | | | CAGATCTGGCAAA’] | depletion syndrome 2’] | |
| NM_020320.3(RARS2): | 147391618 | RARS2 | [‘CATACCYGGCAAGC | [‘Pontocerebellar | 159 |
| c.35A > G (p.Gln12Arg) | | | AATAGCGCGGCGAA’] | hypoplasia type 6’] | |
| NM_206933.2(USH2A): | 151148854 | USH2A | [‘TGTATGTCYACAGAA | [‘Usher syndrome, type | 160 |
| c.12295-2A > G | | | GGACAGAAGCAAA’] | 2A’] | |
| NM_000080.3(CHRNE): | 193919341 | — | [‘AGCGTCYGGATTGG | [‘MYASTHENIC | 161 |
| c.223T > C (p.Trp75Arg) | | | AATCGTGAGTCAAA’] | SYNDROME, | |
| | | | | CONGENITAL, 4B, | |
| | | | | FAST-CHANNEL’] | |
| NM_004329.2(BMPR1A): | 199476089 | BMPR1A | [‘AGATAYGCGTGAGG | [‘Juvenile polyposis | 162 |
| c.1409T > C | | | TTGTGTGTGTCAAA’] | syndrome’] | |
| (p.Met470Thr) | | | | | |
| NM_006214.3(PHYH): | 201578674 | PHYH | [‘ATACCYAAAGGAGA | [‘Refsum disease, adult, | 163 |
| c.135-2A > G | | | AAAAGAATCCCAAA’] | 1’] | |
| NM_013382.5(POMT2): | 267606964 | POMT2 | [‘CTCAYGGGACTTTTG | [‘Congenital muscular | 164 |
| c.2242T > C | | | AGGCCACTGCAAA’] | dystrophy- | |
| (p.Trp748Arg) | | | | dystroglycanopathy with | |
| | | | | mental retardation, type | |
| | | | | B2’] | |
| NM_000084.4(CLCN5): | 273585645 | CLCN5 | [‘GAACATCCYGTGCC | [‘Dent disease 1’] | 165 |
| c.674T > C (p.Leu225Pro) | | | ACTGCTTCAACAAA’] | | |
| NM_005211.3(CSF1R): | 281860279 | CSF1R | [‘AAAYGGCCCAGCCT | [‘Hereditary diffuse | 166 |
| c.2624T > C (p.Met875Thr) | | | GCATTTGCCCCAAA’] | leukoencephalopathy | |
| | | | | with spheroids’] | |
| NM_004614.4(TK2): | 281865497 | TK2 | [‘CCATCYCCATGAGG | [‘Mitochondrial DNA | 167 |
| c.644T > C (p.Leu215Pro) | | | AGTGGCTCATCAAA’] | depletion syndrome 2’] | |
| NM_000155.3(GALT): | 367543254 | GALT | [‘CCAGYGATCATCCCC | [‘Deficiency of | 168 |
| c.336T > C (p.Ser1l2=) | | | TTTTCCAAGCAAA’] | UDPglucose-hexose-1- | |
| | | | | phosphate | |
| | | | | uridylyltransferase’] | |
| NM_022445.3(TPK1): | 371271054 | TPK1 | [‘AYTGGAAGTACTGA | [‘THIAMINE | 169, 170 |
| c.656A > G (p.Asn219Ser) | | | CCAATGTTCCAAA’, | METABOLISM | |
| | | | ‘TAYTGGAAGTACTGA | DYSFUNCTION | |
| | | | CCAATGTTCCAAA’] | SYNDROME 5 | |
| | | | | (EPISODIC | |
| | | | | ENCEPHALOPATHY | |
| | | | | TYPE)’] | |
| NM_001282227.1 (CECR1): | 376785840 | CECR1 | [‘AATCAYAGGACAAG | [‘Polyarteritis nodosa’] | 171 |
| c.1232A > G | | | CCTTTGGCACCAAA’] | | |
| (p.Tyr411Cys) | | | | | |
| NM_004453.3(ETFDH): | 377686388 | ETFDH | [‘TACCYGAGTCCATTT | [‘Glutaric aciduria, type | 172 |
| c.1001T > C | | | AGAGAGTTCCAAA’] | 2’] | |
| (p.Leu334Pro) | | | | | |
| NM_002465.3(MYBPC1): | 387906657 | MYBPC1 | [‘ACGTAYGGGAGTTG | [‘Distal arthrogryposis | 173 |
| c.706T > C | | | CTGAAGAACGCGAA’] | type IB’] | |
| (p.Trp236Arg) | | | | | |
| NM_001256714.1(DNAAF3): | 387907151 | DNAAF3 | [‘ACCTGCYGCGGACC | [‘Kartagener syndrome’, | 174 |
| c.386T > C | | | CTGTCCCGAGCGAA’] | ‘Ciliary dyskinesia, | |
| (p.Leul29Pro) | | | | primary, 2’] | |
| NM_152296.4(ATP1A3): | 387907282 | ATP1A3 | [‘CATCYCACTGGCGTA | [‘Alternating hemiplegia | 175 |
| c.2431T > C | | | CGAGGCTGCCGAA’] | of childhood 2’] | |
| (p.Ser811Pro) | | | | | |
| NM_000344.3(SMN1): | 397514518 | SMN1 | [‘ACTGGAYATGGAAA | [‘Kugelberg-Welander | 176 |
| c.388T > C (p.Tyrl30His) | | | TAGAGAGGAGCAAA’] | disease’] | |
| NM_024531.4(SLC52A2): | 397514538 | SLC52A2 | [‘TGCTGGCACYGGCAT | [‘Brown-Vialetto-Van | 177 |
| c.368T > C | | | GCTGTGCCTCGAA’] | Laere syndrome 2’] | |
| (p.Leu123Pro) | | | | | |
| NM_001006657.1(WDR35): | 397515533 | WDR35 | [‘AGTCYACCTAATGTT | [‘Cranioectodermal | 178 |
| c.1592T > C | | | GGTTTGATTCAAA’] | dysplasia 2’] | |
| (p.Leu531Pro) | | | | | |
| NM_004595.4(SMS): | 397515552 | SMS | [‘ATCAAAATAYAAAA | [‘Snyder Robinson | 179 |
| c.449T > C (p.Ile150Thr) | | | ATTCTACACTCGAA’] | syndrome’] | |
| NM_206933.2(USH2A): | 397518022 | | [‘CAGGAGCAGGYAAA | [‘Usher syndrome, type | 180 |
| c.5857 + 2T > C | | | TACTTATCTTCAAA’] | 2A’] | |
| NM_000212.2(ITGB3): | 398122374 | — | [‘AAACTCCYCATCACC | [‘Platelet-type bleeding | 181 |
| c.2231T > C (p.Leu744Pro) | | | ATCCACGACCGAA’] | disorder 16’] | |
| NM_000019.3(ACAT1): | 398123096 | ACAT1 | [‘CAGTAAAAGGYAGA | [‘Deficiency of acetyl- | 182, 182 |
| c.730 + 2T > C | | | GATAATGTTCCAAA’]; | CoA acetyltransferase’, | |
| | | | [‘CAGTAAAAGGYAGA | ‘not provided’] | |
| | | | GATAATGTTCCAAA’] | | |
| NM_000169.2(GLA): | 398123223 | — | [‘CYCCGACACATCAG | [‘Fabry disease’] | 183, 184 |
| c.899T > C (p.Leu300Pro) | | | CCCTCAAGCCAAA’, | | |
| | | | ‘CCYCCGACACATCAG | | |
| | | | CCCTCAAGCCAAA’] | | |
| NM_014795.3(ZEB2): | 398124282 | ZEB2 | [‘AAAACGGYAAGAAG | [‘Mowat-Wilson | 185 |
| c.73 + 2T > C | | | CAGCCCGAACCAAA’] | syndrome’] | |
| NM_000353.2(TAT): | 587776512 | TAT | [‘TCCCCTTTYTATGGG | [‘Tyrosinemia type 2’] | 186 |
| c.236-5A > G | | | AGGAAAACACAAA’] | | |
| NM_001142519.1(FAM111A): | 587777012 | FAM111A | [‘CCAGAGYATGTCCAT | [‘Kenny-Caffey | 187 |
| c.1531T > C | | | ATGTATACTCAAA’] | syndrome type 2’] | |
| (p.Tyr511His) | | | | | |
| NM_052844.3(WDR34): | 587777098 | WDR34 | [‘CAGATACYTGAGGG | [‘Short-rib thoracic | 188 |
| c.1307A > G | | | AGAGCTGCAGCGAA’] | dysplasia 11 with or | |
| (p.Lys436Arg) | | | | without polydactyly’] | |
| NM_014740.3(EIF4A3): | 587777204 | EIF4A3 | [‘TGTCGTAGAGGYCA | [‘Richieri Costa Pereira | 189 |
| c.809A > G | | | CACAGAGTGTCAAA’] | syndrome’] | |
| (p.Asp270Gly) | | | | | |
| NM_012338.3(TSPAN12): | 587777283 | TSPAN12 | [‘TCCAYAATTTGTCAT | [‘Exudative | 190 |
| c.413A > G | | | CCTGGCTTTCAAA’] | vitreoretinopathy 5’] | |
| (p.Tyr138Cys) | | | | | |
| NM_001159287.1(TPI1): | 587777440 | TPI1 | [‘CGAATYCGTGGACA | [‘Triosephosphate | 191 |
| c.833T > C (p.Phe278Ser) | | | TCATCAATGCCAAA’] | isomerase deficiency’] | |
| NM_017696.2(MCM9): | 587777871 | MCM9 | [‘TAGCAGAAGGYCTA | [‘Premature ovarian | 192 |
| c.1732 + 2T > C | | | TTTCATTCAGCGAA’] | failure 1’, ‘Ovarian | |
| | | | | dysgenesis 4’] | |
| NM_178151.2(DCX): | 587783574 | DCX | [‘CCTCACTGATAYCAC | [‘Heterotopia’] | 193 |
| c.641T > C (p.Ile214Thr) | | | AGAAGCCATCAAA’] | | |
| NM_022455.4(NSD1): | 587784171 | NSD1 | [‘TAATTTCYATATGCT | [‘Sotos syndrome 1’] | 194 |
| c.5989T > C | | | CACCCTAGACAAA’] | | |
| (p.Tyr1997His) | | | | | |
| NM_017653.3(DYM): | 775414124 | DYM | [‘CYATACAAAAAGGA | [‘Dyggve-Melchior- | 195, 196 |
| c.621-2A > G | | | AAAAAAAATCAAA’, | Clausen syndrome’] | |
| | | | ‘ACYATACAAAAAGGA | | |
| | | | AAAAAAAATCAAA’] | | |
| NM_001987.4(ETV6): | 786205155 | ETV6 | [‘TCAGTTGCYTTCTGA | [‘Thrombocytopenia’, | 197 |
| c.1046T > C (p.Leu349Pro) | | | CAGCCGGTACGAA’] | ‘LEUKEMIA, ACUTE | |
| | | | | LYMPHOBLASTIC; | |
| | | | | ALL’] | |
| NM_004370.5(COL12A1): | 796052093 | COL12A1 | [‘GAGCAYTGGGGACG | [‘BETHLEM | 198 |
| c.7001T > C | | | ATAATTTTAACAAA’] | MYOPATHY 2’] | |
| (p.Ile2334Thr) | | | | | |
| NM_014191.3(SCN8A): | 796053222 | SCN8A | [‘CATCTTGCGTCYGAT | [‘not provided’] | 199 |
| c.4889T > C | | | CAAAGGCGCCAAA’] | | |
| (p.Leu1630Pro) | | | | | |

TABLE 2
Human gene mutations that may be corrected by changing a guanineine
(G) to a adenine (A) using GeoCas9 base editors provided herein. The gene name, gene
symbol, and dbSNP database reference number (RS#) are indicated. Also indicated are
exemplary protospacers with their PAM sequences (gRNAs) and the base to be edited, e.g., a
G, indicated by a “R”.The “gRNAs” sequences correspond to SEQ ID NOs: 200-373.
SEQ ID NO(s):

| | RS# | Gene | | | SEQ ID |
|---|---|---|---|---|---|
| Name | (dbSNP) | Symbol | gRNA | Phenotypes | No(s): |
| NM_000487.5(ARSA): | 6151429 | ARSA | [‘TTTGTGCCTGATAAC | [‘Metachromatic | 200, 201 |
| c.*96A > G | | | GTARTAACACC AG’]; | leukodystrophy’, | |
| | | | [‘TTTGTGCCTGATAAC | ‘Arylsulfatase A | |
| | | | GTARTAACACCAG’] | pseudodeficiency’, ‘not | |
| | | | | provided’] | |
| NM_000060.3(BTD): | 28934601 | BTD | [‘TTTGATATATTGTTC | [‘Biotinidase deficiency’] | 202 |
| c.755A > G (p.Asp252Gly) | | | TTTGRCCCTGCCA’] | | |
| NM_005263.3(GFI1): | 28936382 | GFI1 | [‘TTCGGCTGCGACCTC | [‘Neutropenia, | 203 |
| c.1208A > G (p.Lys403Arg) | | | TGTGGGARGGGTT’] | nonimmune chronic | |
| | | | | idiopathic, of adults’] | |
| NM_000527.4(LDLR): | 28942085 | LDLR | [‘TTTGACAACCCCGTC | [‘Familial | 204 |
| c.2483A > G (p.Tyr828Cys) | | | TRTCAGAAGACCA’] | hypercholesterolemia’, | |
| | | | | ‘not provided’] | |
| NM_000060.3(BTD): | 35976361 | BTD | [‘TTTGCTGTTGCCTTT | [‘Biotinidase deficiency’] | 205 |
| c.880A > G (p.Ile294Val) | | | GGCRTCAACGTTC’] | | |
| NM_000249.3(MLH1): | 63750211 | MLH1 | [‘TTTGGAAGTTGTTGG | [‘Hereditary | 206 |
| c.544A > G (p.Argl82Gly) | | | CRGGTACAGTCCA’] | Nonpolyposis Colorectal | |
| | | | | Neoplasms’] | |
| NM_000484.3(APP): | 63751039 | APP | [‘TTTGTTTTCAAGGTG | [‘Alzheimer disease’, | 207 |
| c.2078A > G (p.Glu693Gly) | | | TTCTTTGCAGRAG’] | ‘Alzheimer disease, type | |
| | | | | 1’, ‘Cerebral amyloid | |
| | | | | angiopathy, APP-related’, | |
| | | | | ‘not provided’] | |
| NM_000249.3(MLH1): | 63751094 | MLH1 | [‘TTTGATTTGCCAGTT | [‘Hereditary | 208 |
| c.122A > G (p.Asp41Gly) | | | TAGRTGCAAAATC’] | Nonpolyposis Colorectal | |
| | | | | Neoplasms’] | |
| NM_000463.2(UGTlA1): | 72551348 | — | [‘TTTGGGCAAAATCCC | [‘Crigler-Najjar | 209 |
| c.992A > G (p.Gln331Arg) | | | TCRGACAGTAAGA’] | syndrome, type 11’] | |
| NM_000531.5(GTC): | 72554332 | OTC | [‘TTTGCCTTTATTGCA | [‘Ornithine | 210 |
| c.238A > G (p.Lys80Glu) | | | AGGGRAGTCCTTA’] | carbamoyltransferase | |
| | | | | deficiency’, ‘not | |
| | | | | provided’] | |
| NM_000531.5(OTC): | 72554344 | OTC | [‘TTTGAGAAAAGAAG | [‘not provided’] | 211 |
| c.277A > G (p.Thr93Ala) | | | TACTCGARCAAGAT’] | | |
| NM_000404.2(GLB 1): | 72555368 | GLB1 | [‘TTTGCAGGGTTTGGT | [‘Mucopolysaccharidosis, | 212, 213 |
| c.1498A > G (p.Thr500Ala) | | | TTCTAACCTGRCT’, | MPS-IV-B’] | |
| | | | ‘TTTGGTTTCTAACCTG | | |
| | | | RCTCTCAGTTCC’] | | |
| NM_170784.2(MKKS): | 74315399 | MKKS | [‘TTTGGAGGTTACGTG | [‘Bardet-Biedl syndrome | 214 |
| c.169A > G (p.Thr57Ala) | | | TGTACARCCTCAC’] | 6’] | |
| NM_000311.3(PRNP): | 74315411 | PRNP | [‘TTTGTGCACGACTGC | [‘Genetic prion diseases’, | 215, 216 |
| c.547A > G (p.Thr183Ala) | | | GTCAATATCRCAA’]; | ‘Spongiform | |
| | | | [‘TTTGTGCACGACTGC | encephalopathy with | |
| | | | GTCAATATCRCAA’] | neurop sy chi atric | |
| | | | | features’] | |
| NM_000492.3(CFTR): | 76151804 | CFTR | [‘TTTGTGTTTATGTTA | [‘Cystic fibrosis’] | 217 |
| c.3140-26A > G | | | TTTGCARTGTTTT’] | | |
| NM_000303.2(PMM2): | 80338704 | PMM2 | [‘TTTGATGTCTTTCCT | [‘Carbohydrate-deficient | 218 |
| c.563A > G (p.Asp188Gly) | | | GATGGATGGGRCA’] | glycoprotein syndrome | |
| | | | | type F, ‘not provided’] | |
| NM_206933.2(USH2A): | 80338904 | USH2A | [‘TTTGTATTACGAATT | [‘Retinitis pigmentosa’, | 219 |
| c.14020A > G | | | ATACRGAAGACAA’] | ‘Retinitis pigmentosa 39’] | |
| (p.Arg4674Gly) | | | | | |
| NM_000059.3(BRCA2): | 81002862 | BRCA2 | [‘TTTGTTTTGTTTTCTG | [‘Familial cancer of | 220 |
| c.9118-2A > G | | | TRGGTTTCAGAT’] | breast’, ‘Breast-ovarian | |
| | | | | cancer, familial 2’] | |
| NM_000495.4(COL4A5): | 104886193 | COL4A5 | [‘TTTGGGAATTCCTGG | [‘Alport syndrome, X- | 221 |
| c.2746A > G | | | CAGGRGTGGTGTA’] | linked recessive’] | |
| (p.Ser916Gly) | | | | | |
| NM_000495.4(COL4A5): | 104886341 | COL4A5 | [‘TTTGAACGTTTTCCT | [‘Alport syndrome, X- | 222 |
| c.2042-18A > G | | | TTCAATARCTGCT’] | linked recessive’] | |
| NM_000073.2(CD3G): | 104894199 | CD3G | [‘TTTGCCGGAGGACA | [‘Immunodeficiency 17’] | 223 |
| c.1A > G (p.Met1Val) | | | GAGACTGACRTGGA’] | | |
| NM_000317.2(PTS): | 104894278 | PTS | [‘TTTGGGAAATGCAAC | [‘Hyperphenylalaninemia, | 224 |
| c.139A > G (p.Asn47Asp) | | | AATCCARATGGCC’] | bh4-deficient, a, due to | |
| | | | | partial pts deficiency’] | |
| NM_000448.2(RAG1): | 104894292 | RAG1 | [‘TTTGCTGACAAAGAA | [‘Histiocytic medullary | 225 |
| c.1286A > G (p.Asp429Gly) | | | GAAGGTGGAGRTG’] | reticulosis’] | |
| NM_014239.3(EIF2B2): | 104894425 | EIF2B2 | [‘TTTGTCCAAAGCAGG | [‘Leukoencephalopathy | 226 |
| c.638A > G (p.Glu213Gly) | | | TATTGRGACAACT’] | with vanishing white | |
| | | | | matter’, | |
| | | | | ‘Ovarioleukodystrophy’] | |
| NM_024006.5(VKORC1): | 104894541 | VKORC1 | [‘TTCGCGCGTCTTCTC | [‘Warfarin response’] | 227 |
| c, 172A > G (p.Arg58Gly) | | | CTCCRGGTGTGCA’] | | |
| NM_001128085.1 (ASPA): | 104894551 | — | [‘TTTGGAGGAACCCAT | [‘Spongy degeneration of | 228 |
| c.71A > G (p.Glu24Gly) | | | GGGAATGRGCTAA’] | central nervous system’] | |
| NM_002769.4(PRSS1): | 111033567 | — | [‘TTTGATGATGATGAC | [‘Hereditary pancre- | 229 |
| c.68A > G (p.Lys23Arg) | | | ARGATCGTTGGGG’] | atitis’] | |
| NM_000155.3(GALT): | 111033830 | GALT | [‘TTTGCCCCTTGACAG | [‘Deficiency of | 230 |
| c.574A > G (p.Ser192Gly) | | | GTATGGGCCRGCA’] | UDPglucose-hexose-1- | |
| | | | | phosphate | |
| | | | | uridylyltransferase’] | |
| NM_172107.2(KCNQ2): | 118192238 | KCNQ2 | [‘TTTGGGGTCTCTGTT | [‘Benign familial | 231 |
| c.1764-2A > G | | | CCCGGTRGAGTGG’] | neonatal seizures 1’] | |
| NM_004519.3(KCNQ3): | 118192248 | KCNQ3 | [‘TTTGAGACCTATGCA | [‘Benign familial | 232 |
| c914A > G (p.Asp305Gly) | | | GRTGCCCTGTGGT’] | neonatal seizures 2’] | |
| NM_000785.3(CYP27B1): | 118204012 | CYP27B1 | [‘TTCGGGACGTGGCG | [‘Vitamin D-dependent | 233 |
| c.566A > G (p.Glu189Gly) | | | GGGGRATTTTACAA’] | rickets, type 1’] | |
| NM_015702.2(MMADHC): | 118204046 | MMADHC | [‘TTTGAAACTGATGAA | [‘Homocystinuria, cblD | 234 |
| c.746A > G | | | CGCTRCCGACATT’] | type, variant 1’] | |
| (p.Tyr249Cys) | | | | | |
| NM_014874.3(MFN2): | 119103264 | MFN2 | [‘TTTGGGCTCCAGGTG | [‘Hereditary motor and | 235 |
| c.827A > G (p.Gln276Arg) | | | CGGCGGCRGCACA’] | sensory neuropathy with | |
| | | | | optic atrophy’] | |
| NM_020247.4(ADCK3): | 119468008 | ADCK3 | [‘TTTGGGGCAACGCG | [‘Coenzyme Q10 | 236 |
| c.1541A > G (p.Tyr514Cys) | | | GGAATRTGACAGAT’] | deficiency, primary, 4’] | |
| NM_000197.1(HSD17B3): | 119481079 | HSD17B3 | [‘TTTGCTTCTTTCTGCC | [‘Testosterone 17-beta- | 237 |
| c.389A > G | | | AGTCARCAATGT’] | dehydrogenase | |
| (p.Asn130Ser) | | | | deficiency’] | |
| NM_017780.3(CHD7): | 121434345 | CHD7 | [‘TTTGCCTCTTTACAG | [‘Kallmann syndrome 5’] | 238, 239 |
| c.164A > G (p.His55Arg) | | | CCATCCCTTCRTC’]; | | |
| | | | [‘TTTGCCTCTTTACAG | | |
| | | | CCATCCCTTCRTC’] | | |
| NM_002894.2(RBBP8): | 121434388 | RBBP8 | [‘TTTGGAGCTACCTCT | [‘Carcinoma of pancreas’] | 240 |
| c.1009A > G (p.Lys337Glu) | | | AGTATCRAAAGTG’] | | |
| NM_000430.3(PAFAH1B1): | 121434482 | PAFAH1 | [‘TTTGAACGAACTCTT | [‘Lissencephaly 1’] | 241 |
| c.446A > G | | B1 | AAAGGACRTACAG’] | | |
| (p.His 149 Arg) | | | | | |
| NM_024301.4(FKRP): | 121908110 | FKRP | [‘TTCGTGGCGCAGGCG | [‘Congenital muscular | 242, 243 |
| c.1387A > G (p.AsnAsp) | | | CCTRACAACTACC’]; | dystrophy- | |
| | | | [‘TTCGTGGCGCAGGCG | dystroglycanopathy (with | |
| | | | CCTRACAACTACC’] | or without mental | |
| | | | | retardation) type B5’, | |
| | | | | ‘Limb-girdle muscular | |
| | | | | dystrophy- | |
| | | | | dystroglycanopathy, type | |
| | | | | C5’, ‘Muscular | |
| | | | | dystrophy’, ‘Congenital | |
| | | | | muscular dystrophy- | |
| | | | | dystroglycanopathy with | |
| | | | | brain and eye anomalies | |
| | | | | type A5’, ‘Congenital | |
| | | | | muscular dystrophy- | |
| | | | | dystroglycanopathy | |
| | | | | without mental | |
| | | | | retardation, type B5’, ‘not | |
| | | | | provided’] | |
| NM_014946.3(SPAST): | 121908514 | SPAST | [‘TTTGGTCCACCTGGG | [‘Spastic paraplegia 4, | 244 |
| c.1157A > G (p.Asn386Ser) | | | ARTGGGAAGACAA’] | autosomal dominant’] | |
| NM_006892.3(DNMT3B): | 121908939 | DNMT3B | [‘TTTGGCTTTCCTGTG | [‘Centromeric instability | 245 |
| c.2450A > G | | | CACTACACAGRCG’] | of chromosomes 1, 9 and | |
| (p.Asp817Gly) | | | | 16 and | |
| | | | | immunodeficiency’] | |
| NM_001130978.1(DYSF): | 121908961 | DYSF | [‘TTTGTCTTCTCTCTG | [‘Limb-girdle muscular | 246 |
| c.5264A > G | | | GGGCAGRGGCTGG’] | dystrophy, type 2B’] | |
| (p.Glu1755Gly) | | | | | |
| NM_001040667.2(HSF4): | 121909050 | HSF4 | [‘TTCGGAAGGTGGTG | [‘Cataract, zonular’] | 247 |
| c.256A > G (p.Ile86Val) | | | AGCRTCGAGCAGGG’] | | |
| NM_001363.4(DKC1): | 121912297 | DKC1 | [‘TTTGATAAGCTGAAT | [‘Dyskeratosis congenita | 248 |
| c.196A > G (p.Thr66Ala) | | | GTAAGGRCAACAC’] | X-linked’] | |
| NM_001363.4(DKC1): | 121912305 | DKC1 | [‘TTCGGGTGGAGAAG | [‘Dyskeratosis congenita | 249 |
| c.361A > G (p.Ser121Gly) | | | ACAGGGCACRGTGG’] | X-linked’, ‘Hoyeraal | |
| | | | | Hreidarsson syndrome’] | |
| NM_001943.3(DSG2): | 121913011 | DSG2 | [‘TTCGTATTTTGGATG | [‘Arrhythmogenic right | 250 |
| c.797A > G (p.Asn266Ser) | | | TCARTGACAATAT’] | ventricular | |
| | | | | cardiomyopathy, type | |
| | | | | 10’] | |
| NM_000043.4(FAS): | 121913082 | FAS | [‘TTTGTTCGAAAGAAT | [ ] | 251, 252 |
| c.763A > G (p.Asn255Asp) | | | GGTGTCRATGAAG’, | | |
| | | | ‘TTCGAAAGAATGGTG | | |
| | | | TCRATGAAGCCAA’] | | |
| NM_001127500.1(MET): | 121913246 | MET | [‘TTTGGTCTTGCCAGA | [‘Renal cell carcinoma, | 253 |
| c.3743A > G | | | GACATGTRTGATA’] | papillary, 1’] | |
| (p.Tyr1248Cys) | | | | | |
| NM_007035.3(KERA): | 121917858 | KERA | [‘TTTGAGACTAAATCA | [‘Cornea plana 2’] | 254 |
| c.740A > G (p.Asn247Ser) | | | CARCAAACTGTCA’] | | |
| NM_000536.3(RAG2): | 121917897 | RAG2 | [‘TTTGGACAAAAAGG | [‘Histiocytic medullary | 255 |
| c.115A > G (p.Arg39Gly) | | | CTGGCCCAAARGAT’] | reticulosis’] | |
| NM_000040.1(APGC3): | 121918381 | APOC3 | [‘TTTGGACCCTGAGGT | [ ] | 256 |
| c.280A > G (p.Thr94Ala) | | | CAGACCARCTTCA’] | | |
| NM_021957.3(GYS2): | 121918423 | GYS2 | [‘TTTGAAGTTGCTTGG | [‘Hypoglycemia with | 257 |
| c.116A > G (p.Asn39Ser) | | | GAAGTGACCARTA’] | deficiency of glycogen | |
| | | | | synthetase in the liver’] | |
| NM_002834.3(PTPN11): | 121918466 | PTPN11 | [‘TTTGCCACTTTGGCT | [‘Noonan syndrome’, | 258, 259 |
| c.236A > G (p.Gln79Arg) | | | GAGTTGGTCCRGT’, | ‘Noonan syndrome 1’, | |
| | | | ‘TTTGGCTGAGTTGGT | ‘Rasopathy’, ‘not | |
| | | | CCRGTATTACATG’] | provided’] | |
| NM_000313.3(PROS1): | 121918474 | PROS1 | [‘TTTGTTATGCTTTCA | [‘Protein S deficiency’] | 260 |
| c.586A > G (p.Lys196Glu) | | | AATRAGAAAGATT’] | | |
| NM_001063.3(TF): | 121918678 | TF | [‘TTTGTTTGTTCCGGT | [ ] | 261, 262 |
| c.1936A > G (p.Lys646Glu) | | | CGGAAACCRAGGA’, | | |
| | | | TTTGTTCCGGTCGGA | | |
| | | | AACCRAGGACCTT’] | | |
| NM_004612.3(TGFBR1): | 121918711 | TGFBR1 | [‘TTTGAATCCTTCAAA | [‘Loeys-Dietz syndrome | 263 |
| c.H99A > G | | | CGTGCTGRCATCT’] | 1’] | |
| (p.Asp400Gly) | | | | | |
| NM_000532.4(PCCB): | 121964961 | PCCB | [‘TTTGGATCTGTTTTA | [‘Propionic acidemia’] | 264 |
| c.1304A > G (p.Tyr435Cys) | | | GGCCTRTGGAGGT’] | | |
| NM_000137.2(FAH): | 121965078 | FAH | [‘TTTGCTGTGCCCAAC | [‘Tyrosinemia type 1’] | 265 |
| c.836A > G (p.Gln279Arg) | | | CCGAAGCRGGTAA’] | | |
| NM_000132.3(F8): | 137852420 | F8 | [‘TTCGTTAATATGGAG | [‘Hereditary factor VIII | 266 |
| c.1682A > G (p.Asp561Gly) | | | AGAGRTCTAGCTT’] | deficiency disease’] | |
| NM_004387.3(NKX2- | 137852683 | NKX2-5 | [‘TTCGTGAACTTCGGC | [‘Atrial septal defect 7 | 267 |
| 5):c.896A > G | | | GTCGGGGRCTTGA’] | with or without | |
| (p.Asp299Gly) | | | | atrioventricular | |
| | | | | conduction defects’] | |
| NM_153638.2(PANK2): | 137852965 | PANK2 | [‘TTTGAACCCAAAGAC | [ ] | 268 |
| c.700A > G (p.Thr234Ala) | | | ATCRCTGCTGAAG’] | | |
| NM_001080463.1(DYNC2H1): | 137853026 | DYNC2H1 | [‘TTTGCTTTTGTAGGT | [‘Short-rib thoracic | 269 |
| c.1l284A > G | | | TGCCRTGGGTCAA’] | dysplasia 3 with or | |
| (p.Met3762Val) | | | | without polydactyly’] | |
| NM_031885.3(BBS2): | 137854887 | BBS2 | [‘TTTGGGGTTTTATTT | [‘Bardet-Biedl syndrome | 270 |
| c.472-2A > G | | | TCRGGTTACTGGA’] | 2’] | |
| NM_000199.3(SGSH): | 138504221 | SGSH | [‘TTTGGGTGCTCCGGG | [‘Mucopolysaccharidosis, | 271 |
| c.892T > C (p.Ser298Pro) | | | GRTGACACCAGTA’] | MPS-III-A’, ‘not | |
| | | | | provided’] | |
| NM_004333.4(BRAF): | 180177035 | BRAF | [‘TTTGTCGAAAGCTGC | [‘Noonan syndrome 7’, | 272 |
| c.770A > G (p.Gln257Arg) | | | TTTTCCRGGGTTT’] | ‘Cardiofaciocutaneous | |
| | | | | syndrome’, ‘Rasopathy’, | |
| | | | | ‘not provided’] | |
| NM_003060.3(SLC22A5): | 188698686 | SLC22A5 | [‘TTCGTATAATATTCT | [‘not provided’] | 273 |
| c.694A > C (p.Thr232Pro) | | | CTRCGTTAGGAGT’] | | |
| m.3243A > G | 199474657 | MT-TL1 | [‘TTTGTTAAGATGGCA | [‘Leigh disease’, ‘Cyclical | 274 |
| | | | GRGCCCGGTAATC’] | vomiting syndrome’, | |
| | | | | ‘Juvenile myopathy, | |
| | | | | encephalopathy, lactic | |
| | | | | acidosis AND stroke’, | |
| | | | | ‘Myoclonus with epilepsy | |
| | | | | with ragged red fibers’, | |
| | | | | ‘Cytochrome-c oxidase | |
| | | | | deficiency’, ‘Diabetes- | |
| | | | | deafness syndrome | |
| | | | | maternally transmitted’, | |
| | | | | ‘3-Methylglutaconic | |
| | | | | aciduria’, ‘Age-related | |
| | | | | macular degeneration 2’, | |
| | | | | ‘MERRF/MELAS | |
| | | | | overlap syndrome’] | |
| m.3252A > G | 199474661 | MT-TL1 | [‘TTTGTTAAGATGGCA | [‘Mitochondrial | 275 |
| | | | GAGCCCGGTARTC’] | encephalomyopathy’] | |
| m.3251A > G | 199474662 | MT-TL1 | [‘TTTGTTAAGATGGCA | [ ] | 276 |
| | | | GAGCCCGGTRATC’] | | |
| NM_001018005.1(TPM1): | 199476319 | TPM1 | [‘TTTGCGGAGAGGTCA | [‘Left ventricular | 277 |
| c.742A > G (p.Lys248Glu) | | | GTAACTRAATTGG’] | noncompaction 9’, ‘not | |
| | | | | provided’] | |
| NM_000267.3(NF1): | 267606602 | NF1 | [‘TTTGTCTTTCTCTTTT | [‘Neurofibromatosis, type | 278, 279 |
| c.1642-8A > G | | | TTAAARAATTCA’]; | 1’, ‘Juvenile | |
| | | | [‘TTTGTCTTTCTCTTTT | myelomonocytic | |
| | | | TTAAARAATTCA’] | leukemia’] | |
| NM_198965.1(PTHLH): | 267606987 | PTHLH | [‘TTTGCAGGAGGCATT | [‘Brachydactyly type E2’] | 280 |
| c.534A > G (p.Ter178Trp) | | | GRAATTTTCAGCA’] | | |
| NM_007373.3(SHOC2): | 267607048 | SHOC2 | [‘TTTGTCCAGGCTTGA | [‘Noonan-like syndrome | 281 |
| c.4A > G (p.Ser2Gly) | | | GTCACCATGRGTA’] | with loose anagen hair’, | |
| | | | | ‘Rasopathy’] | |
| NM_018105.2(THAP1): | 267607111 | THAP1 | [‘TTTGTACTGAGCCAC | [‘Dystonia 6, torsion’] | 282 |
| c.266A > G (p.Lys89Arg) | | | ATGACARGGTAAT’] | | |
| NM_003286.2(TOP1): | 267607131 | — | [‘TTTGACTTCCTCGGG | [ ] | 283 |
| c.1598A > G (p.Asp533Gly) | | | AAGGRCTCCATCA’] | | |
| NM_000249.3(MLH1): | 267607760 | MLH1 | [‘TTTGGAAGTTGTTGG | [‘Hereditary | 284 |
| c.545 + 3A > G | | | CAGGTRCAGTCCA’] | Nonpolyposis Colorectal | |
| | | | | Neoplasms’] | |
| NM_000288.3(PEX7): | 267608255 | PEX7 | [‘TTTGCTTTCTAAACA | [‘Phytanic acid storage | 285 |
| c.340-10A > G | | | CTTTTCARTGTTT’] | disease’, ‘Peroxisome | |
| | | | | biogenesis disorder 9B’] | |
| NM_003159.2(CDKL5): | 267608480 | CDKL5 | [‘TTTGGACTTTGCTAT | [‘Early infantile epileptic | 286 |
| c.464-2A > G | | | CTTTCRGGTTTTG’] | encephalopathy 2’, ‘not | |
| | | | | provided’] | |
| NM_212472.2(PRKAR1A: | 281864796 | PRKAR1A | [‘TTTGCAAACTCGTAA | [‘Carney complex, type | 287 |
| c.178-2A > G | | | TTTCTTTCRGGAG’] | 1’] | |
| NM_024312.4(GNPTAB): | 281865019 | GNPTAB | [‘TTTGTTTGCCTGAAT | [‘Pseudo-Hurler | 288 |
| c.3458A > G | | | GACARCATTGACC’] | polydystrophy’] | |
| (p.Asn1153Ser) | | | | | |
| NM_198578.3(LRRK2): | 281865052 | LRRK2 | [‘TTTGGCTGACCTGCC | [‘Parkinson disease 8, | 289 |
| c.5605A > G | | | TAGAAATATTRTG’] | autosomal dominant’] | |
| (p.Met1869Val) | | | | | |
| NM_004614.4(TK2): | 281865495 | TK2 | [‘TTCGGACCAATCCTG | [‘Mitochondrial DNA | 290 |
| c.562A > G (p.Thr188Ala) | | | AGRCTTGTTACCA’] | depletion syndrome 2’] | |
| NM_000495.4(COL4A5): | 281874758 | COL4A5 | [‘TTTGTCTTCTCTTCTT | [‘Alport syndrome, X- | 291 |
| c.610-2A > G | | | RGGGCCCTCCTG’] | linked recessive’] | |
| NM_005359.5(SMAD4): | 281875320 | SMAD4 | [‘TTCGTCGCTTATGCA | [‘Myhre syndrome’, ‘not | 292 |
| c.1500A > G (p.Ile500Met) | | | TRCTCAGGATGAG’] | provided’] | |
| NM_000095.2(CGMP): | 312262901 | COMP | [‘TTCGAGGGCACGTTC | [‘Pseudoachondroplastic | 293 |
| c.1760A > G (p.His587Arg) | | | CRTGTGAACACGG’] | spondyloepiphyseal | |
| | | | | dysplasia syndrome’] | |
| NM_000155.3(GALT): | 367543252 | GALT | [‘TTTGACAACGACTTC | [‘Deficiency of | 294 |
| c.308A > G (p.Gln103Arg) | | | CCAGCTCTGCRGC’] | UDPglucose-hexose-1- | |
| | | | | phosphate | |
| | | | | uridylyltransferase’] | |
| NM_018718.2(CEP41): | 368178632 | CEP41 | [‘TTCGAGCTTCTCAGT | [‘Joubert syndrome 9/15, | 295 |
| c.107T > C (p.Met36Thr) | | | ATATTTAGTCRTA’] | digenic’] | |
| NM_005359.5(SMAD4): | 377767327 | SMAD4 | [‘TTTGTTTTCCCCTTTA | [‘Juvenile polyposis | 296 |
| c.425-6A > G | | | AACARTTAAGAT’] | syndrome’] | |
| NM_002739.3(PRKCG): | 386134157 | PRKCG | [‘TTTGCAGAAAGGGG | [‘Spinocerebellar ataxia | 297 |
| c.76A > G (p.Arg26Gly) | | | GCCCTGRGGCAGAA’] | 14’] | |
| NM_000138.4(FBN1): | 387906622 | FBN1 | [‘TTTGTGCTACAGAAA | [‘Geleophysic dysplasia | 298 |
| c.5096A > G (p.Tyr1699Cys) | | | CTRCTATGCTGAC’] | 2’] | |
| NM_000138.4(FBN1): | 387906626 | FBN1 | [‘TTTGTGCTACAGAAA | [ ] | 299 |
| c.5099A > G (p.Tyr1700Cys) | | | CTACTRTGCTGAC’] | | |
| m.10450A > G | 387906731 | MT-TR | [‘TTCGACTCATTAAAT | [‘Mitochondrial | 300 |
| | | | TRTGATAATCATA’] | encephalomyopathy’] | |
| m.5816A > G | 387906732 | MT-TC | [‘TTTGCAATTCAATAT | [ ] | 301, 302 |
| | | | GAAAATCRCCTCG’]; | | |
| | | | [‘TTTGCAATTCAATAT | | |
| | | | GAAAATCRCCTCG’] | | |
| NM_001184.3(ATR): | 387906797 | ATR | [‘TTTGACTGCTTTTTC | [‘Cutaneous | 303 |
| c.6431A > G (p.Gln2144Arg) | | | ACRATTGATCTCT’] | telangiectasia and cancer | |
| | | | | syndrome, familial’] | |
| NM_006587.3(CORIN): | 387906895 | CORIN | [‘TTTGCCCTACAACAG | [‘Preeclampsia/eclampsia | 304 |
| c.1414A > G (p.Ser472Gly) | | | TACARGTTATCCA’] | 5’] | |
| NM_024700.3(SNIP1): | 387906986 | SNIP1 | [‘TTTGGATTCAGTAGC | [‘Psychomotor | 305 |
| c.1097A > G (p.Glu366Gly) | | | AGAGRATACGTCT’] | retardation, epilepsy, and | |
| | | | | craniofacial | |
| | | | | dysmorphism’] | |
| NM_016952.4(CDON): | 387906997 | CDON | [‘TTTGTTTTCCCTCAA | [‘Holoprosencephaly 11’] | 306 |
| c.2368A > G (p.Thr790Ala) | | | AGGTTCARCATAC’] | | |
| NM_201269.2(ZNF644): | 387907109 | ZNF644 | [‘TTTGGATCAACCTCA | [‘Myopia 21, autosomal | 307 |
| c.2014A > G (p.Ser672Gly) | | | CAATCARGTAGTT’] | dominant’] | |
| NM_016464.4(TMEM138): | 387907132 | TMEM138 | [‘TTTGCCCTCAGCATC | [‘Joubert syndrome 16’] | 308 |
| c.287A > G | | | TCCCTTCRTGTCT’] | | |
| (p.His96Arg) | | | | | |
| NM_021629.3(GNB4):c. | 387907341 | GNB4 | [‘TTTGGGATAGCTATA | [‘Charcot-Marie-Tooth | 309 |
| 265A > G (p.Lys89Glu) | | | CAACAAATRAGGT’] | disease, dominant | |
| | | | | intermediate F’] | |
| NM_032237.4(POMK): | 397509386 | POMK | [‘TTCGTGGCTCCAGAG | [‘Congenital muscular | 310 |
| c.773A > G (p.Gln258Arg) | | | CRACTGTGGCCCT’] | dystrophy- | |
| | | | | dystroglycanopathy with | |
| | | | | brain and eye anomalies, | |
| | | | | type A12’] | |
| NM_181690.2(AKT3): | 397514605 | AKT3 | [‘TTTGTGATGGAATAT | [‘Megalencephaly- | 311 |
| c.686A > G (p.Asn229Ser) | | | GTTARTGGGGGCG’] | polymicrogyria- | |
| | | | | polydactyly- | |
| | | | | hydrocephalus syndrome | |
| | | | | 2’] | |
| NM_000138.4(FBN1): | 397515756 | FBN1 | [‘TTTGTTTTGTTGTGTT | [‘Marfan syndrome’] | 312, 313 |
| c.1148-2A > G | | | TTTCTRGAGGAT’, | | |
| | | | ‘TTTGTTGTGTTTTTCT | | |
| | | | RGAGGATTTCAA’] | | |
| NM_000551.3(VHL): | 397516441 | VHL | [‘TTTGGTTTTTGCCCTT | [‘Von Hippel-Lindau | 314, 315 |
| c.467A > G (p.Tyr156Cys) | | | CCAGTGTRTACT’, | syndrome’] | |
| | | | ‘TTTGCCCTTCCAGTGT | | |
| | | | RTACTCTGAAAG’] | | |
| NM_033056.3(PCDH15): | 397517452 | PCDH15 | [‘TTTGTTTGTTTGTTTT | [‘Usher syndrome, type | 316, 317 |
| c.1998-2A > G | | | GTCACTRGCACG’, | IF’] | |
| | | | ‘TTTGTTTGTTTTGTCA | | |
| | | | CTRGCACGGGGA’] | | |
| NM_000833.4(GRIN2A): | 397518469 | GRIN2A | [‘TTTGTGTTTGTGCTG | [‘Focal epilepsy with | 318 |
| c.1123-2A > G | | | CRGGTGGGCAAGT’] | speech disorder with or | |
| | | | | without mental | |
| | | | | retardation’] | |
| NM_001204316.1 (PRLR): | 398122522 | PRLR | [‘TTCGCTGCAAACCAG | [‘Hyperprolactinemia’] | 319 |
| c.635A > G (p.His212Arg) | | | ACCRTGGATACTG’] | | |
| NM_000059.3(BRCA2): | 398122779 | BRCA2 | [‘TTTGCTTTGTTTTATT | [‘Familial cancer of | 320 |
| c.426-2A > G | | | TTRGTCCTGTTG’] | breast’, ‘Breast-ovarian | |
| | | | | cancer, familial 2’] | |
| NM_000834.3(GRIN2B): | 398122824 | GRIN2B | [‘TTTGTTTGCTTTTTTC | [‘Mental retardation, | 321, 322 |
| c.2172-2A > G | | | CTGTACRGGAAA’, | autosomal dominant 6’] | |
| | | | ‘TTTGCTTTTTTCCTGT | | |
| | | | ACRGGAAACTGG’] | | |
| NM_003688.3(CASK): | 398122844 | CASK | [‘TTTGATCAATTAGAT | [‘FG syndrome 4’, | 323 |
| c.2168A > G (p.Tyr723Cys) | | | CTTGTCACATRTG’] | ‘Mental retardation and | |
| | | | | microcephaly with | |
| | | | | pontine and cerebellar | |
| | | | | hypoplasia’] | |
| NM_130466.3(UBE3B): | 398123022 | UBE3B | [‘TTTGTTCTCACTGTTT | [‘Kaufman | 324 |
| c.545-2A > G | | | TCTTTCTTTRGG’] | oculocerebrofacial | |
| | | | | syndrome’] | |
| NM_000350.2(ABCA4): | 398123339 | ABCA4 | [‘TTTGTTTTGTTTTTCC | [‘Stargardt disease 1’] | 325 |
| c.67-2A > G | | | RGATTCGCTTTG’] | | |
| NC_000007.14: | 483352872 | — | [‘TTTGAGGCCTGTGGT | [‘Isolated growth | 326 |
| g.62535490A > G | | | TGARATGGAAATA’] | hormone deficiency type | |
| | | | | IB’] | |
| NM_001199397.1(NEK1): | 483352906 | NEK1 | [‘TTTGTATCTTTTCATC | [‘Short rib-polydactyly | 327 |
| c.869-2A > G | | | TRGCTAAAAGAC’] | syndrome, Majewski | |
| | | | | type’] | |
| NM_015474.3(SAMHD1): | 515726139 | SAMHD1 | [‘TTTGTGTTGATAAGC | [‘Aicardi Goutieres | 328 |
| c.1106T > C | | | TCTACGGTGTRAA’] | syndrome 5’] | |
| (p.Leu369Ser) | | | | | |
| NM_031229.2(RBCK1): | 566912235 | RBCK1 | [‘TTTGAGGATGATGTC | [‘Polyglucosan body | 329 |
| c.1160A > G (p.Asn387Ser) | | | ARTGAGTTCACCT’] | myopathy 1 with or | |
| | | | | without | |
| | | | | immunodeficiency’] | |
| NM_001024847.2(TGFBR2): | 587776770 | TGFBR2 | [‘TTTGCTGGCTTTCTT | [‘Loeys-Dietz syndrome | 330 |
| c.1472-2A > G | | | CACRGAAGTAAAA’] | 2’] | |
| NM_003094.3(SNRPE): | 587776924 | SNRPE | [‘TTTGTGAAATTCCAC | [‘Hypotrichosis 11’] | 331 |
| c.1A > G (p.Met1Val) | | | CRTGGCGTACCGT’] | | |
| NM_022068.3(PIEZO2): | 587777454 | PIEZO2 | [‘TTTGATCATTGTCTC | [‘Oculomelic | 332 |
| c.8215T > C (p.Ser2739Pro) | | | TGGTGRGCGATAT’] | amyoplasia’] | |
| NM_152384.2(BBS5): | 587777828 | BBS5 | [‘TTTGGAATTTATCCA | [‘Bardet-Biedl syndrome | 333 |
| c.522 + 3A > G | | | GTGATCAGGTRTT’] | 5’] | |
| NM_000136.2(FANCC): | 587779898 | FANCC | [‘TTTGTTCCCTTTCTTA | [‘Hereditary cancer- | 334 |
| c.-78-2A > G | | | CRGATTTAATGT’] | predisposing syndrome’] | |
| NM_058216.2(RAD51C): | 587780835 | RAD51C | [‘TTTGTATATATATTT | [‘Fanconi anemia, | 335 |
| c.1027-2A > G | | | TTTATCTTTCRGC’] | complementation group | |
| | | | | O’] | |
| NM_000060.3(BTD): | 587783004 | BTD | [‘TTCGATGTTCCTCTT | [‘Biotinidase deficiency’] | 336 |
| c.683A > G (p.Asp228Gly) | | | AAAGTGGRTCTCA’] | | |
| NM_003159.2(CDKL5): | 587783072 | CDKL5 | [‘TTCGGACTCTCAAGC | [‘Atypical Rett | 337 |
| c.211A > G (p.Asn71Asp) | | | AGGAARACATTGT’] | syndrome’, ‘not | |
| | | | | provided’] | |
| NM_178151.2(DCX): | 587783518 | DCX | [‘TTTGTCTCTTCTCTTC | [‘Heterotopia’] | 338 |
| c.1027-2A > G | | | TTATRGGACCTG’] | | |
| NM_000252.2(MTM1): | 587783789 | MTM1 | [‘TTTGATTATAATTTT | [‘Severe X-linked | 339 |
| c.1406A > G (p.His469Arg) | | | GGATCRTCTGTAT’] | myotubular myopathy’] | |
| NM_022458.3(LMBR1): | 606231150 | LMBR1 | [‘TTTGATCTTAGTGTT | [‘Triphalangeal thumb’, | 340 |
| c.423 + 5252A > G | | | TATTACAGRAAAT’] | ‘Preaxial polydactyly 2’] | |
| NM_130466.3(UBE3B): | 672601304 | UBE3B | [‘TTTGTGCAAGTTTGC | [‘Kaufman | 341 |
| c.1A > G (p.Met1Val) | | | AAACRTGTTCACC’] | oculocerebrofacial | |
| | | | | syndrome’] | |
| NM_014305.3(TGDS): | 724160004 | TGDS | [‘TTTGTGAAACTGCTT | [‘Catel Manzke | 342 |
| c.269A > G (p.Glu90Gly) | | | TTTGAAACAGRGA’] | syndrome’] | |
| NM_014305.3(TGDS): | 724160005 | TGDS | [‘TTTGTTTTCCAAGAC | [‘Catel Manzke | 343 |
| c.892A > G (p.Asn298Asp) | | | CCACCRATGACAT’] | syndrome’] | |
| NM_001145901.1(SARS2): | 727502784 | SARS2 | [‘TTCGTCCTTGACCGC | [‘Hyperuricemia, | 344 |
| c.1l75A > G | | | AGGGTCCTGGRTA’] | pulmonary hypertension, | |
| (p.Asp392Gly) | | | | renal failure, and | |
| | | | | alkalosis’] | |
| NM_172362.2(KCNH1): | 727502821 | KCNH1 | [‘TTCGGGAATGTGACG | [‘Temple-Baraitser | 345, 346 |
| c.1508A > G (p.Gln503Arg) | | | ACTATTTTCCRAC’]; | syndrome’] | |
| | | | [‘TTCGGGAATGTGACG | | |
| | | | ACTATTTTCCRAC’] | | |
| NM_000257.3(MYH7): | 727504270 | MYH7 | [‘TTTGTGCTGGAGCAG | [‘Cardiomyopathy’, ‘not | 347 |
| c.1496A > G (p.Glu499Gly) | | | GRGGAGTACAAGA’] | specified’] | |
| NM_001110792.1 (MECP2): | 727505391 | MECP2 | [‘TTTGACTTCACGGTA | [‘Rett disorder’] | 348 |
| c.520A > G | | | ACTGGGRGAGGGA’] | | |
| (p.Arg174Gly) | | | | | |
| NM_006343.2(MERTK): | 730880273 | MERTK | [‘TTTGTTTTCATTCAC | [‘Retinitis pigmentosa | 349 |
| c.1605-2A > G | | | CCRGGAATGCATT’] | 38’] | |
| NM_003611.2(OFD1): | 730880283 | OFD1 | [‘TTTGGCAATAATGAA | [‘Retinitis Pigmentosa | 350 |
| c.935 + 706A > G | | | AAGGTAARTTGAT’] | 23’] | |
| NM_004793.3(LONP1): | 730880293 | LONP1 | [‘TTTGTGGAGACATCC | [‘CODAS syndrome’] | 351 |
| c.2353A > G (p.Arg785Gly) | | | CTGRGACGGCCAC’] | | |
| NM_005633.3(SOS1): | 730881044 | SOSI | [‘TTTGCTGTAAATCAA | [‘Rasopathy’] | 352 |
| c.1430A > G (p.Gln477Arg) | | | ATCATGGGCRGCC’] | | |
| NM_012233.2(RAB3GAP1): | 730882183 | RAB3GA | [‘TTTGCATGTATTTCC | [‘Warburg micro | 353 |
| c.649-2A > G | | P1 | TRGGGATGTCCTT’] | syndrome 1’] | |
| NM_016218.2(POLK): | 757103131 | POLK | [‘TTTGTAAAACCTCTA | [‘Malignant tumor of | 354 |
| c.1679A > T (p.Glu560Val) | | | GRAATGTCTCATA’] | prostate’] | |
| NM_003588.3(CUL4B): | 786200913 | CUL4B | [‘TTTGATTTCTTTTTTT | [‘Syndromic X-linked | 355 |
| c.901-2A > G | | | TTCATTGGCRGA’] | mental retardation, | |
| | | | | Cabezas type’] | |
| NM_014946.3(SPAST): | 786204132 | SPAST | [‘TTTGGTCCACCTGGG | [‘Spastic paraplegia 4, | 356 |
| c.1165A > G (p.Thr389Ala) | | | AATGGGAAGRCAA’] | autosomal dominant’] | |
| NM_003816.2(ADAM9): | 786205086 | ADAM9 | [‘TTTGTATACGTGTAA | [ ] | 357 |
| c.411-8A > G | | | TGCARCATTCAGA’] | | |
| NM_003816.2(ADAM9): | 786205151 | ADAM9 | [‘TTTGAATTCTATTTC | [ ] | 358 |
| c.1396-2A > G | | | ACTRGTTCCTTCC’] | | |
| NM_003002.3(SDHD):c. | 786205436 | SDHD | [‘TTTGAATCCTTGCTC | [‘Fatal infantile | 359 |
| 275A > G (p.Asp92Gly) | | | TGCGATGGRCTAT’] | mitochondrial | |
| | | | | cardiomyopathy’] | |
| NM_016218.2(POLK): | 786205692 | POLK | [‘TTTGCCATTGCTAAG | [‘Malignant tumor of | 360 |
| c.1477A > G (p.Lys493Glu) | | | GAATTGCTARAAA’] | prostate’] | |
| NM_001604.5(PAX6): | 794726661 | PAX6 | [‘TTTGCCTCTCTCCTC | [‘Congenital aniridia’] | 361 |
| c.1075-2A > G | | | ACRGCCCCCAGTC’] | | |
| NM_001165963.1(SCN1A): | 794726849 | SCN1A | [‘TTTGACAAACTGTGT | [‘Severe myoclonic | 362 |
| c.433A > G | | | GTTTRTGACAATG’] | epilepsy in infancy’] | |
| (p.Met145Val) | | | | | |
| NM_000138.4(FBN1): | 794728216 | FBN1 | [‘TTTGCTTTTTTCTCCC | [‘Thoracic aortic | 363 |
| c.4337-2A > G | | | TCCCCCCARGAT’] | aneurysms and aortic | |
| | | | | dissections’] | |
| NM_000238.3(KCNH2): | 794728377 | KCNH2 | [‘TTCGGCAACGTCTCT | [‘Cardiac arrhythmia’] | 364, 365 |
| c.1900A > G (p.Thr634Ala) | | | CCCAACRCCAACT’]; | | |
| | | | [‘TTCGGCAACGTCTCT | | |
| | | | CCCAACRCCAACT’] | | |
| NM_198056.2(SCN5A): | 794728886 | SCN5A | [‘TTTGCACTTAGGGGG | [‘not provided’] | 366 |
| c.4453A > G (p.Ile1485Val) | | | CCAGGACRTCTTC’] | | |
| NM_030973.3(MED25): | 794729668 | MED25 | [‘TTCGAGGGGCTCCGC | [‘BASEL-VANAGAITE- | 367 |
| c.116A > G (p.Tyr39Cys) | | | AAGCACTRCCTGC’] | SMIRIN-YOSEF | |
| | | | | SYNDROME’] | |
| NM_006017.2(PROM1): | 796051882 | PROM1 | [‘TTTGCTGTTCTTCAC | [‘Cone-rod dystrophy 2’] | 368 |
| c.2077-521A > G | | | AGCTTTAGGRTAT’] | | |
| NM_000016.5(ACADM): | 796051900 | ACADM | [‘TTTGATGCTTGTTTA | [‘not provided’] | 369 |
| c.329A > G (p.Glu110Gly) | | | ATTAGTGRAGAAT’] | | |
| NM_000833.4(GRIN2A): | 796052544 | GRIN2A | [‘TTCGCTGTCATATTC | [‘not provided’] | 370 |
| c.1930A > G | | | CTGGCTRGCTACA’] | | |
| (p.Ser644Gly) | | | | | |
| NM_021007.2(SCN2A): | 796053169 | SCN2A | [‘TTTGTCTTCCTTGAC | [‘not provided’] | 371, 372 |
| c.387-2A > G | | | GATATTCTRCTTT’]; | | |
| | | | [‘TTTGTCTTCCTTGAC | | |
| | | | GATATTCTRCTTT’] | | |
| NM_021007.2(SCN2A): | 796053173 | SCN2A | [‘TTTGCAATGGCCTCC | [‘not provided’] | 373 |
| c.851A > G (p.Asp284Gly) | | | AGRTAATTCTTCC’] | | |

It will be apparent to those of skill in the art that in order to target a GeoCas9 base editor fusion protein as disclosed herein to a target site, e.g., a site comprising a point mutation to be edited, it is typically necessary to co-express the GeoCas9 fusion protein together with a guide RNA, e.g., an sgRNA. As explained in more detail elsewhere herein, a guide RNA typically comprises a tracrRNA framework allowing for Cas9 binding, and a guide sequence, which confers sequence specificity to the Cas9: nucleic acid editing enzyme/domain fusion protein. In some embodiments, the guide RNA comprises a structure 5′-[guide sequence]-[GUCAUAGUUCCCCUGAGAAAUCAGGGUUACUAUGAUAAGGGCUUUCUGCCUA AGGCAGACUGACCCGCGGCGUUGGGGAUCGCCUGUCGCCCGCUUUUGGGGGC AUUCCCCAUCCUUUUUUU-3′ (SEQ ID NO: 77)]-3′, wherein the guide sequence comprises a sequence that is complementary to the target sequence. The guide sequence is typically 20 nucleotides long. The sequences of suitable guide RNAs for targeting GeoCas9: nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting Cas9: nucleic acid editing enzyme/domain fusion proteins to specific target sequences are provided herein.

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a GeoCas9 domain fused to a cytidine deaminase domain) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase; and the method results in less than 20% indel formation in the nucleic acid. It should be appreciated that in some embodiments, step b is omitted. In some embodiments, the first nucleobase is a cytosine. In some embodiments, the second nucleobase is a deaminated cytosine, or a uracil. In some embodiments, the third nucleobase is a guanine. In some embodiments, the fourth nucleobase is an adenine. In some embodiments, the first nucleobase is a cytosine, the second nucleobase is a deaminated cytosine, or a uracil, the third nucleobase is a guanine, and the fourth nucleobase is an adenine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., C: G->T: A). In some embodiments, the fifth nucleobase is a thymine. In some embodiments, at least 5% of the intended basepairs are edited. In some embodiments, at least 10%, 15%, 20%, 25%, 30%, 35%, 40%, 45%, or 50% of the intended basepairs are edited.

In some embodiments, the ratio of intended products to unintended products in the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand (nicked strand) is hybridized to the guide nucleic acid. In some embodiments, the cut single strand is opposite to the strand comprising the first nucleobase. In some embodiments, the base editor comprises a GeoCas9 domain. In some embodiments, the first base is cytosine, and the second base is not a G, C, A, or T. In some embodiments, the second base is uracil. In some embodiments, the first base is cytosine. In some embodiments, the second base is not a G, C, A, or T. In some embodiments, the second base is uracil. In some embodiments, the base editor inhibits base escision repair of the edited strand. In some embodiments, the base editor protects or binds the non-edited strand. In some embodiments, the base editor comprises UGI activity. In some embodiments, the base editor comprises nickase activity. In some embodiments, the intended edited basepair is upstream of a PAM site (e.g., 5′-NNNNCRAA-3′ (SEQ ID NO: 79)). In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical GeoCas9 PAM site (e.g., 5′-NNNNCRAA-3′ (SEQ ID NO: 79)). In some embodiments, the base editor comprises a linker. In some embodiments, the linker is 1-25 amino acids in length. In some embodiments, the linker is 5-20 amino acids in length. In some embodiments, linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair is within the target window. In some embodiments, the target window comprises the intended edited base pair. In some embodiments, the method is performed using any of the base editors provided herein. In some embodiments, a target window is a deamination window

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited basepair, wherein the efficiency of generating the intended edited basepair is at least 5%. It should be appreciated that in some embodiments, step b is omitted. In some embodiments, at least 5% of the intended basepaires are edited. In some embodiments, at least 10%, 15%, 20%, 25%, 30%, 35%, 40%, 45%, or 50% of the intended basepaires are edited. In some embodiments, the method causes less than 19%, 18%, 16%, 14%, 12%, 10%, 8%, 6%, 4%, 2%, 1%, 0.5%, 0.2%, or less than 0.1% indel formation. In some embodiments, the ratio of intended product to unintended products at the target nucleotide is at least 2:1, 5:1, 10:1, 20:1, 30:1, 40:1, 50:1, 60:1, 70:1, 80:1, 90:1, 100:1, or 200:1, or more. In some embodiments, the ratio of intended point mutation to indel formation is greater than 1:1, 10:1, 50:1, 100:1, 500:1, or 1000:1, or more. In some embodiments, the cut single strand is hybridized to the guide nucleic acid. In some embodiments, the cut single strand is opposite to the strand comprising the first nucleobase. In some embodiments, the first base is cytosine. In some embodiments, the second nucleobase is not G, C, A, or T. In some embodiments, the second base is uracil. In some embodiments, the base editor inhibits base escision repair of the edited strand. In some embodiments, the base editor protects or binds the non-edited strand. In some embodiments, the nucleobase editor comprises UGI activity. In some embodiments, the nucleobase edit comprises nickase activity. In some embodiments, the intended edited basepair is upstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides upstream of the PAM site (e.g., 5′-NNNNCRAA-3′ (SEQ ID NO: 79)). In some embodiments, the intended edited basepair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical GeoCas9 PAM site (e.g., 5′-NNNNCRAA-3′ (SEQ ID NO: 79)). In some embodiments, the nucleobase editor comprises a linker. In some embodiments, the linker is 1-25 amino acids in length. In some embodiments, the linker is 5-20 amino acids in length. In some embodiments, the linker is 10, 11, 12, 13, 14, 15, 16, 17, 18, 19, or 20 amino acids in length. In some embodiments, the target region comprises a target window, wherein the target window comprises the target nucleobase pair. In some embodiments, the target window comprises 1-10 nucleotides. In some embodiments, the target window is 1-9, 1-8, 1-7, 1-6, 1-5, 1-4, 1-3, 1-2, or 1 nucleotides in length. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edited base pair occurs within the target window. In some embodiments, the target window comprises the intended edited base pair. In some embodiments, the nucleobase editor is any one of the base editors provided herein.

### V. Base Editor Efficiency

Some aspects of the disclosure are based on the recognition that any of the base editors provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g. mutate or deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations or deaminations) versus indels. In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method known in the art

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, an number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

Some aspects of the disclosure are based on the recognition that any of the base editors provided herein are capable of efficiently generating an intended mutation, such as a point mutation, in a nucleic acid (e.g. a nucleic acid within a genome of a subject) without generating a significant number of unintended mutations, such as unintended point mutations. In some embodiments, a intended mutation is a mutation that is generated by a specific base editor bound to a gRNA, specifically designed to generate the intended mutation. In some embodiments, the intended mutation is a mutation associated with a disease or disorder. In some embodiments, the intended mutation is a cytosine (C) to thymine (T) point mutation associated with a disease or disorder. In some embodiments, the intended mutation is a guanine (G) to adenine (A) point mutation associated with a disease or disorder. In some embodiments, the intended mutation is a cytosine (C) to thymine (T) point mutation within the coding region of a gene. In some embodiments, the intended mutation is a guanine (G) to adenine (A) point mutation within the coding region of a gene. In some embodiments, the intended mutation is a point mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations: unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more. It should be appreciated that the characteristics of the base editors described in the “Base Editor Efficiency” section, herein, may be applied to any of the fusion proteins, or methods of using the fusion proteins provided herein.

### VI. Pharmaceutical Compositions

Other aspects of the present disclosure relate to pharmaceutical compositions comprising any of the various components described herein (e.g., including, but not limited to, the GeoCas9 proteins, fusion proteins, guide RNAs, and complexes comprising fusion proteins and guide RNAs). In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments, the pharmaceutical composition comprises a ribonucleoprotein complex comprising an RNA-guided nuclease (e.g., GeoCas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions may optionally comprise one or more additional therapeutically active substances.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.). Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

In some embodiments, compositions provided herein are administered to a subject, for example, to a human subject, in order to effect a targeted genomic modification within the subject. In some embodiments, cells are obtained from the subject and contacted with a any of the pharmaceutical compositions provided herein. In some embodiments, cells removed from a subject and contacted ex vivo with a pharmaceutical composition are re-introduced into the subject, optionally after the desired genomic modification has been effected or detected in the cells. Methods of delivering pharmaceutical compositions comprising nucleases are known, and are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties. Although the descriptions of pharmaceutical compositions provided herein are principally directed to pharmaceutical compositions which are suitable for administration to humans, it will be understood by the skilled artisan that such compositions are generally suitable for administration to animals or organisms of all sorts. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, domesticated animals, pets, and commercially relevant mammals such as cattle, pigs, horses, sheep, cats, dogs, mice, and/or rats; and/or birds, including commercially relevant birds such as chickens, ducks, geese, and/or turkeys.

Formulations of the pharmaceutical compositions described herein may be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit.

Pharmaceutical formulations may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro (Lippincott, Williams & Wilkins, Baltimore, MD, 2006; incorporated in its entirety herein by reference) discloses various excipients used in formulating pharmaceutical compositions and known techniques for the preparation thereof. See also PCT application PCT/US2010/055131 (Publication number WO2011053982 A8, filed Nov. 2, 2010), incorporated in its entirety herein by reference, for additional suitable methods, reagents, excipients and solvents for producing pharmaceutical compositions comprising a nuclease. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this disclosure.

In some embodiments, compositions in accordance with the present invention may be used for treatment of any of a variety of diseases, disorders, and/or conditions, including but not limited to one or more of the following: autoimmune disorders (e.g. diabetes, lupus, multiple sclerosis, psoriasis, rheumatoid arthritis); inflammatory disorders (e.g. arthritis, pelvic inflammatory disease); infectious diseases (e.g. viral infections (e.g., HIV, HCV, RSV), bacterial infections, fungal infections, sepsis); neurological disorders (e.g. Alzheimer's disease, Huntington's disease; autism; Duchenne muscular dystrophy); cardiovascular disorders (e.g. atherosclerosis, hypercholesterolemia, thrombosis, clotting disorders, angiogenic disorders such as macular degeneration); proliferative disorders (e.g. cancer, benign neoplasms); respiratory disorders (e.g. chronic obstructive pulmonary disease); digestive disorders (e.g. inflammatory bowel disease, ulcers); musculoskeletal disorders (e.g. fibromyalgia, arthritis); endocrine, metabolic, and nutritional disorders (e.g. diabetes, osteoporosis); urological disorders (e.g. renal disease); psychological disorders (e.g. depression, schizophrenia); skin disorders (e.g. wounds, eczema); blood and lymphatic disorders (e.g. anemia, hemophilia); etc.

In other embodiments, the pharmaceutical composition described herein is delivered in a controlled release system. In one embodiment, a pump may be used (see, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et al., 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic aqueous buffer. Where necessary, the pharmaceutical can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration may be a liquid, e.g., sterile saline, lactated Ringer's or Hank's solution. In addition, the pharmaceutical composition can be in solid forms and re-dissolved or suspended immediately prior to use. Lyophilized forms are also contemplated.

The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et al., Gene Ther. 1999, 6:1438-47). Positively charged lipids such as N-[1-(2,3-dioleoyloxi) propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein may be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile water) for injection. The pharmaceutically acceptable diluent can be used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers may be formed from a variety of materials such as glass or plastic. In some embodiments, the container holds a composition that is effective for treating a disease described herein and may have a sterile access port. For example, the container may be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture may further comprise a second container comprising a pharmaceutically-acceptable buffer, such as phosphate-buffered saline, Ringer's solution, or dextrose solution. It may further include other materials desirable from a commercial and user standpoint, including other buffers, diluents, filters, needles, syringes, and package inserts with instructions for use.

### VII. Delivery Methods

In some aspects, the invention provides methods comprising delivering one or more polynucleotides, such as or one or more vectors as described herein encoding one or more components described herein, one or more transcripts thereof, and/or one or proteins transcribed therefrom, to a host cell. In some aspects, the invention further provides cells produced by such methods, and organisms (such as animals, plants, or fungi) comprising or produced from such cells. In some embodiments, a base editor as described herein in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a base editor to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Bihm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids take advantage of highly evolved processes for targeting a virus to specific cells in the body and trafficking the viral payload to the nucleus. Viral vectors can be administered directly to patients (in vivo) or they can be used to treat cells in vitro, and the modified cells may optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a viruses can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700). In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

Packaging cells are typically used to form virus particles that are capable of infecting a host cell. Such cells include 293 cells, which package adenovirus, and w2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences. The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

### VIII. Kits, Vectors, Cells

Some aspects of this disclosure provide kits comprising a nucleic acid construct, comprising (a) a nucleotide sequence encoding a GeoCas9 protein or a GeoCas9 fusion protein as provided herein; and (b) a heterologous promoter that drives expression of the sequence of (a). In some embodiments, the kit further comprises an expression construct encoding a guide RNA backbone, wherein the construct comprises a cloning site positioned to allow the cloning of a nucleic acid sequence identical or complementary to a target sequence into the guide RNA backbone.

Some aspects of this disclosure provide polynucleotides encoding a GeoCas9 protein of a fusion protein as provided herein. Some aspects of this disclosure provide vectors comprising such polynucleotides. In some embodiments, the vector comprises a heterologous promoter driving expression of polynucleotide.

Some aspects of this disclosure provide cells comprising a GeoCas9 protein, a fusion protein, a nucleic acid molecule encoding the fusion protein, a complex comprise the GeoCas9 protein and the gRNA, and/or a vector as provided herein.

The description of exemplary embodiments of the GeoCas9 system is provided for illustration purposes only and not meant to be limiting. Additional GeoCas9 systems, e.g., variations of the exemplary systems described in detail above, are also embraced by this disclosure. Some aspects of this disclosure provide kits comprising a nucleic acid construct comprising a nucleotide sequence encoding a GeoCas9 fusion protein editor described herein. In some embodiments, the nucleotide sequence comprises a heterologous promoter that drives expression of the GeoCas9 fusion proteins described herein.

Some aspects of this disclosure provide kits comprising a nucleic acid construct comprising a nucleotide sequence encoding a GeoCas9 fusion protein described herein. In some embodiments, the nucleotide sequence comprises a heterologous promoter that drives expression of the GeoCas9 fusion proteins.

Some aspects of this disclosure provide kits comprising a nucleic acid construct, comprising (a) a nucleotide sequence encoding a GeoCas9 (e.g., a GeoCas9 nickase) fused to a nucleic acid editing domain and (b) a heterologous promoter that drives expression of the sequence of (a). In some embodiments, the kit further comprises an expression construct encoding a guide RNA, or wherein the nucleic acid construct comprising the GeoCas9 fusion further comprises the nucleic acid sequence encoding the guide RNA. The promoters driving expression of the GeoCas9 fusion protein and the guide RNA can be the same of different.

Some aspects of this disclosure provide cells comprising any of the constructs disclosed herein. In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A 172, A20, A253, A431, A-549, ALC, B16, B35, BCP-1 cells, BEAS-2B, bEnd.3, BHK-21, BR 293. BxPC3. C3H-10T1/2, C6/36, Cal-27, CHO, CHO-7, CHO—IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalc1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK 11, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which more than one members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

### EXAMPLES

### Example 1—Construction of GeoCas9 Base Editors

### Methods

Plasmids encoding GeoBE3 or GeoBE4, and a plasmid encoding a specific gRNA are transfected into HEK293T cells using Lipofectamine 2000 according to manufacturer's protocol. After 48 h, the cells were harvested and the genomic DNA was extracted. Target site amplicons were analyzed by Illumina high-throughput sequencing. Base editing efficiency was calculated using a custom Matlab program as used previously (Komor et al 2016).

### Results

GeoBE3 and GeoBE4 can mediate efficient base editing on several genomic loci in HEK293T cells. GeoBE3 and GeoBE4 exhibits comparable base editing efficiencies. GeoBE3 and GeoBE4 has a wider window of deamination as evidenced by significant base editing occurring around the target site that is usually untargetable using SpCas9-based base editors. Base editing efficiencies varied from ˜0 to 35%. See FIG. 3.

GeoBE3 and GeoBE4 expands the targeting scope of base editing by providing access to additional PAM sites within the genome.

### Exemplary Sequences

GeoCas9 single guide RNA (X denotes target sequence) 5′ >3′
(SEQ ID NO: 435)
5′XXXXXXXXXXXXXXXXXXXXXXGTCATAGTTCCCCTGAGAAATCAG
GGTTACTATGATAAGGGCTTTCTGCCTAAGGCAGACTGACCCGCGGCGTTGGGGA
TCGCCTGTCGCCCGCTTTTGGCGGGCATTCCCCATCCTTTTTTT-3′
wherein X is A, U, C, or G.
GeoCas9 nickase (D8A)
(SEQ ID NO: 2)
RYKIGLAIGITSVGWAVMNLDIPRIEDLGVRIFDRAENPQTGESLALPRRLA
RSARRRLRRRKHRLERIRRLVIREGILTKEELDKLFEEKHEIDVWQLRVEALDRKLNN
DELARVLLHLAKRRGFKSNRKSERSNKENSTMLKHIEENRAILSSYRTVGEMIVKDP
KFALHKRNKGENYTNTIARDDLEREIRLIFSKQREFGNMSCTEEFENEYITIWASQRP
VASKDDIEKKVGFCTFEPKEKRAPKATYTFQSFIAWEHINKLRLISPSGARGLTDEER
RLLYEQAFQKNKITYHDIRTLLHLPDDTYFKGIVYDRGESRKQNENIRFLELDAYHQI
RKAVDKVYGKGKSSSFLPIDFDTFGYALTLFKDDADIHSYLRNEYEQNGKRMPNLA
NKVYDNELIEELLNLSFTKFGHLSLKALRSILPYMEQGEVYSSACERAGYTFTGPKKK
QKTMLLPNIPPIANPVVMRALTQARKVVNAIIKKYGSPVSIHIELARDLSQTFDERRKT
KKEQDENRKKNETAIRQLMEYGLTLNPTGHDIVKFKLWSEQNGRCAYSLQPIEIERL
LEPGYVEVDHVIPYSRSLDDSYTNKVLVLTRENREKGNRIPAEYLGVGTERWQQFET
FVLTNKQFSKKKRDRLLRLHYDENEETEFKNRNLNDTRYISRFFANFIREHLKFAESD
DKQKVYTVNGRVTAHLRSRWEFNKNREESDLHHAVDAVIVACTTPSDIAKVTAFYQ
RREQNKELAKKTEPHFPQPWPHFADELRARLSKHPKESIKALNLGNYDDQKLESLQP
VFVSRMPKRSVTGAAHQETLRRYVGIDERSGKIQTVVKTKLSEIKLDASGHFPMYGK
ESDPRTYEAIRQRLLEHNNDPKKAFQEPLYKPKKNGEPGPVIRTVKIIDTKNQVIPLND
GKTVAYNSNIVRVDVFEKDGKYYCVPVYTMDIMKGILPNKAIEPNKPYSEWKEMTE
DYTFRFSLYPNDLIRIELPREKTVKTAAGEEINVKDVFVYYKTIDSANGGLELISHDHR
FSLRGVGSRTLKRFEKYQVDVLGNIYKVRGEKRVGLASSAHSKPGKTIRPLQSTRD
GeoBE3
(SEQ ID NO: 80)
MSSETGPVAVDPTLRRRIEPHEFEVFFDPRELRKETCLLYEINWGGRHSIWR
HTSQNTNKHVEVNFIEKFTTERYFCPNTRCSITWFLSWSPCGECSRAITEFLSRYPHVT
LFIYIARLYHHADPRNRQGLRDLISSGVTIQIMTEQESGYCWRNFVNYSPSNEAHWPR
YPHLWVRLYVLELYCIILGLPPCLNILRRKQPQLTFFTIALQSCHYQRLPPHILWATGL
KSGSETPGTSESATPESRYKIGLAIGITSVGWAVMNLDIPRIEDLGVRIFDRAENPQTG
ESLALPRRLARSARRRLRRRKHRLERIRRLVIREGILTKEELDKLFEEKHEIDVWQLR
VEALDRKLNNDELARVLLHLAKRRGFKSNRKSERSNKENSTMLKHIEENRAILSSYR
TVGEMIVKDPKFALHKRNKGENYTNTIARDDLEREIRLIFSKQREFGNMSCTEEFENE
YITIWASQRPVASKDDIEKKVGFCTFEPKEKRAPKATYTFQSFIA WEHINKLRLISPSG
ARGLTDEERRLLYEQAFQKNKITYHDIRTLLHLPDDTYFKGIVYDRGESRKQNENIRF
LELDAYHQIRKAVDKVYGKGKSSSFLPIDFDTFGYALTLFKDDADIHSYLRNEYEQN
GKRMPNLANKVYDNELIEELLNLSFTKFGHLSLKALRSILPYMEQGEVYSSACERAG
YTFTGPKKKQKTMLLPNIPPIANPVVMRALTQARKVVNAIIKKYGSPVSIHIELARDL
SQTFDERRKTKKEQDENRKKNETAIRQLMEYGLTLNPTGHDIVKFKLWSEQNGRCA
YSLQPIEIERLLEPGYVEVDHVIPYSRSLDDSYTNKVLVLTRENREKGNRIPAEYLGV
GTERWQQFETFVLTNKQFSKKKRDRLLRLHYDENEETEFKNRNLNDTRYISRFFANF
IREHLKFAESDDKQKVYTVNGRVTAHLRSRWEFNKNREESDLHHAVDAVIVACTTP
SDIAKVTAFYQRREQNKELAKKTEPHFPQPWPHFADELRARLSKHPKESIKALNLGN
YDDQKLESLQPVFVSRMPKRSVTGAAHQETLRRYVGIDERSGKIQTVVKTKLSEIKL
DASGHFPMYGKESDPRTYEAIRQRLLEHNNDPKKAFQEPLYKPKKNGEPGPVIRTVK
IIDTKNQVIPLNDGKTVAYNSNIVRVDVFEKDGKYYCVPVYTMDIMKGILPNKAIEPN
KPYSEWKEMTEDYTFRFSLYPNDLIRIELPREKTVKTAAGEEINVKDVFVYYKTIDSA
NGGLELISHDHRFSLRGVGSRTLKRFEKYQVDVLGNIYKVRGEKRVGLASSAHSKPG
KTIRPLQSTRDSGGSTNLSDIIEKETGKQLVIQESILMLPEEVEEVIGNKPESDILVHTA
YDESTDENVMLLTSDAPEYKPWALVIQDSNGENKIKMLSGGSPKKKRKV
GeoBE4
(SEQ ID NO: 81)
MSSETGPVAVDPTLRRRIEPHEFEVFFDPRELRKETCLLYEINWGGRHSIWR
HTSQNTNKHVEVNFIEKFTTERYFCPNTRCSITWFLSWSPCGECSRAITEFLSRYPHVT
LFIYIARLYHHADPRNRQGLRDLISSGVTIQIMTEQESGYCWRNFVNYSPSNEAHWPR
YPHLWVRLYVLELYCIILGLPPCLNILRRKQPQLTFFTIALQSCHYQRLPPHILWATGL
KSGGSSGGSSGSETPGTSESATPESSGGSSGGSRYKIGLAIGITSVGWAVMNLDIPRIE
DLGVRIFDRAENPQTGESLALPRRLARSARRRLRRRKHRLERIRRLVIREGILTKEELD
KLFEEKHEIDVWQLR VEALDRKLNNDELARVLLHLAKRRGFKSNRKSERSNKENST
MLKHIEENRAILSSYRTVGEMIVKDPKFALHKRNKGENYTNTIARDDLEREIRLIFSK
QREFGNMSCTEEFENEYITIWASQRPVASKDDIEKKVGFCTFEPKEKRAPKATYTFQS
FIAWEHINKLRLISPSGARGLTDEERRLLYEQAFQKNKITYHDIRTLLHLPDDTYFKGI
VYDRGESRKQNENIRFLELDAYHQIRKAVDKVYGKGKSSSFLPIDFDTFGYALTLFK
DDADIHSYLRNEYEQNGKRMPNLANKVYDNELIEELLNLSFTKFGHLSLKALRSILPY
MEQGEVYSSACERAGYTFTGPKKKQKTMLLPNIPPIANPVVMRALTQARKVVNAIIK
KYGSPVSIHIELARDLSQTFDERRKTKKEQDENRKKNETAIRQLMEYGLTLNPTGHDI
VKFKLWSEQNGRCAYSLQPIEIERLLEPGYVEVDHVIPYSRSLDDSYTNKVLVLTREN
REKGNRIPAEYLGVGTERWQQFETFVLINKQFSKKKRDRLLRLHYDENEETEFKNR
NLNDTRYISRFFANFIREHLKFAESDDKQKVYTVNGRVTAHLRSRWEFNKNREESDL
HHAVDAVIVACTTPSDIAKVTAFYQRREQNKELAKKTEPHFPQPWPHFADELRARLS
KHPKESIKALNLGNYDDQKLESLQPVFVSRMPKRSVTGAAHQETLRRYVGIDERSGK
IQTVVKTKLSEIKLDASGHFPMYGKESDPRTYEAIRQRLLEHNNDPKKAFQEPLYKPK
KNGEPGPVIRTVKIIDTKNQVIPLNDGKTVAYNSNIVRVDVFEKDGKYYCVPVYTMD
IMKGILPNKAIEPNKPYSEWKEMTEDYTFRFSLYPNDLIRIELPREKTVKTAAGEEINV
KDVFVYYKTIDSANGGLELISHDHRFSLRGVGSRTLKRFEKYQVDVLGNIYKVRGEK
RVGLASSAHSKPGKTIRPLQSTRDSGGSGGSGGSTNLSDIIEKETGKQLVIQESILMLP
EEVEEVIGNKPESDIL VHTAYDESTDENVMLLTSDAPEYKPWAL VIQDSNGENKIKM
LSGGSGGSGGSTNLSDIIEKETGKQLVIQESILMLPEEVEEVIGNKPESDILVHTAYDES
TDENVMLLTSDAPEYKPWALVIQDSNGENKIKMLSGGSPKKKRKV.

1-137. (canceled)

138. A polynucleotide encoding a fusion protein comprising an amino acid sequence that is at least 85% identical to the amino acid sequence of any one of SEQ ID NOS: 80-81.

139. A vector comprising a polynucleotide of claim 138.

140. (canceled)

141. A cell comprising the polynucleotide of claim 138.

142. A cell comprising the vector of claim 139.

143. (canceled)

144. A pharmaceutical composition comprising the polynucleotide of claim 138 and a pharmaceutically acceptable carrier.

145. A pharmaceutical composition comprising the vector of claim 139 and a pharmaceutically acceptable carrier.

146. The polynucleotide of claim 138, wherein the fusion protein comprises an amino acid sequence that is at least 90% identical to the amino acid sequence of any one of SEQ ID NOs: 80-81.

147. The polynucleotide of claim 138, wherein the fusion protein comprises an amino acid sequence that is at least 95% identical to the amino acid sequence of any one of SEQ ID NOS: 80-81.

148. The polynucleotide of claim 138, wherein the fusion protein comprises an amino acid sequence that is at least 96% identical to the amino acid sequence of any one of SEQ ID NOS: 80-81.

149. The polynucleotide of claim 138, wherein the fusion protein comprises an amino acid sequence that is at least 97% identical to the amino acid sequence of any one of SEQ ID NOs: 80-81.

150. The polynucleotide of claim 138, wherein the fusion protein comprises an amino acid sequence that is at least 98% identical to the amino acid sequence of any one of SEQ ID NOS: 80-81.

151. The polynucleotide of claim 138, wherein the fusion protein comprises an amino acid sequence that is at least 99% identical to the amino acid sequence of any one of SEQ ID NOs: 80-81.

152. The polynucleotide of claim 138, wherein the fusion protein comprises the amino acid sequence of SEQ ID NO: 80.

153. The polynucleotide of claim 138, wherein the fusion protein comprises the amino acid sequence of SEQ ID NO: 81.

154. A method comprising expressing a fusion protein from the polynucleotide of claim 138, wherein the fusion protein is bound to a guide RNA (gRNA) that targets the fusion protein to a nucleic acid molecule.

155. The method of claim 154, wherein the nucleic acid molecule comprises a target sequence associated with a disease or disorder.

156. The method of claim 154, wherein the nucleic acid molecule comprises a point mutation associated with a disease or disorder.

157. The method of claim 156, wherein the activity of the fusion protein results in a correction of the point mutation.

158. The method of claim 154, wherein the contacting is performed in vivo in a subject.

159. The method of claim 154, wherein the contacting is performed in vitro.


SEQUENCE LISTING (436 sequences)

SEQ ID NO: 1 - protein, 1368 aa, Streptococcus pyogenes [sequence omitted]

SEQ ID NO: 2 - protein, 1086 aa, synthetic construct
  REGION 1..1086: note = Synthetic
RYKIGLAIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 3 - protein, 1086 aa, synthetic construct
  REGION 1..1086: note = Synthetic
RYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRD

SEQ ID NO: 4 - DNA, 3249 bp, unidentified
  source 1..3249: mol_type = genomic DNA; note = Bacillus thermodenitrificans
atgaagtataaaatcggtcttgctatcggcattacgtctatcggttgggctgtcattaatttggacattcctcgcatcgaagatttaggtgtccgcatttttgacagagcggaaaacccgaaaaccggggagtcactagctcttccacgtcgcctcgcccgctccgcccgacgtcgtctgcggcgtcgcaaacatcgactggagcgcattcgccgcctgttcgtccgcgaaggaattttaacgaaggaagagctgaacaagctgtttgaaaaaaagcacgaaatcgacgtctggcagcttcgtgttgaagcactggatcgaaaactaaataacgatgaattagcccgcatccttcttcatctggctaaacggcgtggatttagatccaaccgcaagagtgagcgcaccaacaaagaaaacagtacgatgctcaaacatattgaagaaaaccaatccattctttcaagttaccgaacggttgcagaaatggttgtcaaggatccgaaattttccctgcacaagcgtaataaagaggataattacaccaacactgttgcccgcgacgatcttgaacgggaaatcaaactgattttcgccaaacagcgcgaatatgggaacatcgtttgcacagaagcatttgaacacgagtatatttccatttgggcatcgcaacgcccttttgcttctaaggatgatatcgagaaaaaagtcggtttctgtacgtttgagcctaaagaaaaacgcgcgccaaaagcaacatacacattccagtccttcaccgtctgggaacatattaacaaacttcgtcttgtctccccgggaggcatccgggcactaaccgatgatgaacgtcgtcttatatacaagcaagcatttcataaaaataaaatcaccttccatgatgttcgaacattgcttaacttgcctgacgacacccgttttaaaggtcttttatatgaccgaaacaccacgctgaaggaaaatgagaaagttcgcttccttgaactcggcgcctatcataaaatacggaaagcgatcgacagcgtctatggcaaaggagcagcaaaatcatttcgtccgattgattttgatacatttggctacgcattaacgatgtttaaagacgacaccgacattcgcagttacttgcgaaacgaatacgaacaaaatggaaaacgaatggaaaatctagcggataaagtctatgatgaagaattgattgaagaacttttaaacttatcgttttctaagtttggtcatctatcccttaaagcgcttcgcaacatccttccatatatggaacaaggcgaagtctactcaaccgcttgtgaacgagcaggatatacatttacagggccaaagaaaaaacagaaaacggtattgctgccgaacattccgccgatcgccaatccggtcgtcatgcgcgcactgacacaggcacgcaaagtggtcaatgccattatcaaaaagtacggctcaccggtctccatccatatcgaactggcccgggaactatcacaatcctttgatgaacgacgtaaaatgcagaaagaacaggaaggaaaccgaaagaaaaacgaaactgccattcgccaacttgttgaatatgggctgacgctcaatccaactgggcttgacattgtgaaattcaaactatggagcgaacaaaacggaaaatgtgcctattcactccaaccgatcgaaatcgagcggttgctcgaaccaggctatacagaagtcgacgctgtgattccatacagccgaagcttggacgatagctataccaataaagttcttgtgttgacaaaggagaaccgtgaaaaaggaaaccgcaccccagctgaatatttaggattaggctcagaacgttggcaacagttcgagacgtttgtcttgacaaataagcagttttcgaaaaagaagcgggatcgactccttcggcttcattacgatgaaaacgaagaaaatgagtttaaaaatcgtaatctaaatgatacccgttatatctcacgcttcttggctaactttattcgcgaacatctcaaattcgccgacagcgatgacaaacaaaaagtatacacggtcaacggccgtattaccgcccatttacgcagccgttggaattttaacaaaaaccgggaagaatcgaatttgcatcatgccgtcgatgctgccatcgtcgcctgcacaacgccgagcgatatcgcccgagtcaccgccttctatcaacggcgcgaacaaaacaaagaactgtccaaaaagacggatccgcagtttccgcagccttggccgcactttgctgatgaactgcaggcgcgtttatcaaaaaatccaaaggagagtataaaagctctcaatcttggaaattatgataacgagaaactcgaatcgttgcagccggtttttgtctcccgaatgccgaagcggagcataacaggagcggctcatcaagaaacattgcggcgttatatcggcatcgacgaacggagcggaaaaatacagacggtcgtcaaaaagaaactatccgagatccaactggataaaacaggtcatttcccaatgtacgggaaagaaagcgatccaaggacatatgaagccattcgccaacggttgcttgaacataacaatgacccaaaaaaggcgtttcaagagcctctgtataaaccgaagaagaacggagaactaggtcctatcatccgaacaatcaaaatcatcgatacgacaaatcaagttattccgctcaacgatggcaaaacagtcgcctacaacagcaacatcgtgcgggtcgacgtctttgagaaagatggcaaatattattgtgtccctatctatacaatagatatgatgaaagggatcttgccaaacaaggcgatcgagccgaacaaaccgtactctgagtggaaggaaatgacggaggactatacattccgattcagtctatacccaaatgatcttatccgtatcgaatttccccgagaaaaaacaataaagactgctgtgggggaagaaatcaaaattaaggatctgttcgcctattatcaaaccatcgactcctccaatggagggttaagtttggttagccatgataacaacttttcgctccgcagcatcggttcaagaaccctcaaacgattcgagaaataccaagtagatgtgctaggcaacatctacaaagtgagaggggaaaagagagttggggtggcgtcatcttctcattcgaaagccggggaaactatccgtccgttataa

SEQ ID NO: 5 - protein, 1082 aa, unidentified
  source 1..1082: note = Bacillus thermodenitrificans
MKYKIGLDIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 6 - protein, 1087 aa, unidentified
  source 1..1087: note = Bacillus thermoamyloliquefaciens
MRYKIGLDIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAAIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKTGETVRPLQSTRD

SEQ ID NO: 7 - protein, 1082 aa, synthetic construct
  REGION 1..1082: note = Synthetic
MKYKIGLAIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDAVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 8 - protein, 1082 aa, synthetic construct
  REGION 1..1082: note = Synthetic
MKYKIGLAIGITSIGWAVINLDIPRIEDLGVRIFDRAENPKTGESLALPRRLARSARRRLRRRKHRLERIRRLFVREGILTKEELNKLFEKKHEIDVWQLRVEALDRKLNNDELARILLHLAKRRGFRSNRKSERTNKENSTMLKHIEENQSILSSYRTVAEMVVKDPKFSLHKRNKEDNYTNTVARDDLEREIKLIFAKQREYGNIVCTEAFEHEYISIWASQRPFASKDDIEKKVGFCTFEPKEKRAPKATYTFQSFTVWEHINKLRLVSPGGIRALTDDERRLIYKQAFHKNKITFHDVRTLLNLPDDTRFKGLLYDRNTTLKENEKVRFLELGAYHKIRKAIDSVYGKGAAKSFRPIDFDTFGYALTMFKDDTDIRSYLRNEYEQNGKRMENLADKVYDEELIEELLNLSFSKFGHLSLKALRNILPYMEQGEVYSTACERAGYTFTGPKKKQKTVLLPNIPPIANPVVMRALTQARKVVNAIIKKYGSPVSIHIELARELSQSFDERRKMQKEQEGNRKKNETAIRQLVEYGLTLNPTGLDIVKFKLWSEQNGKCAYSLQPIEIERLLEPGYTEVDHVIPYSRSLDDSYTNKVLVLTKENREKGNRTPAEYLGLGSERWQQFETFVLTNKQFSKKKRDRLLRLHYDENEENEFKNRNLNDTRYISRFLANFIREHLKFADSDDKQKVYTVNGRITAHLRSRWNFNKNREESNLHHAVDAAIVACTTPSDIARVTAFYQRREQNKELSKKTDPQFPQPWPHFADELQARLSKNPKESIKALNLGNYDNEKLESLQPVFVSRMPKRSITGAAHQETLRRYIGIDERSGKIQTVVKKKLSEIQLDKTGHFPMYGKESDPRTYEAIRQRLLEHNNDPKKAFQEPLYKPKKNGELGPIIRTIKIIDTTNQVIPLNDGKTVAYNSNIVRVDVFEKDGKYYCVPIYTIDMMKGILPNKAIEPNKPYSEWKEMTEDYTFRFSLYPNDLIRIEFPREKTIKTAVGEEIKIKDLFAYYQTIDSSNGGLSLVSHDNNFSLRSIGSRTLKRFEKYQVDVLGNIYKVRGEKRVGVASSSHSKAGETIRPL

SEQ ID NO: 9 - protein, 124 aa, synthetic construct
  REGION 1..124: note = Synthetic
  REGION 5..124: note = may or may not be present
GGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 10 - protein, 155 aa, synthetic construct
  REGION 1..155: note = Synthetic
  REGION 6..155: note = may or may not be present
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 11 - protein, 31 aa, synthetic construct
  REGION 1..31: note = Synthetic
  REGION 2..31: note = may or may not be present
GGGGGGGGGGGGGGGGGGGGGGGGGGGGGGG

SEQ ID NO: 12 - protein, 155 aa, synthetic construct
  REGION 1..155: note = Synthetic
  REGION 6..155: note = may or may not be present
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 13 - protein, 93 aa, synthetic construct
  REGION 1..93: note = Synthetic
  REGION 4..93: note = may or may not be present
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 14 - protein, 124 aa, synthetic construct
  REGION 1..124: note = Synthetic
  REGION 5..124: note = may or may not be present
SGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGSSGGS

SEQ ID NO: 15 - protein, 16 aa, synthetic construct
  REGION 1..16: note = Synthetic
SGSETPGTSESATPES

SEQ ID NO: 16 - protein, 97 aa, synthetic construct
  REGION 1..97: note = Synthetic
  REGION 8..97: note = may or may not be present
SGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGS

SEQ ID NO: 17 - protein, 32 aa, synthetic construct
  REGION 1..32: note = Synthetic
SGGSSGGSSGSETPGTSESATPESSGGSSGGS

SEQ ID NO: 18 - protein, 62 aa, synthetic construct
  REGION 1..62: note = Synthetic
  VARIANT 1: note = X can be any naturally occurring amino acid
  REGION 3..62: note = may or may not be present
  VARIANT 3: note = X can be any naturally occurring amino acid
  VARIANT 5: note = X can be any naturally occurring amino acid
  VARIANT 7: note = X can be any naturally occurring amino acid
  VARIANT 9: note = X can be any naturally occurring amino acid
  VARIANT 11: note = X can be any naturally occurring amino acid
  VARIANT 13: note = X can be any naturally occurring amino acid
  VARIANT 15: note = X can be any naturally occurring amino acid
  VARIANT 17: note = X can be any naturally occurring amino acid
  VARIANT 19: note = X can be any naturally occurring amino acid
  VARIANT 21: note = X can be any naturally occurring amino acid
  VARIANT 23: note = X can be any naturally occurring amino acid
  VARIANT 25: note = X can be any naturally occurring amino acid
  VARIANT 27: note = X can be any naturally occurring amino acid
  VARIANT 29: note = X can be any naturally occurring amino acid
  VARIANT 31: note = X can be any naturally occurring amino acid
  VARIANT 33: note = X can be any naturally occurring amino acid
  VARIANT 35: note = X can be any naturally occurring amino acid
  VARIANT 37: note = X can be any naturally occurring amino acid
  VARIANT 39: note = X can be any naturally occurring amino acid
  VARIANT 41: note = X can be any naturally occurring amino acid
  VARIANT 43: note = X can be any naturally occurring amino acid
  VARIANT 45: note = X can be any naturally occurring amino acid
  VARIANT 47: note = X can be any naturally occurring amino acid
  VARIANT 49: note = X can be any naturally occurring amino acid
  VARIANT 51: note = X can be any naturally occurring amino acid
  VARIANT 53: note = X can be any naturally occurring amino acid
  VARIANT 55: note = X can be any naturally occurring amino acid
  VARIANT 57: note = X can be any naturally occurring amino acid
  VARIANT 59: note = X can be any naturally occurring amino acid
  VARIANT 61: note = X can be any naturally occurring amino acid
XPXPXPXPXPXPXPXPXPXPXPXPXPXPXPXPXPXPXPXPXPXPXPXPXPXPXPXPXPXPXP

SEQ ID NO: 19 - protein, 24 aa, synthetic construct
  REGION 1..24: note = Synthetic
  REGION 4..24: note = may or may not be present
GGSGGSGGSGGSGGSGGSGGSGGS

SEQ ID NO: 20 - protein, 48 aa, synthetic construct
  REGION 1..48: note = Synthetic
  REGION 4..48: note = may or may not be present
GGSGGSGGSGGSGGSGGSGGSGGSGGSGGSGGSGGSGGSGGSGGSGGS

SEQ ID NO: 21 - protein, 34 aa, synthetic construct
  REGION 1..34: note = Synthetic
  REGION 8..34: note = may or may not be present
SGGSGGSGGSGGSGGSGGSGGSGGSGGSGGSGGS

SEQ ID NO: 22 - protein, 13 aa, synthetic construct
  REGION 1..13: note = Synthetic
  REGION 8..13: note = may or may not be present
SGGSGGSGGSGGS

SEQ ID NO: 23 - protein, 11 aa, synthetic construct
  REGION 1..11: note = Synthetic
SPKKKRKVEAS

SEQ ID NO: 24 - protein, 10 aa, synthetic construct
  REGION 1..10: note = Synthetic
  REGION 8..10: note = may or may not be present
SGGSGGSGGS

SEQ ID NO: 25 - protein, 198 aa, Homo sapiens [sequence omitted]

SEQ ID NO: 26 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 27 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 28 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 29 - protein, 429 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 30 - protein, 429 aa, Rattus rattus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 31 - protein, 370 aa, synthetic construct
  REGION 1..370: note = Synthetic
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 32 - protein, 384 aa, synthetic construct
  REGION 1..384: note = Synthetic
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 33 - protein, 377 aa, synthetic construct
  REGION 1..377: note = Synthetic
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 34 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 35 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 36 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 37 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 38 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 39 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 40 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 41 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 42 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 43 - protein, 229 aa, Rattus rattus [sequence omitted]

SEQ ID NO: 44 - protein, 239 aa, Rattus rattus
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 45 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWFCDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 46 - protein, 202 aa, synthetic construct
  REGION 1..202: note = Synthetic
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 47 - protein, 185 aa, unidentified
  source 1..185: note = Bos bovis
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 48 - protein, 396 aa, Rattus rattus
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFYWRKKFQKGLCTLWRSGIHVDVMDLPQFADCWTNFVNPQRPFRPWNELEKNSWRIQRRLRRIKESWGL

SEQ ID NO: 49 - protein, 227 aa, unidentified
  source 1..227: note = Bos bovis
DGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAEIRFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 50 - protein, 490 aa, synthetic construct
  REGION 1..490: note = Synthetic
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 51 - protein, 210 aa, synthetic construct
  REGION 1..210: note = Synthetic
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 52 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 53 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 54 - protein, 224 aa, Rattus rattus
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 55 - protein, 224 aa, unidentified
  source 1..224: note = Bos bovis
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 56 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 57 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQNQEN

SEQ ID NO: 58 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQ

SEQ ID NO: 59 - protein, 184 aa, Homo sapiens
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISIMTYSEFKHCWDTFVDHQGCPFQPWDGLDEHSQDLSGRLRAILQ

SEQ ID NO: 60 - protein, 84 aa, synthetic construct
  REGION 1..84: note = Synthetic
MTNLSDIIEKETGKQLVIQESILMLPEEVEEVIGNKPESDILVHTAYDESTDENVMLLTSDAPEYKPWALVIQDSNGENKIKML

SEQ ID NO: 61 - protein, 181 aa, unidentified
  REGION 1..181: note = Erwinia tasmaniensis
MASRGVNKVILVGNLGQDPEVRYMPNGGAVANITLATSESWRDKQTGETKEKTEWHRVVLFGKLAEVAGEYLRKGSQVYIEGALQTRKWTDQAGVEKYTTEVVVNVGGTMQMLGGRSQGGGASAGGQNGGSNNGWGQPQQPQGGNQFSGGAQQQARPQQQPQQNNAPANNEPPIDFDDDIP

SEQ ID NO: 62 - protein, 209 aa, synthetic construct
  REGION 1..209: note = Synthetic
MAGAQDFVPHTADLAELAAAAGECRGCGLYRDATQAVFGAGGRSARIMMIGEQPGDKEDLAGLPFVGPAGRLLDRALEAADIDRDALYVTNAVKHFKFTRAAGGKRRIHKTPSRTEVVACRPWLIAEMTSVEPDVVVLLGATAAKALLGNDFRVTQHRGEVLHVDDVPGDPALVATVHPSSLLRGPKEERESAFAGLVDDLRVAADVRP

SEQ ID NO: 63 - protein, 313 aa, Homo sapiens
MIGQKTLYSFFSPSPARKRHAPSPEPAVQGTGVAGVPEESGDAAAIPAKKAPAGQEEPGTPPSSPLSAEQLDRIQRNKAAALLRLAARNVPVGFGESWKKHLSGEFGKPYFIKLMGFVAEERKHYTVYPPPHQVFTWTQMCDIKDVKVVILGQEPYHGPNQAHGLCFSVQRPVPPPPSLENIYKELSTDIEDFVHPGHGDLSGWAKQGVLLLNAVLTVRAHQANSHKERGWEQFTDAVVSWLNQNSNGLVFLLWGSYAQKKGSAIDRKRHHVLQTAHPSPLSVYRGFFGCRHFSKTNELLQKSGKKPIDWKEL

SEQ ID NO: 64 - protein, 148 aa, Homo sapiens
MFRRPVLQVLRQFVRHESETTTSLVLERSLNRVHLLGRVGQDPVLRQVEGKNPVTIFSLATNEMWRSGDSEVYQLGDVSQKTTWHRISVFRPGLRDVAYQYVKKGSRIYLEGKIDYGEYMDKNNVRRQATTIIADNIIFLSDQTKEKE

SEQ ID NO: 65 - protein, 388 aa, Mus musculus
MFAKGKGSAVPSDGQAREKLALYVYEYLLHVGAQKSAQTFLSEIRWEKNITLGEPPGFLHSWWCVFWDLYCAAPERRDTCEHSSEAKAFHDYSAAAAPSPVLGNIPPNDGMPGGPIPPGFFQGPPGSQPSPHAQPPPHNPSSMMGPHSQPFMSPRYAGGPRPPIRMGNQPPGGVPGTQPLLPNSMDPTRQQGHPNMGGSMQRMNPPRGMGPMGPGPQNYGSGMRPPPNSLGPAMPGINMGPGAGRPWPNPNSANSIPYSSSSPGTYVGPPGGGGPPGTPIMPSPADSTNSSDNIYTMINPVPPGGSRSNFPMGPGSDGPMGGMGGMEPHHMNGSLGSGDIDGLPKNSPNNISGISNPPGTPRDDGELGGNFLHSFQNDNYSPSMTMSV

SEQ ID NO: 66 - protein, 616 aa, synthetic construct
  REGION 1..616: note = Synthetic
MVGQLSEGAIAAIMQKGDTNIKPILQVINIRPITTGNSPPRYRLLMSDGLNTLSSFMLATQLNPLVEEEQLSSNCVCQIHRFIVNTLKDGRRVVILMELEVLKSAEAVGVKIGNPVPYNEGLGQPQVAPPAPAASPAASSRPQPQNGSSGMGSTVSKAYGASKTFGKAAGPSLSHTSGGTQSKVVPIASLTPYQSKWTICARVTNKSQIRTWSNSRGEGKLFSLELVDESGEIRATAFNEQVDKFFPLIEVNKVYYFSKGTLKIANKQFTAVKNDYEMTFNNETSVMPCEDDHHLPTVQFDFTGIDDLENKSKDSLVDIIGICKSYEDATKITVRSNNREVAKRNIYLMDTSGKVVTATLWGEDADKFDGSRQPVLAIKGARVSDFGGRSLSVLSSSTIIANPDIPEAYKLRGWFDAEGQALDGVSISDLKSGGVGGSNTNWKTLYEVKSENLGQGDKPDYFSSVATVVYLRKENCMYQACPTQDCNKKVIDQQNGLYRCEKCDTEFPNFKYRMILSVNIADFQENQWVTCFQESAEAILGQNAAYLGELKDKNEQAFEEVFQNANFRSFIFRVRVKVETYNDESRIKATVMDVKPVDYREYGRRLVMSIRRSALM

SEQ ID NO: 67 - protein, 270 aa, synthetic construct
  REGION 1..270: note = Synthetic
MWNSGFESYGSSSYGGAGGYTQSPGGFGSPAPSQAEKKSRARAQHIVPCTISQLLSATLVDEVFRIGNVEISQVTIVGIIRHAEKAPTNIVYKIDDMTAAPMDVRQWVDTDDTSSENTVVPPETYVKVAGHLRSFQNKKSLVAFKIMPLEDMNEFTTHILEVINAHMVLSKANSQPSAGRAPISNPGMSEAGNFGGNSFMPANGLTVAQNQVLNLIKACPRPEGLNFQDLKNQLKHMSVSSIKQAVDFLSNEGHIYSTVDDDHFKSTDAE

SEQ ID NO: 68 - protein, 121 aa, synthetic construct
  REGION 1..121: note = Synthetic
MVDMMDLPRSRINAGMLAQFIDKPVCFVGRLEKIHPTGKMFILSDGEGKNGTIELMEPLDEEISGIVEVVGRVTAKATILCTSYVQFKEDSHPFDLGLYNEAVKIIHDFPQFYPLGIVQHD

SEQ ID NO: 69 - protein, 172 aa, Bacillus subtilis
MLNRVVLVGRLTKDPELRYTPNGAAVATFTLAVNRTFTNQSGEREADFINCVTWRRQAENVANFLKKGSLAGVDGRLQTRNYENQQGQRVFVTEVQAESVQFLEPKNGGGSGSGGYNEGNSGGGQYFGGGQNDNPFGGNQNNQRRNQGNSFNDDPFANDGKPIDISDDDLPF

SEQ ID NO: 70 - protein, 199 aa, unidentified
  source 1..199: note = Streptomyces coelicolor
MAGETVITVVGNLVDDPELRFTPSGAAVAKFRVASTPRTFDRQTNEWKDGESLFLTCSVWRQAAENVAESLQRGMRVIVQGRLKQRSYEDREGVKRTVYELDVDEVGASLRSATAKVTKTSGQGRGGQGGYGGGGGGQGGGGWGGGPGGGQQGGGAPADDPWATGGAPAGGQQGGGGQGGGGWGGGSGGGGGYSDEPPF

SEQ ID NO: 71 - protein, 156 aa, Streptococcus pneumoniae
MINNVVLVGRMTRDAELRYTPSNVAVATFTLAVNRTFKSQNGEREADFINVVMWRQQAENLANWAKKGSLIGVTGRIQTRSYDNQQGQRVYVTEVVAENFQMLESRSVREGHTGGAYSAPTANYSAPTNSVPDFSRNENPFGATNPLDISDDDLPF

SEQ ID NO: 72 - protein, 1196 aa, synthetic construct
  REGION 1..1196: note = Synthetic
METKPKTATTIKVPPGPLGYVYARACPSEGIELLALLSARSGDSDVAVAPLVVGLTVESGFEANVAVVVGSRTTGLGGTAVSLKLTPSHYSSSVYVFHGGRHLDPSTQAPNLTRLCERARRHFGFSDYTPRPGDLKHETTGEALCERLGLDPDRALLYLVVTEGFKEAVCINNTFLHLGGSDKVTIGGAEVHRIPVYPLQLFMPDFSRVIAEPFNANHRSIGENFTYPLPFFNRPLNRLLFEAVVGPAAVALRCRNVDAVARAAAHLAFDENHEGAALPADITFTAFEASQGKTPRGGRDGGGKGPAGGFEQRLASVMAGDAALALESIVSMAVFDEPPTDISAWPLFEGQDTAAARANAVGAYLARAAGLVGAMVFSTNSALHLTEVDDAGPADPKDHSKPSFYRFFLVPGTHVAANPQVDREGHVVPGFEGRPTAPLVGGTQEFAGEHLAMLCGFSPALLAKMLFYLERCDGGVIVGRQEMDVFRYVADSNQTDVPCNLCTFDTRHACVHTTLMRLRARHPKFASAARGAIGVFGTMNSMYSDCDVLGNYAAFSALKRADGSETARTIMQETYRAATERVMAELETLQYVDQAVPTAMGRLETIITNREALHTVVNNVRQVVDREVEQLMRNLVEGRNFKFRDGLGEANHAMSLTLDPYACGPCPLLQLLGRRSNLAVYQDLALSQCHGVFAGQSVEGRNFRNQFQPVLRRRVMDMFNNGFLSAKTLTVALSEGAAICAPSLTAGQTAPAESSFEGDVARVTLGFPKELRVKSRVLFAGASANASEAAKARVASLQSAYQKPDKRVDILLGPLGFLLKQFHAAIFPNGKPPGSNQPNPQWFWTALQRNQLPARLLSREDIETIAFIKKFSLDYGAINFINLAPNNVSELAMYYMANQILRYCDHSTYFINTLTAIIAGSRRPPSVQAAAAWSAQGGAGLEAGARALMDAVDAHPGAWTSMFASCNLLRPVMAARPMVVLGLSISKYYGMAGNDRVFQAGNWASLMGGKNACPLLIFDRTRKFVLACPRAGFVCAASSLGGGAHESSLCEQLRGIISEGGAAVASSVFVATVKSLGPRTQQLQIEDWLALLEDEYLSEEMMELTARALERGNGEWSTDAALEVAHEAEALVSQLGNAGEVFNFGDFGCEDDNATPFGGPGAPGPAFAGRKRAFHGDDPFGEGPPDKKGDLTLDML

SEQ ID NO: 73 - protein, 124 aa, synthetic construct
  REGION 1..124: note = Synthetic
MENTNIVKATFDTETLEGQIKIFNAQTGGGQSFKNLPDGTIIEANAIAQYKQVSDTYGDAKEETVTTIFAADGSLYSAISKTVAEAASDLIDLVTRHKLETFKVKVVQGTSSKGNVFFSLQLSL

SEQ ID NO: 74 - protein, 162 aa, synthetic construct
  REGION 1..162: note = Synthetic
MASVNKVILVGNLGADPETRYLPSGDAISNIRLATTDRYKDKASGEMKESTEWHRVSFFGRLAEIVDEYLRKGAPVYIEGRIRTRKWQDNAGQDRYTTEIVAEKMQMLGDRRDGGERQQRAPQQQQQRTQRNGYADATGRAQPSQRPAAGGGFDEMDDDIPF

SEQ ID NO: 75 - protein, 7 aa, synthetic construct
  REGION 1..7: note = Synthetic
PKKKRKV

SEQ ID NO: 76 - protein, 30 aa, synthetic construct
  REGION 1..30: note = Synthetic
MDSLLMNRRKFLYQFKNVRWAKGRRETYLC

SEQ ID NO: 77 - RNA, 123 nt, unidentified
  source 1..123: mol_type = genomic RNA; note = Bacillus thermoamyloliquefaciens
gtcatagttcccctgagaaatcagggttactatgataagggctttctgcctaaggcagactgacccgcggcgttggggatcgcctgtcgcccgcttttggcgggcattccccatccttttttt

SEQ ID NO: 78 - RNA, 87 nt, synthetic construct
  misc_feature 1..87: note = Synthetic
  source 1..87: mol_type = other RNA
gcaagccccgagaaacagggacagaaagggccgccaaggcagacgacccgcggcgggggacgccgcgcccgcggcgggcaccccacc

SEQ ID NO: 79 - protein, 8 aa, synthetic construct
  REGION 1..8: note = Synthetic
NNNNCRAA

SEQ ID NO: 80 - protein, 1429 aa, synthetic construct
  REGION 1..1429: note = Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGSETPGTSESATPESRYKIGLAIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRDSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 81 - protein, 1544 aa, synthetic construct
  REGION 1..1544: note = Synthetic
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQLTFFTIALQSCHYQRLPPHILWATGLKSGGSSGGSSGSETPGTSESATPESSGGSSGGSRYKIGLAIGITSVGWAVMNLDIPRIEDLGVRIFDRAENPQTGESLALPRRLARSARRRLRRRKHRLERIRRLVIREGILTKEELDKLFEEKHEIDVWQLRVEALDRKLNNDELARVLLHLAKRRGFKSNRKSERSNKENSTMLKHIEENRAILSSYRTVGEMIVKDPKFALHKRNKGENYTNTIARDDLEREIRLIFSKQREFGNMSCTEEFENEYITIWASQRPVASKDDIEKKVGFCTFEPKEKRAPKATYTFQSFIAWEHINKLRLISPSGARGLTDEERRLLYEQAFQKNKITYHDIRTLLHLPDDTYFKGIVYDRGESRKQNENIRFLELDAYHQIRKAVDKVYGKGKSSSFLPIDFDTFGYALTLFKDDADIHSYLRNEYEQNGKRMPNLANKVYDNELIEELLNLSFTKFGHLSLKALRSILPYMEQGEVYSSACERAGYTFTGPKKKQKTMLLPNIPPIANPVVMRALTQARKVVNAIIKKYGSPVSIHIELARDLSQTFDERRKTKKEQDENRKKNETAIRQLMEYGLTLNPTGHDIVKFKLWSEQNGRCAYSLQPIEIERLLEPGYVEVDHVIPYSRSLDDSYTNKVLVLTRENREKGNRIPAEYLGVGTERWQQFETFVLTNKQFSKKKRDRLLRLHYDENEETEFKNRNLNDTRYISRFFANFIREHLKFAESDDKQKVYTVNGRVTAHLRSRWEFNKNREESDLHHAVDAVIVACTTPSDIAKVTAFYQRREQNKELAKKTEPHFPQPWPHFADELRARLSKHPKESIKALNLGNYDDQKLESLQPVFVSRMPKRSVTGAAHQETLRRYVGIDERSGKIQTVVKTKLSEIKLDASGHFPMYGKESDPRTYEAIRQRLLEHNNDPKKAFQEPLYKPKKNGEPGPVIRTVKIIDTKNQVIPLNDGKTVAYNSNIVRVDVFEKDGKYYCVPVYTMDIMKGILPNKAIEPNKPYSEWKEMTEDYTFRFSLYPNDLIRIELPREKTVKTAAGEEINVKDVFVYYKTIDSANGGLELISHDHRFSLRGVGSRTLKRFEKYQVDVLGNIYKVRGEKRVGLASSAHSKPGKTIRPLQSTRDSGGSGGSGGSTNLSDIIEKETGKQLVIQESILMLPEEVEEVIGNKPESDILVHTAYDESTDENVMLLTSDAPEYKPWALVIQDSNGENKIKMLSGGSGGSGGSTNLSDIIEKETGKQLVIQESILMLPEEVEEVIGNKPESDILVHTAYDESTDENVMLLTSDAPEYKPWALVIQDSNGENKIKMLSGGSPKKKRKV

SEQ ID NO: 82 - DNA, 28 bp, synthetic construct
  misc_feature 1..28: note = Synthetic
  source 1..28: mol_type = other DNA
tctgcagatcaytggggtggatcccgaa

SEQ ID NO: 83 - DNA, 28 bp, synthetic construct
  misc_feature 1..28: note = Synthetic
  source 1..28: mol_type = other DNA
tgccgctctyggtgaagatgaccccgaa

SEQ ID NO: 84 - DNA, 28 bp, synthetic construct
  misc_feature 1..28: note = Synthetic
  source 1..28: mol_type = other DNA
catccygggccggaggagcccgtgcaaa

SEQ ID NO: 85 - DNA, 27 bp, synthetic construct
  misc_feature 1..27: note = Synthetic
  source 1..27: mol_type = other DNA
ayggcatctcgtggagatagcaccaaa

SEQ ID NO: 86 - DNA, 28 bp, synthetic construct
  misc_feature 1..28: note = Synthetic
  source 1..28: mol_type = other DNA
aayggcatctcgtggagatagcaccaaa

SEQ ID NO: 87 - DNA, 28 bp, synthetic construct
  misc_feature 1..28: note = Synthetic
  source 1..28: mol_type = other DNA
tagggattaytggagctcctttctcaaa

SEQ ID NO: 88 - DNA, 28 bp, synthetic construct
  misc_feature 1..28: note = Synthetic
  source 1..28: mol_type = other DNA
ccaytccagtgtaaaacttgtcagcgaa

SEQ ID NO: 89 - DNA, 28 bp, synthetic construct
  misc_feature 1..28: note = Synthetic
  source 1..28: mol_type = other DNA
cccctyccccacactatctcaatgcaaa

SEQ ID NO: 90 - DNA, 28 bp, synthetic construct
  misc_feature 1..28: note = Synthetic
  source 1..28: mol_type = other DNA
ctgtgtgcyggcccatcactttggcaaa

SEQ ID NO: 91 - DNA, 28 bp, synthetic construct
  misc_feature 1..28: note = Synthetic
  source 1..28: mol_type = other DNA
aattctaggcyatatacaactacgcaaa

SEQ ID NO: 92 - DNA, 28 bp, synthetic construct
  misc_feature 1..28: note = Synthetic
  source 1..28: mol_type = other DNA
tcttcyccaacttcacggctgtcccgaa

SEQ ID NO: 93 - DNA, 28 bp, synthetic construct
  misc_feature 1..28: note = Synthetic
  source 1..28: mol_type = other DNA
aaaggaatgccytgttcatcaaagcgaa

SEQ ID NO: 94 - DNA, 28 bp, synthetic construct
  misc_feature 1..28: note = Synthetic
  source 1..28: mol_type = other DNA
aggacttayacctgcgaattatgtcaaa

SEQ ID NO: 95 - DNA, 26 bp, synthetic construct
  misc_feature 1..26: note = Synthetic
  source 1..26: mol_type = other DNA
yaaaagatgtgtatgttgattccaaa

SEQ ID NO: 96 - DNA, 28 bp, synthetic construct
  misc_feature 1..28: note = Synthetic
  source 1..28: mol_type = other DNA
tcyaaaagatgtgtatgttgattccaaa

SEQ ID NO: 97 - DNA, 27 bp, synthetic construct
  misc_feature 1..27: note = Synthetic
  source 1..27: mol_type = other DNA
cyaaaagatgtgtatgttgattccaaa

SEQ ID NO: 98 - DNA, 28 bp, synthetic construct
  misc_feature 1..28: note = Synthetic
  source 1..28: mol_type = other DNA
ccagcaygggcaccagcctcagcccgaa

SEQ ID NO: 99 - DNA, 27 bp, synthetic construct
  misc_feature 1..27: note = Synthetic
  source 1..27: mol_type = other DNA
gyttgtaaatattttcttctctccaaa

SEQ ID NO: 100 - DNA, 28 bp, synthetic construct
  misc_feature 1..28: note = Synthetic
  source 1..28: mol_type = other DNA
ggyttgtaaatattttcttctctccaaa

SEQ ID NO: 101 - DNA, 28 bp, synthetic construct
  misc_feature 1..28: note = Synthetic
  source 1..28: mol_type = other DNA
ctgcgtccagyttcagagaggaggcaaa

SEQ ID NO: 102 - DNA, 28 bp, synthetic construct
  misc_feature 1..28: note = Synthetic
  source 1..28: mol_type = other DNA
aaatacayatgtactaagaattttcaaa

SEQ ID NO: 103 - DNA, 28 bp, synthetic construct
  misc_feature 1..28: note = Synthetic
  source 1..28: mol_type = other DNA
ccgacttayagccagtaatgtcaccgaa

SEQ ID NO: 104 - DNA, 28 bp, synthetic construct
  misc_feature 1..28: note = Synthetic
  source 1..28: mol_type = other DNA
ttaaaagcayccgtggactatatccgaa

SEQ ID NO: 105 - DNA, 28 bp, synthetic construct
  misc_feature 1..28: note = Synthetic
  source 1..28: mol_type = other DNA
aagygattgcagcagtgaaatgggcaaa

SEQ ID NO: 106 - DNA, 28 bp, synthetic construct
  misc_feature 1..28: note = Synthetic
  source 1..28: mol_type = other DNA
agccaygggcggggcagtgcctttcaaa

SEQ ID NO: 107 - DNA, 28 bp, synthetic construct
  misc_feature 1..28: note = Synthetic
  source 1..28: mol_type = other DNA
ctggaatacttytgtagaaaaccacgaa

SEQ ID NO: 108 - DNA, 28 bp, synthetic construct
  misc_feature 1..28: note = Synthetic
  source 1..28: mol_type = other DNA
gtgytctccatgttcgaacagacccaaa

SEQ ID NO: 109 - DNA, 28 bp, synthetic construct
  misc_feature 1..28: note = Synthetic
  source 1..28: mol_type = other DNA
gccatytgcctctgggtaagttgccaaa

SEQ ID NO: 110 - DNA, 27 bp, synthetic construct
  misc_feature 1..27: note = Synthetic
  source 1..27: mol_type = other DNA
cyggctcagcagattgcaaactgcaaa

SEQ ID NO: 111 - DNA, 28 bp, synthetic construct
  misc_feature 1..28: note = Synthetic
  source 1..28: mol_type = other DNA
acyggctcagcagattgcaaactgcaaa

SEQ ID NO: 112 - DNA, 28 bp, synthetic construct
  misc_feature 1..28: note = Synthetic
  source 1..28: mol_type = other DNA
ttcgcaaacyggctcagcagattgcaaa

SEQ ID NO: 113 - DNA, 28 bp, synthetic construct
  misc_feature 1..28: note = Synthetic
  source 1..28: mol_type = other DNA
cattcygcaatccatgcataagtacaaa

SEQ ID NO: 114 - DNA, 28 bp, synthetic construct
  misc_feature 1..28: note = Synthetic
  source 1..28: mol_type = other DNA
ttcgatttgcygtaccaagtgccacaaa

SEQ ID NO: 115 - DNA, 28 bp, synthetic construct
  misc_feature 1..28: note = Synthetic
  source 1..28: mol_type = other DNA
tatcyacaaaagtaccaagaattacaaa

SEQ ID NO: 116 - DNA, 28 bp, synthetic construct
  misc_feature 1..28: note = Synthetic
  source 1..28: mol_type = other DNA
taaacyggagaaaacctatacaatcaaa

SEQ ID NO: 117 - DNA, 28 bp, synthetic construct
  misc_feature 1..28: note = Synthetic
  source 1..28: mol_type = other DNA
acttccttaytcaaacaaacttttcaaa

SEQ ID NO: 118 - DNA, 28 bp, synthetic construct
  misc_feature 1..28: note = Synthetic
  source 1..28: mol_type = other DNA
gcatcytccggatctttgaggggacaaa

SEQ ID NO: 119 - DNA, 28 bp, synthetic construct
  misc_feature 1..28: note = Synthetic
  source 1..28: mol_type = other DNA
ttatttgcytggaagtgattataccgaa

SEQ ID NO: 120 - DNA, 28 bp, synthetic construct
  misc_feature 1..28: note = Synthetic
  source 1..28: mol_type = other DNA
ggtatggtyttctaatcgaagggccaaa

SEQ ID NO: 121 - DNA, 28 bp, synthetic construct
  misc_feature 1..28: note = Synthetic
  source 1..28: mol_type = other DNA
gcacyggaagttgggacatacagtcaaa

SEQ ID NO: 122 - DNA, 28 bp, synthetic construct
  misc_feature 1..28: note = Synthetic
  source 1..28: mol_type = other DNA
agaccatgcaayacagctcaagatcgaa

SEQ ID NO: 123 - DNA, 28 bp, synthetic construct
  misc_feature 1..28: note = Synthetic
  source 1..28: mol_type = other DNA
tgggaagacaygtaccaggggagacaaa

SEQ ID NO: 124 - DNA, 28 bp, synthetic construct
  misc_feature 1..28: note = Synthetic
  source 1..28: mol_type = other DNA
tcttgaagaytgatagaacagaaccaaa

SEQ ID NO: 125 - DNA, 28 bp, synthetic construct
  misc_feature 1..28: note = Synthetic
  source 1..28: mol_type = other DNA
ggaacgaygggccttcaacttcagcgaa

SEQ ID NO: 126 - DNA, 27 bp, synthetic construct
  misc_feature 1..27: note = Synthetic
  source 1..27: mol_type = other DNA
tytggagtgataccacaggtgagcaaa

SEQ ID NO: 127 - DNA, 28 bp, synthetic construct
  misc_feature 1..28: note = Synthetic
  source 1..28: mol_type = other DNA
ggaacgaygggccttcaacttcagcgaa

SEQ ID NO: 128 - DNA, 28 bp, synthetic construct
  misc_feature 1..28: note = Synthetic
  source 1..28: mol_type = other DNA
ctytggagtgataccacaggtgagcaaa

SEQ ID NO: 129 - DNA, 28 bp, synthetic construct
  misc_feature 1..28: note = Synthetic
  source 1..28: mol_type = other DNA
gaatccaagayggcgggatccaggcaaa

SEQ ID NO: 130 - DNA, 27 bp, synthetic construct
  misc_feature 1..27: note = Synthetic
  source 1..27: mol_type = other DNA
cyttgcagactttgggaaccacacaaa

SEQ ID NO: 131 - DNA, 28 bp, synthetic construct
  misc_feature 1..28: note = Synthetic
  source 1..28: mol_type = other DNA
tcyttgcagactttgggaaccacacaaa

SEQ ID NO: 132 - DNA, 28 bp, synthetic construct
  misc_feature 1..28: note = Synthetic
  source 1..28: mol_type = other DNA
tttctycggcttagaggtgactggcaaa

SEQ ID NO: 133 - DNA, 27 bp, synthetic construct
  misc_feature 1..27: note = Synthetic
  source 1..27: mol_type = other DNA
tycttcggcttagaggtgactggcaaa

SEQ ID NO: 134 - DNA, 28 bp, synthetic construct
  misc_feature 1..28: note = Synthetic
  source 1..28: mol_type = other DNA
ttycttcggcttagaggtgactggcaaa

SEQ ID NO: 135 - DNA, 28 bp, synthetic construct
  misc_feature 1..28: note = Synthetic
  source 1..28: mol_type = other DNA
tgggtgyccaataagaccgaaggccgaa

SEQ ID NO: 136 - DNA, 28 bp, synthetic construct
  misc_feature 1..28: note = Synthetic
  source 1..28: mol_type = other DNA
ccaacayaatgcagatggaagaagcaaa

SEQ ID NO: 137 - DNA, 28 bp, synthetic construct
  misc_feature 1..28: note = Synthetic
  source 1..28: mol_type = other DNA
taaggaccygcagcaccggctggacgaa

SEQ ID NO: 138 - DNA, 28 bp, synthetic construct
  misc_feature 1..28: note = Synthetic
  source 1..28: mol_type = other DNA
aatagagtgcyaaaagacccaaaacaaa

SEQ ID NO: 139 - DNA, 28 bp, synthetic construct
  misc_feature 1..28: note = Synthetic
  source 1..28: mol_type = other DNA
tgaatccaagygtcctctgatggtcaaa

SEQ ID NO: 140 - DNA, 28 bp, synthetic construct
  misc_feature 1..28: note = Synthetic
  source 1..28: mol_type = other DNA
gatatacaaagyggaaatagacaccaaa

SEQ ID NO: 141 - DNA, 28 bp, synthetic construct
  misc_feature 1..28: note = Synthetic
  source 1..28: mol_type = other DNA
ggaggaayttgtagaagggatatacaaa

SEQ ID NO: 142 - DNA, 28 bp, synthetic construct
  misc_feature 1..28: note = Synthetic
  source 1..28: mol_type = other DNA
ggaggaayttgtagaagggatatacaaa

SEQ ID NO: 143 - DNA, 28 bp, synthetic construct
  misc_feature 1..28: note = Synthetic
  source 1..28: mol_type = other DNA
gatayacaaagtggaaatagacaccaaa

SEQ ID NO: 144 - DNA, 27 bp, synthetic construct
  misc_feature 1..27: note = Synthetic
  source 1..27: mol_type = other DNA
cyggacttttcctccaaagaagtcaaa

SEQ ID NO: 145 - DNA, 28 bp, synthetic construct
  misc_feature 1..28: note = Synthetic
  source 1..28: mol_type = other DNA
ccyggacttttcctccaaagaagtcaaa

SEQ ID NO: 146 - DNA, 28 bp, synthetic construct
  misc_feature 1..28: note = Synthetic
  source 1..28: mol_type = other DNA
caatcgctaccyggacttttcctccaaa

SEQ ID NO: 147 - DNA, 28 bp, synthetic construct
  misc_feature 1..28: note = Synthetic
  source 1..28: mol_type = other DNA
gccytgatgctctcagcaagaaagcgaa

SEQ ID NO: 148 - DNA, 28 bp, synthetic construct
  misc_feature 1..28: note = Synthetic
  source 1..28: mol_type = other DNA
cgaaytcgtggacatcatcaatgccaaa

SEQ ID NO: 149 - DNA, 28 bp, synthetic construct
  misc_feature 1..28: note = Synthetic
  source 1..28: mol_type = other DNA
cagtgggctgcyggtctaccagggcaaa

SEQ ID NO: 150 - DNA, 28 bp, synthetic construct
  misc_feature 1..28: note = Synthetic
  source 1..28: mol_type = other DNA
acactcccttyccaaggaaaagatcgaa

SEQ ID NO: 151 - DNA, 28 bp, synthetic construct
  misc_feature 1..28: note = Synthetic
  source 1..28: mol_type = other DNA
tggctccaaccytctgtccatctgcaaa

SEQ ID NO: 152 - DNA, 28 bp, synthetic construct
  misc_feature 1..28: note = Synthetic
  source 1..28: mol_type = other DNA
atgtcaacyggattaaggaaaaaacaaa

SEQ ID NO: 153 - DNA, 28 bp, synthetic construct
  misc_feature 1..28: note = Synthetic
  source 1..28: mol_type = other DNA
cctggacyccgtgcagcctgtaagcaaa

SEQ ID NO: 154 - DNA, 28 bp, synthetic construct
  misc_feature 1..28: note = Synthetic
  source 1..28: mol_type = other DNA
gatacaytgactatgctgaatttgcaaa

SEQ ID NO: 155 - DNA, 28 bp, synthetic construct
  misc_feature 1..28: note = Synthetic
  source 1..28: mol_type = other DNA
agccagtcgtcygggattgtattccaaa

SEQ ID NO: 156 - DNA, 28 bp, synthetic construct
  misc_feature 1..28: note = Synthetic
  source 1..28: mol_type = other DNA
tctcyccagacccactgctgctctcaaa

SEQ ID NO: 157 - DNA, 28 bp, synthetic construct
  misc_feature 1..28: note = Synthetic
  source 1..28: mol_type = other DNA
gccayttgcctctgggtaagttgccaaa

SEQ ID NO: 158 - DNA, 28 bp, synthetic construct
  misc_feature 1..28: note = Synthetic
  source 1..28: mol_type = other DNA
ataytgccctcgacacagatctggcaaa

SEQ ID NO: 159 - DNA, 28 bp, synthetic construct
  misc_feature 1..28: note = Synthetic
  source 1..28: mol_type = other DNA
cataccyggcaagcaatagcgcggcgaa

SEQ ID NO: 160 - DNA, 28 bp, synthetic construct
  misc_feature 1..28: note = Synthetic
  source 1..28: mol_type = other DNA
tgtatgtcyacagaaggacagaagcaaa

SEQ ID NO: 161 - DNA, 28 bp, synthetic construct
  misc_feature 1..28: note = Synthetic
  source 1..28: mol_type = other DNA
agcgtcyggattggaatcgtgagtcaaa

SEQ ID NO: 162 - DNA, 28 bp, synthetic construct
  misc_feature 1..28: note = Synthetic
  source 1..28: mol_type = other DNA
agataygcgtgaggttgtgtgtgtcaaa

SEQ ID NO: 163 - DNA, 28 bp, synthetic construct
  misc_feature 1..28: note = Synthetic
  source 1..28: mol_type = other DNA
ataccyaaaggagaaaaagaatcccaaa

SEQ ID NO: 164 - DNA, 28 bp, synthetic construct
  misc_feature 1..28: note = Synthetic
  source 1..28: mol_type = other DNA
ctcaygggacttttgaggccactgcaaa

SEQ ID NO: 165 - DNA, 28 bp, synthetic construct
  misc_feature 1..28: note = Synthetic
  source 1..28: mol_type = other DNA
gaacatccygtgccactgcttcaacaaa

SEQ ID NO: 166 - DNA, 28 bp, synthetic construct
  misc_feature 1..28: note = Synthetic
  source 1..28: mol_type = other DNA
aaayggcccagcctgcatttgccccaaa

SEQ ID NO: 167 - DNA, 28 bp, synthetic construct
  misc_feature 1..28: note = Synthetic
  source 1..28: mol_type = other DNA
ccatcyccatgaggagtggctcatcaaa

SEQ ID NO: 168 - DNA, 28 bp, synthetic construct
  misc_feature 1..28: note = Synthetic
  source 1..28: mol_type = other DNA
ccagygatcatccccttttccaagcaaa

SEQ ID NO: 169 - DNA, 27 bp, synthetic construct
  misc_feature 1..27: note = Synthetic
  source 1..27: mol_type = other DNA
aytggaagtactgaccaatgttccaaa

SEQ ID NO: 170 - DNA, 28 bp, synthetic construct
  misc_feature 1..28: note = Synthetic
  source 1..28: mol_type = other DNA
taytggaagtactgaccaatgttccaaa

SEQ ID NO: 171 - DNA, 28 bp, synthetic construct
  misc_feature 1..28: note = Synthetic
  source 1..28: mol_type = other DNA
aatcayaggacaagcctttggcaccaaa

SEQ ID NO: 172 - DNA, 28 bp, synthetic construct
  misc_feature 1..28: note = Synthetic
  source 1..28: mol_type = other DNA
taccygagtccatttagagagttccaaa

SEQ ID NO: 173 - DNA, 28 bp, synthetic construct
  misc_feature 1..28: note = Synthetic
  source 1..28: mol_type = other DNA
acgtaygggagttgctgaagaacgcgaa

SEQ ID NO: 174 - DNA, 28 bp, synthetic construct
  misc_feature 1..28: note = Synthetic
  source 1..28: mol_type = other DNA
acctgcygcggaccctgtcccgagcgaa

SEQ ID NO: 175 - DNA, 28 bp, synthetic construct
  misc_feature 1..28: note = Synthetic
  source 1..28: mol_type = other DNA
catcycactggcgtacgaggctgccgaa

SEQ ID NO: 176 - DNA, 28 bp, synthetic construct
  misc_feature 1..28: note = Synthetic
  source 1..28: mol_type = other DNA
actggayatggaaatagagaggagcaaa

SEQ ID NO: 177 - DNA, 28 bp, synthetic construct
  misc_feature 1..28: note = Synthetic
  source 1..28: mol_type = other DNA
tgctggcacyggcatgctgtgcctcgaa

SEQ ID NO: 178 - DNA, 28 bp, synthetic construct
  misc_feature 1..28: note = Synthetic
  source 1..28: mol_type = other DNA
agtcyacctaatgttggtttgattcaaa

SEQ ID NO: 179 - DNA, 28 bp, synthetic construct
  misc_feature 1..28: note = Synthetic
  source 1..28: mol_type = other DNA
atcaaaatayaaaaattctacactcgaa

SEQ ID NO: 180 - DNA, 28 bp, synthetic construct
  misc_feature 1..28: note = Synthetic
  source 1..28: mol_type = other DNA
caggagcaggyaaatacttatcttcaaa

SEQ ID NO: 181 - DNA, 28 bp, synthetic construct
  misc_feature 1..28: note = Synthetic
  source 1..28: mol_type = other DNA
aaactccycatcaccatccacgaccgaa

SEQ ID NO: 182 - DNA, 28 bp, synthetic construct
  misc_feature 1..28: note = Synthetic
  source 1..28: mol_type = other DNA
cagtaaaaggyagagataatgttccaaa

SEQ ID NO: 183 - DNA, 27 bp, synthetic construct
  misc_feature 1..27: note = Synthetic
  source 1..27: mol_type = other DNA
cyccgacacatcagccctcaagccaaa

SEQ ID NO: 184 - DNA, 28 bp, synthetic construct
  misc_feature 1..28: note = Synthetic
  source 1..28: mol_type = other DNA
ccyccgacacatcagccctcaagccaaa

SEQ ID NO: 185 - DNA, 28 bp, synthetic construct
  misc_feature 1..28: note = Synthetic
  source 1..28: mol_type = other DNA
aaaacggyaagaagcagcccgaaccaaa

SEQ ID NO: 186 - DNA, 28 bp, synthetic construct
  misc_feature 1..28: note = Synthetic
  source 1..28: mol_type = other DNA
tcccctttytatgggaggaaaacacaaa

SEQ ID NO: 187 - DNA, 28 bp, synthetic construct
  misc_feature 1..28: note = Synthetic
  source 1..28: mol_type = other DNA
ccagagyatgtccatatgtatactcaaa

SEQ ID NO: 188 - DNA, 28 bp, synthetic construct
  misc_feature 1..28: note = Synthetic
  source 1..28: mol_type = other DNA
cagatacytgagggagagctgcagcgaa

SEQ ID NO: 189 - DNA, 28 bp, synthetic construct
  misc_feature 1..28: note = Synthetic
  source 1..28: mol_type = other DNA
tgtcgtagaggycacacagagtgtcaaa

SEQ ID NO: 190 - DNA, 28 bp, synthetic construct
  misc_feature 1..28: note = Synthetic
  source 1..28: mol_type = other DNA
tccayaatttgtcatcctggctttcaaa

SEQ ID NO: 191 - DNA, 28 bp, synthetic construct
  misc_feature 1..28: note = Synthetic
  source 1..28: mol_type = other DNA
cgaatycgtggacatcatcaatgccaaa

SEQ ID NO: 192 - DNA, 28 bp, synthetic construct
  misc_feature 1..28: note = Synthetic
  source 1..28: mol_type = other DNA
tagcagaaggyctatttcattcagcgaa

SEQ ID NO: 193 - DNA, 28 bp, synthetic construct
  misc_feature 1..28: note = Synthetic
  source 1..28: mol_type = other DNA
cctcactgataycacagaagccatcaaa

SEQ ID NO: 194 - DNA, 28 bp, synthetic construct
  misc_feature 1..28: note = Synthetic
  source 1..28: mol_type = other DNA
taatttcyatatgctcaccctagacaaa

SEQ ID NO: 195 - DNA, 27 bp, synthetic construct
  misc_feature 1..27: note = Synthetic
  source 1..27: mol_type = other DNA
cyatacaaaaaggaaaaaaaaatcaaa

SEQ ID NO: 196 - DNA, 28 bp, synthetic construct
  misc_feature 1..28: note = Synthetic
  source 1..28: mol_type = other DNA
acyatacaaaaaggaaaaaaaaatcaaa

SEQ ID NO: 197 - DNA, 28 bp, synthetic construct
  misc_feature 1..28: note = Synthetic
  source 1..28: mol_type = other DNA
tcagttgcyttctgacagccggtacgaa

SEQ ID NO: 198 - DNA, 28 bp, synthetic construct
  misc_feature 1..28: note = Synthetic
  source 1..28: mol_type = other DNA
gagcaytggggacgataattttaacaaa

SEQ ID NO: 199 - DNA, 28 bp, synthetic construct
  misc_feature 1..28: note = Synthetic
  source 1..28: mol_type = other DNA
catcttgcgtcygatcaaaggcgccaaa

SEQ ID NO: 200 - DNA, 28 bp, synthetic construct
  misc_feature 1..28: note = Synthetic
  source 1..28: mol_type = other DNA
tttgtgcctgataacgtartaacaccag

SEQ ID NO: 201 - DNA, 28 bp, synthetic construct
  misc_feature 1..28: note = Synthetic
  source 1..28: mol_type = other DNA
tttgtgcctgataacgtartaacaccag

SEQ ID NO: 202 - DNA, 28 bp, synthetic construct
  misc_feature 1..28: note = Synthetic
  source 1..28: mol_type = other DNA
tttgatatattgttctttgrccctgcca

SEQ ID NO: 203 - DNA, 28 bp, synthetic construct
  misc_feature 1..28: note = Synthetic
  source 1..28: mol_type = other DNA
ttcggctgcgacctctgtgggargggtt

SEQ ID NO: 204 - DNA, 28 bp, synthetic construct
  misc_feature 1..28: note = Synthetic
  source 1..28: mol_type = other DNA
tttgacaaccccgtctrtcagaagacca

SEQ ID NO: 205 - DNA, 28 bp, synthetic construct
  misc_feature 1..28: note = Synthetic
  source 1..28: mol_type = other DNA
tttgctgttgcctttggcrtcaacgttc

SEQ ID NO: 206 - DNA, 28 bp, synthetic construct
  misc_feature 1..28: note = Synthetic
  source 1..28: mol_type = other DNA
tttggaagttgttggcrggtacagtcca

SEQ ID NO: 207 - DNA, 28 bp, synthetic construct
  misc_feature 1..28: note = Synthetic
  source 1..28: mol_type = other DNA
tttgttttcaaggtgttctttgcagrag

SEQ ID NO: 208 - DNA, 28 bp, synthetic construct
  misc_feature 1..28: note = Synthetic
  source 1..28: mol_type = other DNA
tttgatttgccagtttagrtgcaaaatc

SEQ ID NO: 209 - DNA, 28 bp, synthetic construct
  misc_feature 1..28: note = Synthetic
  source 1..28: mol_type = other DNA
tttgggcaaaatccctcrgacagtaaga

SEQ ID NO: 210 - DNA, 28 bp, synthetic construct
  misc_feature 1..28: note = Synthetic
  source 1..28: mol_type = other DNA
tttgcctttattgcaagggragtcctta

SEQ ID NO: 211 - DNA, 28 bp, synthetic construct
  misc_feature 1..28: note = Synthetic
  source 1..28: mol_type = other DNA
tttgagaaaagaagtactcgarcaagat

SEQ ID NO: 212 - DNA, 28 bp, synthetic construct
  misc_feature 1..28: note = Synthetic
  source 1..28: mol_type = other DNA
tttgcagggtttggtttctaacctgrct

SEQ ID NO: 213 - DNA, 28 bp, synthetic construct
  misc_feature 1..28: note = Synthetic
  source 1..28: mol_type = other DNA
tttggtttctaacctgrctctcagttcc

SEQ ID NO: 214 - DNA, 28 bp, synthetic construct
  misc_feature 1..28: note = Synthetic
  source 1..28: mol_type = other DNA
tttggaggttacgtgtgtacarcctcac

SEQ ID NO: 215 - DNA, 28 bp, synthetic construct
  misc_feature 1..28: note = Synthetic
  source 1..28: mol_type = other DNA
tttgtgcacgactgcgtcaatatcrcaa

SEQ ID NO: 216 - DNA, 28 bp, synthetic construct
  misc_feature 1..28: note = Synthetic
  source 1..28: mol_type = other DNA
tttgtgcacgactgcgtcaatatcrcaa

SEQ ID NO: 217 - DNA, 28 bp, synthetic construct
  misc_feature 1..28: note = Synthetic
  source 1..28: mol_type = other DNA
tttgtgtttatgttatttgcartgtttt

SEQ ID NO: 218 - DNA, 28 bp, synthetic construct
  misc_feature 1..28: note = Synthetic
  source 1..28: mol_type = other DNA
tttgatgtctttcctgatggatgggrca

SEQ ID NO: 219 - DNA, 28 bp, synthetic construct
  misc_feature 1..28: note = Synthetic
  source 1..28: mol_type = other DNA
tttgtattacgaattatacrgaagacaa

SEQ ID NO: 220 - DNA, 28 bp, synthetic construct
  misc_feature 1..28: note = Synthetic
  source 1..28: mol_type = other DNA
tttgttttgttttctgtrggtttcagat

SEQ ID NO: 221 - DNA, 28 bp, synthetic construct
  misc_feature 1..28: note = Synthetic
  source 1..28: mol_type = other DNA
tttgggaattcctggcaggrgtggtgta

SEQ ID NO: 222 - DNA, 28 bp, synthetic construct
  misc_feature 1..28: note = Synthetic
  source 1..28: mol_type = other DNA
tttgaacgttttcctttcaatarctgct

SEQ ID NO: 223 - DNA, 28 bp, synthetic construct
  misc_feature 1..28: note = Synthetic
  source 1..28: mol_type = other DNA
tttgccggaggacagagactgacrtgga

SEQ ID NO: 224 - DNA, 28 bp, synthetic construct
  misc_feature 1..28: note = Synthetic
  source 1..28: mol_type = other DNA
tttgggaaatgcaacaatccaratggcc

SEQ ID NO: 225 - DNA, 28 bp, synthetic construct
  misc_feature 1..28: note = Synthetic
  source 1..28: mol_type = other DNA
tttgctgacaaagaagaaggtggagrtg

SEQ ID NO: 226 - DNA, 28 bp, synthetic construct
  misc_feature 1..28: note = Synthetic
  source 1..28: mol_type = other DNA
tttgtccaaagcaggtattgrgacaact

SEQ ID NO: 227 - DNA, 28 bp, synthetic construct
  misc_feature 1..28: note = Synthetic
  source 1..28: mol_type = other DNA
ttcgcgcgtcttctcctccrggtgtgca

SEQ ID NO: 228 - DNA, 28 bp, synthetic construct
  misc_feature 1..28: note = Synthetic
  source 1..28: mol_type = other DNA
tttggaggaacccatgggaatgrgctaa

SEQ ID NO: 229 - DNA, 28 bp, synthetic construct
  misc_feature 1..28: note = Synthetic
  source 1..28: mol_type = other DNA
tttgatgatgatgacargatcgttgggg

SEQ ID NO: 230 - DNA, 28 bp, synthetic construct
  misc_feature 1..28: note = Synthetic
  source 1..28: mol_type = other DNA
tttgccccttgacaggtatgggccrgca

SEQ ID NO: 231 - DNA, 28 bp, synthetic construct
  misc_feature 1..28: note = Synthetic
  source 1..28: mol_type = other DNA
tttggggtctctgttcccggtrgagtgg

SEQ ID NO: 232 - DNA, 28 bp, synthetic construct
  misc_feature 1..28: note = Synthetic
  source 1..28: mol_type = other DNA
tttgagacctatgcagrtgccctgtggt

SEQ ID NO: 233 - DNA, 28 bp, synthetic construct
  misc_feature 1..28: note = Synthetic
  source 1..28: mol_type = other DNA
ttcgggacgtggcgggggrattttacaa

SEQ ID NO: 234 - DNA, 28 bp, synthetic construct
  misc_feature 1..28: note = Synthetic
  source 1..28: mol_type = other DNA
tttgaaactgatgaacgctrccgacatt

SEQ ID NO: 235 - DNA, 28 bp, synthetic construct
  misc_feature 1..28: note = Synthetic
  source 1..28: mol_type = other DNA
tttgggctccaggtgcggcggcrgcaca

SEQ ID NO: 236 - DNA, 28 bp, synthetic construct
  misc_feature 1..28: note = Synthetic
  source 1..28: mol_type = other DNA
tttggggcaacgcgggaatrtgacagat

SEQ ID NO: 237 - DNA, 28 bp, synthetic construct
  misc_feature 1..28: note = Synthetic
  source 1..28: mol_type = other DNA
tttgcttctttctgccagtcarcaatgt

SEQ ID NO: 238 - DNA, 28 bp, synthetic construct
  misc_feature 1..28: note = Synthetic
  source 1..28: mol_type = other DNA
tttgcctctttacagccatcccttcrtc

SEQ ID NO: 239 - DNA, 28 bp, synthetic construct
  misc_feature 1..28: note = Synthetic
  source 1..28: mol_type = other DNA
tttgcctctttacagccatcccttcrtc

SEQ ID NO: 240 - DNA, 28 bp, synthetic construct
  misc_feature 1..28: note = Synthetic
  source 1..28: mol_type = other DNA
tttggagctacctctagtatcraaagtg

SEQ ID NO: 241 - DNA, 28 bp, synthetic construct
  misc_feature 1..28: note = Synthetic
  source 1..28: mol_type = other DNA
tttgaacgaactcttaaaggacrtacag

SEQ ID NO: 242 - DNA, 28 bp, synthetic construct
  misc_feature 1..28: note = Synthetic
  source 1..28: mol_type = other DNA
ttcgtggcgcaggcgcctracaactacc

SEQ ID NO: 243 - DNA, 28 bp, synthetic construct
  misc_feature 1..28: note = Synthetic
  source 1..28: mol_type = other DNA
ttcgtggcgcaggcgcctracaactacc

SEQ ID NO: 244 - DNA, 28 bp, synthetic construct
  misc_feature 1..28: note = Synthetic
  source 1..28: mol_type = other DNA
tttggtccacctgggartgggaagacaa

SEQ ID NO: 245 - DNA, 28 bp, synthetic construct
  misc_feature 1..28: note = Synthetic
  source 1..28: mol_type = other DNA
tttggctttcctgtgcactacacagrcg

SEQ ID NO: 246 - DNA, 28 bp, synthetic construct
  misc_feature 1..28: note = Synthetic
  source 1..28: mol_type = other DNA
tttgtcttctctctggggcagrggctgg

SEQ ID NO: 247 - DNA, 28 bp, synthetic construct
  misc_feature 1..28: note = Synthetic
  source 1..28: mol_type = other DNA
ttcggaaggtggtgagcrtcgagcaggg

SEQ ID NO: 248 - DNA, 28 bp, synthetic construct
  misc_feature 1..28: note = Synthetic
  source 1..28: mol_type = other DNA
tttgataagctgaatgtaaggrcaacac

SEQ ID NO: 249 - DNA, 28 bp, synthetic construct
  misc_feature 1..28: note = Synthetic
  source 1..28: mol_type = other DNA
ttcgggtggagaagacagggcacrgtgg

SEQ ID NO: 250 - DNA, 28 bp, synthetic construct
  misc_feature 1..28: note = Synthetic
  source 1..28: mol_type = other DNA
ttcgtattttggatgtcartgacaatat

SEQ ID NO: 251 - DNA, 28 bp, synthetic construct
  misc_feature 1..28: note = Synthetic
  source 1..28: mol_type = other DNA
tttgttcgaaagaatggtgtcratgaag

SEQ ID NO: 252 - DNA, 28 bp, synthetic construct
  misc_feature 1..28: note = Synthetic
  source 1..28: mol_type = other DNA
ttcgaaagaatggtgtcratgaagccaa

SEQ ID NO: 253 - DNA, 28 bp, synthetic construct
  misc_feature 1..28: note = Synthetic
  source 1..28: mol_type = other DNA
tttggtcttgccagagacatgtrtgata

SEQ ID NO: 254 - DNA, 28 bp, synthetic construct
  misc_feature 1..28: note = Synthetic
  source 1..28: mol_type = other DNA
tttgagactaaatcacarcaaactgtca

SEQ ID NO: 255 - DNA, 28 bp, synthetic construct
  misc_feature 1..28: note = Synthetic
  source 1..28: mol_type = other DNA
tttggacaaaaaggctggcccaaargat

SEQ ID NO: 256 - DNA, 28 bp, synthetic construct
  misc_feature 1..28: note = Synthetic
  source 1..28: mol_type = other DNA
tttggaccctgaggtcagaccarcttca

SEQ ID NO: 257 - DNA, 28 bp, synthetic construct
  misc_feature 1..28: note = Synthetic
  source 1..28: mol_type = other DNA
tttgaagttgcttgggaagtgaccarta

SEQ ID NO: 258 - DNA, 28 bp, synthetic construct
  misc_feature 1..28: note = Synthetic
  source 1..28: mol_type = other DNA
tttgccactttggctgagttggtccrgt

SEQ ID NO: 259 - DNA, 28 bp, synthetic construct
  misc_feature 1..28: note = Synthetic
  source 1..28: mol_type = other DNA
tttggctgagttggtccrgtattacatg

SEQ ID NO: 260 - DNA, 28 bp, synthetic construct
  misc_feature 1..28: note = Synthetic
  source 1..28: mol_type = other DNA
tttgttatgctttcaaatragaaagatt

SEQ ID NO: 261 - DNA, 28 bp, synthetic construct
  misc_feature 1..28: note = Synthetic
  source 1..28: mol_type = other DNA
tttgtttgttccggtcggaaaccragga

SEQ ID NO: 262 - DNA, 28 bp, synthetic construct
  misc_feature 1..28: note = Synthetic
  source 1..28: mol_type = other DNA
tttgttccggtcggaaaccraggacctt

SEQ ID NO: 263 - DNA, 28 bp, synthetic construct
  misc_feature 1..28: note = Synthetic
  source 1..28: mol_type = other DNA
tttgaatccttcaaacgtgctgrcatct

SEQ ID NO: 264 - DNA, 28 bp, synthetic construct
  misc_feature 1..28: note = Synthetic
  source 1..28: mol_type = other DNA
tttggatctgttttaggcctrtggaggt

SEQ ID NO: 265 - DNA, 28 bp, synthetic construct
  misc_feature 1..28: note = Synthetic
  source 1..28: mol_type = other DNA
tttgctgtgcccaacccgaagcrggtaa

SEQ ID NO: 266 - DNA, 28 bp, synthetic construct
  misc_feature 1..28: note = Synthetic
  source 1..28: mol_type = other DNA
ttcgttaatatggagagagrtctagctt

SEQ ID NO: 267 - DNA, 28 bp, synthetic construct
  misc_feature 1..28: note = Synthetic
  source 1..28: mol_type = other DNA
ttcgtgaacttcggcgtcggggrcttga

SEQ ID NO: 268 - DNA, 28 bp, synthetic construct
  misc_feature 1..28: note = Synthetic
  source 1..28: mol_type = other DNA
tttgaacccaaagacatcrctgctgaag

SEQ ID NO: 269 - DNA, 28 bp, synthetic construct
  misc_feature 1..28: note = Synthetic
  source 1..28: mol_type = other DNA
tttgcttttgtaggttgccrtgggtcaa

SEQ ID NO: 270 - DNA, 28 bp, synthetic construct
  misc_feature 1..28: note = Synthetic
  source 1..28: mol_type = other DNA
tttggggttttattttcrggttactgga

SEQ ID NO: 271 - DNA, 28 bp, synthetic construct
  misc_feature 1..28: note = Synthetic
  source 1..28: mol_type = other DNA
tttgggtgctccggggrtgacaccagta

SEQ ID NO: 272 - DNA, 28 bp, synthetic construct
  misc_feature 1..28: note = Synthetic
  source 1..28: mol_type = other DNA
tttgtcgaaagctgcttttccrgggttt

SEQ ID NO: 273 - DNA, 28 bp, synthetic construct
  misc_feature 1..28: note = Synthetic
  source 1..28: mol_type = other DNA
ttcgtataatattctctrcgttaggagt

SEQ ID NO: 274 - DNA, 28 bp, synthetic construct
  misc_feature 1..28: note = Synthetic
  source 1..28: mol_type = other DNA
tttgttaagatggcagrgcccggtaatc

SEQ ID NO: 275 - DNA, 28 bp, synthetic construct
  misc_feature 1..28: note = Synthetic
  source 1..28: mol_type = other DNA
tttgttaagatggcagagcccggtartc

SEQ ID NO: 276 - DNA, 28 bp, synthetic construct
  misc_feature 1..28: note = Synthetic
  source 1..28: mol_type = other DNA
tttgttaagatggcagagcccggtratc

SEQ ID NO: 277 - DNA, 28 bp, synthetic construct
  misc_feature 1..28: note = Synthetic
  source 1..28: mol_type = other DNA
tttgcggagaggtcagtaactraattgg

SEQ ID NO: 278 - DNA, 28 bp, synthetic construct
  misc_feature 1..28: note = Synthetic
  source 1..28: mol_type = other DNA
tttgtctttctcttttttaaaraattca

SEQ ID NO: 279 - DNA, 28 bp, synthetic construct
  misc_feature 1..28: note = Synthetic
  source 1..28: mol_type = other DNA
tttgtctttctcttttttaaaraattca

SEQ ID NO: 280 - DNA, 28 bp, synthetic construct
  misc_feature 1..28: note = Synthetic
  source 1..28: mol_type = other DNA
tttgcaggaggcattgraattttcagca

SEQ ID NO: 281 - DNA, 28 bp, synthetic construct
  misc_feature 1..28: note = Synthetic
  source 1..28: mol_type = other DNA
tttgtccaggcttgagtcaccatgrgta

SEQ ID NO: 282 - DNA, 28 bp, synthetic construct
  misc_feature 1..28: note = Synthetic
  source 1..28: mol_type = other DNA
tttgtactgagccacatgacarggtaat

SEQ ID NO: 283 - DNA, 28 bp, synthetic construct
  misc_feature 1..28: note = Synthetic
  source 1..28: mol_type = other DNA
tttgacttcctcgggaaggrctccatca

SEQ ID NO: 284 - DNA, 28 bp, synthetic construct
  misc_feature 1..28: note = Synthetic
  source 1..28: mol_type = other DNA
tttggaagttgttggcaggtrcagtcca

SEQ ID NO: 285 - DNA, 28 bp, synthetic construct
  misc_feature 1..28: note = Synthetic
  source 1..28: mol_type = other DNA
tttgctttctaaacacttttcartgttt

SEQ ID NO: 286 - DNA, 28 bp, synthetic construct
  misc_feature 1..28: note = Synthetic
  source 1..28: mol_type = other DNA
tttggactttgctatctttcrggttttg

SEQ ID NO: 287 - DNA, 28 bp, synthetic construct
  misc_feature 1..28: note = Synthetic
  source 1..28: mol_type = other DNA
tttgcaaactcgtaatttctttcrggag

SEQ ID NO: 288 - DNA, 28 bp, synthetic construct
  misc_feature 1..28: note = Synthetic
  source 1..28: mol_type = other DNA
tttgtttgcctgaatgacarcattgacc

SEQ ID NO: 289 - DNA, 28 bp, synthetic construct
  misc_feature 1..28: note = Synthetic
  source 1..28: mol_type = other DNA
tttggctgacctgcctagaaatattrtg

SEQ ID NO: 290 - DNA, 28 bp, synthetic construct
  misc_feature 1..28: note = Synthetic
  source 1..28: mol_type = other DNA
ttcggaccaatcctgagrcttgttacca

SEQ ID NO: 291 - DNA, 28 bp, synthetic construct
  misc_feature 1..28: note = Synthetic
  source 1..28: mol_type = other DNA
tttgtcttctcttcttrgggccctcctg

SEQ ID NO: 292 - DNA, 28 bp, synthetic construct
  misc_feature 1..28: note = Synthetic
  source 1..28: mol_type = other DNA
ttcgtcgcttatgcatrctcaggatgag

SEQ ID NO: 293 - DNA, 28 bp, synthetic construct
  misc_feature 1..28: note = Synthetic
  source 1..28: mol_type = other DNA
ttcgagggcacgttccrtgtgaacacgg

SEQ ID NO: 294 - DNA, 28 bp, synthetic construct
  misc_feature 1..28: note = Synthetic
  source 1..28: mol_type = other DNA
tttgacaacgacttcccagctctgcrgc

SEQ ID NO: 295 - DNA, 28 bp, synthetic construct
  misc_feature 1..28: note = Synthetic
  source 1..28: mol_type = other DNA
ttcgagcttctcagtatatttagtcrta

SEQ ID NO: 296 - DNA, 28 bp, synthetic construct
  misc_feature 1..28: note = Synthetic
  source 1..28: mol_type = other DNA
tttgttttcccctttaaacarttaagat

SEQ ID NO: 297 - DNA, 28 bp, synthetic construct
  misc_feature 1..28: note = Synthetic
  source 1..28: mol_type = other DNA
tttgcagaaagggggccctgrggcagaa

SEQ ID NO: 298 - DNA, 28 bp, synthetic construct
  misc_feature 1..28: note = Synthetic
  source 1..28: mol_type = other DNA
tttgtgctacagaaactrctatgctgac

SEQ ID NO: 299 - DNA, 28 bp, synthetic construct
  misc_feature 1..28: note = Synthetic
  source 1..28: mol_type = other DNA
tttgtgctacagaaactactrtgctgac

SEQ ID NO: 300 - DNA, 28 bp, synthetic construct
  misc_feature 1..28: note = Synthetic
  source 1..28: mol_type = other DNA
ttcgactcattaaattrtgataatcata

SEQ ID NO: 301 - DNA, 28 bp, synthetic construct
  misc_feature 1..28: note = Synthetic
  source 1..28: mol_type = other DNA
tttgcaattcaatatgaaaatcrcctcg

SEQ ID NO: 302 - DNA, 28 bp, synthetic construct
  misc_feature 1..28: note = Synthetic
  source 1..28: mol_type = other DNA
tttgcaattcaatatgaaaatcrcctcg

SEQ ID NO: 303 - DNA, 28 bp, synthetic construct
  misc_feature 1..28: note = Synthetic
  source 1..28: mol_type = other DNA
tttgactgctttttcacrattgatctct

SEQ ID NO: 304 - DNA, 28 bp, synthetic construct
  misc_feature 1..28: note = Synthetic
  source 1..28: mol_type = other DNA
tttgccctacaacagtacargttatcca

SEQ ID NO: 305 - DNA, 28 bp, synthetic construct
  misc_feature 1..28: note = Synthetic
  source 1..28: mol_type = other DNA
tttggattcagtagcagagratacgtct

SEQ ID NO: 306 - DNA, 28 bp, synthetic construct
  misc_feature 1..28: note = Synthetic
  source 1..28: mol_type = other DNA
tttgttttccctcaaaggttcarcatac

SEQ ID NO: 307 - DNA, 28 bp, synthetic construct
  misc_feature 1..28: note = Synthetic
  source 1..28: mol_type = other DNA
tttggatcaacctcacaatcargtagtt

SEQ ID NO: 308 - DNA, 28 bp, synthetic construct
  misc_feature 1..28: note = Synthetic
  source 1..28: mol_type = other DNA
tttgccctcagcatctcccttcrtgtct

SEQ ID NO: 309 - DNA, 28 bp, synthetic construct
  misc_feature 1..28: note = Synthetic
  source 1..28: mol_type = other DNA
tttgggatagctatacaacaaatraggt

SEQ ID NO: 310 - DNA, 28 bp, synthetic construct
  misc_feature 1..28: note = Synthetic
  source 1..28: mol_type = other DNA
ttcgtggctccagagcractgtggccct

SEQ ID NO: 311 - DNA, 28 bp, synthetic construct
  misc_feature 1..28: note = Synthetic
  source 1..28: mol_type = other DNA
tttgtgatggaatatgttartgggggcg

SEQ ID NO: 312 - DNA, 28 bp, synthetic construct
  misc_feature 1..28: note = Synthetic
  source 1..28: mol_type = other DNA
tttgttttgttgtgtttttctrgaggat

SEQ ID NO: 313 - DNA, 28 bp, synthetic construct
  misc_feature 1..28: note = Synthetic
  source 1..28: mol_type = other DNA
tttgttgtgtttttctrgaggatttcaa

SEQ ID NO: 314 - DNA, 28 bp, synthetic construct
  misc_feature 1..28: note = Synthetic
  source 1..28: mol_type = other DNA
tttggtttttgcccttccagtgtrtact

SEQ ID NO: 315 - DNA, 28 bp, synthetic construct
  misc_feature 1..28: note = Synthetic
  source 1..28: mol_type = other DNA
tttgcccttccagtgtrtactctgaaag

SEQ ID NO: 316 - DNA, 28 bp, synthetic construct
  misc_feature 1..28: note = Synthetic
  source 1..28: mol_type = other DNA
tttgtttgtttgttttgtcactrgcacg

SEQ ID NO: 317 - DNA, 28 bp, synthetic construct
  misc_feature 1..28: note = Synthetic
  source 1..28: mol_type = other DNA
tttgtttgttttgtcactrgcacgggga

SEQ ID NO: 318 - DNA, 28 bp, synthetic construct
  misc_feature 1..28: note = Synthetic
  source 1..28: mol_type = other DNA
tttgtgtttgtgctgcrggtgggcaagt

SEQ ID NO: 319 - DNA, 28 bp, synthetic construct
  misc_feature 1..28: note = Synthetic
  source 1..28: mol_type = other DNA
ttcgctgcaaaccagaccrtggatactg

SEQ ID NO: 320 - DNA, 28 bp, synthetic construct
  misc_feature 1..28: note = Synthetic
  source 1..28: mol_type = other DNA
tttgctttgttttattttrgtcctgttg

SEQ ID NO: 321 - DNA, 28 bp, synthetic construct
  misc_feature 1..28: note = Synthetic
  source 1..28: mol_type = other DNA
tttgtttgcttttttcctgtacrggaaa

SEQ ID NO: 322 - DNA, 28 bp, synthetic construct
  misc_feature 1..28: note = Synthetic
  source 1..28: mol_type = other DNA
tttgcttttttcctgtacrggaaactgg

SEQ ID NO: 323 - DNA, 28 bp, synthetic construct
  misc_feature 1..28: note = Synthetic
  source 1..28: mol_type = other DNA
tttgatcaattagatcttgtcacatrtg

SEQ ID NO: 324 - DNA, 28 bp, synthetic construct
  misc_feature 1..28: note = Synthetic
  source 1..28: mol_type = other DNA
tttgttctcactgttttctttctttrgg

SEQ ID NO: 325 - DNA, 28 bp, synthetic construct
  misc_feature 1..28: note = Synthetic
  source 1..28: mol_type = other DNA
tttgttttgtttttccrgattcgctttg

SEQ ID NO: 326 - DNA, 28 bp, synthetic construct
  misc_feature 1..28: note = Synthetic
  source 1..28: mol_type = other DNA
tttgaggcctgtggttgaratggaaata

SEQ ID NO: 327 - DNA, 28 bp, synthetic construct
  misc_feature 1..28: note = Synthetic
  source 1..28: mol_type = other DNA
tttgtatcttttcatctrgctaaaagac

SEQ ID NO: 328 - DNA, 28 bp, synthetic construct
  misc_feature 1..28: note = Synthetic
  source 1..28: mol_type = other DNA
tttgtgttgataagctctacggtgtraa

SEQ ID NO: 329 - DNA, 28 bp, synthetic construct
  misc_feature 1..28: note = Synthetic
  source 1..28: mol_type = other DNA
tttgaggatgatgtcartgagttcacct

SEQ ID NO: 330 - DNA, 28 bp, synthetic construct
  misc_feature 1..28: note = Synthetic
  source 1..28: mol_type = other DNA
tttgctggctttcttcacrgaagtaaaa

SEQ ID NO: 331 - DNA, 28 bp, synthetic construct
  misc_feature 1..28: note = Synthetic
  source 1..28: mol_type = other DNA
tttgtgaaattccaccrtggcgtaccgt

SEQ ID NO: 332 - DNA, 28 bp, synthetic construct
  misc_feature 1..28: note = Synthetic
  source 1..28: mol_type = other DNA
tttgatcattgtctctggtgrgcgatat

SEQ ID NO: 333 - DNA, 28 bp, synthetic construct
  misc_feature 1..28: note = Synthetic
  source 1..28: mol_type = other DNA
tttggaatttatccagtgatcaggtrtt

SEQ ID NO: 334 - DNA, 28 bp, synthetic construct
  misc_feature 1..28: note = Synthetic
  source 1..28: mol_type = other DNA
tttgttccctttcttacrgatttaatgt

SEQ ID NO: 335 - DNA, 28 bp, synthetic construct
  misc_feature 1..28: note = Synthetic
  source 1..28: mol_type = other DNA
tttgtatatatattttttatctttcrgc

SEQ ID NO: 336 - DNA, 28 bp, synthetic construct
  misc_feature 1..28: note = Synthetic
  source 1..28: mol_type = other DNA
ttcgatgttcctcttaaagtggrtctca

SEQ ID NO: 337 - DNA, 28 bp, synthetic construct
  misc_feature 1..28: note = Synthetic
  source 1..28: mol_type = other DNA
ttcggactctcaagcaggaaracattgt

SEQ ID NO: 338 - DNA, 28 bp, synthetic construct
  misc_feature 1..28: note = Synthetic
  source 1..28: mol_type = other DNA
tttgtctcttctcttcttatrggacctg

SEQ ID NO: 339 - DNA, 28 bp, synthetic construct
  misc_feature 1..28: note = Synthetic
  source 1..28: mol_type = other DNA
tttgattataattttggatcrtctgtat

SEQ ID NO: 340 - DNA, 28 bp, synthetic construct
  misc_feature 1..28: note = Synthetic
  source 1..28: mol_type = other DNA
tttgatcttagtgtttattacagraaat

SEQ ID NO: 341 - DNA, 28 bp, synthetic construct
  misc_feature 1..28: note = Synthetic
  source 1..28: mol_type = other DNA
tttgtgcaagtttgcaaacrtgttcacc

SEQ ID NO: 342 - DNA, 28 bp, synthetic construct
  misc_feature 1..28: note = Synthetic
  source 1..28: mol_type = other DNA
tttgtgaaactgctttttgaaacagrga

SEQ ID NO: 343 - DNA, 28 bp, synthetic construct
  misc_feature 1..28: note = Synthetic
  source 1..28: mol_type = other DNA
tttgttttccaagacccaccratgacat

SEQ ID NO: 344 - DNA, 28 bp, synthetic construct
  misc_feature 1..28: note = Synthetic
  source 1..28: mol_type = other DNA
ttcgtccttgaccgcagggtcctggrta

SEQ ID NO: 345 - DNA, 28 bp, synthetic construct
  misc_feature 1..28: note = Synthetic
  source 1..28: mol_type = other DNA
ttcgggaatgtgacgactattttccrac

SEQ ID NO: 346 - DNA, 28 bp, synthetic construct
  misc_feature 1..28: note = Synthetic
  source 1..28: mol_type = other DNA
ttcgggaatgtgacgactattttccrac

SEQ ID NO: 347 - DNA, 28 bp, synthetic construct
  misc_feature 1..28: note = Synthetic
  source 1..28: mol_type = other DNA
tttgtgctggagcaggrggagtacaaga

SEQ ID NO: 348 - DNA, 28 bp, synthetic construct
  misc_feature 1..28: note = Synthetic
  source 1..28: mol_type = other DNA
tttgacttcacggtaactgggrgaggga

SEQ ID NO: 349 - DNA, 28 bp, synthetic construct
  misc_feature 1..28: note = Synthetic
  source 1..28: mol_type = other DNA
tttgttttcattcacccrggaatgcatt

SEQ ID NO: 350 - DNA, 28 bp, synthetic construct
  misc_feature 1..28: note = Synthetic
  source 1..28: mol_type = other DNA
tttggcaataatgaaaaggtaarttgat

SEQ ID NO: 351 - DNA, 28 bp, synthetic construct
  misc_feature 1..28: note = Synthetic
  source 1..28: mol_type = other DNA
tttgtggagacatccctgrgacggccac

SEQ ID NO: 352 - DNA, 28 bp, synthetic construct
  misc_feature 1..28: note = Synthetic
  source 1..28: mol_type = other DNA
tttgctgtaaatcaaatcatgggcrgcc

SEQ ID NO: 353 - DNA, 28 bp, synthetic construct
  misc_feature 1..28: note = Synthetic
  source 1..28: mol_type = other DNA
tttgcatgtatttcctrgggatgtcctt

SEQ ID NO: 354 - DNA, 28 bp, synthetic construct
  misc_feature 1..28: note = Synthetic
  source 1..28: mol_type = other DNA
tttgtaaaacctctagraatgtctcata

SEQ ID NO: 355 - DNA, 28 bp, synthetic construct
  misc_feature 1..28: note = Synthetic
  source 1..28: mol_type = other DNA
tttgatttctttttttttcattggcrga

SEQ ID NO: 356 - DNA, 28 bp, synthetic construct
  misc_feature 1..28: note = Synthetic
  source 1..28: mol_type = other DNA
tttggtccacctgggaatgggaagrcaa

SEQ ID NO: 357 - DNA, 28 bp, synthetic construct
  misc_feature 1..28: note = Synthetic
  source 1..28: mol_type = other DNA
tttgtatacgtgtaatgcarcattcaga

SEQ ID NO: 358 - DNA, 28 bp, synthetic construct
  misc_feature 1..28: note = Synthetic
  source 1..28: mol_type = other DNA
tttgaattctatttcactrgttccttcc

SEQ ID NO: 359 - DNA, 28 bp, synthetic construct
  misc_feature 1..28: note = Synthetic
  source 1..28: mol_type = other DNA
tttgaatccttgctctgcgatggrctat

SEQ ID NO: 360 - DNA, 28 bp, synthetic construct
  misc_feature 1..28: note = Synthetic
  source 1..28: mol_type = other DNA
tttgccattgctaaggaattgctaraaa

SEQ ID NO: 361 - DNA, 28 bp, synthetic construct
  misc_feature 1..28: note = Synthetic
  source 1..28: mol_type = other DNA
tttgcctctctcctcacrgcccccagtc

SEQ ID NO: 362 - DNA, 28 bp, synthetic construct
  misc_feature 1..28: note = Synthetic
  source 1..28: mol_type = other DNA
tttgacaaactgtgtgtttrtgacaatg

SEQ ID NO: 363 - DNA, 28 bp, synthetic construct
  misc_feature 1..28: note = Synthetic
  source 1..28: mol_type = other DNA
tttgcttttttctccctccccccargat

SEQ ID NO: 364 - DNA, 28 bp, synthetic construct
  misc_feature 1..28: note = Synthetic
  source 1..28: mol_type = other DNA
ttcggcaacgtctctcccaacrccaact

SEQ ID NO: 365 - DNA, 28 bp, synthetic construct
  misc_feature 1..28: note = Synthetic
  source 1..28: mol_type = other DNA
ttcggcaacgtctctcccaacrccaact

SEQ ID NO: 366 - DNA, 28 bp, synthetic construct
  misc_feature 1..28: note = Synthetic
  source 1..28: mol_type = other DNA
tttgcacttagggggccaggacrtcttc

SEQ ID NO: 367 - DNA, 28 bp, synthetic construct
  misc_feature 1..28: note = Synthetic
  source 1..28: mol_type = other DNA
ttcgaggggctccgcaagcactrcctgc

SEQ ID NO: 368 - DNA, 28 bp, synthetic construct
  misc_feature 1..28: note = Synthetic
  source 1..28: mol_type = other DNA
tttgctgttcttcacagctttaggrtat

SEQ ID NO: 369 - DNA, 28 bp, synthetic construct
  misc_feature 1..28: note = Synthetic
  source 1..28: mol_type = other DNA
tttgatgcttgtttaattagtgragaat

SEQ ID NO: 370 - DNA, 28 bp, synthetic construct
  misc_feature 1..28: note = Synthetic
  source 1..28: mol_type = other DNA
ttcgctgtcatattcctggctrgctaca

SEQ ID NO: 371 - DNA, 28 bp, synthetic construct
  misc_feature 1..28: note = Synthetic
  source 1..28: mol_type = other DNA
tttgtcttccttgacgatattctrcttt

SEQ ID NO: 372 - DNA, 28 bp, synthetic construct
  misc_feature 1..28: note = Synthetic
  source 1..28: mol_type = other DNA
tttgtcttccttgacgatattctrcttt

SEQ ID NO: 373 - DNA, 28 bp, synthetic construct
  misc_feature 1..28: note = Synthetic
  source 1..28: mol_type = other DNA
tttgcaatggcctccagrtaattcttcc

SEQ ID NO: 374 - protein, 18 aa, synthetic construct
  REGION 1..18: note = Synthetic
KRTADGSEFESPKKKRKV

SEQ ID NO: 375 - protein, 17 aa, synthetic construct
  REGION 1..17: note = Synthetic
KRTADGSEFEPKKKRKV

SEQ ID NO: 376 - DNA, 125 bp, synthetic construct
  misc_feature 1..125: note = Synthetic
  misc_difference 1..8: note = n is a, c, g, or t
  source 1..125: mol_type = other DNA
nnnnnnnngtttttgtactctcaagatttagaaataaatcttgcagaagctacaaagataaggcttcatgccgaaatcaacaccctgtcattttatggcagggtgttttcgttatttaatttttt

SEQ ID NO: 377 - DNA, 121 bp, synthetic construct
  misc_feature 1..121: note = Synthetic
  misc_difference 1..18: note = n is a, c, g, or t
  source 1..121: mol_type = other DNA
nnnnnnnnnnnnnnnnnngtttttgtactctcagaaatgcagaagctacaaagataaggcttcatgccgaaatcaacaccctgtcattttatggcagggtgttttcgttatttaatttttt

SEQ ID NO: 378 - DNA, 109 bp, synthetic construct
  misc_feature 1..109: note = Synthetic
  misc_difference 1..20: note = n is a, c, g, or t
  source 1..109: mol_type = other DNA
nnnnnnnnnnnnnnnnnnnngtttttgtactctcagaaatgcagaagctacaaagataaggcttcatgccgaaatcaacaccctgtcattttatggcagggtgtttttt

SEQ ID NO: 379 - DNA, 102 bp, synthetic construct
  misc_feature 1..102: note = Synthetic
  misc_difference 1..20: note = n is a, c, g, or t
  source 1..102: mol_type = other DNA
nnnnnnnnnnnnnnnnnnnngttttagagctagaaatagcaagttaaaataaggctagtccgttatcaacttgaaaaagtggcaccgagtcggtgctttttt

SEQ ID NO: 380 - DNA, 88 bp, synthetic construct
  misc_feature 1..88: note = Synthetic
  misc_difference 1..20: note = n is a, c, g, or t
  source 1..88: mol_type = other DNA
nnnnnnnnnnnnnnnnnnnngttttagagctagaaatagcaagttaaaataaggctagtccgttatcaacttgaaaaagtgttttttt

SEQ ID NO: 381 - DNA, 76 bp, synthetic construct
  misc_feature 1..76: note = Synthetic
  misc_difference 1..20: note = n is a, c, g, or t
  source 1..76: mol_type = other DNA
nnnnnnnnnnnnnnnnnnnngttttagagctagaaatagcaagttaaaataaggctagtccgttatcatttttttt

SEQ ID NO: 382 - RNA, 81 nt, synthetic construct
  misc_feature 1..81: note = Synthetic
  source 1..81: mol_type = other RNA
gttttagagctagaaatagcaagttaaaataaggctagtccgttatcaacttgaaaaagtggcaccgagtcggtgcttttt

SEQ ID NO: 383 - protein, 36 aa, synthetic construct
  REGION 1..36: note = Synthetic
  VARIANT 2: note = X can be any naturally occurring amino acid
  VARIANT 4..29: note = X can be any naturally occurring amino acid
  REGION 26..29: note = may or may not be present
  VARIANT 32..35: note = X can be any naturally occurring amino acid
  REGION 34..35: note = may or may not be present
HXEXXXXXXXXXXXXXXXXXXXXXXXXXXPCXXXXC

SEQ ID NO: 384 - protein, 20 aa, synthetic construct
  REGION 1..20: note = Synthetic
NNNNAAASSWWSSTTTNNNN

SEQ ID NO: 385 - protein, 343 aa, synthetic construct
  REGION 1..343: note = Synthetic
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 386 - protein, 423 aa, synthetic construct
  REGION 1..423: note = Synthetic
MPQFGILCKTPPKVLVRQFVERFERPSGEKIALCAAELTYLCWMITHNGTAIKRATFMSYNTIISNSLSFDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI

SEQ ID NO: 387 - protein, 144 aa, synthetic construct
  REGION 1..144: note = Synthetic
MRLFGYARVSTSQQSLDIQVRALKDAGVKANRIFTDKASGSSSDRKGLDLLRMKVEEGDVILVKKLDRLGRDTADMIQLIKEFDAQGVSIRFIDDGISTDGEMGKMVVTILSAVAQAERQRILERTNEGRQEAMAKGVVFGRKR

SEQ ID NO: 388 - protein, 144 aa, synthetic construct
  REGION 1..144: note = Synthetic
MRLFGYARVSTSQQSLDIQVRALKDAGVKANRIFTDKASGSSSDRKGLDLLRMKVEEGDVILVKKLDRLGRDTADMIQLIKEFDAQGVSIRFIDDGISTDGEMGKMVVTILSAVAQAERQRILQRTNEGRQEAMAKGVVFGRKR

SEQ ID NO: 389 - protein, 144 aa, synthetic construct
  REGION 1..144: note = Synthetic
MRLFGYARVSTSQQSLDIQVRALKDAGVKANRIFTDKASGSSSDRKGLDLLRMKVEEGDVILVKKLDRLGRDTADMIQLIKEFDAQGVSIRFIDDGISTDGYMGKMVVTILSAVAQAERQRILQRTNEGRQEAMAKGVVFGRKR

SEQ ID NO: 390 - protein, 147 aa, synthetic construct
  REGION 1..147: note = Synthetic
MAKIGYARVSSKEQNLDRQLQALQGVSKVFSDKLSGQSVERPQLQAMLNYIREGDIVVVTELDRLGRNNKELTELMNAIQQKGATLEVLDLPSMNGIEDENLRRLINNLVIELYKYQAESERKRIKERQAQGIEIAKSKGKFKGRQH

SEQ ID NO: 391 - protein, 147 aa, synthetic construct
  REGION 1..147: note = Synthetic
MAKIGYARVSSKEQNLDRQLQALQGVSKVFSDKLSGQSVERPQLQAMLNYIREGDIVVVTELDRLGRNNKELTELMNAIQQKGATLEVLDLPSMDGIEDENLRRLINNLVIELYKYQAESERKRIKERQAQGIEIAKSKGKFKGRQH

SEQ ID NO: 392 - protein, 150 aa, synthetic construct
  REGION 1..150: note = Synthetic
MIIGYARVSSLDQNLERQLENLKTFGAEKIFTEKQSGKSIENRPILQKALNFVRMGDRFIVESIDRLGRNYNEVIHTVNYLKDKEVQLMITSLPMMNEVIGNPLLDKFMKDLIIQILAMVSEQERNESKRRQAQGIQVAKEKGVYKGRPL

SEQ ID NO: 393 - protein, 150 aa, synthetic construct
  REGION 1..150: note = Synthetic
MIIGYARVSSLDQNLERQLENLKTFGAEKIFTEKQSGKSIENRPILQKALNFVRMGDRFIVESIDRLGRNYNEVIHTVNYLKDKEVRLMITSLPMMNEVIGNPLLDKFMKDLIIRILAMVSEQERNESKRRQAQGIQVAKEKGVYKGRPL

SEQ ID NO: 394 - protein, 144 aa, synthetic construct
  REGION 1..144: note = Synthetic
MRLFGYARVSTSQQSLDLQVRALKDAGVKANRIFTDKASGSSTDREGLDLLRMKVKEGDVILVKKLDRLGRDTADMLQLIKEFDAQGVAVRFIDDGISTDGDMGQMVVTILSAVAQAERRRILERTNEGRQEAKLKGIKFGRRR

SEQ ID NO: 395 - protein, 144 aa, synthetic construct
  REGION 1..144: note = Synthetic
MRLFGYARVSTSQQSLDLQVRALKDAGVKANRIFTDKASGSSTDREGLDLLRMKVKEGDVILVKKLDRLSRDTADMLQLIKEFDAQGVAVRFIDDGISTDGYMGQMVVTILSAVAQAERRRILQRTNEGRQEAKLKGIKFGRRR

SEQ ID NO: 396 - protein, 142 aa, synthetic construct
  REGION 1..142: note = Synthetic
MATIGYIRVSTIDQNIDLQRNALTSANCDRIFEDRISGKIANRPGLKRALKYVNKGDTLVVWKLDRLGRSVKNLVALISELHERGAHFHSLTDSIDTSSAMGRFFFHVMSALAEMERELIVERTLAGLAAARAQGRLGGRPV

SEQ ID NO: 397 - protein, 142 aa, synthetic construct
  REGION 1..142: note = Synthetic
MATIGYIRVSTIDQNIDLQRNALTSANCDRIFEDRISGKIANRPGLKRALKYVNKGDTLVVWKLDRLGRSVKNLVALISELHERGAHFHSLTDSIDTSSAMGRFFFYVMSALAEMERELIVERTLAGLAAARAQGRLGGRPV

SEQ ID NO: 398 - protein, 608 aa, synthetic construct
  REGION 1..608: note = Synthetic
MDTYAGAYDRQSRERENSSAASPATQRSANEDKAADLQREVERDGGRFRFVGHFSEAPGTSAFGTAERPEFERILNECRAGRLNMIIVYDVSRFSRLKVMDAIPIVSELLALGVTIVSTQEGVFRQGNVMDLIHLIMRLDASHKESSLKSAKILDTKNLQRELGGYVGGKAPYGFELVSETKEITRNGRMVNVVINKLAHSTTPLTGPFEFEPDVIRWWWREIKTHKHLPFKPGSQAAIHPGSITGLCKRMDADAVPTRGETIGKKTASSAWDPATVMRILRDPRIAGFAAEVIYKKKPDGTPTTKIEGYRIQRDPITLRPVELDCGPIIEPAEWYELQAWLDGRGRGKGLSRGQAILSAMDKLYCECGAVMTSKRGEESIKDSYRCRRRKVVDPSAPGQHEGTCNVSMAALDKFVAERIFNKIRHAEGDEETLALLWEAARRFGKLTEAPEKSGERANLVAERADALNALEELYEDRAAGAYDGPVGRKHFRKQQAALTLRQQGAEERLAELEAAEAPKLPLDQWFPEDADADPTGPKSWWGRASVDDKRVFVGLFVDKIVVTKSTTGRGQGTPIEKRASITWAKPPTDDDEDDAQDGTEDVAATGA

SEQ ID NO: 399 - DNA, 34 bp, synthetic construct
  misc_feature 1..34: note = Synthetic
  source 1..34: mol_type = other DNA
ataacttcgtatagcatacattatacgaagttat

SEQ ID NO: 400 - DNA, 34 bp, synthetic construct
  misc_feature 1..34: note = Synthetic
  source 1..34: mol_type = other DNA
gaagttcctattctctagaaagtataggaacttc

SEQ ID NO: 401 - protein, 173 aa, synthetic construct
  REGION 1..173: note = Synthetic
AKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGI

SEQ ID NO: 402 - protein, 174 aa, synthetic construct
  REGION 1..174: note = Synthetic
MAKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGI

SEQ ID NO: 403 - protein, 174 aa, Escherichia coli
MAKPAKRIKSAAAAYVPQNRDAVITDIKRIGDLQREASRLETEMNDAIAEITEKFAARIAPIKTDIETLSKGVQGWCEANRDELTNGGKVKTANLVTGDVSWRVRPPSVSIRGMDAVMETLERLGLQRFVRTKQEINKEAILLEPKAVAGVAGITVKSGIEDFSIIPFEQEAGI

SEQ ID NO: 404 - protein, 174 aa, Escherichia coli
MAKPAKRIKSAAAAYVPQNRDAVITDIKRIGDLQREASRLETEMNDAIAEITEKFAARIAPLKTDIETLSKGVQGWCEANRDELTNGGKVKTANLVTGDVSWRVRPPSVSIRGMDAVMETLERLGLQRFIRTKQEINKEAILLEPKAVAGVAGITVKSGIEDFSIIPFEQEAGI

SEQ ID NO: 405 - protein, 174 aa, Escherichia coli
MAKPAKRIKSAAAAYVPQNRDAVITDIKRIGDLQREASRLETEMNDAIAEITEKFAARIAPIKTDIETLSKGVQGWCEANRDELTNGGKVKTANLITGDVSWRVRPPSVSIRGMDAVMETLERLGLQRFIRTKQEINKEAILLEPKAVAGVAGITVKSGIEDFSIIPFEQEAGI

SEQ ID NO: 406 - DNA, 31 bp, synthetic construct
  misc_feature 1..31: note = Synthetic
  source 1..31: mol_type = other DNA
tctctgcgtactgattggaacatccgcgaaa

SEQ ID NO: 407 - DNA, 31 bp, synthetic construct
  misc_feature 1..31: note = Synthetic
  source 1..31: mol_type = other DNA
gctgcagaagggattccatgaggtgcgcgaa

SEQ ID NO: 408 - DNA, 30 bp, synthetic construct
  misc_feature 1..30: note = Synthetic
  source 1..30: mol_type = other DNA
gcggggcgggccagcctgaatagctgcaaa

SEQ ID NO: 409 - DNA, 31 bp, synthetic construct
  misc_feature 1..31: note = Synthetic
  source 1..31: mol_type = other DNA
gctggccctgtaaaggaaactggaacacaaa

SEQ ID NO: 410 - DNA, 31 bp, synthetic construct
  misc_feature 1..31: note = Synthetic
  source 1..31: mol_type = other DNA
gtggttccagaaccggaggacaaagtacaaa

SEQ ID NO: 411 - DNA, 30 bp, synthetic construct
  misc_feature 1..30: note = Synthetic
  source 1..30: mol_type = other DNA
tcacatcaaccggtggcgcattgccacgaa

SEQ ID NO: 412 - DNA, 30 bp, synthetic construct
  misc_feature 1..30: note = Synthetic
  source 1..30: mol_type = other DNA
gaaggtgtggttccagaaccggaggacaaa

SEQ ID NO: 413 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 414 - protein, 178 aa, Escherichia coli
MRRAFITGVFFLSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 415 - protein, 160 aa, Staphylococcus aureus
MGSHMTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 416 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 417 - protein, 183 aa, unidentified
  source 1..183: note = Salmonella typhimurium
MPPAFITGVTSLSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 418 - protein, 164 aa, Shewanella putrefaciens
MDEYWMQVAMQMAEKAEAAGEVPVGAVLVKDGQQIATGYNLSISQHDPTAHAEILCLRSAGKKLENYRLLDATLYITLEPCAMCAGAMVHSRIARVVYGARDEKTGAAGTVVNLLQHPAFNHQVEVTSGVLAEACSAQLSRFFKRRRDEKKALKLAQRAQQGIE

SEQ ID NO: 419 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEITSGVLAEECSQKLSTFFQKRREEKKIEKALLKSLSDK

SEQ ID NO: 420 - protein, 161 aa, unidentified
  source 1..161: note = Caulobacter crescentus
MRTDESEDQDHRMMRLALDAARAAAEAGETPVGAVILDPSTGEVIATAGNGPIAAHDPTAHAEIAAMRAAAAKLGNYRLTDLTLVVTLEPCAMCAGAISHARIGRVVFGADDPKGGAVVHGPKFFAQPTCHWRPEVTGGVLADESADLLRGFFRARRKAKI

SEQ ID NO: 421 - protein, 179 aa, Geobacter sulfurreducens
MSSLKKTPIRDDAYWMGKAIREAAKAAARDEVPIGAVIVRDGAVIGRGHNLREGSNDPSAHAEMIAIRQAARRSANWRLTGATLYVTLEPCLMCMGAIILARLERVVFGCYDPKGGAAGSLYDLSADPRLNHQVRLSPGVCQEECGTMLSDFFRDLRRRKKAKATPALFIDERKVPPEP

SEQ ID NO: 422 - protein, 167 aa, synthetic construct
  REGION 1..167: note = Synthetic
MSEVEFSHEYWMRHALTLAKRAWDEREVPVGAVLVHNNRVIGEGWNRPIGRHDPTAHAAIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 423 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 424 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGWRNAKTGAAGSLMDVLHYPGMNHRVEITEGILADECAALLCYFFRMPRQVFNAQKKAQSSTD

SEQ ID NO: 425 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRWAKTGAAGSLMDVLHYPGMNHRVEITEGILADECAALLCYFFRMPRQVFNAQKKAQSSTD

SEQ ID NO: 426 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRQAKTGAAGSLMDVLHYPGMNHRVEITEGILADECAALLCYFFRMPRQVFNAQKKAQSSTD

SEQ ID NO: 427 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGFRNAKTGAAGSLMDVLHYPGMNHRVEITEGILADECAALLCYFFRMPRQVFNAQKKAQSSTD

SEQ ID NO: 428 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGQRNAKTGAAGSLMDVLHYPGMNHRVEITEGILADECAALLCYFFRMPRQVFNAQKKAQSSTD

SEQ ID NO: 429 - protein, 167 aa, synthetic construct
  REGION 1..167: note = Synthetic
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGMRNAKTGAAGSLMDVLHYPGMNHRVEITEGILADECAALLCYFFRMPRQVFNAQKKAQSSTD

SEQ ID NO: 430 - protein, 167 aa, synthetic construct
  REGION 1..167: note = Synthetic
MSEVEFSHEYWMRHALTLAKRARDEREVPVGAVLVLNNRVIGEGWNFAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 431 - protein, 167 aa, synthetic construct
  REGION 1..167: note = Synthetic
MSEVEFSHEYWMRHALTLAKRARDEREVPVGAVLVLNNRVIGEGWNWAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 432 - protein, 167 aa, synthetic construct
  REGION 1..167: note = Synthetic
MSEVEFSHEYWMRHALTLAKRARDEREVPVGAVLVLNNRVIGEGWNQAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 433 - protein, 167 aa, synthetic construct
  REGION 1..167: note = Synthetic
MSEVEFSHEYWMRHALTLAKRARDEREVPVGAVLVLNNRVIGEGWNMAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 434 - protein, 167 aa, synthetic construct
  REGION 1..167: note = Synthetic
MSEVEFSHEYWMRHALTLAKRAWDEREVPVGAVLVHNNRVIGEGWNRPIGRHDPTAHAQIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 435 - DNA, 145 bp, synthetic construct
  misc_feature 1..145: note = Synthetic
  misc_difference 1..22: note = n is a, c, g, or t
  source 1..145: mol_type = other DNA
nnnnnnnnnnnnnnnnnnnnnngtcatagttcccctgagaaatcagggttactatgataagggctttctgcctaaggcagactgacccgcggcgttggggatcgcctgtcgcccgcttttggcgggcattccccatccttttttt

SEQ ID NO: 436 - protein, 22 aa, synthetic construct
  REGION 1..22: note = Synthetic
GGSSGSETPGTSESATPESGGS